(19) United States
(12) Patent Application Publication
(10) Pub. No.: US 2025/0195511 A1
(43) Pub. Date: Jun. 19, 2025

(54) INDAZOLE DERIVATIVES USEFUL AS INHIBITORS OF NOD-LIKE RECEPTOR PROTEIN 3

(71) Applicant: Merck Sharp & Dohme LLC, Rahway, NJ (US)

(72) Inventors: Zachary G. Brill, San Francisco, CA (US); Donna A.A.W. Hayes, San Francisco, CA (US); Tom M. Lam, San Francisco, CA (US); Kyle S. McClymont, San Diego, CA (US); Bryan S. Matsuura, San Francisco, CA (US); Rohan Rajiv Merchant, Burlingame, CA (US); Anilkumar G. Nair, Morganville, NJ (US); Ning Qi, Somerset, NJ (US)

(73) Assignee: Merck Sharp & Dohme LLC, Rahway, NJ (US)

(21) Appl. No.: 18/978,580

(22) Filed: Dec. 12, 2024

Related U.S. Application Data
(60) Provisional application No. 63/610,214, filed on Dec. 14, 2023.

(51) Int. Cl. A61K 31/4985; C07D 487/04

(52) U.S. Cl. CPC A61K 31/4985; C07D 487/04

(57) ABSTRACT

Novel compounds of Formula (I), and the pharmaceutically acceptable salts thereof, are inhibitors of NLRP3 and may be useful in the treatment, prevention, management, amelioration, control, and suppression of diseases mediated by NLPR3. The compounds of the present invention may be useful in the treatment, prevention or management of diseases, disorders and conditions mediated by NLRP3 such as, but not limited to, obesity, gout, pseudogout, CAPS, NASH, MASH, fibrosis, osteoarthritis, atherosclerosis, heart failure, idiopathic pericarditis, myocarditis, atopic dermatitis, hidradenitis suppurativa, inflammatory bowel disease, cancer, Alzheimer's Disease, Parkinson's Disease, dementia with Lewy bodies (DLB), and traumatic brain injury.

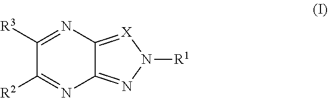

### CROSS-REFERENCE TO RELATED APPLICATIONS

This application claims the benefit of U.S. Provisional Patent Application Ser. No. 63/610,214, which was filed on Dec. 14, 2023, the entire contents of which are incorporated by reference herein.

### FIELD

This disclosure relates generally to indazole derivative compounds that modulate or inhibit nucleotide-binding oligomerization domain (NOD)-like receptor protein 3 and that may be useful for therapy and/or prophylactic treatment.

### BACKGROUND

Inflammasomes function as central signaling hubs of the innate immune system. They are multi-protein complexes assembled after activation of intracellular pattern recognition receptors (PRRs) by a variety of pathogen-associated molecular patterns (PAMPs) or danger-associated molecular patterns (DAMPs). It has been shown that inflammasomes can be formed by nucleotide-binding oligomerization domain (NOD)-like receptors (NLRs) and Pyrin and HIN200-domain-containing proteins (Van Opdenbosch N and Lamkanfi M. Immunity, 2019 Jun. 18; 50(6):1352-1364). Inflammasome activation triggers a cascade of events that releases pro-inflammatory cytokines and promotes an inflammatory form of cell death called pyroptosis induced by the activation of Gasdermin. Pyroptosis is a unique form of inflammatory cell death that leads to the release of not only cytokines but also other intracellular components that promote a broader immune response both of the innate and of the acquired immune system. Thus, inflammasome activation is a major regulator of the inflammatory cascade.

The (NOD)-like receptor protein 3 (NLRP3) inflammasome is the most well-studied of all the inflammasomes. NLRP3 can be activated by numerous stimuli including environmental crystals, pollutants, host-derived DAMPs and protein aggregates (Tartey S and Kanneganti T D. Immunology, 2019 April; 156(4):329-338). Danger-associated molecular patterns that engage NLRP3 include uric acid and cholesterol crystals that cause gout and atherosclerosis, amyloid-P fibrils that are neurotoxic in Alzheimer's disease, and asbestos particles that cause mesothelioma (Kelley et al., Int J Mol Sci, 2019 Jul. 6; 20 (13)). Additionally, NLRP3 is activated by infectious agents, such as Vibrio cholerae, fungal pathogens, such as Aspergillus fumigatus and Candida albicans, adenoviruses, influenza A virus and SARS-CoV-2 (Tartey and Kanneganti, 2019 (see above); Fung et al. Emerg Microbes Infect, 2020 Mar. 14; 9(1):558-570).

The NLRP3 activation mechanism in humans remains unclear. It has been suggested that the NLRP3 inflammasome requires regulation at both the transcriptional and the post-transcriptional level (Yang Yet al., Cell Death Dis, 2019 Feb. 12; 10(2): 128). The NOD-like receptor protein 3 (NLRP3) is a protein-coding gene that encodes a protein consisting of a N-terminal pyrin domain, a nucleotide-binding site domain (NBD), and a leucine-rich repeat (LRR) motif on the C-terminal (Inoue et al., Immunology, 2013, 139, 11-18; Sharif et al., Nature, 2019 June; 570 (7761): 338-343).

In response to sterile inflammatory danger signals PAMPs or DAMPs, NLRP3 interacts with the adaptor protein, apoptosis-associated speck-like protein containing a caspase recruitment domain (ASC), and with the protease caspase-1 to form the NLRP3 inflammasome. Upon activation, procaspase-1 undergoes autoproteolysis and cleaves gasdermin D (Gsdmd) to produce the N-terminal Gsdmd molecule that leads to pore-formation in the plasma membrane and results in a lytic form of cell death called pyroptosis. Alternatively, caspase-1 cleaves the pro-inflammatory cytokines pro-IL-1β and pro-IL-18 to allow release of its biological active form (Kelley et al., 2019—supra). The NLRP3 inflammasome activation results in the release of the inflammatory cytokines IL-1β (interleukin-1β) and IL-18 (interleukin-18), which when dysregulated can lead to a number of diseases.

Dysregulation of the NLRP3 inflammasome or its downstream mediators are associated with numerous immune diseases, inflammatory diseases, auto-immune diseases, and auto-inflammatory diseases. Activation of the NLRP3 inflammasome has been linked to the following diseases and disorders: Cryopyrin-associated Periodic Syndromes; sickle cell disease; systemic lupus erythematosus; allodynia; graft versus host disease; hepatic disorders including non-alcoholic steatohepatitis (NASH), chronic liver disease, viral hepatitis, alcoholic steatohepatitis, and alcoholic liver disease; inflammatory bowel diseases including Crohn's disease and ulcerative colitis; inflammatory joint disorders including gout, pseudogout, arthropathy, osteoarthritis, rheumatoid arthritis; additional rheumatic diseases including dermatomyositis, Still's disease, and juvenile idiopathic arthritis. kidney related diseases including hyperoxaluria, lupus nephritis, hypertensive nephropathy, hemodialysis related inflammation, diabetic nephropathy, and diabetic kidney disease, and other inflammatory diseases (Miyamae T. Paediatr Drugs, 2012 Apr. 1, 14(2): 109-17; Szabo G and Petrasek J. Nat Rev Gastroenterol Hepatol, 2015 July; 12(7): 387-400; Zhen Y and Zhang H. Front Immunol, 2019 Feb. 28; 10:276; Vande Walle L et al., Nature, 2014 Aug. 7; 512 (7512): 69-73; Knauf et al., Kidney Int, 2013 November; 84(5):895-901; Krishnan et al., Br J Pharmacol, 2016 February; 1 73(4):752-65); Shahzad et al., Kidney Int, 2015 January; 87(1):74-84; Jankovic, et al. J Exp Med. 2013 Sep. 23; 210(10):1899-910.). The onset and progression of neuroinflammation-related disorders, such as brain infection, acute injury, multiple sclerosis, amyotrophic lateral sclerosis, and additional neurodegenerative diseases such as Parkinson's and Alzheimer's diseases have also been linked to NLRP3 inflammasome activation (Sarkar et al., NPJ Parkinsons Dis, 2017 Oct. 17; 3:30).

Cardiovascular and metabolic disorders such as atherosclerosis, type I and type II diabetes and diabetes complications including nephropathy and retinopathy, peripheral artery disease, acute heart failure, and hypertension have been associated with NLRP3 (Ridker et al., CANTOS Trial Group. N Engl J Med, 2017 Sep. 21; 377(12):1119-1131; and Toldo S and Abbate A, Nat Rev Cardiol, 2018 April; 15(4):203-214). NLRP3 associated skin diseases include wound healing and scar formation; inflammatory skin diseases such as acne, atopic dermatitis, hidradenitis suppurativa, and psoriasis (Kelly et al., Br J Dermatol, 2015 December; 1 73(6)). NLRP3 inflammasome activity has also been linked to respiratory conditions such as asthma, sarcoidosis, acute respiratory distress syndrome, Severe Acute Respiratory Syndrome (SARS) (Nieto-Torres et al., Virology, 2015 November; 485:330-9)); and ocular diseases including age-related macular degeneration (AMD) and diabetic retinopathy (Doyle et al., Nat Med, 2012 May; 18(5):791-8). Cancers linked to NLRP3 include myeloproliferative neoplasms, leukemias, myelodysplastic syndromes, myelofibrosis, lung cancer, and colon cancer (Ridker et al., Lancet, 2017 Oct. 21; 390(10105): 1833-1842; Derangere et al., Cell Death Differ. 2014 December; 21(12): 1914-24; Basiorka et al., Lancet Haematol, 2018 September; 5(9): e393-e402, Zhang et al., Hum Immunol, 2018 January; 79(1):57-62).

Immune diseases and inflammatory disorders are typically difficult to diagnose or treat efficiently and effectively. Most treatments include treatment of the symptoms, slowing down disease progression, lifestyle changes, and surgery.

There remains a need for inhibitors of NLRP3 to provide new treatments for diseases and disorders associated with NLRP3 inflammasome activation and dysregulation. The compounds of the present invention are useful for the treatment and prevention of diseases, disorders, and conditions mediated by formation and propagation of the NLRP3 inflammasome.

NLRP3 inhibitors are disclosed in the following publications: Nat. 2022, 1; Cell. 2021, 184, 1; J. Mol. Biol. 2021, 433, 167308; J. Med. Chem. 2021, 64, 101; Nat. Chem. Biol. 2019, 15, 556; Nat. 2019, 570, 338; Nat. Chem. Biol. 2019, 15, 560; PLOS Biol. 2019, 1; Nat. Med. 2015, 21, 248; Cell. 2014, 156, 1193; Nat. Immunol. 2014, 15, 738; PNAS. 2007, 104, 8041; Nat. 2006, 440, 9; Immunity. 2006, 24, 317. Several patent applications describe NLRP3 inhibitors, including WO 2021/239885, WO 2021/209552, WO 2021/209539, WO 2021/193897, WO 2020/018975, WO 2020/037116, WO 2020/021447, WO 2020/010143, WO 2019/079119, WO 2019/0166621, WO 2019/121691, U.S. Pat. No. 11,319,319, and US 2020/0361898.

### SUMMARY

The present disclosure relates to novel compounds of structural Formula (I):

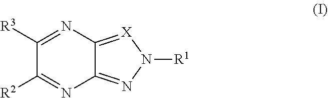

and pharmaceutically acceptable salts thereof.

The compounds of structural Formula (I), and embodiments thereof, are inhibitors of NOD-like receptor protein 3 (NLRP3) and may be useful in the treatment and prevention of diseases, disorders and conditions mediated by NLRP3 such as, but not limited to obesity, gout, pseudogout (chondrocalcinosis), cryopyrin-associated periodic syndromes (CAPS), non-alcoholic steatohepatitis (NASH), metabolic dysfunction-associated steatohepatitis (MASH), fibrosis, heart failure, idiopathic pericarditis, atopic dermatitis, inflammatory bowel disease, Alzheimer's Disease, Parkinson's Disease, dementia with Lewy bodies (DLB), and traumatic brain injury.

The present invention also relates to pharmaceutical compositions comprising the compounds of the present invention and a pharmaceutically acceptable carrier.

The present invention also relates to methods for the treatment, management, prevention, alleviation, amelioration, suppression, or control of disorders, diseases, and conditions that may be responsive to inhibition of the NLRP3 receptor in a subject in need thereof by administering the compounds and pharmaceutical compositions of the present invention.

The present invention also relates to the use of compounds of the present invention for manufacture of a medicament useful in treating diseases, disorders, and conditions that may be responsive to the inhibition of the NLRP3 receptor.

The present invention is also concerned with treatment or prevention of these diseases, disorders, and conditions by administering the compounds of the present invention in combination with a therapeutically effective amount of another agent that may be useful to treat the disease, disorder, and condition. The invention is further concerned with processes for preparing the compounds of this invention.

The summary of the technology described above is non-limiting and other features and advantages of the technology will be apparent from the following detailed description, and from the claims.

### DETAILED DESCRIPTION

The present disclosure is directed to compounds of Formula (I):

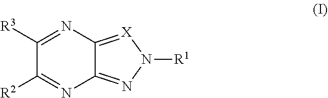

or a pharmaceutically acceptable salt thereof, wherein
X is selected from:
   1) N, and
   2) CR⁵;
R¹ is selected from:
   1) C₁₋₆ alkyl,
   2) C₀₋₃ alkylene-(monocyclic C₃₋₈ cycloalkyl),
   3) C₀₋₃ alkylene-(bicyclic C₄₋₈ cycloalkyl),
   4) C₀₋₃ alkylene-(C₅₋₁₀ spiro-cycloalkyl),
   5) C₀₋₃ alkylene-(tricyclic C₆₋₉ cycloalkyl),
   6) C₀₋₃ alkylene-(cubyl),
   7) C₀₋₃ alkylene-(3- to 9-membered monocyclic heterocycloalkyl containing 1, 2, or 3 ring heteroatoms selected from N, S, O, and P),
   8) C₀₋₃ alkylene-(4- to 9-membered bicyclic heterocycloalkyl containing 1, 2, or 3 ring heteroatoms selected from N, S, O, and P),
   9) C₀₋₃ alkylene-(5- to 9-membered spirocyclic heterocycloalkyl containing 1, 2, or 3 ring heteroatoms selected from N, S, O, and P),
   10) C₀₋₃ alkylene-(5- to 10-membered heteroaryl containing 1, 2, or 3 ring heteroatoms selected from N, S, O, and P),
   11) NH—C₁₋₆ alkyl,
   12) NH—C₀₋₃ alkylene-(monocyclic C₃₋₈ cycloalkyl),
   13) NH—C₀₋₃ alkylene-(bicyclic C₄₋₈ cycloalkyl),
   14) NH—C₀₋₃ alkylene-(C₅₋₁₀ spiro-cycloalkyl), and
   15) NH—C₀₋₃ alkylene-(tricyclic C₆₋₉ cycloalkyl),
   wherein said R¹ group is unsubstituted or substituted with 1 to 5 substituents independently selected from R⁶;
R² is phenyl unsubstituted or substituted by 1, 2, 3, or 4 substituents independently selected from:
   1) OH,
   2) halo,
   3) C₁₋₆ alkyl, and
   4) C₁₋₆ haloalkyl containing from 1 to 3 independently selected halogens;
R³ is selected from:
   1) H,
   2) halo, and
   3) C₁₋₆ alkyl,
R⁵ is selected from:
   1) H,
   2) OH,
   3) CN,
   4) C₁₋₆ alkyl,
   5) C₁₋₆ alkoxy, and
   6) C₃₋₇ cycloalkyl,
   wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
      1) OH,
      2) halo,
      3) CN,
      4) NR^{a}₂;
      wherein each R^{a} is independently selected from H and C₁₋₆ alkyl; and each R⁶ is independently selected from:
   1) OH,
   2) halo,
   3) CN,
   4) oxo,

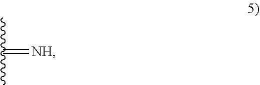 [SMILES: *=N]

6) C₁₋₆ alkyl, which may be straight or branched and which is unsubstituted or substituted with 1 to 4 substituents selected from OH, halo, CN, oxo, and C₁₋₆ alkoxy,
7) C₁₋₆ alkoxy, which may be straight or branched and which is unsubstituted or substituted with 1 to 4 substituents selected from OH, halo, CN, oxo, and C₁₋₆ alkoxy,
8) C₃₋₇ cycloalkyl, which is unsubstituted or substituted with 1 to 4 substituents selected from OH, halo, CN, oxo, C₁₋₃ alkyl, C₁₋₃ haloalkyl, and C₁₋₆ alkoxy,
9) 3- to 9-membered monocyclic heterocycloalkyl containing 1, 2, or 3 ring heteroatoms selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy,
10) C₅₋₇ aryl, which is unsubstituted or substituted with 1 to 4 substituents selected from OH, halo, CN, oxo, and C₁₋₆ alkoxy,
11) methylene-C₅₋₇ aryl, which is unsubstituted or substituted with 1 to 4 substituents selected from OH, halo, CN, oxo, and C₁₋₆ alkoxy, and
12) 5- to 10-membered heteroaryl containing at least one ring heteroatom selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy.

The invention has numerous embodiments, which are summarized below. The invention includes the compounds as shown and also includes individual diastereoisomers, enantiomers, and epimers of the compounds, and mixtures of diastereoisomers and/or enantiomers thereof including racemic mixtures.

A first embodiment is directed to compounds of Formula (I) in which X is selected from N and CR⁵.

In a first aspect of this embodiment, X is N.

In a second aspect of the embodiment, X is CR⁵, R⁵ is selected from H, OH, CN, C₁₋₆ alkyl, C₁₋₆ alkoxy, and C₃₋₇ cycloalkyl, and the R⁵ C₁₋₃ alkyl, R⁵ C₁₋₃ alkoxy, or R⁵ C₃₋₄ cycloalkyl is unsubstituted or substituted with 1 to 5 substituents independently selected from OH, halo, and NR^{a}₂, where each R^{a} is independently selected from H and C₁₋₆ alkyl.

In instances of this second aspect of the first embodiment, X is CR⁵, R⁵ is selected from H, OH, CN, C₁₋₆ alkyl, C₁₋₆ alkoxy, and C₃₋₇ cycloalkyl, and wherein the R⁵ C₁₋₆ alkyl, R⁵ C₁₋₆ alkoxy, or R⁵ C₃₋₇ cycloalkyl is unsubstituted or substituted with 1 to 5 substituents independently selected from OH, halo, and NR^{a}₂, where each R^{a} is independently selected from H and C₁₋₆ alkyl. In specific instances of this second aspect of the first embodiment, R⁵ is selected from H, OH, CN, methyl, ethyl, isopropyl, cyclopropyl, cyclobutyl, and methoxy, and the R⁵ methyl, ethyl, isopropyl, cyclopropyl, cyclobutyl, and methoxy is unsubstituted or substituted with 1 to 2 substituents independently selected from OH, halo, and NR^{a}₂, where each R^{a} is independently selected from H and C₁₋₆ alkyl. In instances of this second aspect of the first embodiment, R⁵ is selected from H, OH, CN, methyl, ethyl, isopropyl, cyclopropyl, cyclobutyl, and methoxy, and the R⁵ methyl, ethyl, isopropyl, cyclopropyl, cyclobutyl, and methoxy is unsubstituted or substituted with 1 to 2 substituents independently selected from OH, F, and NH₂. In instances of this second aspect of the first embodiment, R⁵ is selected from H, OH, CN, CH₃, CH₂OH, CF₂H, CH₂CH₃, CHOHCH₃, CH(NH₂)CH₃, C(CH₃)₂OH, OCH₃, OCF₂H, cyclopropyl, and cyclobutyl. In specific aspects of the first embodiment, X is selected from CH, C—OH, C—CN, C—CH₃, C—CH₂OH, C—CF₂H, C—CH₂CH₃, C—CHOHCH₃, C—CH(NH₂)CH₃, C—C(CH₃)₂OH, C—OCH₃, C—OCF₂H, C-cyclopropyl, and C-cyclobutyl. In instances of this second aspect of the first embodiment, R⁵ is selected from H, CH₃, and CHOHCH₃. In specific aspects of the first embodiment, X is selected from CH, C—CH₃, and C—CHOHCH₃.

A second embodiment is directed to compounds of Formula (I) in which R¹ is selected from: C₁₋₆ alkyl, C₀₋₃ alkylene-(monocyclic C₃₋₈ cycloalkyl), C₀₋₃ alkylene-(bicyclic C₄₋₈ cycloalkyl), C₀₋₃ alkylene-(C₅₋₁₀ spiro-cycloalkyl), C₀₋₃ alkylene-(tricyclic C₆₋₉ cycloalkyl), C₀₋₃ alkylene-(cubyl), C₀₋₃ alkylene-(3- to 9-membered monocyclic heterocycloalkyl containing 1, 2, or 3 ring heteroatoms selected from N, S, O, and P), C₀₋₃ alkylene-(4- to 9-membered bicyclic heterocycloalkyl containing 1, 2, or 3 ring heteroatoms selected from N, S, O, and P), C₀₋₃ alkylene-(5- to 9-membered spirocyclic heterocycloalkyl containing 1, 2, or 3 ring heteroatoms selected from N, S, O, and P), C₀₋₃ alkylene-(5- to 10-membered heteroaryl containing 1, 2, or 3 ring heteroatoms selected from N, S, O, and P), NH—C₁₋₆ alkyl, NH—C₀₋₃ alkylene-(monocyclic C₃₋₈ cycloalkyl), NH—C₀₋₃ alkylene-(bicyclic C₄₋₈ cycloalkyl), NH—C₀₋₃ alkylene-(C₅₋₁₀ spiro-cycloalkyl), and NH—C₀₋₃ alkylene-(tricyclic C₆₋₉ cycloalkyl). Aspects of this second embodiment are directed to compounds of Formula (I) in which R¹ is selected from: C₁₋₆ alkyl, C₀₋₃ alkylene-(monocyclic C₃₋₈ cycloalkyl), C₀₋₃ alkylene-(bicyclic C₄₋₈ cycloalkyl), C₀₋₃ alkylene-(C₅₋₁₀ spiro-cycloalkyl), C₀₋₃ alkylene-(tricyclic C₆₋₉ cycloalkyl), C₀₋₃ alkylene-(3- to 9-membered monocyclic heterocycloalkyl containing 1, 2, or 3 ring heteroatoms selected from N, S, O, and P), C₀₋₃ alkylene-(4- to 9-membered bicyclic heterocycloalkyl containing 1, 2, or 3 ring heteroatoms selected from N, S, O, and P), C₀₋₃ alkylene-(5- to 9-membered spirocyclic heterocycloalkyl containing 1, 2, or 3 ring heteroatoms selected from N, S, O, and P), and C₀₋₃ alkylene-(5- to 10-membered heteroaryl containing 1, 2, or 3 ring heteroatoms selected from N, S, O, and P), wherein said R¹ group is unsubstituted or substituted with 1 to 5 substituents independently selected from R⁶. In aspects of this second embodiment, R¹ is selected from: C₁₋₄ alkyl, monocyclic C₃₋₆ cycloalkyl, methylene-(monocyclic C₃₋₆ cycloalkyl), bicyclic C₅₋₈ cycloalkyl, tricyclic C₇ cycloalkyl, 4- to 7-membered monocyclic heterocycloalkyl containing 1 or 2 ring heteroatoms selected from N, S, and O, methylene-(4- to 7-membered monocyclic heterocycloalkyl containing 1 or 2 ring heteroatoms selected from N, S, and O), 4- to 9-membered bicyclic heterocycloalkyl containing 1, 2, or 3 ring heteroatoms selected from N, S, and O, methylene-(4- to 9-membered bicyclic heterocycloalkyl containing 1, 2, or 3 ring heteroatoms selected from N, S, and O), 6- to 9-membered spirocyclic heterocycloalkyl containing 1, 2, or 3 ring heteroatoms selected from N, S, and O), 5-membered heteroaryl containing 1 or 2 ring heteroatoms selected from N, S, and O, and methylene-(5-membered heteroaryl containing 1 or 2 ring heteroatoms selected from N, S, and O), wherein said R¹ group is unsubstituted or substituted with 1 to 5 substituents independently selected from R⁶.

In aspects of this second embodiment, each R⁶ is selected independently from OH, halo, CN, oxo,

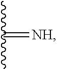 [SMILES: *=N]

C₁₋₆ alkyl, which may be straight or branched and which is unsubstituted or substituted with 1 to 4 substituents selected from OH, halo, CN, oxo, and C₁₋₆ alkoxy, C₁₋₆ alkoxy, which may be straight or branched and which is unsubstituted or substituted with 1 to 4 substituents selected from OH, halo, CN, oxo, and C₁₋₆ alkoxy, C₃₋₇ cycloalkyl, which is unsubstituted or substituted with 1 to 4 substituents selected from OH, halo, CN, oxo, C₁₋₃ alkyl, C₁₋₃ haloalkyl, and C₁₋₆ alkoxy, 3- to 9-membered monocyclic heterocycloalkyl containing 1, 2, or 3 ring heteroatoms selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy, C₅₋₇ aryl, which is unsubstituted or substituted with 1 to 4 substituents selected from OH, halo, CN, oxo, and C₁₋₆ alkoxy, methylene-C₅₋₇ aryl, which is unsubstituted or substituted with 1 to 4 substituents selected from OH, halo, CN, oxo, and C₁₋₆ alkoxy, and 5- to 10-membered heteroaryl containing at least one ring heteroatom selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy.

In aspects of this second embodiment, each R¹ is selected from

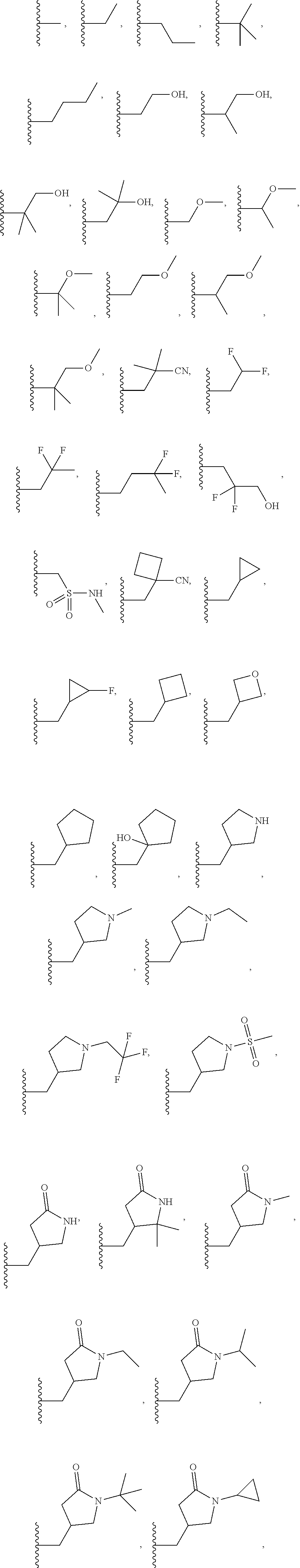

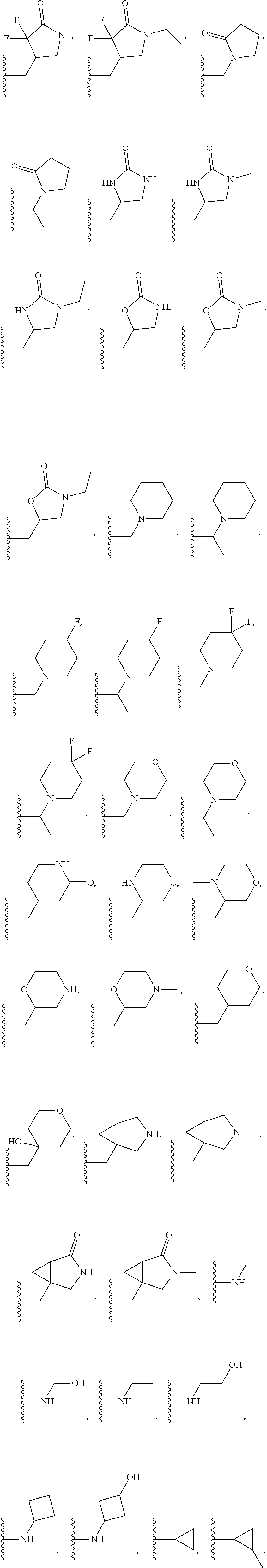

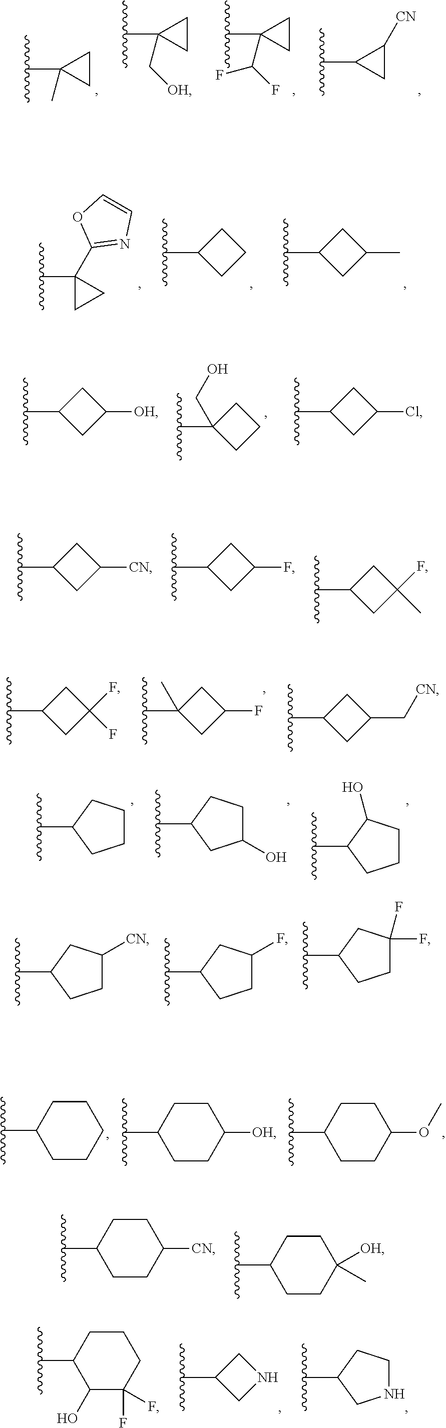

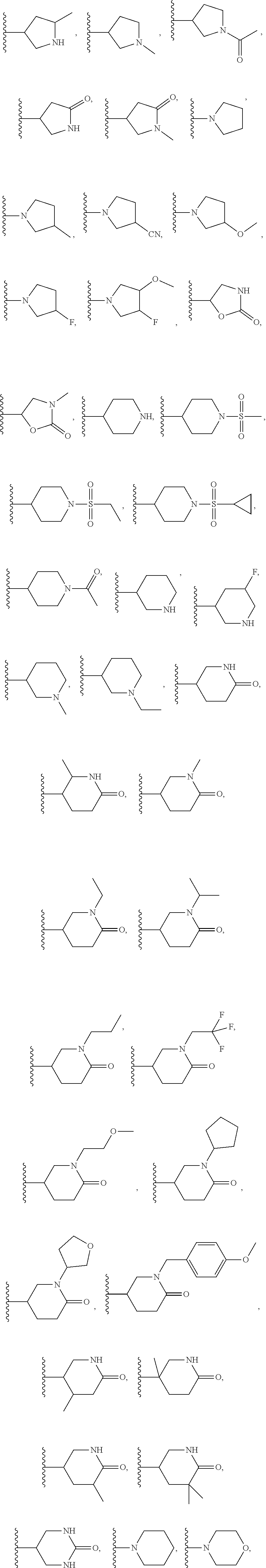

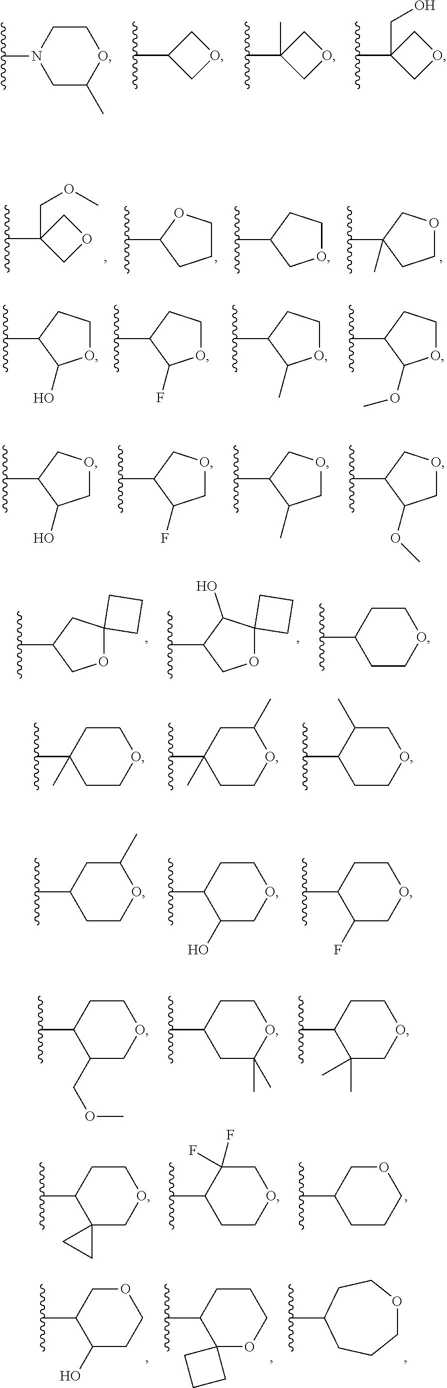

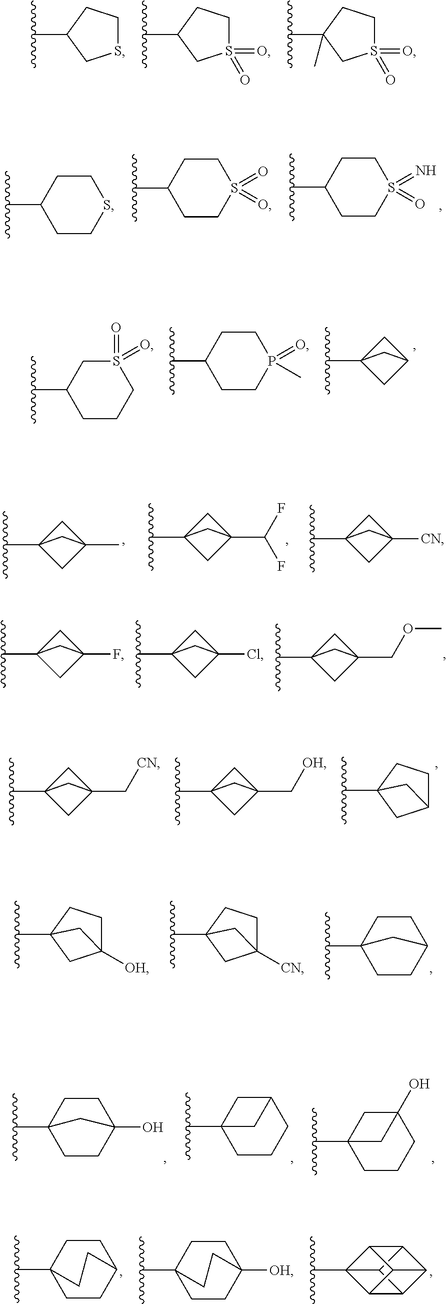

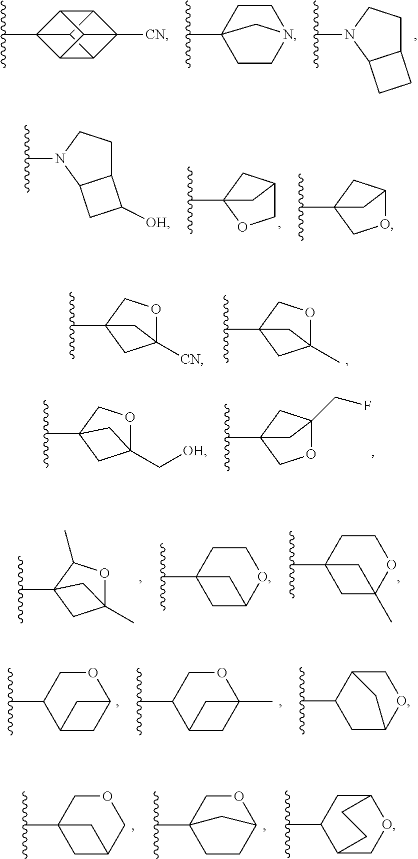

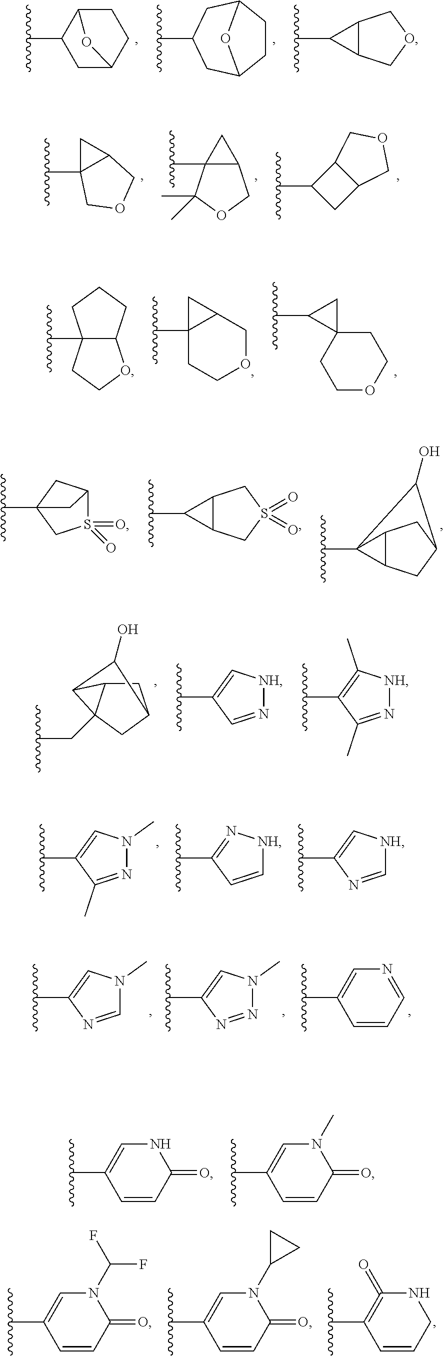

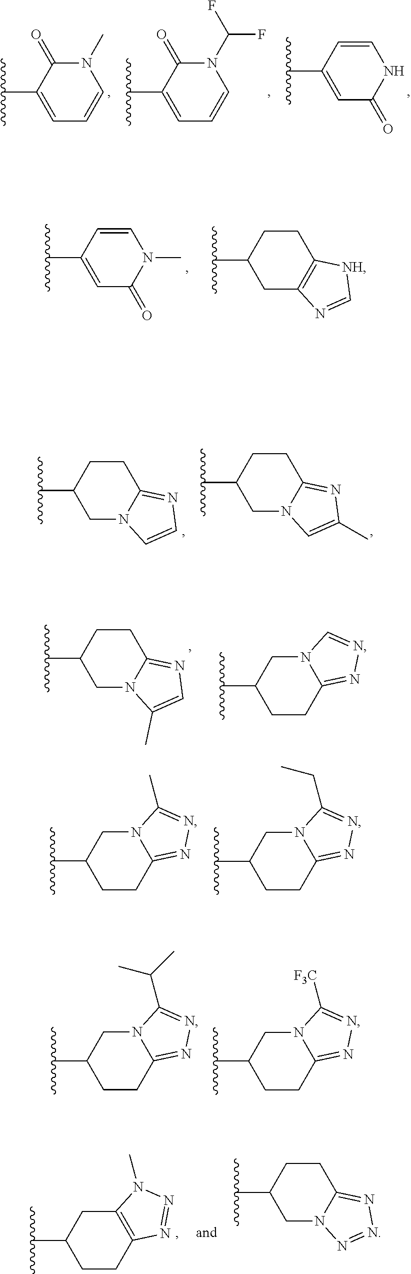

In certain aspects of this second embodiment each R¹ is selected from:

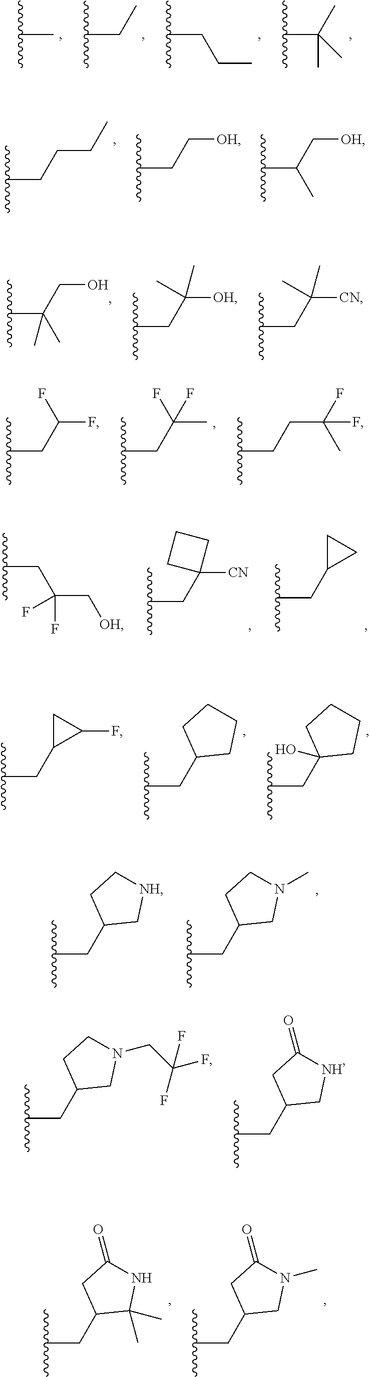

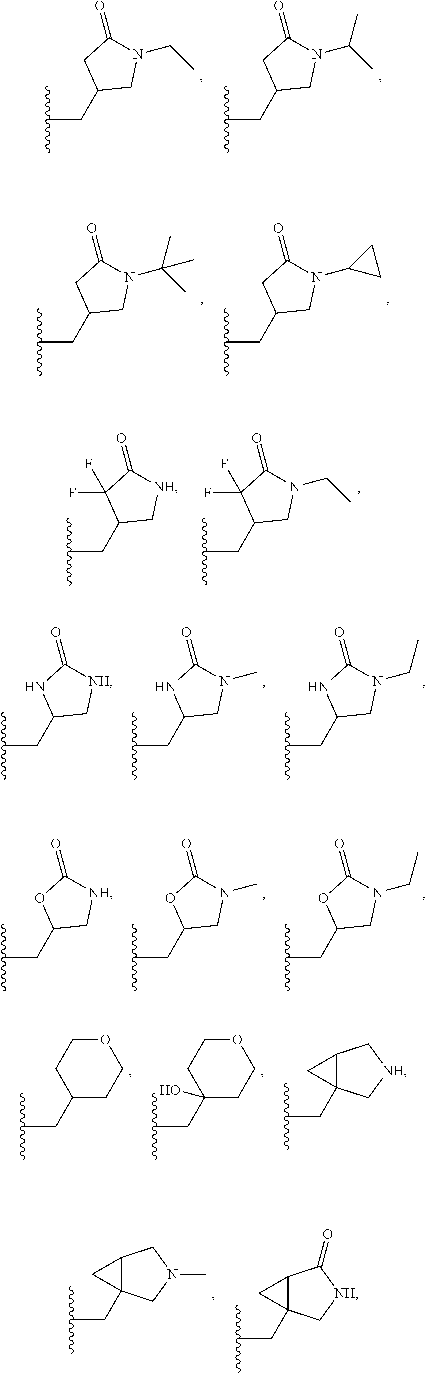

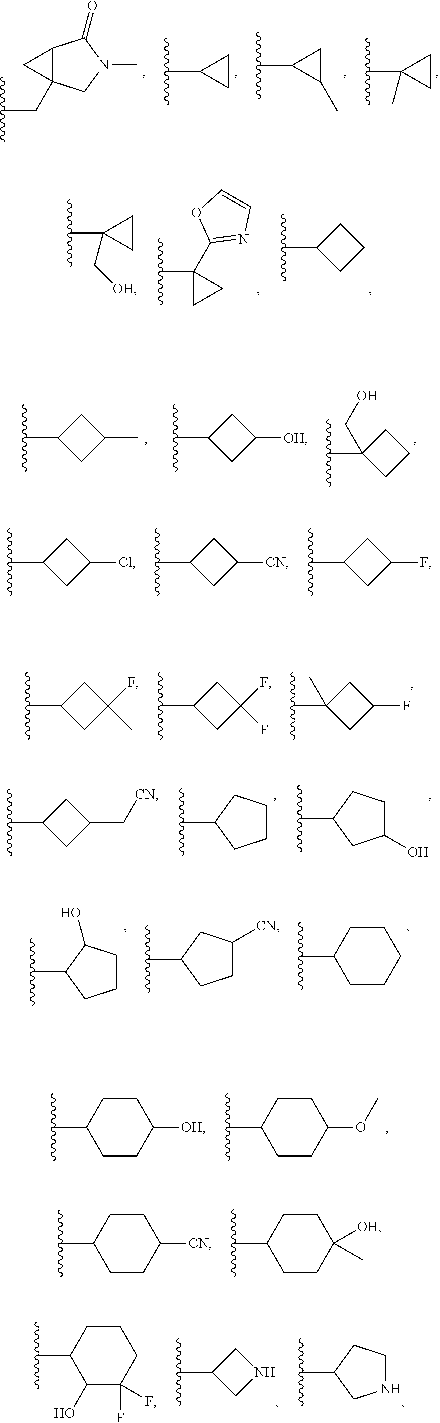

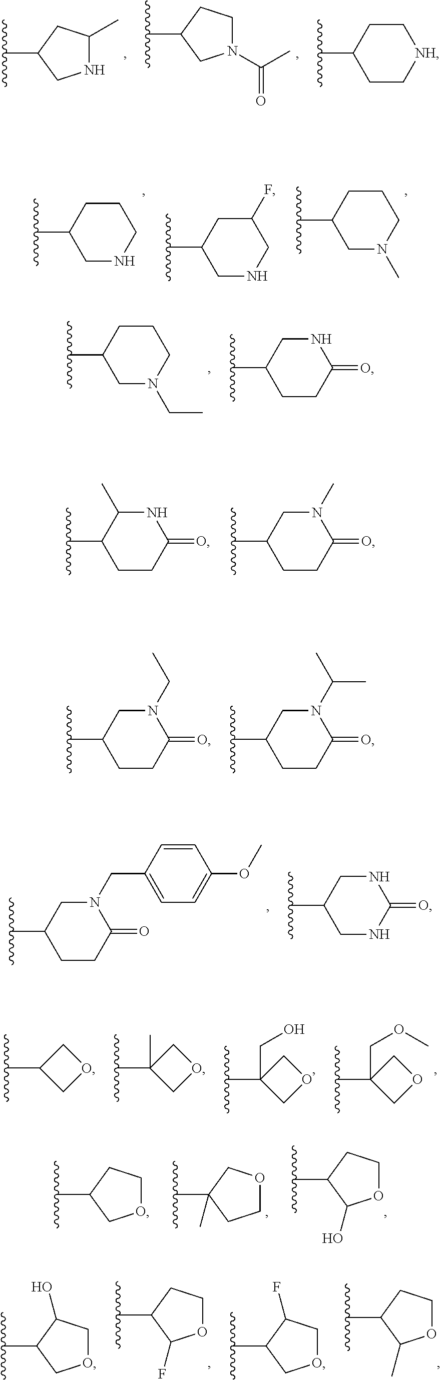

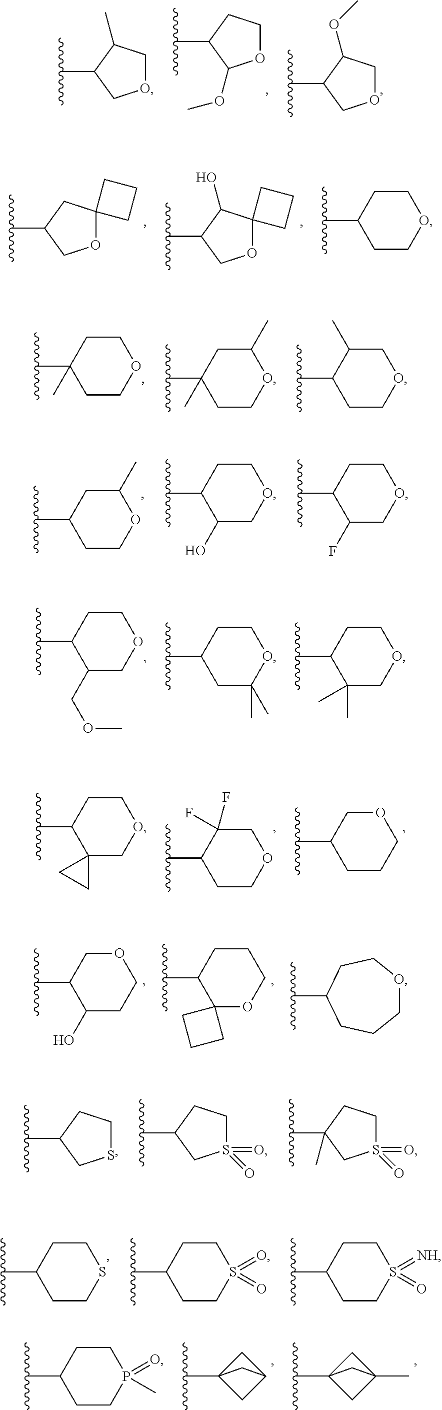

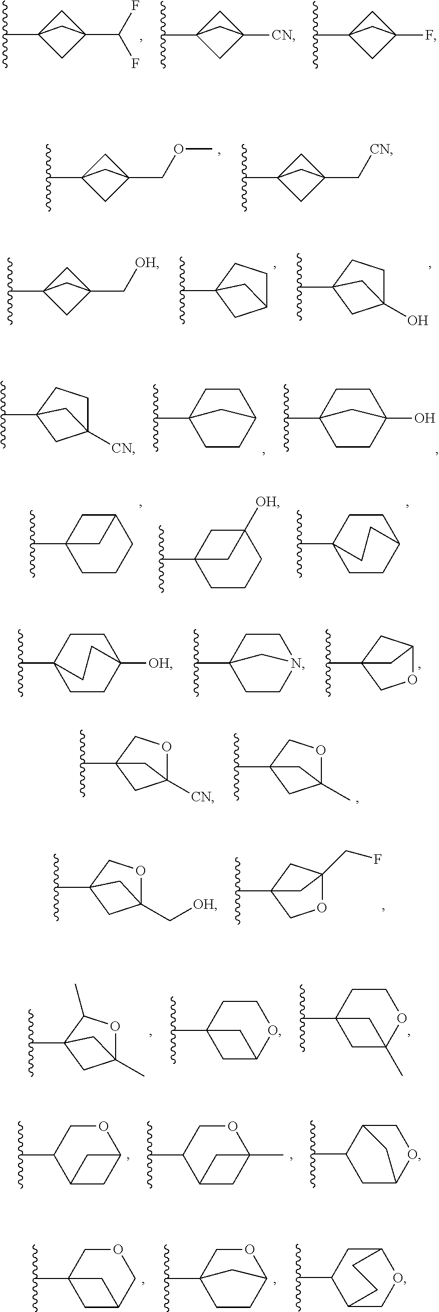

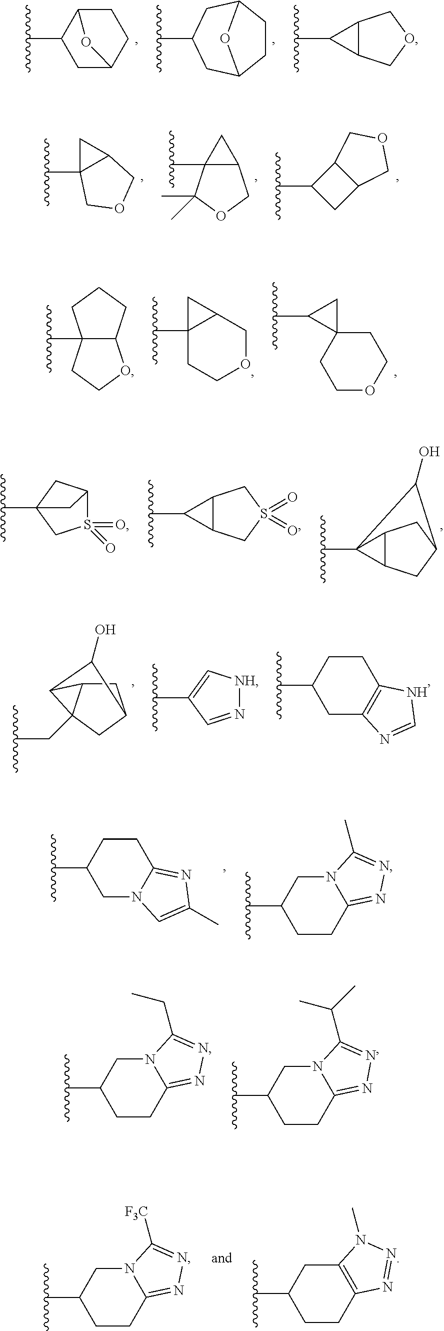

In certain aspects of this second embodiment, each R¹ is selected from

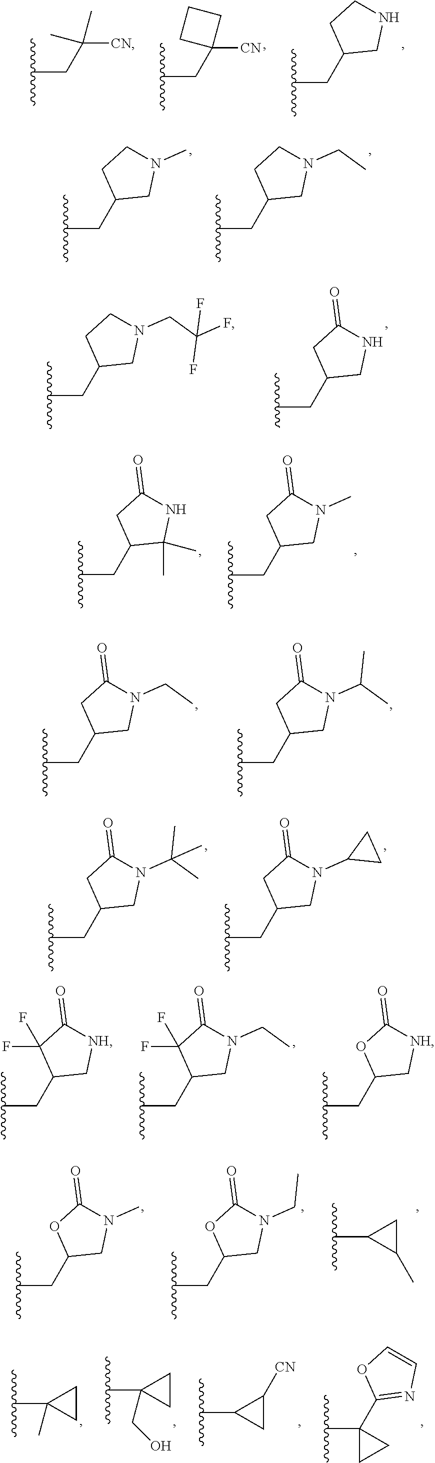

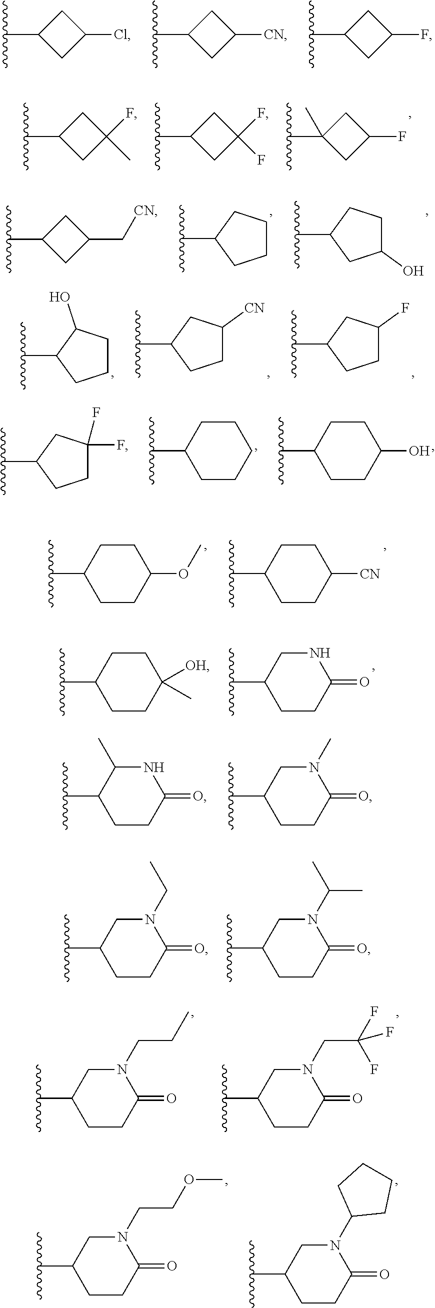

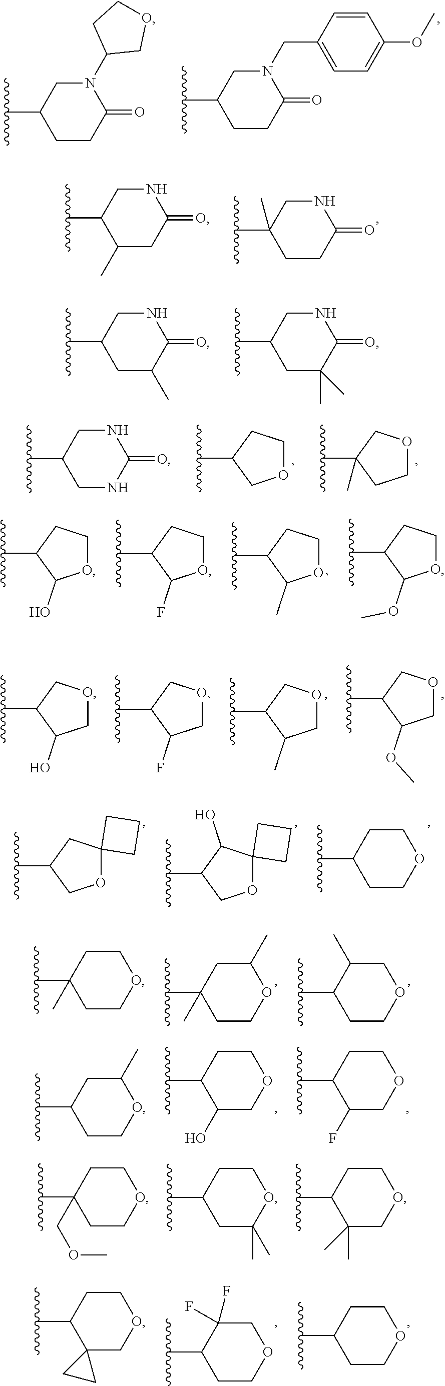

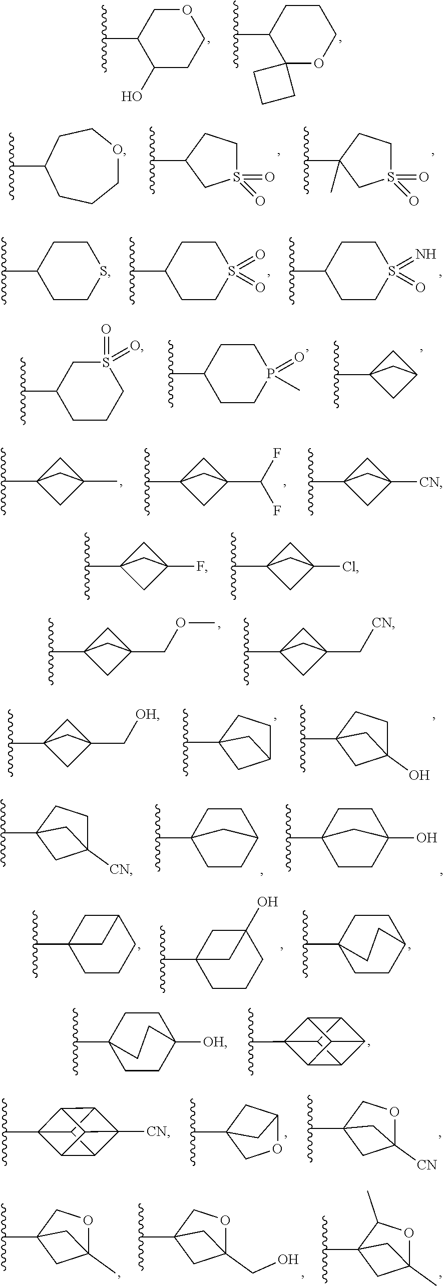

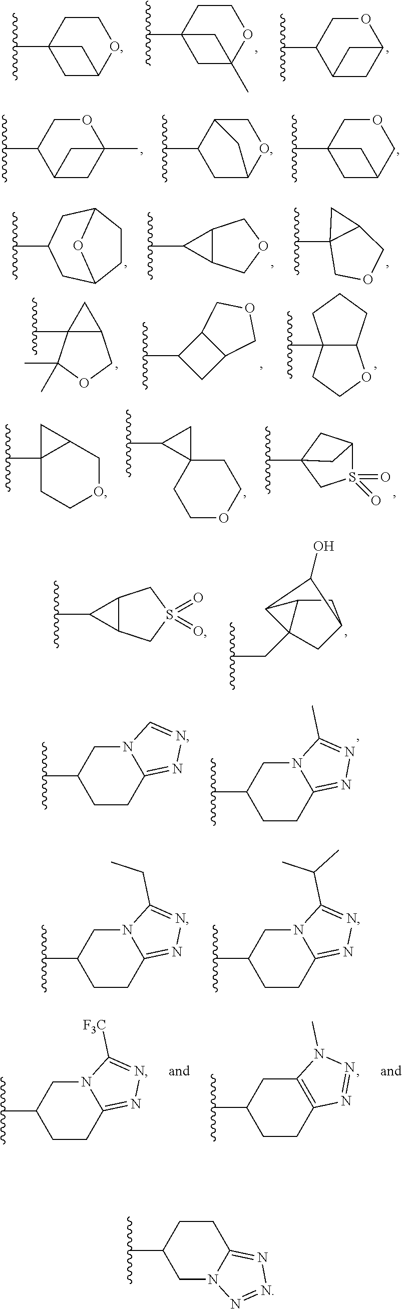

In certain further aspects of this second embodiment, each R¹ is selected from

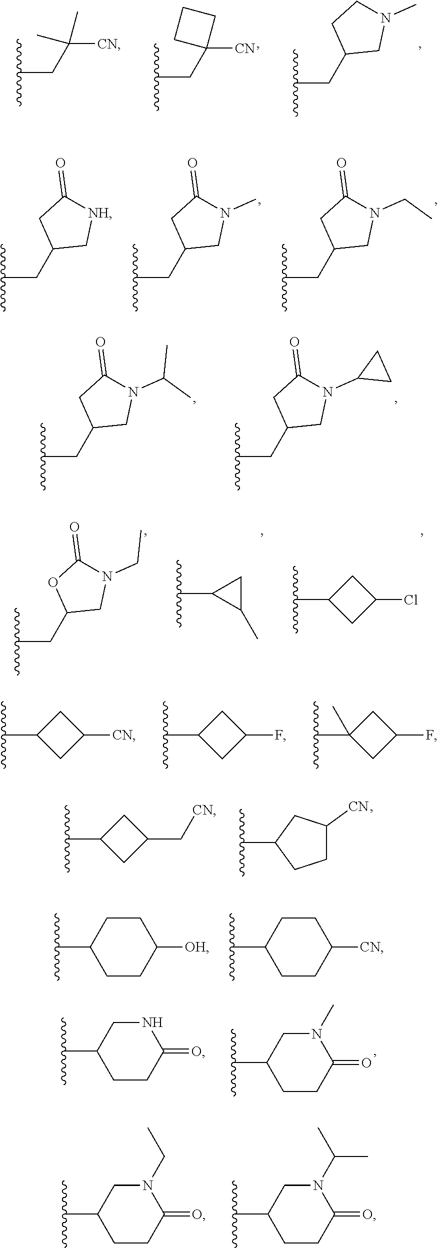

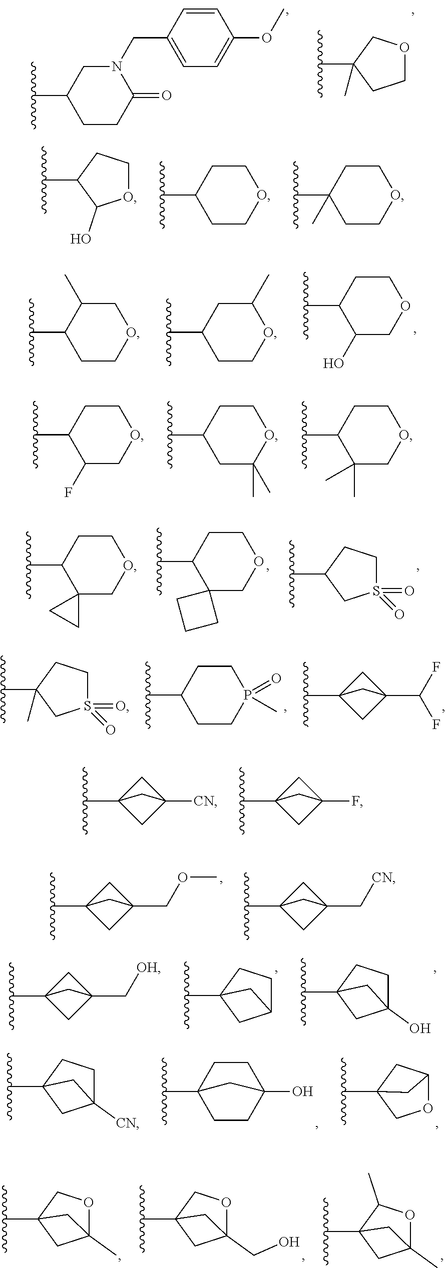

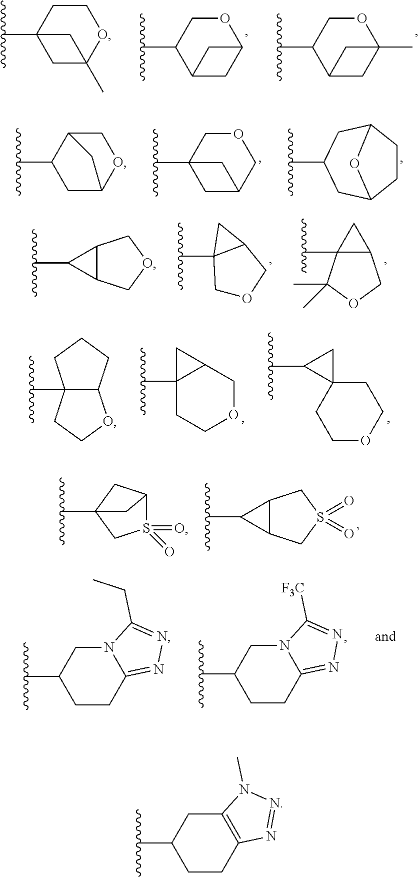

In particular aspects, each R¹ is selected from:

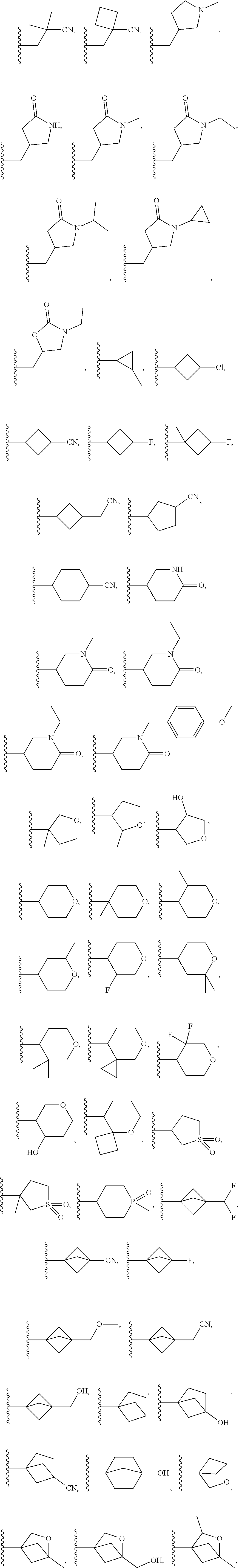

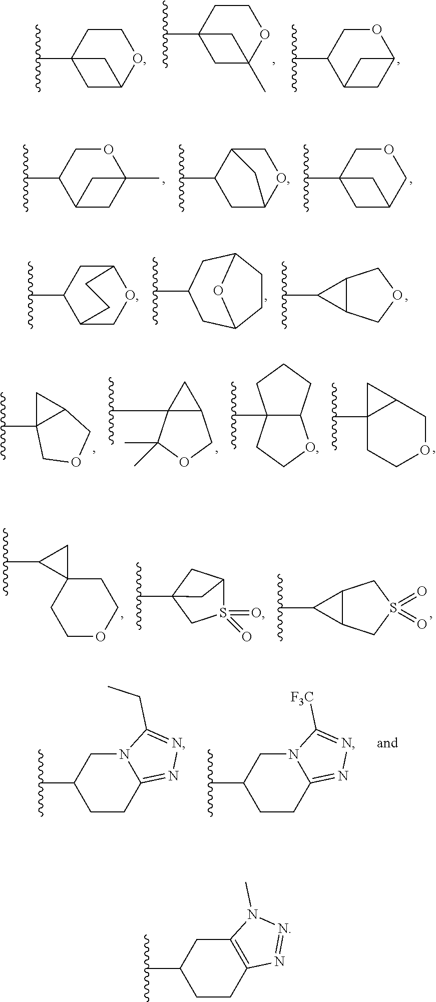

In more particular aspects, each R¹ is selected from:

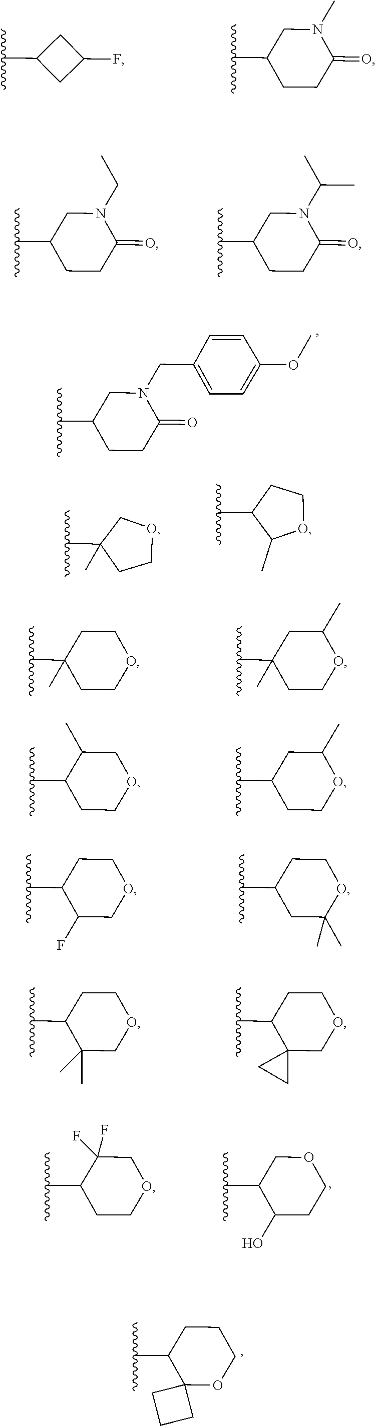

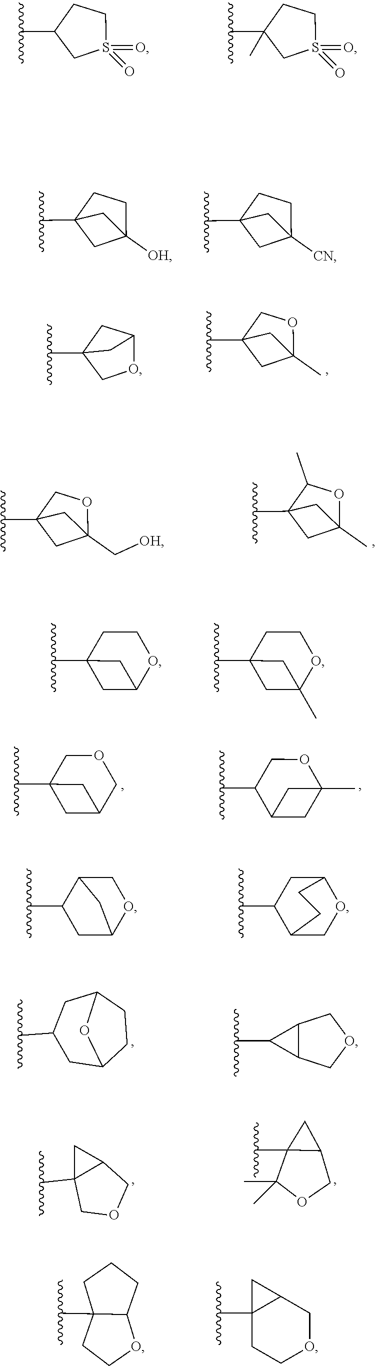

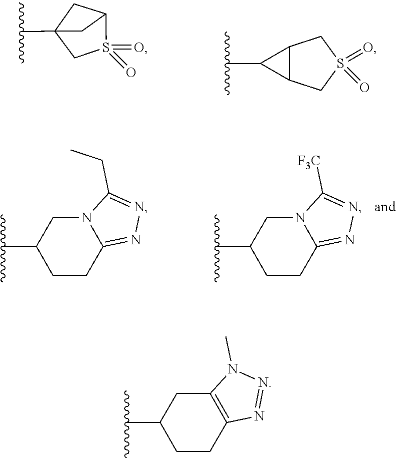 [SMILES: *C12CC(C1)S(=O)(=O)C2 ; *C1C2CS(=O)(=O)CC12 ; *C1CCc2nnc(CC)n2C1 ; *C1CCc2nnc(C(F)(F)F)n2C1 ; *C1CCc2nnn(C)c2C1]

In still more particular aspects, each R¹ is selected from:

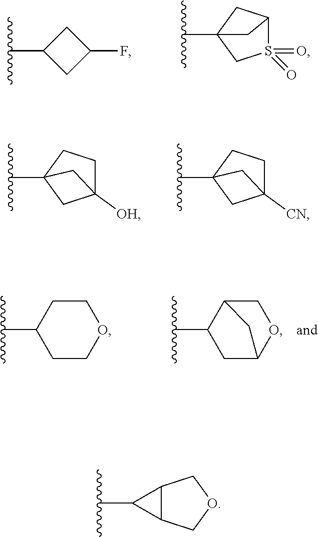

In even more particular aspects, each R¹ is selected from:

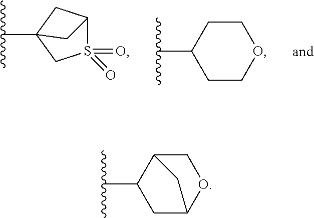

A third embodiment is directed to compounds of Formula (I) in which R² is phenyl substituted by from 0 to 3 substituents independently selected from OH, halo, C₁₋₆ alkyl, and C₁₋₆ haloalkyl containing from 1 to 3 independently selected halogens. In aspects of this third embodiment, R² is phenyl substituted by 2 or 3 substituents independently selected from: OH, halo, C₁₋₆ alkyl, and C₁₋₆ haloalkyl containing from 1 to 3 independently selected halogens. In particular aspects, R² is phenyl substituted by 2 or 3 substitutents independently selected from: OH, C₁, CH₃, and CF₃. In more particular aspects, R² is phenyl substituted by OH and CF₃. In still more particular aspects, R² is phenyl substituted by OH, CH₃, and CF₃. In additional particular aspects, R² is phenyl substituted by OH, CH₃, and Cl. In specific aspects, R² is selected from:

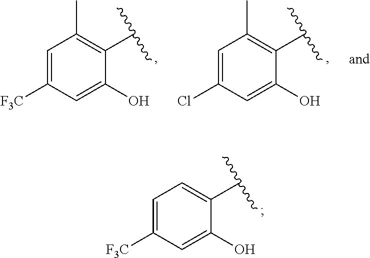

In more specific aspects, R² is

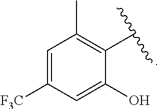 [SMILES: *c1c(C)cc(C(F)(F)F)cc1O]

A fourth embodiment is directed to compounds of Formula (I) in which R³ is selected from H, halo, and C₁₋₆ alkyl. In aspects of this fourth embodiment, R³ is selected from H and C₁₋₆ alkyl. In specific aspects of this embodiment, R³ is H. In other specific aspects, R³ is C₁₋₆ alkyl. In specific instances of these aspects, the R³ C₁₋₆ alkyl is selected from methyl, ethyl, propyl, and isopropyl. In specific instances, R³ C₁₋₆ alkyl is methyl. In specific instances, R³ C₁₋₆ alkyl is ethyl. In specific instances, R³ C₁₋₆ alkyl is propyl. In specific instances, R³ C₁₋₆ alkyl is isopropyl.

Embodiments of the disclosure are directed to compounds of Formula (I):

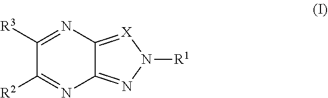

or a pharmaceutically acceptable salt thereof, wherein
X is N;
R¹ is selected from:
   1) C₁₋₄ alkyl,
   2) monocyclic C₃₋₆ cycloalkyl,
   3) methylene-(monocyclic C₃₋₆ cycloalkyl),
   4) bicyclic C₅₋₈ cycloalkyl,
   5) 5- to 6-membered monocyclic heterocycloalkyl containing 1 or 2 ring heteroatoms selected from N, S, and O,
   6) methylene-(5- to 6-membered monocyclic heterocycloalkyl containing 1 or 2 ring heteroatoms selected from N, S, and O),
   7) 6- to 9-membered bicyclic heterocycloalkyl containing 1, 2, or 3 ring heteroatoms selected from N, S, and O,
   8) methylene-(4- to 9-membered bicyclic heterocycloalkyl containing 1, 2, or 3 ring heteroatoms selected from N, S, and O), and
wherein said R¹ group is unsubstituted or substituted with 1 to 5 substituents independently selected from R⁶;
R² is phenyl unsubstituted or substituted by 1, 2, 3, or 4 substituents independently selected from:
   1) OH,
   2) halo,
   3) C₁₋₂ alkyl, and
   4) C₁₋₂ haloalkyl containing from 1 to 3 independently selected halogens;
R³ is selected from:
   1) H,
   2) C₁₋₆ alkyl; and
each R⁶ is independently selected from:
   1) OH,
   2) F,
   3) CN,
   4) oxo,
   5) C₁₋₃ alkyl, which may be straight or branched and which is unsubstituted or substituted with 1 to 4 substituents selected from OH, F, CN, oxo, and OCH₃,
   6) C₃ cycloalkyl, which is unsubstituted, and
   7) methylene-C₅₋₇ aryl, which is unsubstituted or substituted with 1 to 4 substituents selected from OH, F, CN, oxo, and OCH₃.

Embodiments of the disclosure are directed to compounds of Formula (I):

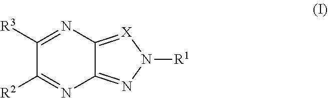

or a pharmaceutically acceptable salt thereof, wherein
X is N;
R¹ is selected from:

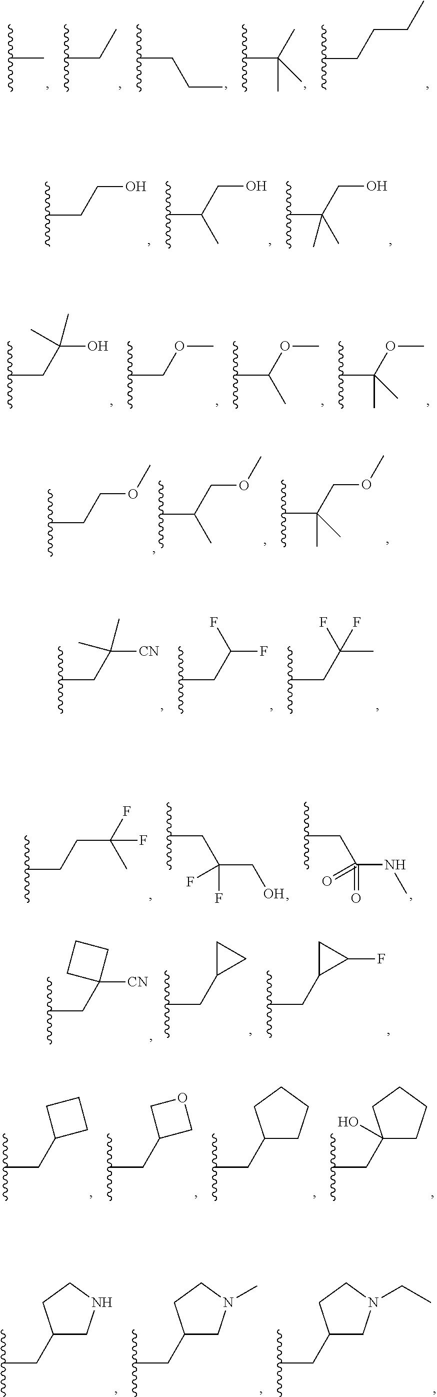

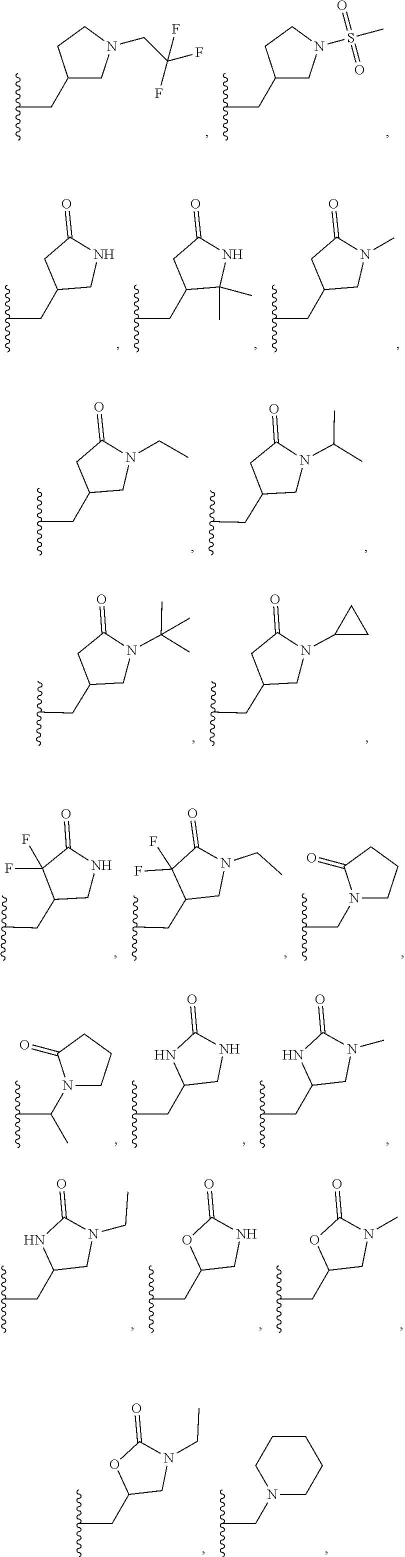

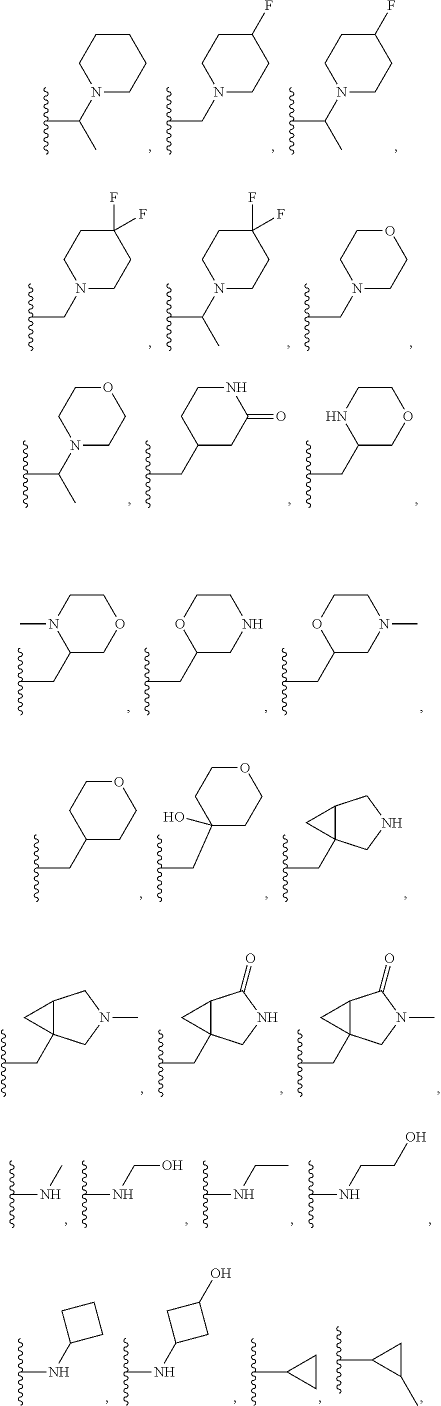

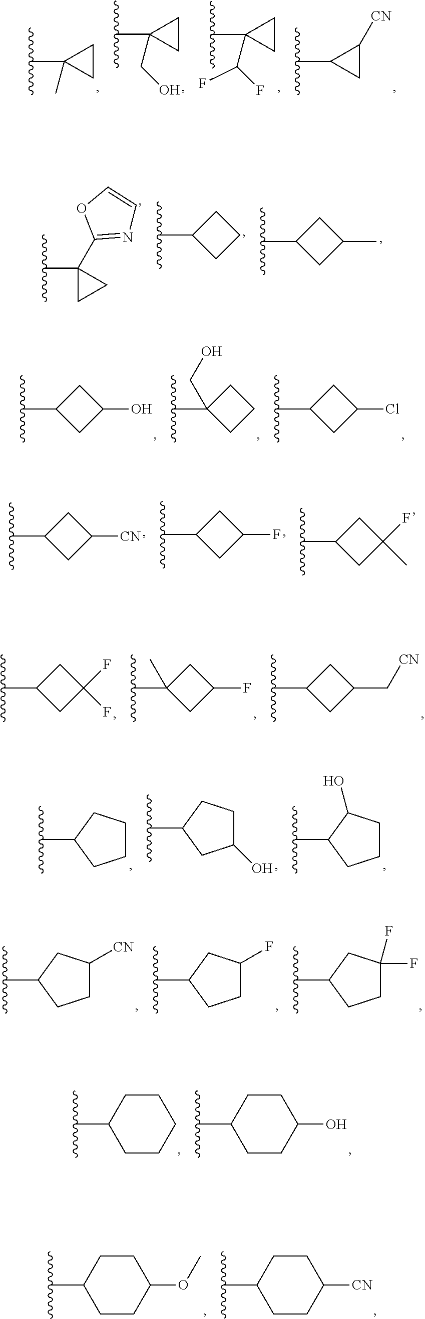

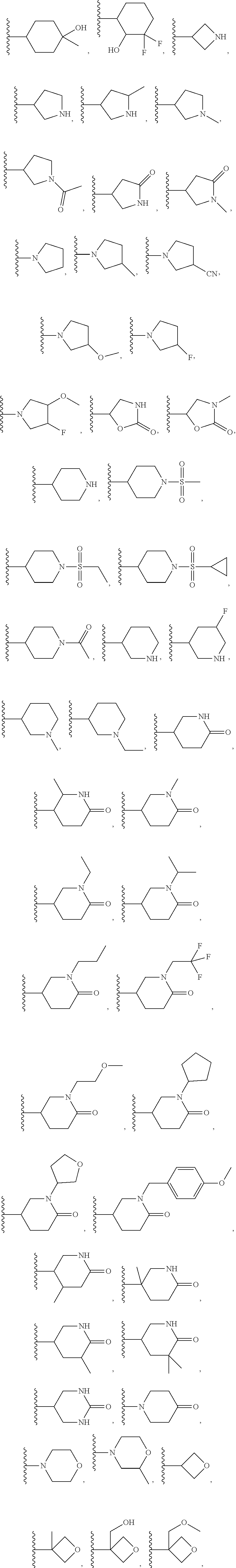

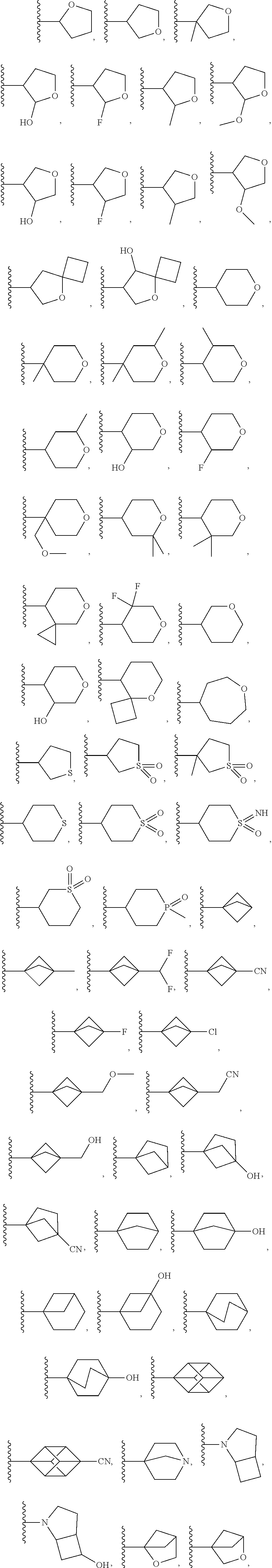

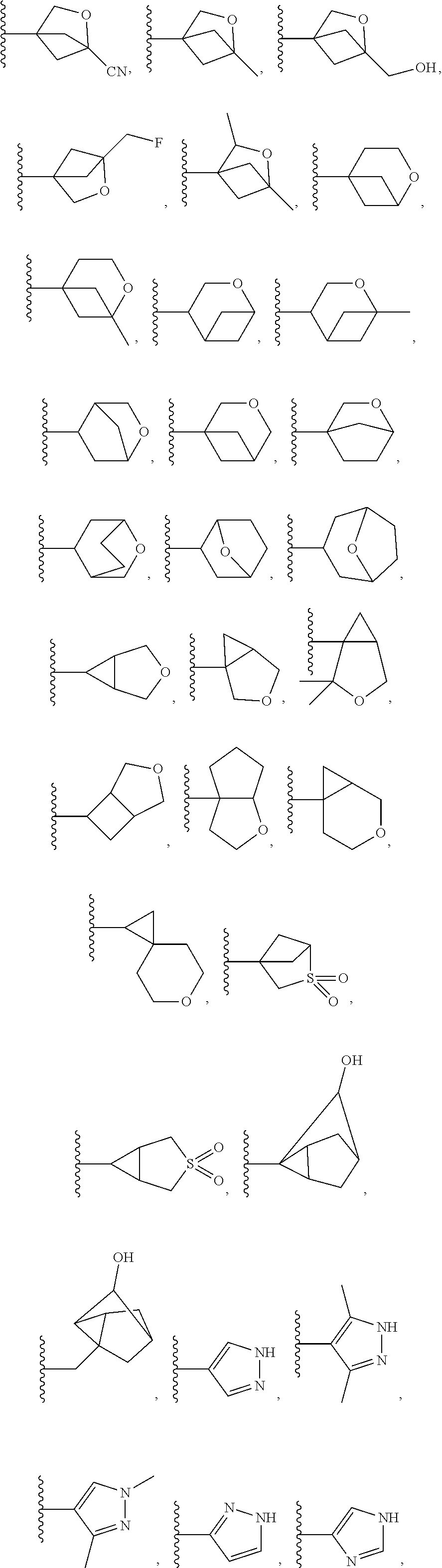

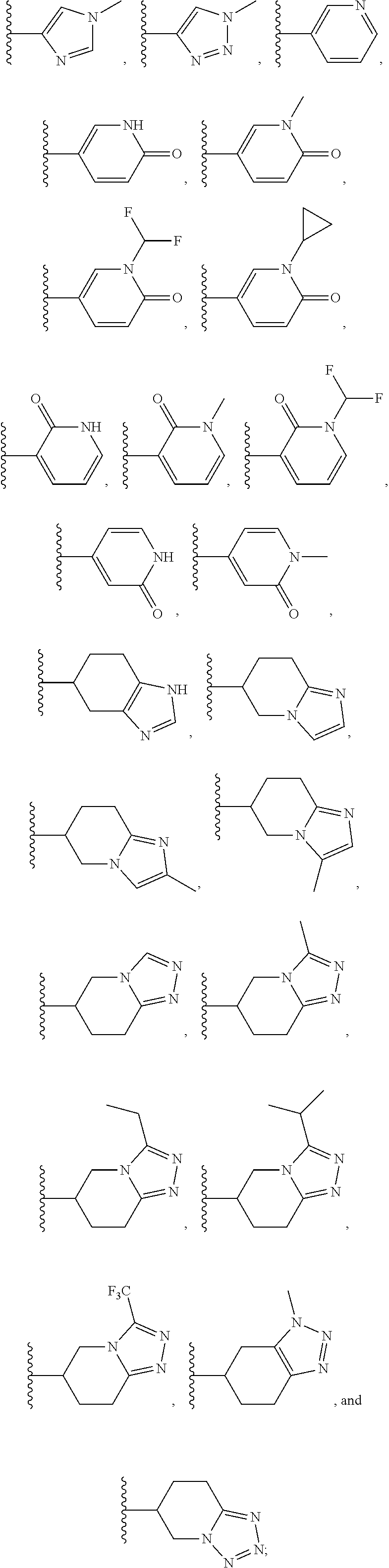

R² is selected from:

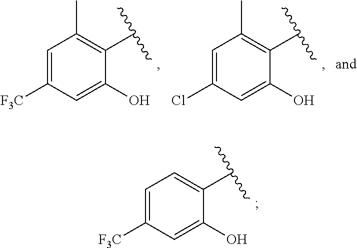

R³ is selected from:
   1) H,
   2) C₁₋₆ alkyl.

Embodiments of the disclosure are directed to compounds of Formula (I):

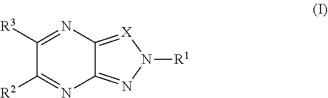

or a pharmaceutically acceptable salt thereof, wherein
X is N;
R¹ is selected from:

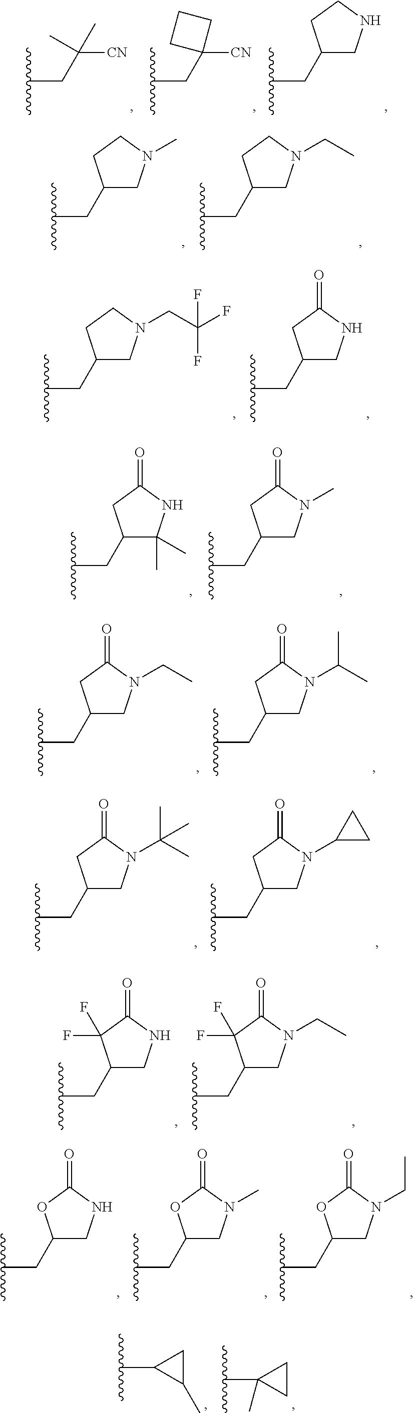

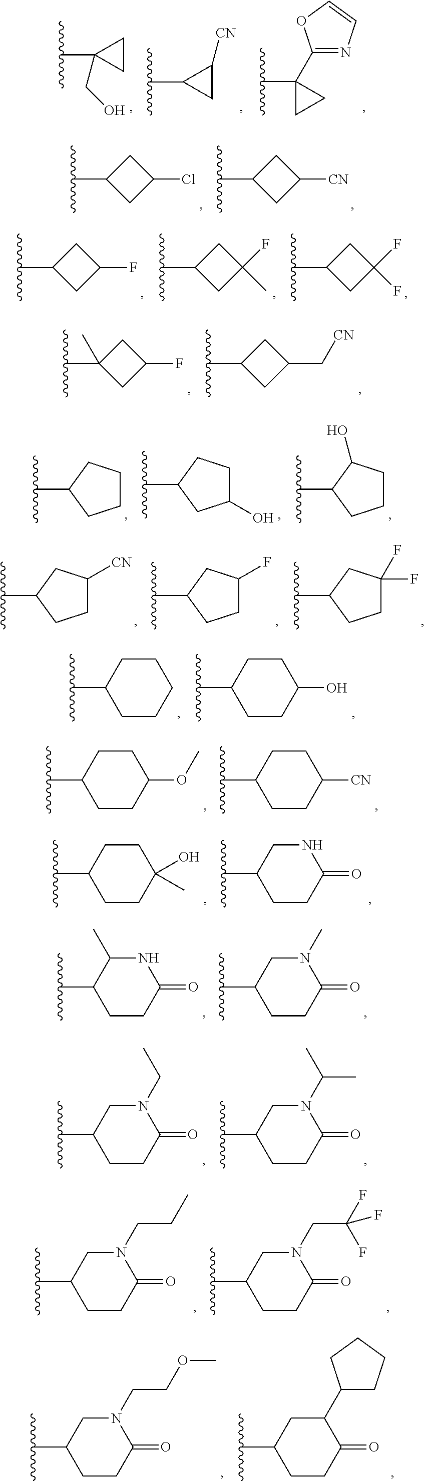

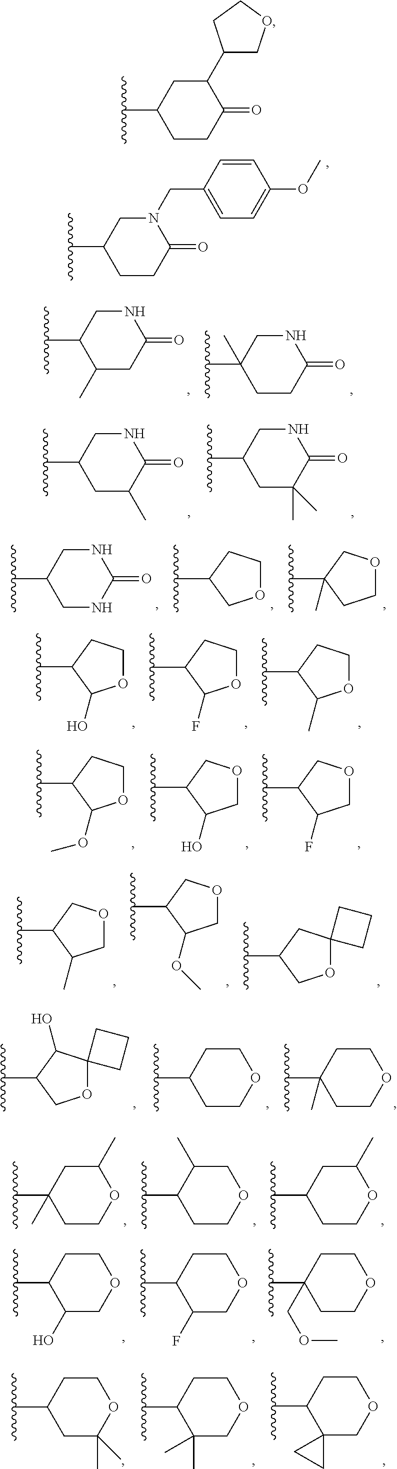

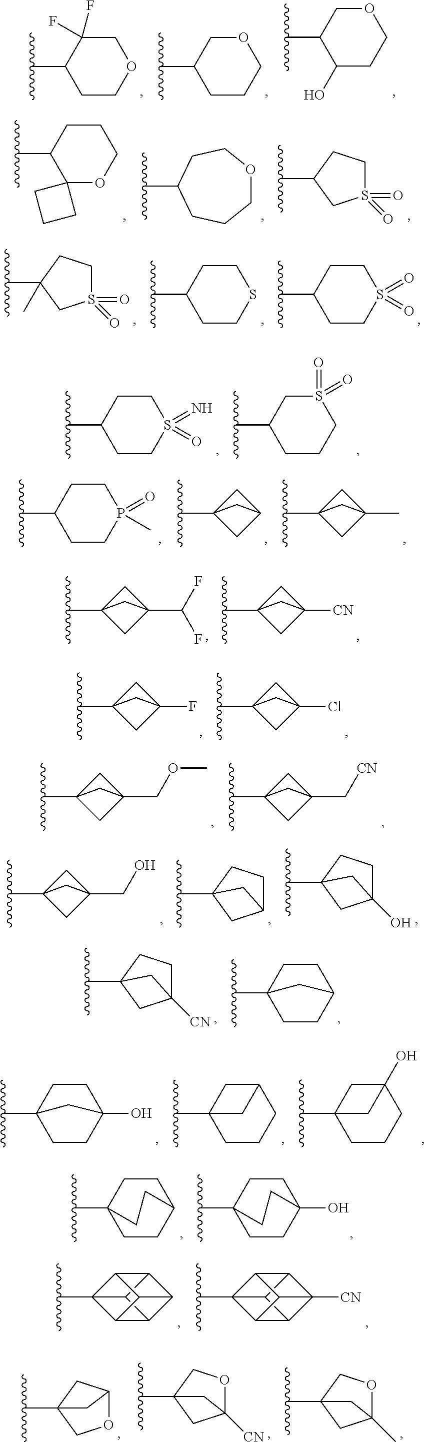

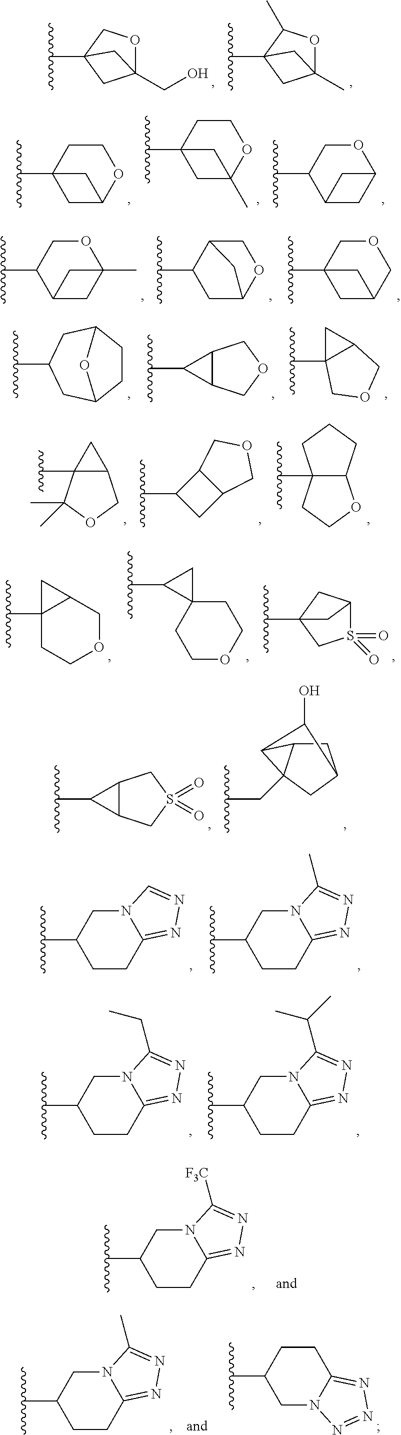

R² is selected from:

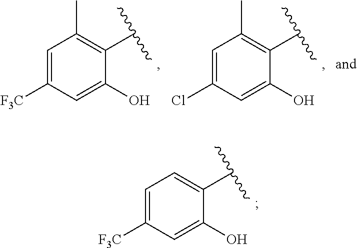

R³ is selected from:
   1) H,
   2) C₁₋₆ alkyl.

Embodiments of the disclosure are directed to compounds of Formula (I):

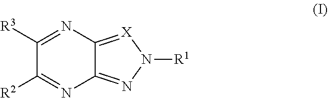

or a pharmaceutically acceptable salt thereof, wherein
X is N;
R¹ is selected from:

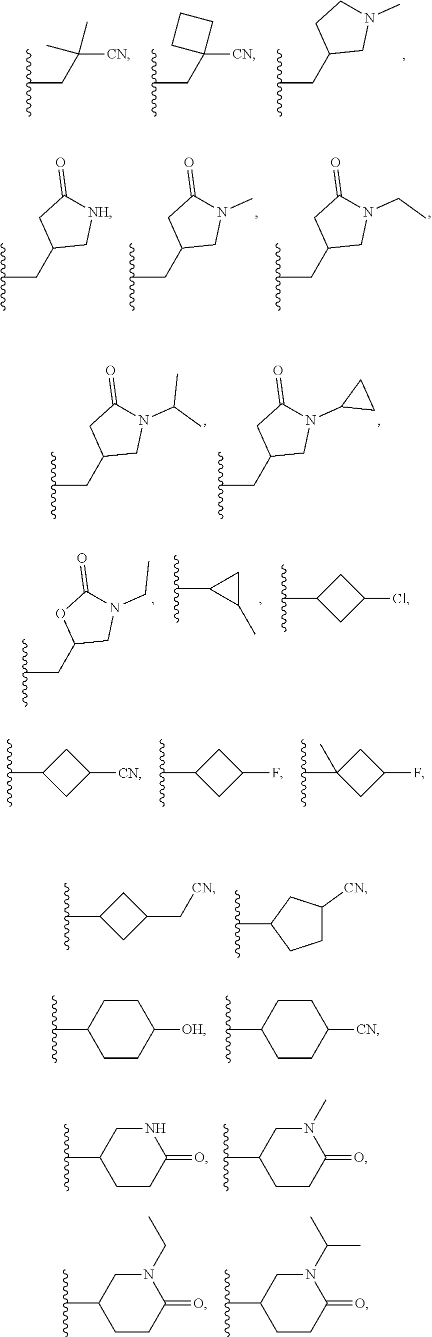

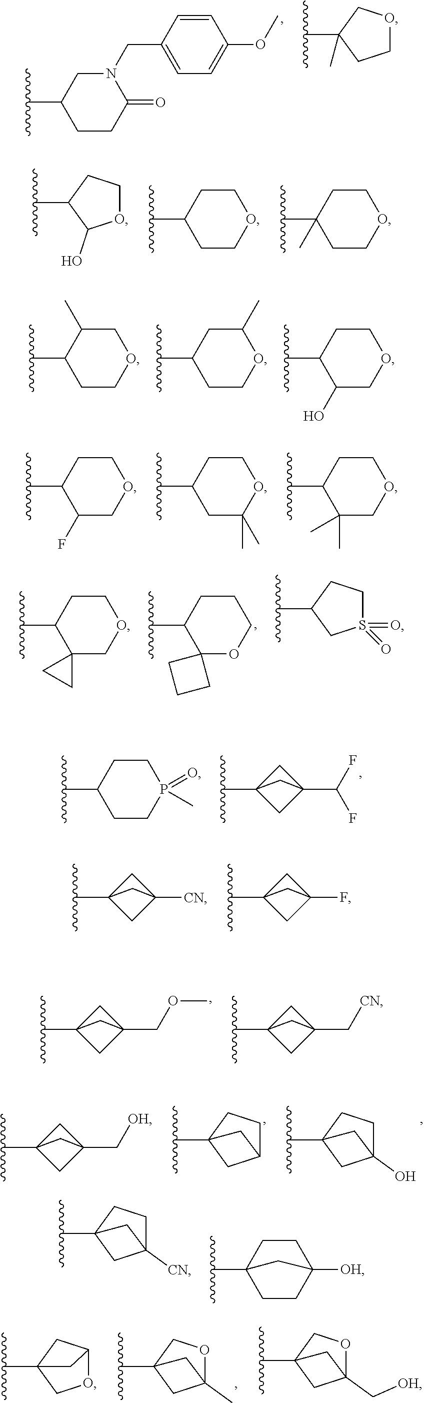

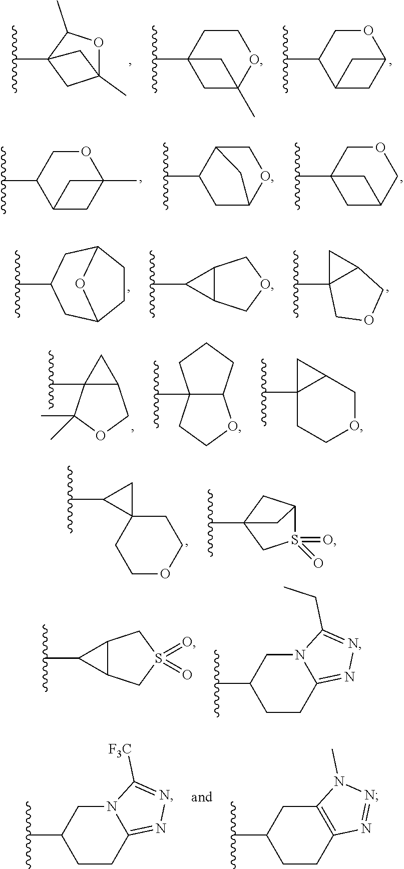

R² is selected from:

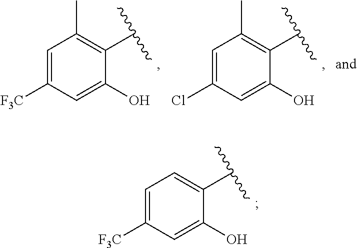

R³ is selected from:
   1) H,
   2) methyl.

Embodiments of the disclosure are directed to compounds of Formula (I):

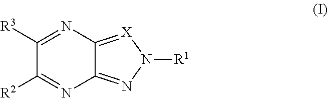

or a pharmaceutically acceptable salt thereof, wherein
X is CR⁵;
R¹ is selected from:
   1) C₁₋₄ alkyl,
   2) monocyclic C₃₋₆ cycloalkyl,
   3) methylene-(monocyclic C₃₋₆ cycloalkyl),
   4) bicyclic C₅₋₈ cycloalkyl,
   5) 5- to 6-membered monocyclic heterocycloalkyl containing 1 or 2 ring heteroatoms selected from N, S, and O,
   6) methylene-(5- to 6-membered monocyclic heterocycloalkyl containing 1 or 2 ring heteroatoms selected from N, S, and O),
   7) 6- to 9-membered bicyclic heterocycloalkyl containing 1, 2, or 3 ring heteroatoms selected from N, S, and O,
   8) methylene-(4- to 9-membered bicyclic heterocycloalkyl containing 1, 2, or 3 ring heteroatoms selected from N, S, and O), and
wherein said R¹ group is unsubstituted or substituted with 1 to 5 substituents independently selected from R⁶;
R² is phenyl unsubstituted or substituted by 1, 2, 3, or 4 substituents independently selected from:
   1) OH,
   2) halo,
   3) C₁₋₂ alkyl, and
   4) C₁₋₂ haloalkyl containing from 1 to 3 independently selected halogens;
R³ is selected from:
   1) H,
   2) C₁₋₆ alkyl;
R⁵ is selected from:
   1) H,
   2) OH,
   3) CN,
   4) C₁₋₃ alkyl,
   5) C₁₋₃ alkoxy, and
   6) C₃₋₄ cycloalkyl,
   wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
      1) OH,
      2) C₁,
      3) F,
      4) CN,
      5) NH₂, and
      6) CH₃; and
each R⁶ is independently selected from:
   1) OH,
   2) F,
   3) CN,
   4) oxo,
   5) C₁₋₃ alkyl, which may be straight or branched and which is unsubstituted or substituted with 1 to 4 substituents selected from OH, F, CN, oxo, and OCH₃,
   6) C₃ cycloalkyl, which is unsubstituted, and
   7) methylene-C₅₋₇ aryl, which is unsubstituted or substituted with 1 to 4 substituents selected from OH, F, CN, oxo, and OCH₃.

Embodiments of the disclosure are directed to compounds of Formula (I):

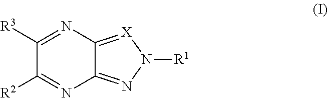

or a pharmaceutically acceptable salt thereof, wherein
X is CR⁵;
R¹ is selected from:

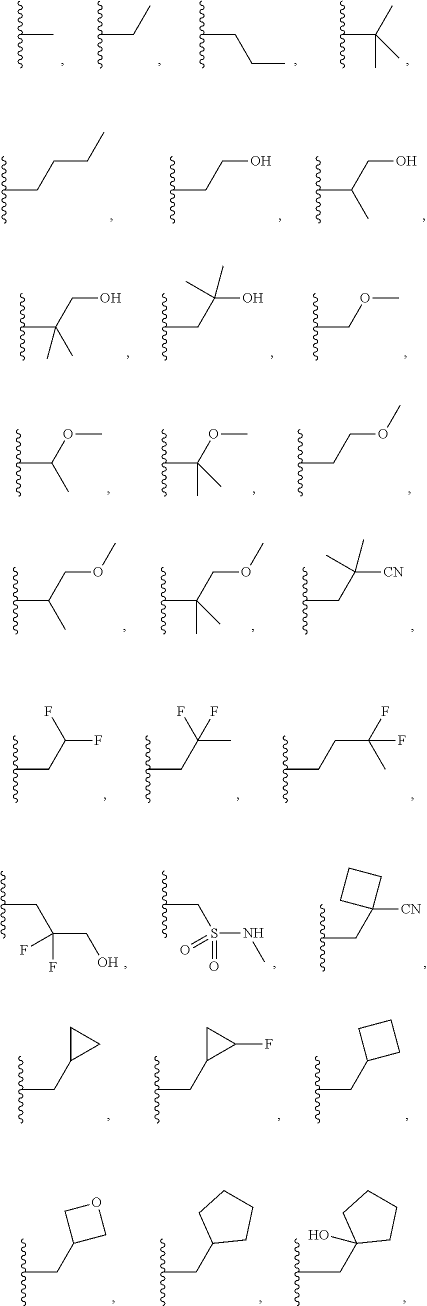

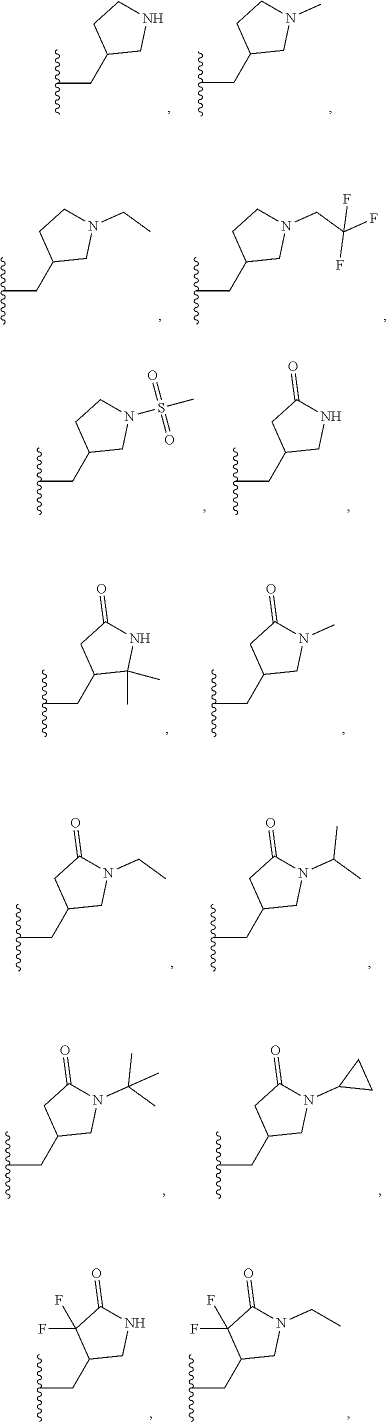

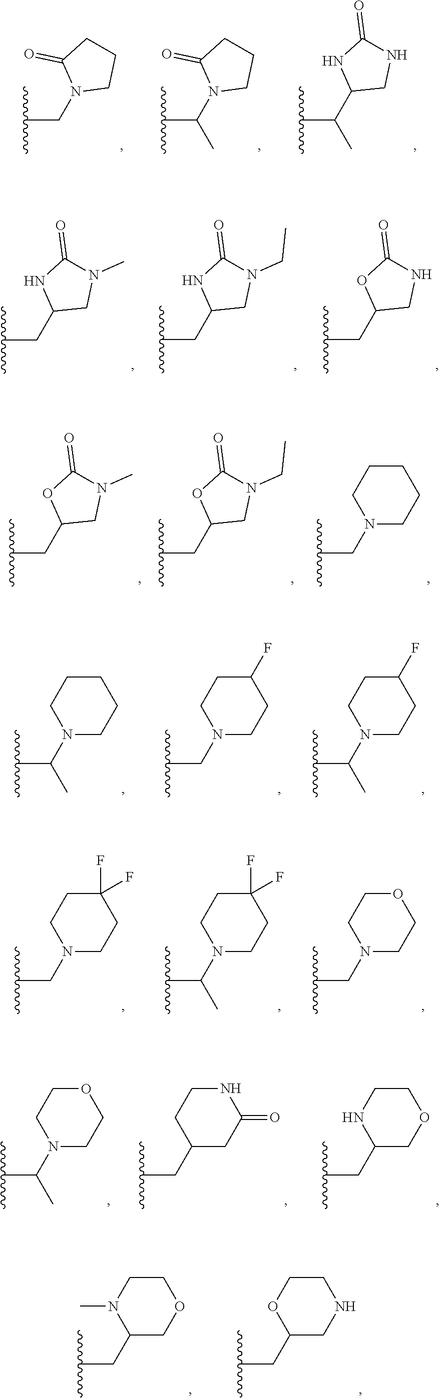

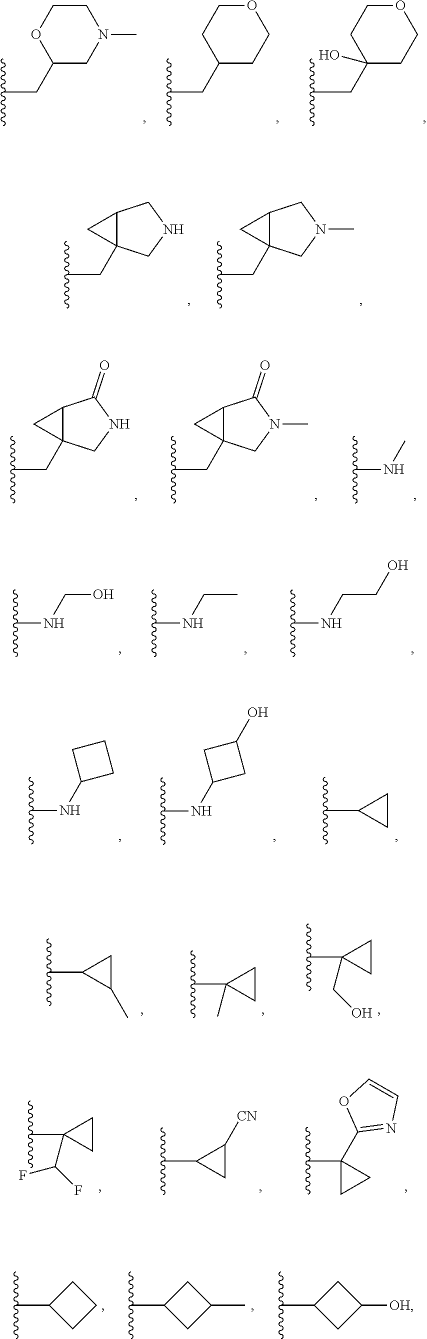

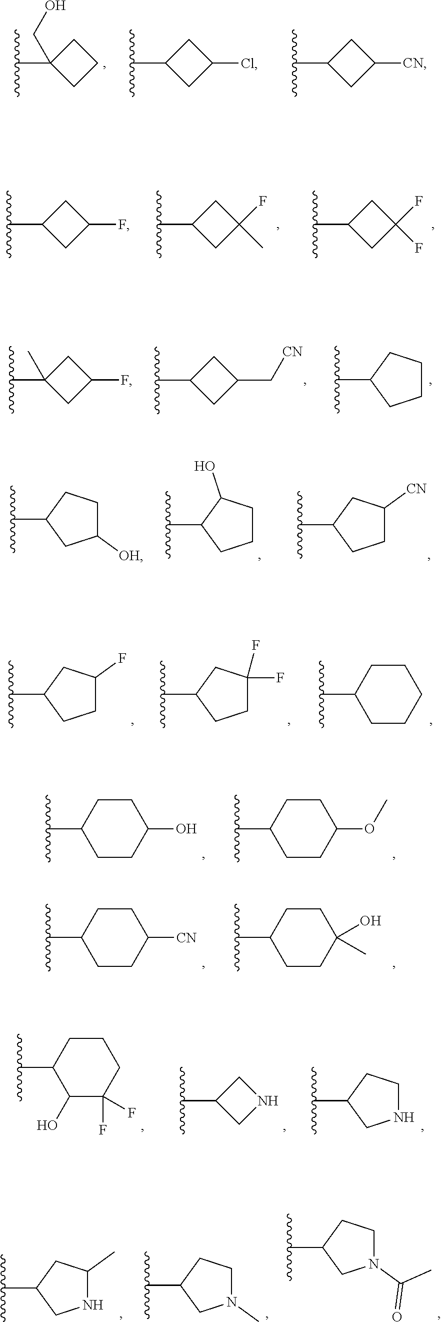

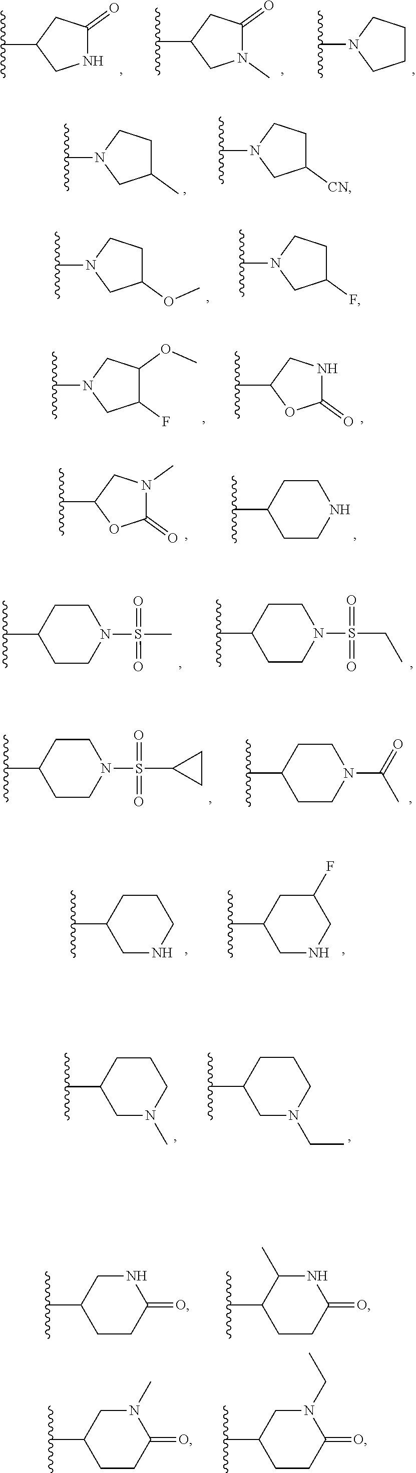

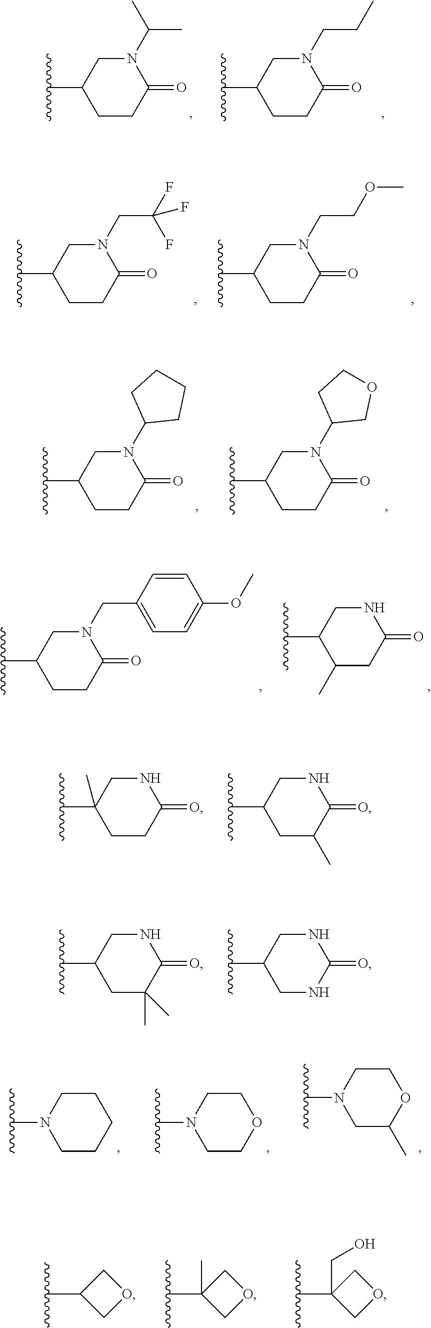

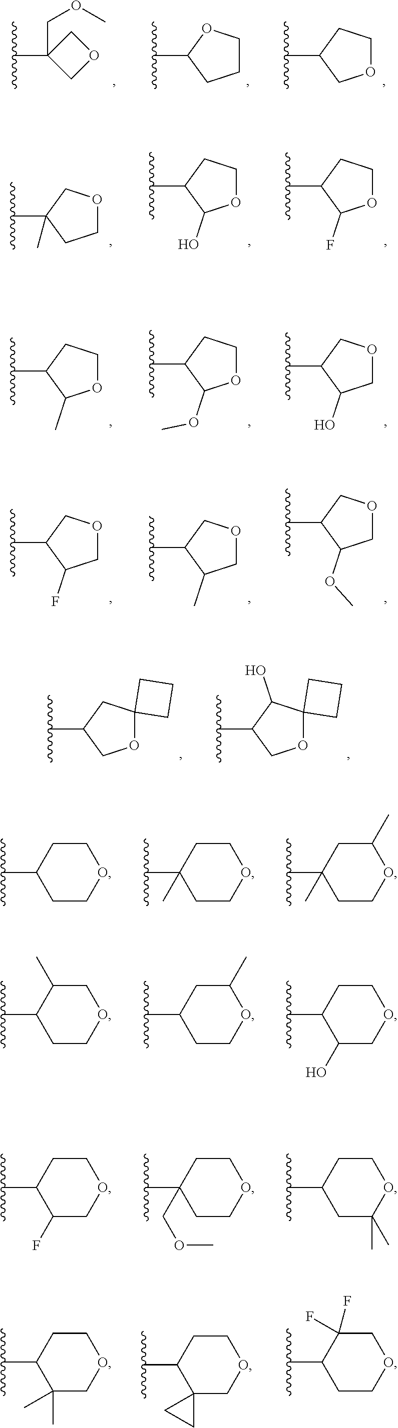

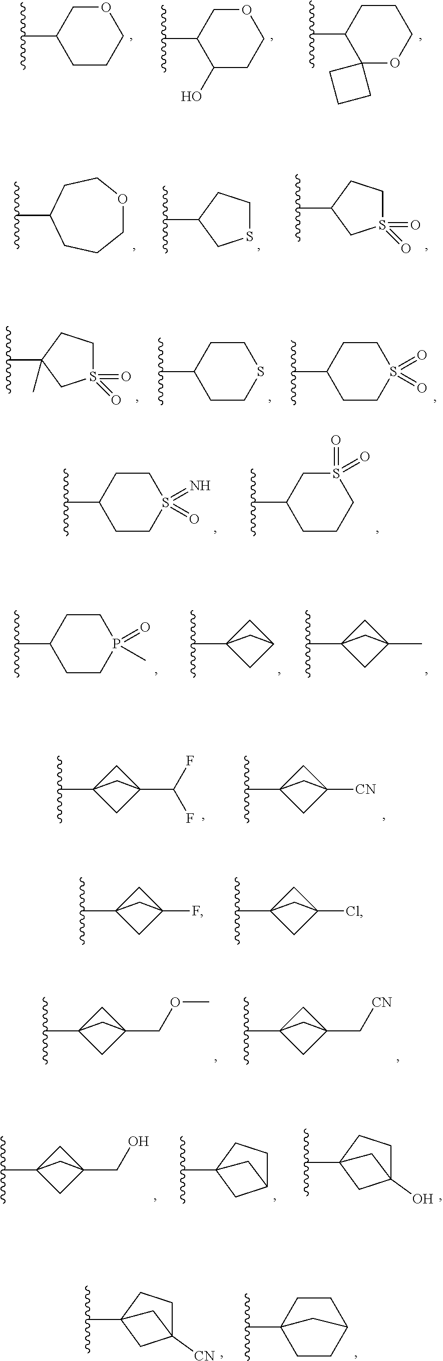

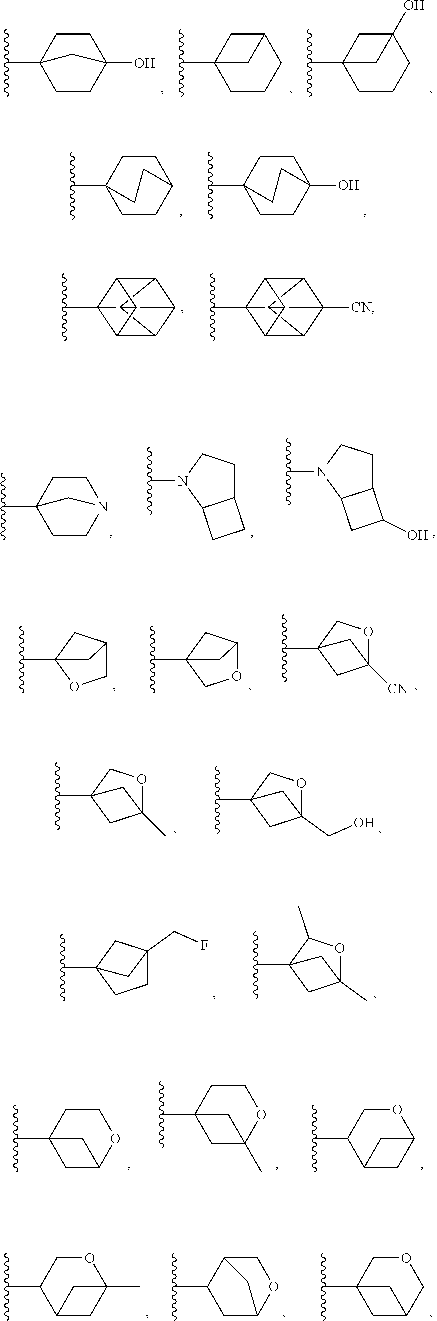

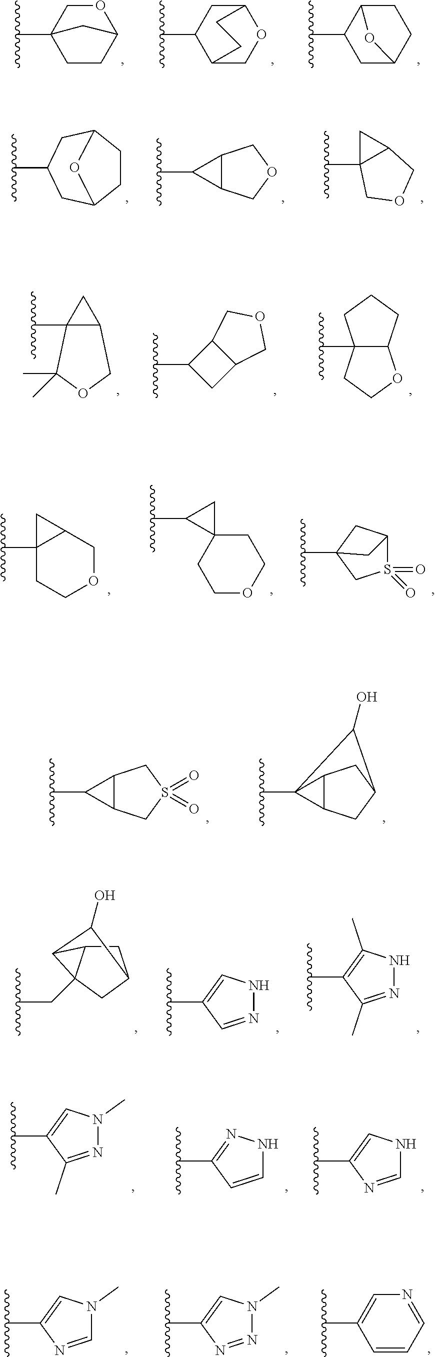

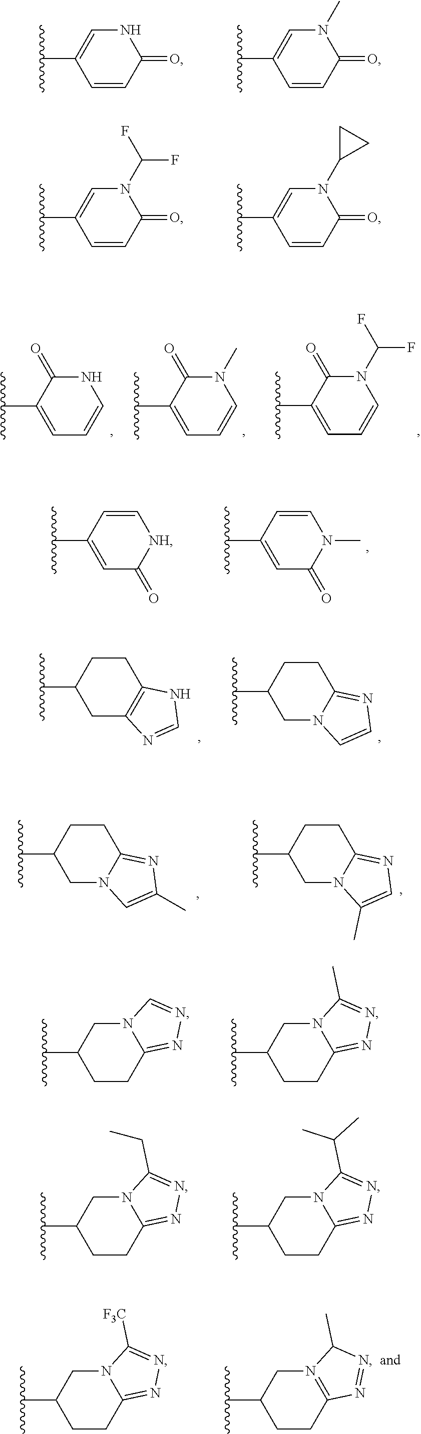

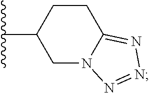 [SMILES: *C1CCc2nnnn2C1]

R² is selected from:

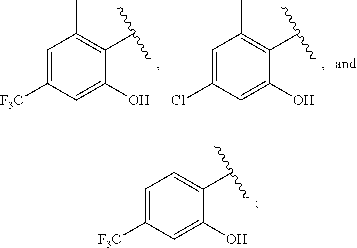

R³ is selected from:
   1) H,
   2) C₁₋₆ alkyl;
R⁵ is selected from:
   1) H,
   2) OH,
   3) CN,
   4) C₁₋₃ alkyl,
   5) C₁₋₃ alkoxy, and
   6) C₃₋₄ cycloalkyl,
   wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
      1) OH,
      2) C₁,
      3) F,
      4) CN,
      5) NH₂, and
      6) CH₃.

Embodiments of the disclosure are directed to compounds of Formula (I):

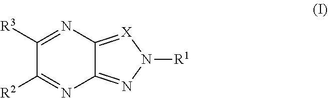

or a pharmaceutically acceptable salt thereof, wherein
X is CR⁵;
R¹ is selected from:

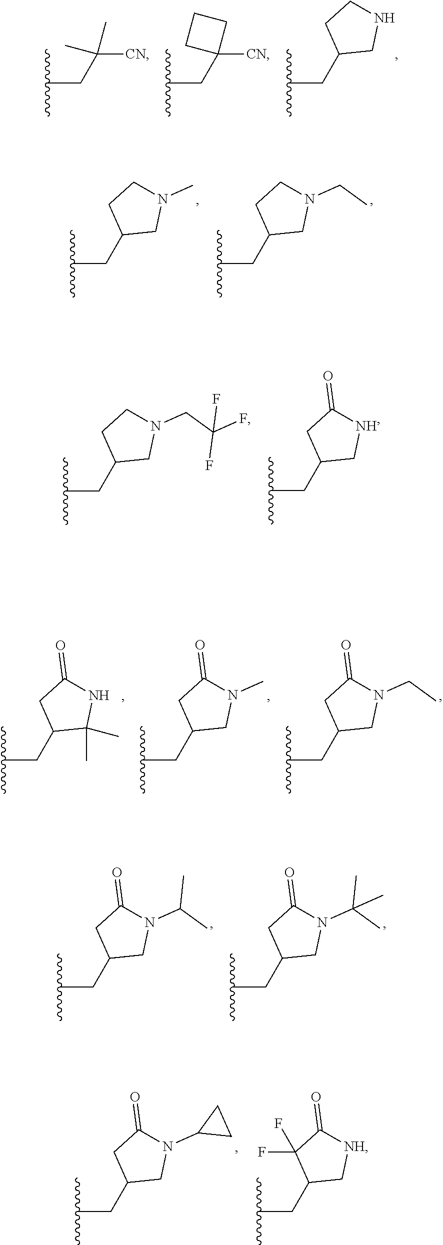

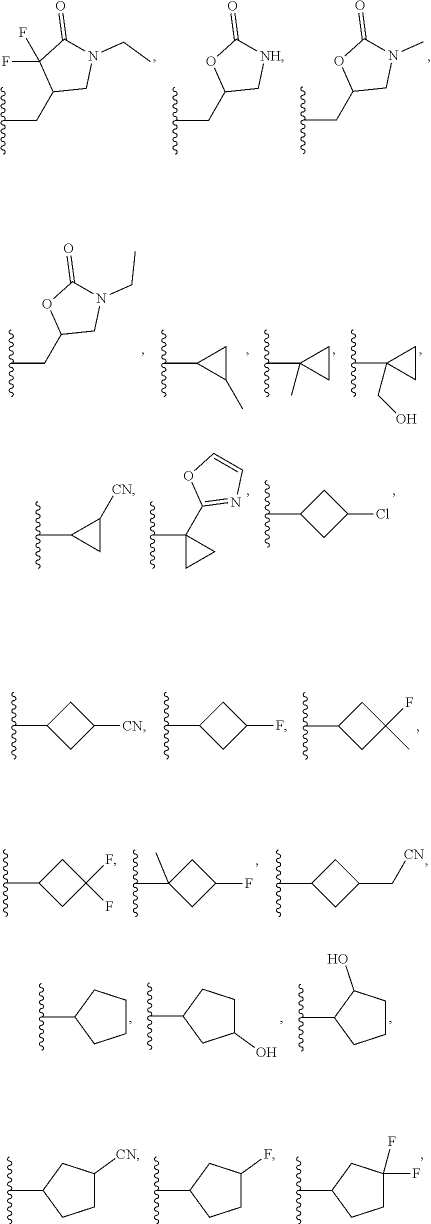

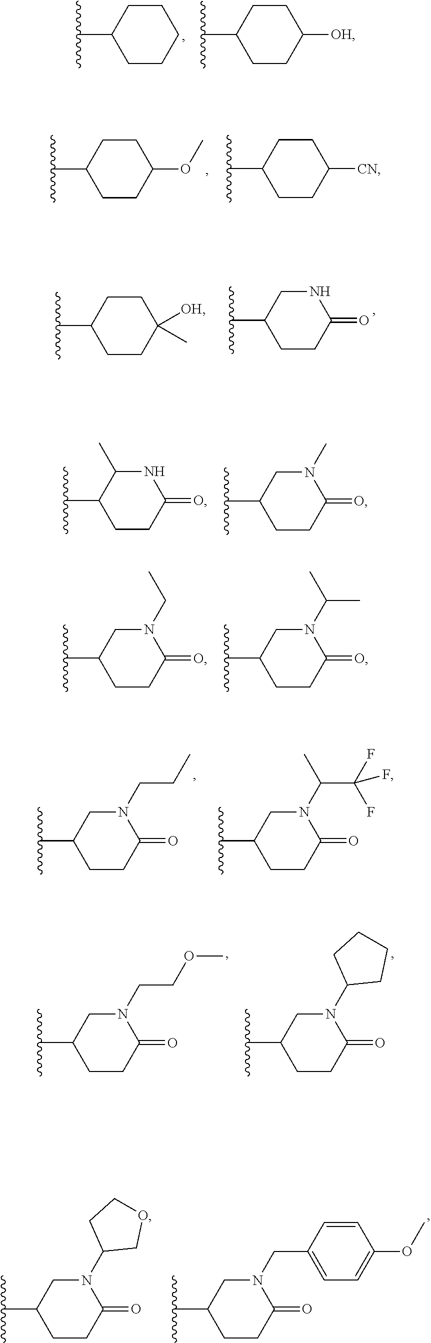

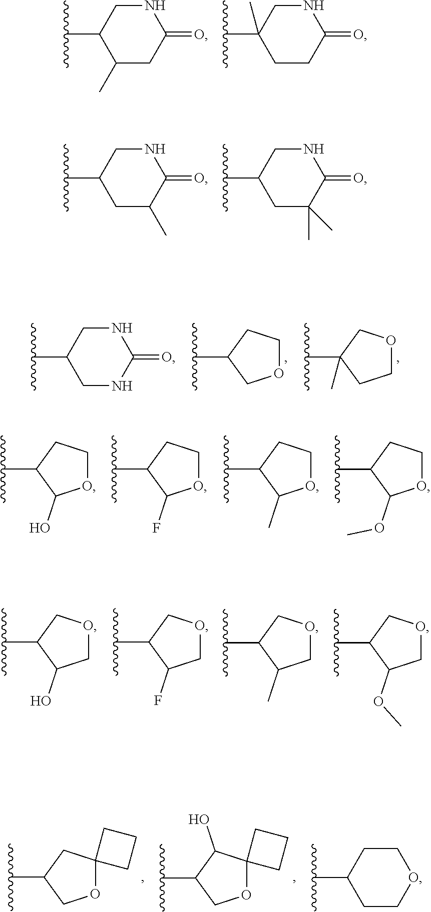

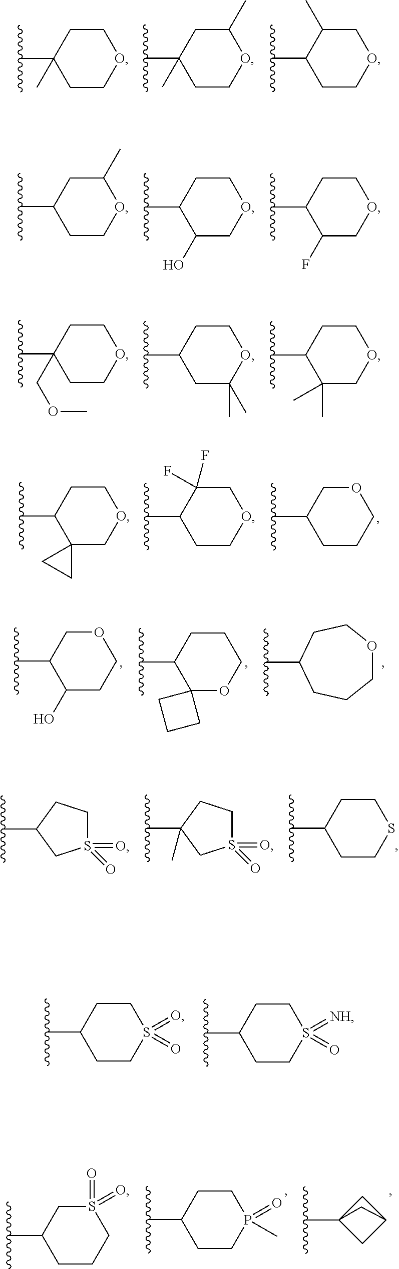

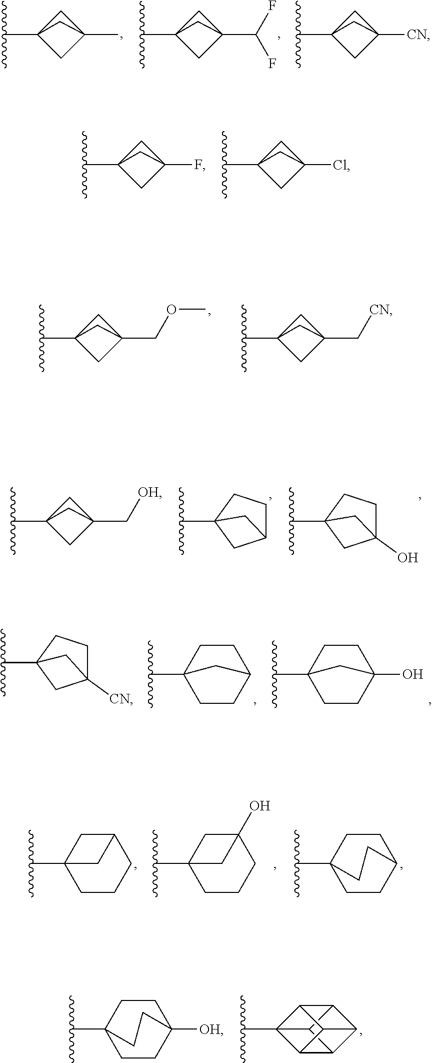

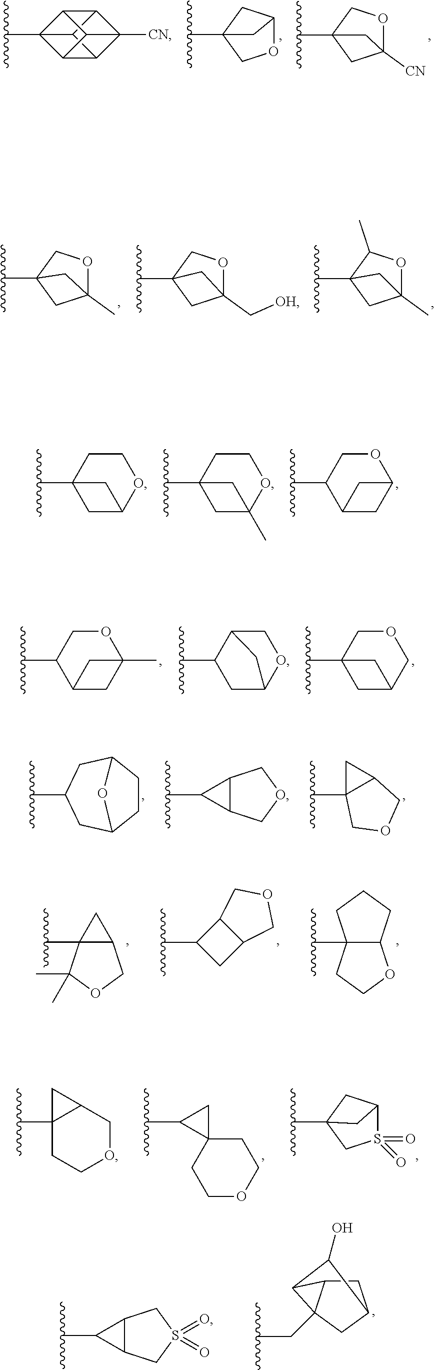

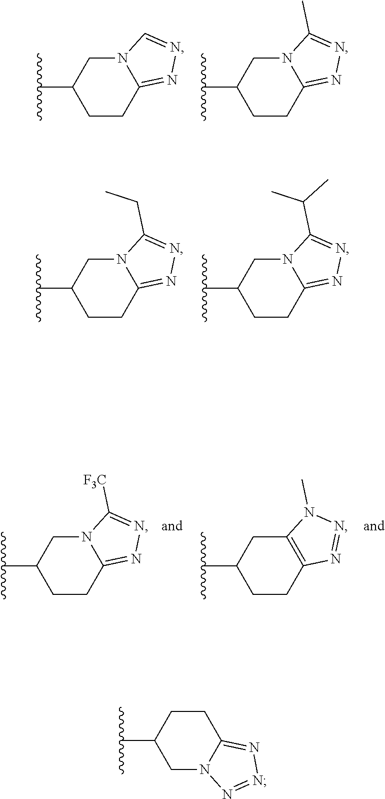

R² is selected from:

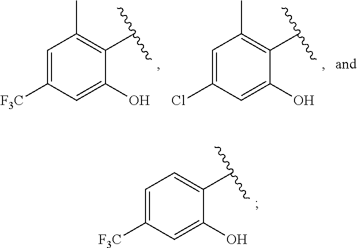

R³ is selected from:
   1) H,
   2) C₁₋₆ alkyl;
R⁵ is selected from the group:
   1) H,
   2) CH₃, and
   3) CH(OH)(CH₃).

Embodiments of the disclosure are directed to compounds of Formula (I):

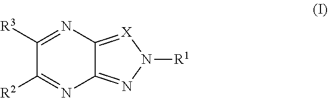

or a pharmaceutically acceptable salt thereof, wherein
X is CR⁵;
R¹ is selected from:

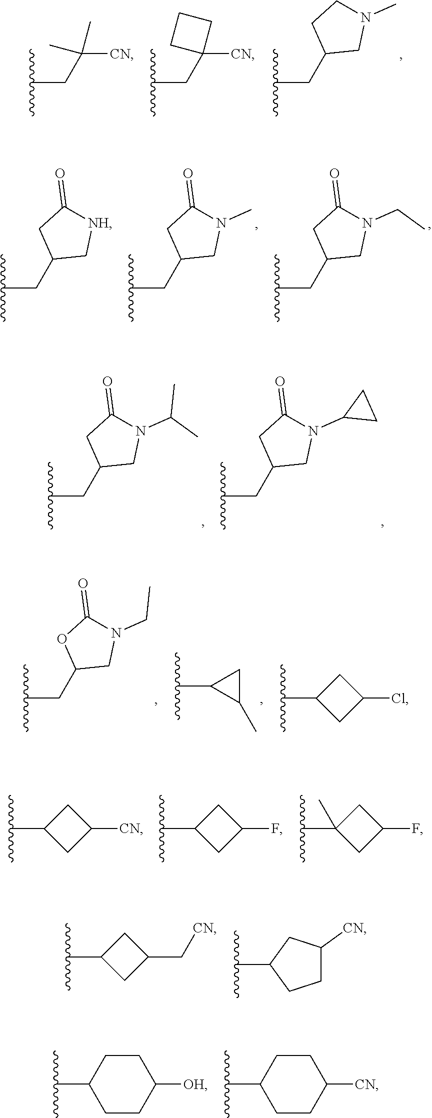

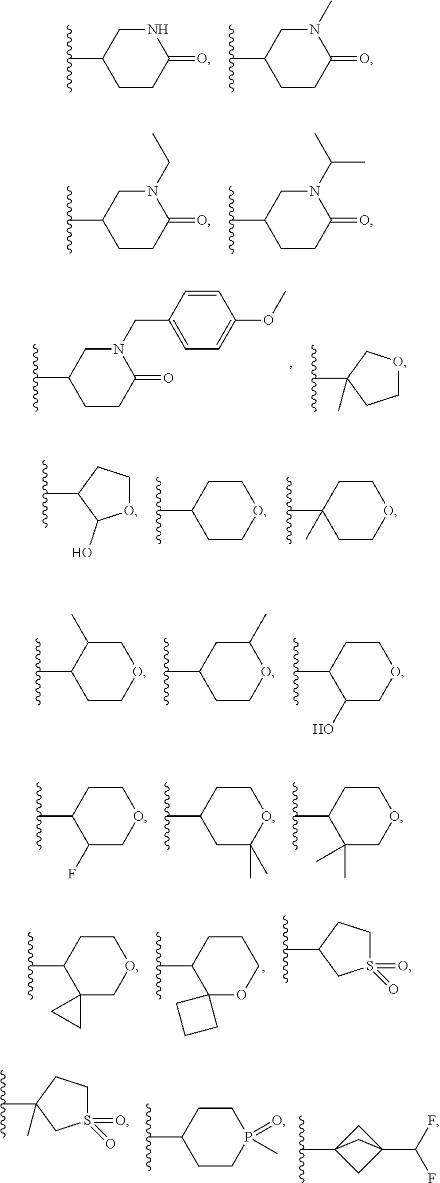

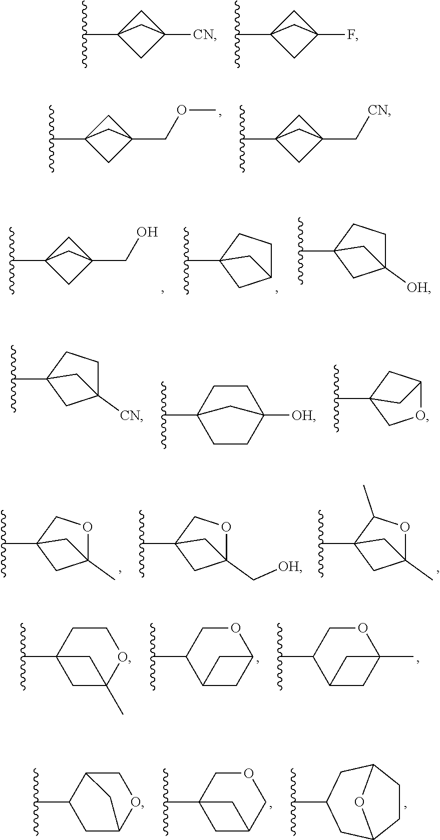

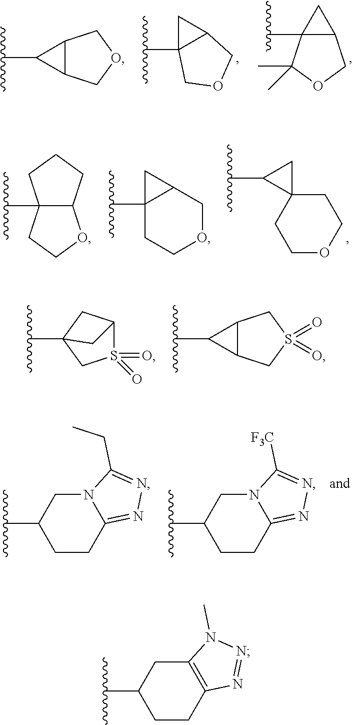

R² is selected from:

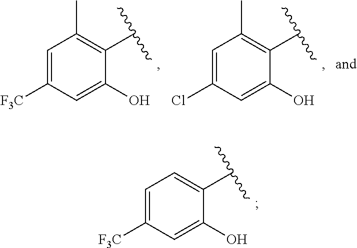

R³ is selected from:
   1) H,
   2) methyl; and
R⁵ is selected from the group:
   1) H,
   2) CH₃, and
   3) CH(OH)(CH₃).

Embodiments of the disclosure are directed to compounds of Formula (I):

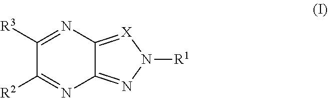

or a pharmaceutically acceptable salt thereof, wherein
X is CR⁵;
R¹ is selected from:

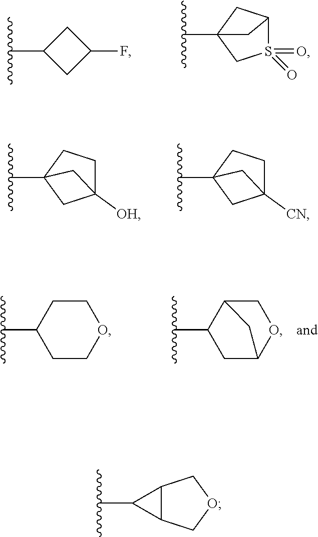

R² is selected from:

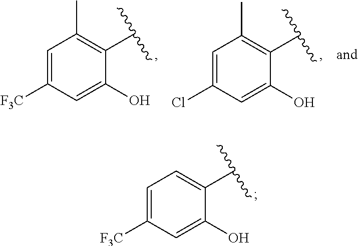

R³ is selected from:
   1) H,
   2) methyl; and
R⁵ is selected from the group:
   1) H,
   2) CH₃, and
   3) CH(OH)(CH₃).

Embodiments of the disclosure are directed to compounds of Formula (I):

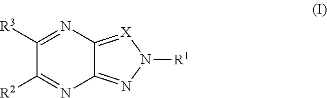

or a pharmaceutically acceptable salt thereof, wherein
X is CR⁵;
R¹ is selected from:

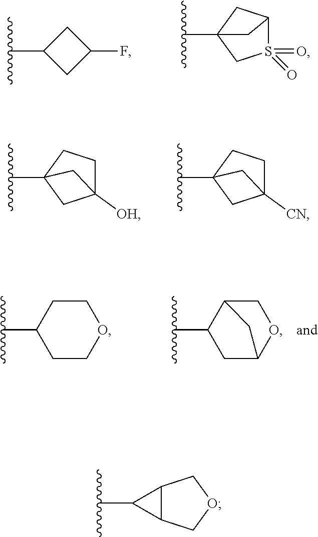

R² is selected from:

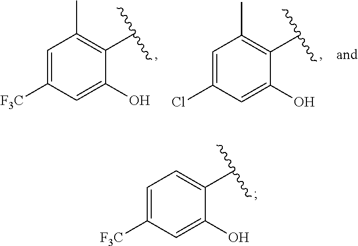

R³ is selected from:
   1) H, and
   2) CH₃; and
R⁵ is selected from:
   1) H,
   2) CH₃, and
   3) CHOHCH₃.

Embodiments of the disclosure are directed to compounds of Formula (I):

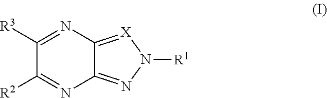

or a pharmaceutically acceptable salt thereof, wherein
X is CR⁵;
R¹ is selected from:

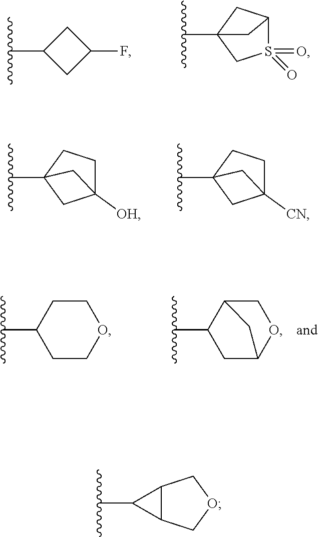

R² is

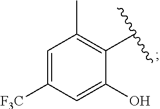 [SMILES: *c1c(C)cc(C(F)(F)F)cc1O]

R³ is selected from:
   1) H, and
   2) CH₃; and
R⁵ is selected from:
   1) H,
   2) CH₃, and
   3) CHOHCH₃.

Embodiments of the disclosure are directed to compounds of Formula (I):

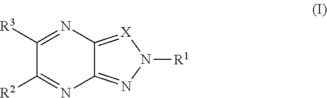

or a pharmaceutically acceptable salt thereof, wherein
X is CR⁵;
R¹ is selected from:

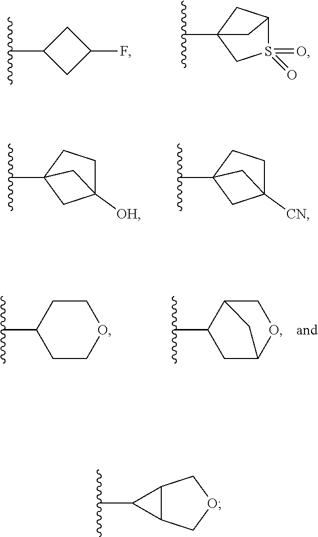

R² is

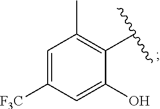 [SMILES: *c1c(C)cc(C(F)(F)F)cc1O]

R³ is H; and
R⁵ is H.

Embodiments of the disclosure are directed to compounds of Formula (I):

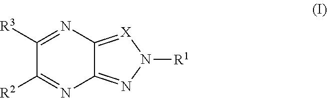

or a pharmaceutically acceptable salt thereof, wherein
X is CR⁵;
R¹ is

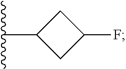 [SMILES: *C1CC(F)C1]

R² is

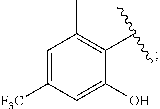 [SMILES: *c1c(C)cc(C(F)(F)F)cc1O]

R³ is H; and
R⁵ is H.

Embodiments of the disclosure are directed to compounds of Formula (I):

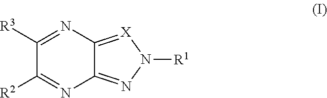

or a pharmaceutically acceptable salt thereof, wherein
X is CR⁵;
R¹ is

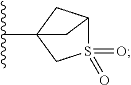 [SMILES: *C12CC(C1)S(=O)(=O)C2]

R² is

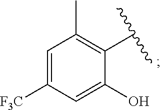 [SMILES: *c1c(C)cc(C(F)(F)F)cc1O]

R³ is H; and
R⁵ is H.

Embodiments of the disclosure are directed to compounds of Formula (I):

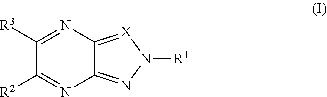

or a pharmaceutically acceptable salt thereof, wherein
X is CR⁵;
R¹ is

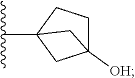 [SMILES: *C12CCC(O)(C1)C2]

R² is

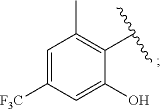 [SMILES: *c1c(C)cc(C(F)(F)F)cc1O]

R³ is H; and
R⁵ is H.

Embodiments of the disclosure are directed to compounds of Formula (I):

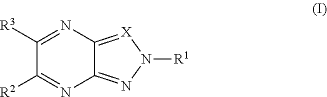

or a pharmaceutically acceptable salt thereof, wherein
X is CR⁵;
R¹ is

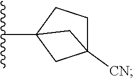 [SMILES: *C12CCC(C#N)(C1)C2]

R² is

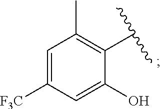 [SMILES: *c1c(C)cc(C(F)(F)F)cc1O]

R³ is H; and
R⁵ is H.

Embodiments of the disclosure are directed to compounds of Formula (I):

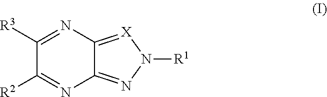

or a pharmaceutically acceptable salt thereof, wherein
X is CR⁵;
R¹ is

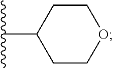 [SMILES: *C1CCOCC1]

R² is

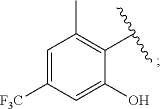 [SMILES: *c1c(C)cc(C(F)(F)F)cc1O]

R³ is H; and
R⁵ is H.

Embodiments of the disclosure are directed to compounds of Formula (I):

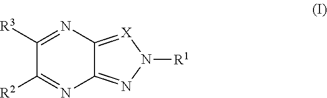

or a pharmaceutically acceptable salt thereof, wherein
X is CR⁵;
R¹ is

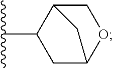 [SMILES: *C1CC2CC1CO2]

R² is

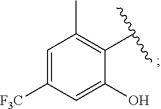 [SMILES: *c1c(C)cc(C(F)(F)F)cc1O]

R³ is H; and
R⁵ is H.

Embodiments of the disclosure are directed to compounds of Formula (I):

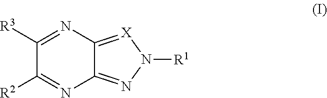

or a pharmaceutically acceptable salt thereof, wherein
X is CR⁵;
R¹ is

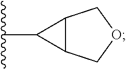 [SMILES: *C1C2COCC12]

R² is

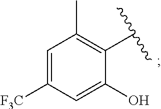 [SMILES: *c1c(C)cc(C(F)(F)F)cc1O]

R³ is H; and
R⁵ is H.

Illustrative, but non-limiting, examples of compounds of embodiments that are useful as inhibitors of the NLRP3 are the following compounds:
1) 3-methyl-2-(2-(tetrahydro-2H-pyran-4-yl)-2H-pyrazolo[3,4-b]pyrazin-6-yl)-5-(trifluoromethyl)phenol,
2) 4-(6-(2-hydroxy-6-methyl-4-(trifluoromethyl)phenyl)-2H-pyrazolo[3,4-b]pyrazin-2-yl)-2-thiabicyclo[2.1.1]hexane 2,2-dioxide,
3) 2-(2-((1S,4R,5R)-2-oxabicyclo[2.2.1]heptan-5-yl)-2H-pyrazolo[3,4-b]pyrazin-6-yl)-3-methyl-5-(trifluoromethyl)phenol,
4) 2-(2-((1R,4S,5S)-2-oxabicyclo[2.2.1]heptan-5-yl)-2H-pyrazolo[3,4-b]pyrazin-6-yl)-3-methyl-5-(trifluoromethyl)phenol,
5) (5S)-5-[6-[2-hydroxy-6-methyl-4-(trifluoromethyl)phenyl]pyrazolo[3,4-b]pyrazin-2-yl]-1-methyl-piperidin-2-one,
6) (R)-3-methyl-2-(2-((1-methylpyrrolidin-3-yl)methyl)-2H-pyrazolo[3,4-b]pyrazin-6-yl)-5-(trifluoromethyl)phenol,
7) (R)-4-((6-(2-hydroxy-6-methyl-4-(trifluoromethyl)phenyl)-2H-pyrazolo[3,4-b]pyrazin-2-yl)methyl)pyrrolidin-2-one,
8) 3-(6-(2-hydroxy-6-methyl-4-(trifluoromethyl)phenyl)-2H-pyrazolo[3,4-b]pyrazin-2-yl)bicyclo[1.1.1]pentane-1-carbonitrile,
9) 2-(2-((1R,5S,6S)-3-oxabicyclo[3.1.0]hexan-6-yl)-2H-pyrazolo[3,4-b]pyrazin-6-yl)-3-methyl-5-(trifluoromethyl)phenol,
10) 2-(2-((1R,5S,6R)-3-oxabicyclo[3.1.0]hexan-6-yl)-2H-pyrazolo[3,4-b]pyrazin-6-yl)-3-methyl-5-(trifluoromethyl)phenol,
11) (1S,4S)-4-(6-(2-hydroxy-6-methyl-4-(trifluoromethyl)phenyl)-2H-pyrazolo[3,4-b]pyrazin-2-yl)cyclohexane-1-carbonitrile,
12) 3-(6-(2-hydroxy-6-methyl-4-(trifluoromethyl)phenyl)-2H-pyrazolo[3,4-b]pyrazin-2-yl)-2,2-dimethylpropanenitrile,
13) 1-((6-(2-hydroxy-6-methyl-4-(trifluoromethyl)phenyl)-2H-pyrazolo[3,4-b]pyrazin-2-yl)methyl)cyclobutane-1-carbonitrile,
14) 2-(2-(3-oxabicyclo[3.1.1]heptan-1-yl)-2H-pyrazolo[3,4-b]pyrazin-6-yl)-3-methyl-5-(trifluoromethyl)phenol,
15) 3-methyl-2-(2-(1-methyl-2-oxabicyclo[3.1.1]heptan-5-yl)-2H-pyrazolo[3,4-b]pyrazin-6-yl)-5-(trifluoromethyl)phenol,
16) 2-(2-((1R,3R)-3-fluorocyclobutyl)-2H-pyrazolo[3,4-b]pyrazin-6-yl)-3-methyl-5-(trifluoromethyl)phenol,
17) 2-(2-(2-oxabicyclo[2.1.1]hexan-4-yl)-2H-pyrazolo[3,4-b]pyrazin-6-yl)-3-methyl-5-(trifluoromethyl)phenol,
18) 2-((1s,3s)-3-(6-(2-hydroxy-6-methyl-4-(trifluoromethyl)phenyl)-2H-pyrazolo[3,4-b]pyrazin-2-yl)cyclobutyl)acetonitrile,
19) (R and S)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
20) (R)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
21) (S)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
22) (1S,3R and 1R,3S)-3-(6-(2-hydroxy-6-methyl-4-(trifluoromethyl)phenyl)-2H-pyrazolo[3,4-b]pyrazin-2-yl)cyclopentane-1-carbonitrile,
23) (1S,3R)-3-(6-(2-hydroxy-6-methyl-4-(trifluoromethyl)phenyl)-2H-pyrazolo[3,4-b]pyrazin-2-yl)cyclopentane-1-carbonitrile,
24) (1R,3S)-3-(6-(2-hydroxy-6-methyl-4-(trifluoromethyl)phenyl)-2H-pyrazolo[3,4-b]pyrazin-2-yl)cyclopentane-1-carbonitrile,
25) 2-(2-((3aS,6aR and 3aR,6aS)-hexahydro-3aH-cyclopenta[b]furan-3a-yl)-2H-pyrazolo[3,4-b]pyrazin-6-yl)-3-methyl-5-(trifluoromethyl)phenol,
26) 2-(2-((3aS,6aR)-hexahydro-3aH-cyclopenta[b]furan-3a-yl)-2H-pyrazolo[3,4-b]pyrazin-6-yl)-3-methyl-5-(trifluoromethyl)phenol,
27) 2-(2-((3aR,6aS)-hexahydro-3aH-cyclopenta[b]furan-3a-yl)-2H-pyrazolo[3,4-b]pyrazin-6-yl)-3-methyl-5-(trifluoromethyl)phenol,
28) (S and R)-2-(2-(5-oxaspiro[3.5]nonan-9-yl)-2H-pyrazolo[3,4-b]pyrazin-6-yl)-3-methyl-5-(trifluoromethyl)phenol,
29) (S)-2-(2-(5-oxaspiro[3.5]nonan-9-yl)-2H-pyrazolo[3,4-b]pyrazin-6-yl)-3-methyl-5-(trifluoromethyl)phenol,
30) (R)-2-(2-(5-oxaspiro[3.5]nonan-9-yl)-2H-pyrazolo[3,4-b]pyrazin-6-yl)-3-methyl-5-(trifluoromethyl)phenol,
31) (S and R)-2-(2-(6-oxaspiro[2.5]octan-1-yl)-2H-pyrazolo[3,4-b]pyrazin-6-yl)-3-methyl-5-(trifluoromethyl)phenol,
32) (S)-2-(2-(6-oxaspiro[2.5]octan-1-yl)-2H-pyrazolo[3,4-b]pyrazin-6-yl)-3-methyl-5-(trifluoromethyl)phenol,
33) (R)-2-(2-(6-oxaspiro[2.5]octan-1-yl)-2H-pyrazolo[3,4-b]pyrazin-6-yl)-3-methyl-5-(trifluoromethyl)phenol,
34) (S and R)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
35) (S)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
36) (R)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
37) (S and R)-2-(2-(2-oxabicyclo[3.1.1]heptan-4-yl)-2H-pyrazolo[3,4-b]pyrazin-6-yl)-3-methyl-5-(trifluoromethyl)phenol,
38) (S)-2-(2-(2-oxabicyclo[3.1.1]heptan-4-yl)-2H-pyrazolo[3,4-b]pyrazin-6-yl)-3-methyl-5-(trifluoromethyl)phenol,
39) (R)-2-(2-(2-oxabicyclo[3.1.1]heptan-4-yl)-2H-pyrazolo[3,4-b]pyrazin-6-yl)-3-methyl-5-(trifluoromethyl)phenol,
40) 4-[6-[2-hydroxy-6-methyl-4-(trifluoromethyl)phenyl]pyrazolo[3,4-b]pyrazin-2-yl]bicyclo[2.1.1]hexan-1-ol,
41) 3-methyl-2-[2-(1-methyl-2-oxabicyclo[2.1.1]hexan-4-yl)pyrazolo[3,4-b]pyrazin-6-yl]-5-(trifluoromethyl)phenol,
42) 4-[6-[2-hydroxy-6-methyl-4-(trifluoromethyl)phenyl]pyrazolo[3,4-b]pyrazin-2-yl]norboman-1-ol,
43) 3-methyl-2-[2-[(3R)-3-methyl-1,1-dioxo-thiolan-3-yl]pyrazolo[3,4-b]pyrazin-6-yl]-5-(trifluoromethyl)phenol,
44) 2-[2-[(3R)-1,1-dioxothiolan-3-yl]pyrazolo[3,4-b]pyrazin-6-yl]-3-methyl-5-(trifluoromethyl)phenol,
45) 2-[2-[(3S)-1,1-dioxothiolan-3-yl]pyrazolo[3,4-b]pyrazin-6-yl]-3-methyl-5-(trifluoromethyl)phenol,
46) (3R,4S)-4-[6-[2-hydroxy-6-methyl-4-(trifluoromethyl)phenyl]pyrazolo[3,4-b]pyrazin-2-yl]tetrahydrofuran-3-ol,
47) (3S,4R)-4-[6-[2-hydroxy-6-methyl-4-(trifluoromethyl)phenyl]pyrazolo[3,4-b]pyrazin-2-yl]tetrahydrofuran-3-ol,
48) (3R,4R)-3-[6-[2-hydroxy-6-methyl-4-(trifluoromethyl)phenyl]pyrazolo[3,4-b]pyrazin-2-yl]tetrahydropyran-4-ol,
49) (3S,4S)-3-[6-[2-hydroxy-6-methyl-4-(trifluoromethyl)phenyl]pyrazolo[3,4-b]pyrazin-2-yl]tetrahydropyran-4-ol,
50) (1R,3R)-3-(6-(2-hydroxy-6-methyl-4-(trifluoromethyl)phenyl)-2H-pyrazolo[3,4-b]pyrazin-2-yl)cyclobutane-1-carbonitrile,
51) 2-[2-[3-(methoxymethyl)-1-bicyclo[1.1.1]pentanyl]pyrazolo[3,4-b]pyrazin-6-yl]-3-methyl-5-(trifluoromethyl)phenol,
52) 2-[2-(3-methoxy-1-bicyclo[1.1.1]pentanyl)pyrazolo[3,4-b]pyrazin-6-yl]-3-methyl-5-(trifluoromethyl)phenol,
53) 2-[2-[3-(difluoromethyl)-1-bicyclo[1.1.1]pentanyl]pyrazolo[3,4-b]pyrazin-6-yl]-3-methyl-5-(trifluoromethyl)phenol,
54) (1R,4R)-4-(6-(2-hydroxy-6-methyl-4-(trifluoromethyl)phenyl)-2H-pyrazolo[3,4-b]pyrazin-2-yl)cyclohexane-1-carbonitrile,
55) 2-[2-[1-(hydroxymethyl)-2-oxabicyclo[2.1.1]hexan-4-yl]pyrazolo[3,4-b]pyrazin-6-yl]-3-methyl-5-(trifluoromethyl)phenol,
56) 2-[2-(1-bicyclo[2.1.1]hexanyl)pyrazolo[3,4-b]pyrazin-6-yl]-3-methyl-5-(trifluoromethyl)phenol,
57) 2-[2-[3-(hydroxymethyl)-1-bicyclo[1.1.1]pentanyl]pyrazolo[3,4-b]pyrazin-6-yl]-3-methyl-5-(trifluoromethyl)phenol,
58) 2-(2-((1S,4S)-4-hydroxycyclohexyl)-2H-pyrazolo[3,4-b]pyrazin-6-yl)-3-methyl-5-(trifluoromethyl)phenol,
59) 3-methyl-2-[2-(8-oxabicyclo[3.2.1]octan-3-yl)pyrazolo[3,4-b]pyrazin-6-yl]-5-(trifluoromethyl)phenol,
60) 2-[3-[6-[2-hydroxy-6-methyl-4-(trifluoromethyl)phenyl]pyrazolo[3,4-b]pyrazin-2-yl]-1-bicyclo[1.1.1]pentanyl]acetonitrile,
61) 4-[6-[2-hydroxy-6-methyl-4-(trifluoromethyl)phenyl]pyrazolo[3,4-b]pyrazin-2-yl]bicyclo[2.1.1]hexane-1-carbonitrile,
62) 2-[2-(3-fluoro-1-bicyclo[1.1.1]pentanyl)pyrazolo[3,4-b]pyrazin-6-yl]-3-methyl-5-(trifluoromethyl)phenol,
63) 2-(2-((1R,3R)-3-chlorocyclobutyl)-2H-pyrazolo[3,4-b]pyrazin-6-yl)-3-methyl-5-(trifluoromethyl)phenol,
64) 3-methyl-2-[2-[(1R,2R)-2-methylcyclopropyl]pyrazolo[3,4-b]pyrazin-6-yl]-5-(trifluoromethyl)phenol,
65) 3-methyl-2-[2-(4-methyltetrahydropyran-4-yl)pyrazolo[3,4-b]pyrazin-6-yl]-5-(trifluoromethyl)phenol,
66) 3-methyl-2-[2-[(1S,2S)-2-methylcyclopropyl]pyrazolo[3,4-b]pyrazin-6-yl]-5-(trifluoromethyl)phenol,
67) (1R,5S,6S)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
68) (1R,5S,6R)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
69) 5-chloro-2-(2-((1R,3R)-3-fluorocyclobutyl)-2H-pyrazolo[3,4-b]pyrazin-6-yl)-3-methylphenol,
70) 2-(2-((1R,5S,6S)-3-oxabicyclo[3.1.0]hexan-6-yl)-2H-pyrazolo[3,4-b]pyrazin-6-yl)-5-chloro-3-methylphenol,
71) 2-(2-((1R,5S,6R)-3-oxabicyclo[3.1.0]hexan-6-yl)-2H-pyrazolo[3,4-b]pyrazin-6-yl)-5-chloro-3-methylphenol,
72) (R)-4-((6-(2-hydroxy-6-methyl-4-(trifluoromethyl)phenyl)-2H-pyrazolo[3,4-b]pyrazin-2-yl)methyl)-1-methylpyrrolidin-2-one,
73) (S)-4-((6-(2-hydroxy-6-methyl-4-(trifluoromethyl)phenyl)-2H-pyrazolo[3,4-b]pyrazin-2-yl)methyl)-1-methylpyrrolidin-2-one,
74) (R)-5-(6-(2-hydroxy-6-methyl-4-(trifluoromethyl)phenyl)-2H-pyrazolo[3,4-b]pyrazin-2-yl)-1-isopropylpiperidin-2-one,
75) (S)-5-(6-(2-hydroxy-6-methyl-4-(trifluoromethyl)phenyl)-2H-pyrazolo[3,4-b]pyrazin-2-yl)-1-isopropylpiperidin-2-one,
76) (R)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
77) (S)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
78) (R)-1-ethyl-5-(6-(2-hydroxy-6-methyl-4-(trifluoromethyl)phenyl)-2H-pyrazolo[3,4-b]pyrazin-2-yl)piperidin-2-one,
79) (S)-1-ethyl-5-(6-(2-hydroxy-6-methyl-4-(trifluoromethyl)phenyl)-2H-pyrazolo[3,4-b]pyrazin-2-yl)piperidin-2-one,
80) (R)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
81) (S)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
82) (R)-3-methyl-2-(2-(1-methyl-4,5,6,7-tetrahydro-1H-benzo[d][1,2,3]triazol-6-yl)-2H-pyrazolo[3,4-b]pyrazin-6-yl)-5-(trifluoromethyl)phenol,
83) (S)-3-methyl-2-(2-(1-methyl-4,5,6,7-tetrahydro-1H-benzo[d][1,2,3]triazol-6-yl)-2H-pyrazolo[3,4-b]pyrazin-6-yl)-5-(trifluoromethyl)phenol,
84) (R)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
85) (S)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
86) (R)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
87) (S)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
88) 2-(2-((1R,3R)-3-fluoro-1-methylcyclobutyl)-2H-pyrazolo[3,4-b]pyrazin-6-yl)-3-methyl-5-(trifluoromethyl)phenol,
89) 2-(2-((1S,3S)-3-fluoro-1-methylcyclobutyl)-2H-pyrazolo[3,4-b]pyrazin-6-yl)-3-methyl-5-(trifluoromethyl)phenol,
90) 2-(2-((1R,5R)-2,2-dimethyl-3-oxabicyclo[3.1.0]hexan-1-yl)-2H-pyrazolo[3,4-b]pyrazin-6-yl)-3-methyl-5-(trifluoromethyl)phenol,
91) 2-(2-((1S,5S)-2,2-dimethyl-3-oxabicyclo[3.1.0]hexan-1-yl)-2H-pyrazolo[3,4-b]pyrazin-6-yl)-3-methyl-5-(trifluoromethyl)phenol,
92) (S)-3-methyl-2-(2-(3-methyltetrahydrofuran-3-yl)-2H-pyrazolo[3,4-b]pyrazin-6-yl)-5-(trifluoromethyl)phenol,
93) (R)-3-methyl-2-(2-(3-methyltetrahydrofuran-3-yl)-2H-pyrazolo[3,4-b]pyrazin-6-yl)-5-(trifluoromethyl)phenol,
94) (R)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
95) (S)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
96) (1R,4R)-4-(6-(2-hydroxy-6-methyl-4-(trifluoromethyl)phenyl)-2H-pyrazolo[3,4-b]pyrazin-2-yl)-1-methylphosphinane 1-oxide,
97) (1S,4S)-4-(6-(2-hydroxy-6-methyl-4-(trifluoromethyl)phenyl)-2H-pyrazolo[3,4-b]pyrazin-2-yl)-1-methylphosphinane 1-oxide,
98) 2-(2-((1R,5R)-3-oxabicyclo[3.1.0]hexan-1-yl)-2H-pyrazolo[3,4-b]pyrazin-6-yl)-3-methyl-5-(trifluoromethyl)phenol,
99) 2-(2-((1S,5S)-3-oxabicyclo[3.1.0]hexan-1-yl)-2H-pyrazolo[3,4-b]pyrazin-6-yl)-3-methyl-5-(trifluoromethyl)phenol,
100) 3-methyl-2-(2-((3R,4R)-3-methyltetrahydro-2H-pyran-4-yl)-2H-pyrazolo[3,4-b]pyrazin-6-yl)-5-(trifluoromethyl)phenol,
101) 3-methyl-2-(2-((3S,4S)-3-methyltetrahydro-2H-pyran-4-yl)-2H-pyrazolo[3,4-b]pyrazin-6-yl)-5-(trifluoromethyl)phenol,
102) 3-methyl-2-(2-((3R,4S)-3-methyltetrahydro-2H-pyran-4-yl)-2H-pyrazolo[3,4-b]pyrazin-6-yl)-5-(trifluoromethyl)phenol,
103) 3-methyl-2-(2-((3S,4R)-3-methyltetrahydro-2H-pyran-4-yl)-2H-pyrazolo[3,4-b]pyrazin-6-yl)-5-(trifluoromethyl)phenol,
104) 3-methyl-2-(2-((2R,4S)-2-methyltetrahydro-2H-pyran-4-yl)-2H-pyrazolo[3,4-b]pyrazin-6-yl)-5-(trifluoromethyl)phenol,
105) 3-methyl-2-(2-((2S,4R)-2-methyltetrahydro-2H-pyran-4-yl)-2H-pyrazolo[3,4-b]pyrazin-6-yl)-5-(trifluoromethyl)phenol,
106) 2-(2-((1R,6S)-3-oxabicyclo[4.1.0]heptan-6-yl)-2H-pyrazolo[3,4-b]pyrazin-6-yl)-3-methyl-5-(trifluoromethyl)phenol,
107) 2-(2-((1S,6R)-3-oxabicyclo[4.1.0]heptan-6-yl)-2H-pyrazolo[3,4-b]pyrazin-6-yl)-3-methyl-5-(trifluoromethyl)phenol,
108) 3-methyl-2-(2-((2S,4S)-2-methyltetrahydro-2H-pyran-4-yl)-2H-pyrazolo[3,4-b]pyrazin-6-yl)-5-(trifluoromethyl)phenol,
109) 3-methyl-2-(2-((2R,4R)-2-methyltetrahydro-2H-pyran-4-yl)-2H-pyrazolo[3,4-b]pyrazin-6-yl)-5-(trifluoromethyl)phenol,
110) (R)-2-(2-(2,2-dimethyltetrahydro-2H-pyran-4-yl)-2H-pyrazolo[3,4-b]pyrazin-6-yl)-3-methyl-5-(trifluoromethyl)phenol,
111) (S)-2-(2-(2,2-dimethyltetrahydro-2H-pyran-4-yl)-2H-pyrazolo[3,4-b]pyrazin-6-yl)-3-methyl-5-(trifluoromethyl)phenol,
112) (R)-2-(2-(3,3-dimethyltetrahydro-2H-pyran-4-yl)-2H-pyrazolo[3,4-b]pyrazin-6-yl)-3-methyl-5-(trifluoromethyl)phenol,
113) (S)-2-(2-(3,3-dimethyltetrahydro-2H-pyran-4-yl)-2H-pyrazolo[3,4-b]pyrazin-6-yl)-3-methyl-5-(trifluoromethyl)phenol,
114) (R)-2-(2-(5-oxaspiro[2.5]octan-8-yl)-2H-pyrazolo[3,4-b]pyrazin-6-yl)-3-methyl-5-(trifluoromethyl)phenol,
115) (S)-2-(2-(5-oxaspiro[2.5]octan-8-yl)-2H-pyrazolo[3,4-b]pyrazin-6-yl)-3-methyl-5-(trifluoromethyl)phenol,
116) 2-(2-((3S,4S)-3-fluorotetrahydro-2H-pyran-4-yl)-2H-pyrazolo[3,4-b]pyrazin-6-yl)-3-methyl-5-(trifluoromethyl)phenol,
117) 2-(2-((3R,4R)-3-fluorotetrahydro-2H-pyran-4-yl)-2H-pyrazolo[3,4-b]pyrazin-6-yl)-3-methyl-5-(trifluoromethyl)phenol,
118) 2-(2-((3R,4S)-3-fluorotetrahydro-2H-pyran-4-yl)-2H-pyrazolo[3,4-b]pyrazin-6-yl)-3-methyl-5-(trifluoromethyl)phenol,
119) 2-(2-((3S,4R)-3-fluorotetrahydro-2H-pyran-4-yl)-2H-pyrazolo[3,4-b]pyrazin-6-yl)-3-methyl-5-(trifluoromethyl)phenol,
120) (R)-2-(2-(3,3-difluorotetrahydro-2H-pyran-4-yl)-2H-pyrazolo[3,4-b]pyrazin-6-yl)-3-methyl-5-(trifluoromethyl)phenol,
121) (S)-5-(6-(2-hydroxy-6-methyl-4-(trifluoromethyl)phenyl)-3-methyl-2H-pyrazolo[3,4-b]pyrazin-2-yl)-1-methylpiperidin-2-one,
122) (R and S)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
123) (R)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
124) (R)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
125) (R)-5-(6-(2-hydroxy-6-methyl-4-(trifluoromethyl)phenyl)-3-methyl-2H-pyrazolo[3,4-b]pyrazin-2-yl)piperidin-2-one,
126) (S)-5-(6-(2-hydroxy-6-methyl-4-(trifluoromethyl)phenyl)-3-methyl-2H-pyrazolo[3,4-b]pyrazin-2-yl)piperidin-2-one,
127) (R)-3-ethyl-5-((6-(2-hydroxy-6-methyl-4-(trifluoromethyl)phenyl)-2H-pyrazolo[3,4-b]pyrazin-2-yl)methyl)oxazolidin-2-one,
128) (R)-5-(6-(2-hydroxy-4-(trifluoromethyl)phenyl)-5-methyl-2H-pyrazolo[3,4-b]pyrazin-2-yl)-1-methylpiperidin-2-one,
129) (S)-5-(6-(2-hydroxy-4-(trifluoromethyl)phenyl)-5-methyl-2H-pyrazolo[3,4-b]pyrazin-2-yl)-1-methylpiperidin-2-one,
130) (S)-1-cyclopropyl-4-((6-(2-(ethoxymethoxy)-6-methyl-4-(trifluoromethyl)phenyl)-3-((S)-1-hydroxyethyl)-2H-pyrazolo[3,4-b]pyrazin-2-yl)methyl)pyrrolidin-2-one,
131) (S)-1-cyclopropyl-4-((6-(2-(ethoxymethoxy)-6-methyl-4-(trifluoromethyl)phenyl)-3-((R)-1-hydroxyethyl)-2H-pyrazolo[3,4-b]pyrazin-2-yl)methyl)pyrrolidin-2-one,
132) (R)-1-cyclopropyl-4-((6-(2-(ethoxymethoxy)-6-methyl-4-(trifluoromethyl)phenyl)-3-((R)-1-hydroxyethyl)-2H-pyrazolo[3,4-b]pyrazin-2-yl)methyl)pyrrolidin-2-one,
133) (R)-1-cyclopropyl-4-((6-(2-(ethoxymethoxy)-6-methyl-4-(trifluoromethyl)phenyl)-3-((S)-1-hydroxyethyl)-2H-pyrazolo[3,4-b]pyrazin-2-yl)methyl)pyrrolidin-2-one,
134) (S)-5-(6-(2-hydroxy-6-methyl-4-(trifluoromethyl)phenyl)-3-((S)-1-hydroxyethyl)-2H-pyrazolo[3,4-b]pyrazin-2-yl)-1-isopropylpiperidin-2-one,
135) (R)-5-(6-(2-hydroxy-6-methyl-4-(trifluoromethyl)phenyl)-3-((S)-1-hydroxyethyl)-2H-pyrazolo[3,4-b]pyrazin-2-yl)-1-isopropylpiperidin-2-one,
136) (R)-5-(6-(2-hydroxy-6-methyl-4-(trifluoromethyl)phenyl)-3-((R)-1-hydroxyethyl)-2H-pyrazolo[3,4-b]pyrazin-2-yl)-1-isopropylpiperidin-2-one,
137) (S)-5-(6-(2-hydroxy-6-methyl-4-(trifluoromethyl)phenyl)-3-((R)-1-hydroxyethyl)-2H-pyrazolo[3,4-b]pyrazin-2-yl)-1-isopropylpiperidin-2-one, and
138) 2-(2-((1R,3R)-3-fluorocyclobutyl)-2H-[1,2,3]triazolo[4,5-b]pyrazin-5-yl)-3-methyl-5-(trifluoromethyl)phenol,
   and pharmaceutically acceptable salts thereof.

Particular examples of compounds of embodiments include:
1) 3-methyl-2-(2-(tetrahydro-2H-pyran-4-yl)-2H-pyrazolo[3,4-b]pyrazin-6-yl)-5-(trifluoromethyl)phenol;
2) 4-(6-(2-hydroxy-6-methyl-4-(trifluoromethyl)phenyl)-2H-pyrazolo[3,4-b]pyrazin-2-yl)-2-thiabicyclo[2.1.1]hexane 2,2-dioxide;
3) 2-(2-((1R,5S,6r)-3-oxabicyclo[3.1.0]hexan-6-yl)-2H-pyrazolo[3,4-b]pyrazin-6-yl)-3-methyl-5-(trifluoromethyl)phenol;
4) 2-(2-((1r,3r)-3-fluorocyclobutyl)-2H-pyrazolo[3,4-b]pyrazin-6-yl)-3-methyl-5-(trifluoromethyl)phenol;
5) 4-[6-[2-hydroxy-6-methyl-4-(trifluoromethyl)phenyl]pyrazolo[3,4-b]pyrazin-2-yl]bicyclo[2.1.1]hexan-1-ol; and
6) 4-[6-[2-hydroxy-6-methyl-4-(trifluoromethyl)phenyl]pyrazolo[3,4-b]pyrazin-2-yl]bicyclo[2.1.1]hexane-1-carbonitrile;
   and pharmaceutically acceptable salts thereof.

In particular, exemplary compounds of an embodiment are 3-methyl-2-(2-(tetrahydro-2H-pyran-4-yl)-2H-pyrazolo[3,4-b]pyrazin-6-yl)-5-(trifluoromethyl)phenol, and pharmaceutically acceptable salts thereof.

In particular, exemplary compounds of an embodiment are 4-(6-(2-hydroxy-6-methyl-4-(trifluoromethyl)phenyl)-2H-pyrazolo[3,4-b]pyrazin-2-yl)-2-thiabicyclo[2.1.1]hexane 2,2-dioxide, and pharmaceutically acceptable salts thereof.

In particular, exemplary compounds of an embodiment are 2-(2-((1R,5S,6r)-3-oxabicyclo[3.1.0]hexan-6-yl)-2H-pyrazolo[3,4-b]pyrazin-6-yl)-3-methyl-5-(trifluoromethyl)phenol, and pharmaceutically acceptable salts thereof.

In particular, exemplary compounds of an embodiment are 2-(2-((1r,3r)-3-fluorocyclobutyl)-2H-pyrazolo[3,4-b]pyrazin-6-yl)-3-methyl-5-(trifluoromethyl)phenol, and pharmaceutically acceptable salts thereof.

In particular, exemplary compounds of an embodiment are 4-[6-[2-hydroxy-6-methyl-4-(trifluoromethyl)phenyl]pyrazolo[3,4-b]pyrazin-2-yl]bicyclo[2.1.1]hexan-1-ol, and pharmaceutically acceptable salts thereof.

In particular, exemplary compounds of an embodiment are 4-[6-[2-hydroxy-6-methyl-4-(trifluoromethyl)phenyl]pyrazolo[3,4-b]pyrazin-2-yl]bicyclo[2.1.1]hexane-1-carbonitrile, and pharmaceutically acceptable salts thereof.

Illustrative, but non-limiting, examples of compounds of embodiments that are useful as inhibitors of the NLRP3 are compounds of the following structures:

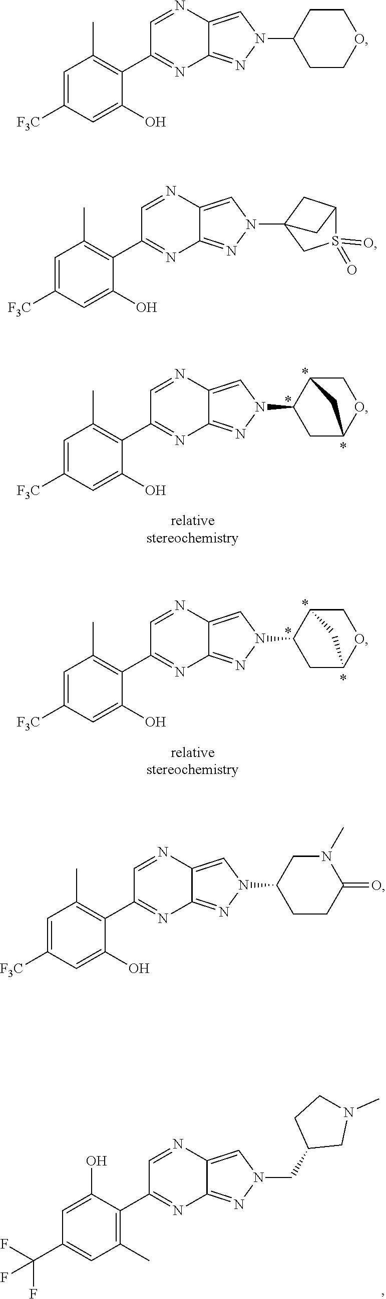 [SMILES: Cc1cc(C(F)(F)F)cc(O)c1-c1cnc2cn(C3CCOCC3)nc2n1 ; Cc1cc(C(F)(F)F)cc(O)c1-c1cnc2cn(C34CC(C3)S(=O)(=O)C4)nc2n1 ; Cc1cc(C(F)(F)F)cc(O)c1-c1cnc2cn([C@@H]3C[C@@H]4C[C@H]3CO4)nc2n1 ; Cc1cc(C(F)(F)F)cc(O)c1-c1cnc2cn([C@H]3C[C@H]4C[C@@H]3CO4)nc2n1 ; Cc1cc(C(F)(F)F)cc(O)c1-c1cnc2cn([C@H]3CCC(=O)N(C)C3)nc2n1 ; Cc1cc(C(F)(F)F)cc(O)c1-c1cnc2cn(C[C@@H]3CCN(C)C3)nc2n1]

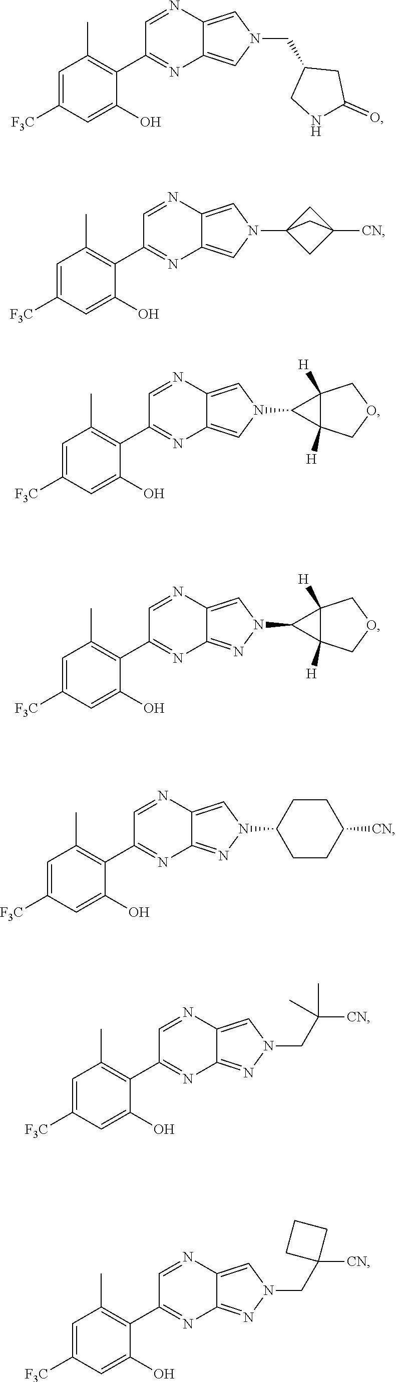

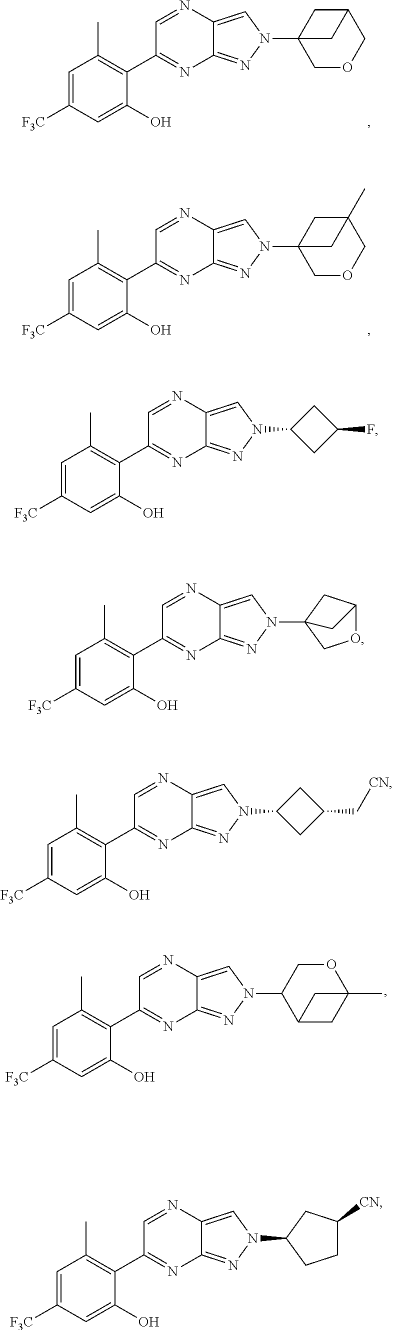

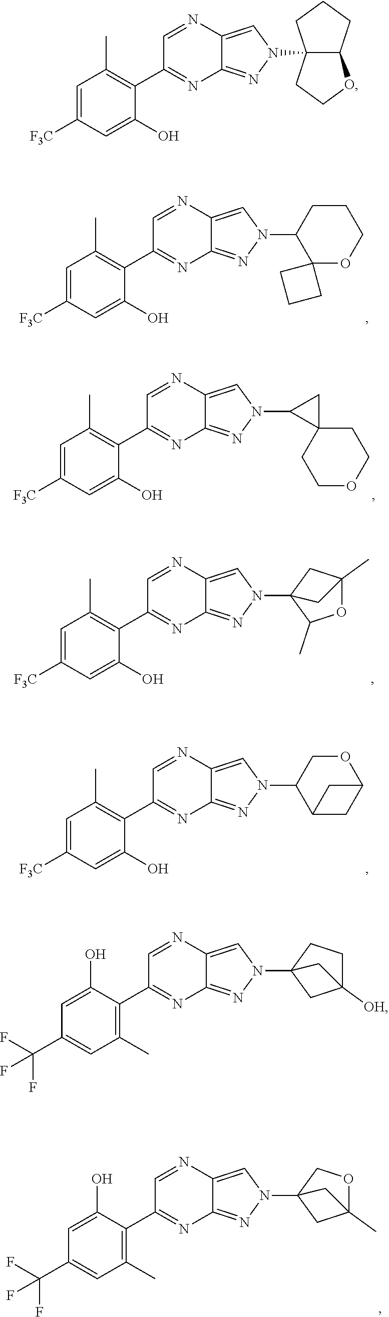

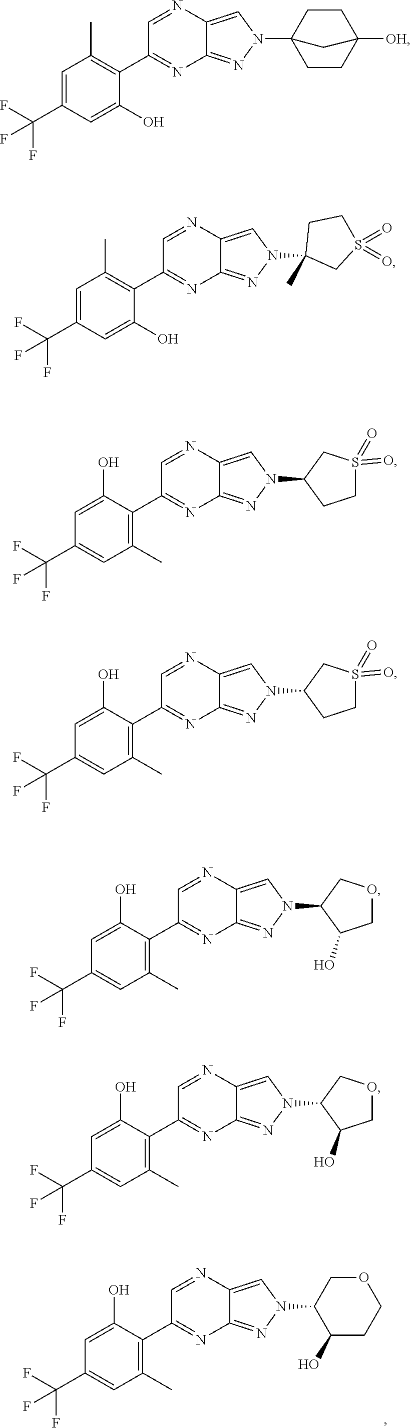

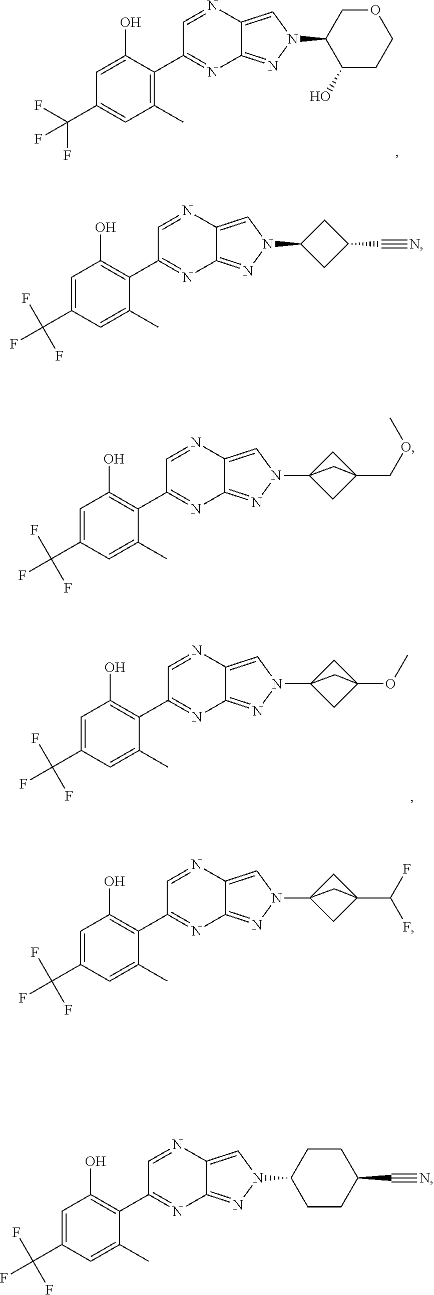 [SMILES: Cc1cc(C(F)(F)F)cc(O)c1-c1cnc2cn([C@H]3COCC[C@@H]3O)nc2n1 ; Cc1cc(C(F)(F)F)cc(O)c1-c1cnc2cn([C@H]3C[C@H](C#N)C3)nc2n1 ; COCC12CC(n3cc4ncc(-c5c(C)cc(C(F)(F)F)cc5O)nc4n3)(C1)C2 ; COC12CC(n3cc4ncc(-c5c(C)cc(C(F)(F)F)cc5O)nc4n3)(C1)C2 ; Cc1cc(C(F)(F)F)cc(O)c1-c1cnc2cn(C34CC(C(F)F)(C3)C4)nc2n1 ; Cc1cc(C(F)(F)F)cc(O)c1-c1cnc2cn([C@H]3CC[C@H](C#N)CC3)nc2n1]

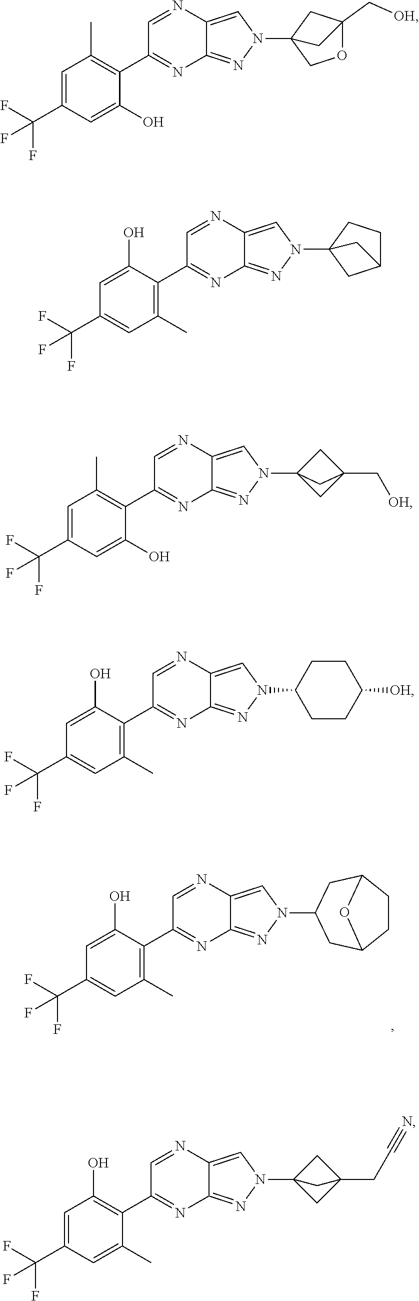 [SMILES: Cc1cc(C(F)(F)F)cc(O)c1-c1cnc2cn(C34COC(CO)(C3)C4)nc2n1 ; Cc1cc(C(F)(F)F)cc(O)c1-c1cnc2cn(C34CCC(C3)C4)nc2n1 ; Cc1cc(C(F)(F)F)cc(O)c1-c1cnc2cn(C34CC(CO)(C3)C4)nc2n1 ; Cc1cc(C(F)(F)F)cc(O)c1-c1cnc2cn([C@H]3CC[C@@H](O)CC3)nc2n1 ; Cc1cc(C(F)(F)F)cc(O)c1-c1cnc2cn(C3CC4CCC(C3)O4)nc2n1 ; Cc1cc(C(F)(F)F)cc(O)c1-c1cnc2cn(C34CC(CC#N)(C3)C4)nc2n1]

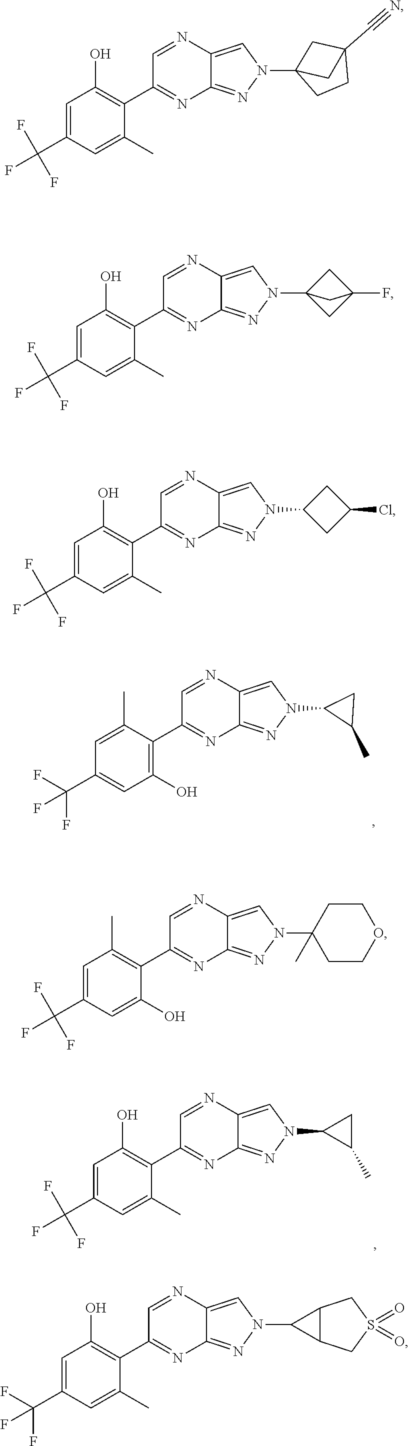

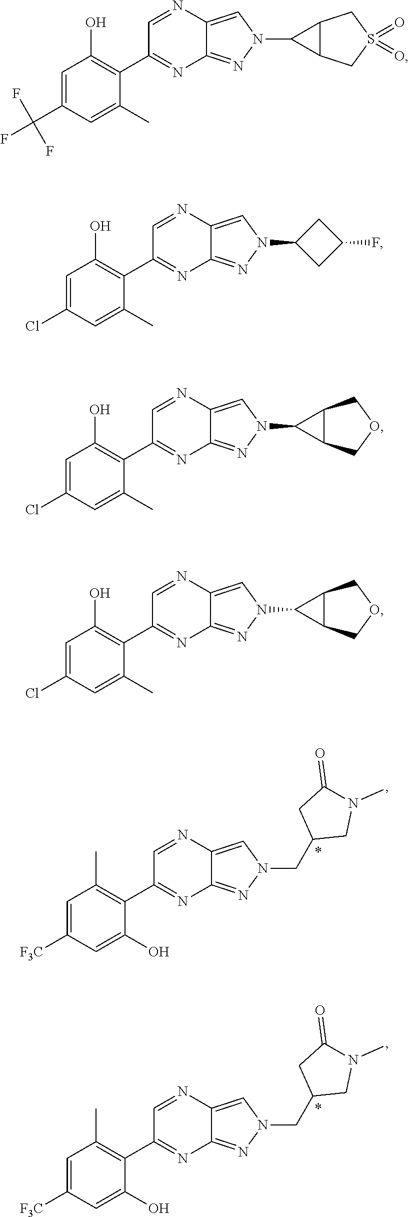 [SMILES: Cc1cc(C(F)(F)F)cc(O)c1-c1cnc2cn(C3C4CS(=O)(=O)CC43)nc2n1 ; Cc1cc(Cl)cc(O)c1-c1cnc2cn([C@H]3C[C@H](F)C3)nc2n1 ; Cc1cc(Cl)cc(O)c1-c1cnc2cn([C@@H]3[C@@H]4COC[C@@H]43)nc2n1 ; Cc1cc(Cl)cc(O)c1-c1cnc2cn([C@H]3[C@@H]4COC[C@@H]43)nc2n1 ; Cc1cc(C(F)(F)F)cc(O)c1-c1cnc2cn(CC3CC(=O)N(C)C3)nc2n1 ; Cc1cc(C(F)(F)F)cc(O)c1-c1cnc2cn(CC3CC(=O)N(C)C3)nc2n1]

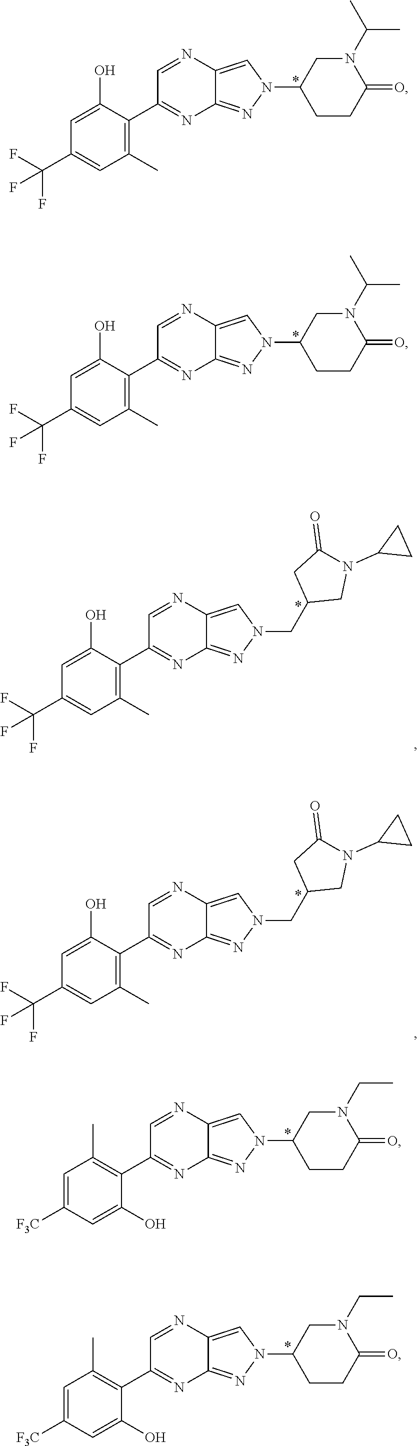 [SMILES: Cc1cc(C(F)(F)F)cc(O)c1-c1cnc2cn(C3CCC(=O)N(C(C)C)C3)nc2n1 ; Cc1cc(C(F)(F)F)cc(O)c1-c1cnc2cn(C3CCC(=O)N(C(C)C)C3)nc2n1 ; Cc1cc(C(F)(F)F)cc(O)c1-c1cnc2cn(CC3CC(=O)N(C4CC4)C3)nc2n1 ; Cc1cc(C(F)(F)F)cc(O)c1-c1cnc2cn(CC3CC(=O)N(C4CC4)C3)nc2n1 ; CCN1CC(n2cc3ncc(-c4c(C)cc(C(F)(F)F)cc4O)nc3n2)CCC1=O ; CCN1CC(n2cc3ncc(-c4c(C)cc(C(F)(F)F)cc4O)nc3n2)CCC1=O]

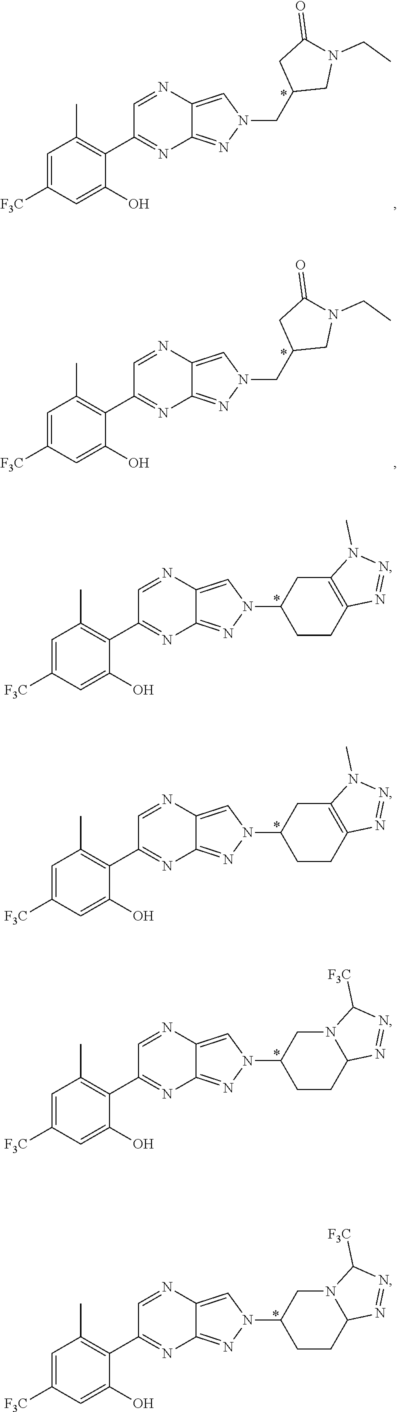 [SMILES: CCN1CC(Cn2cc3ncc(-c4c(C)cc(C(F)(F)F)cc4O)nc3n2)CC1=O ; CCN1CC(Cn2cc3ncc(-c4c(C)cc(C(F)(F)F)cc4O)nc3n2)CC1=O ; Cc1cc(C(F)(F)F)cc(O)c1-c1cnc2cn(C3CCc4nnn(C)c4C3)nc2n1 ; Cc1cc(C(F)(F)F)cc(O)c1-c1cnc2cn(C3CCc4nnn(C)c4C3)nc2n1 ; Cc1cc(C(F)(F)F)cc(O)c1-c1cnc2cn(C3CCC4N=NC(C(F)(F)F)N4C3)nc2n1 ; Cc1cc(C(F)(F)F)cc(O)c1-c1cnc2cn(C3CCC4N=NC(C(F)(F)F)N4C3)nc2n1]

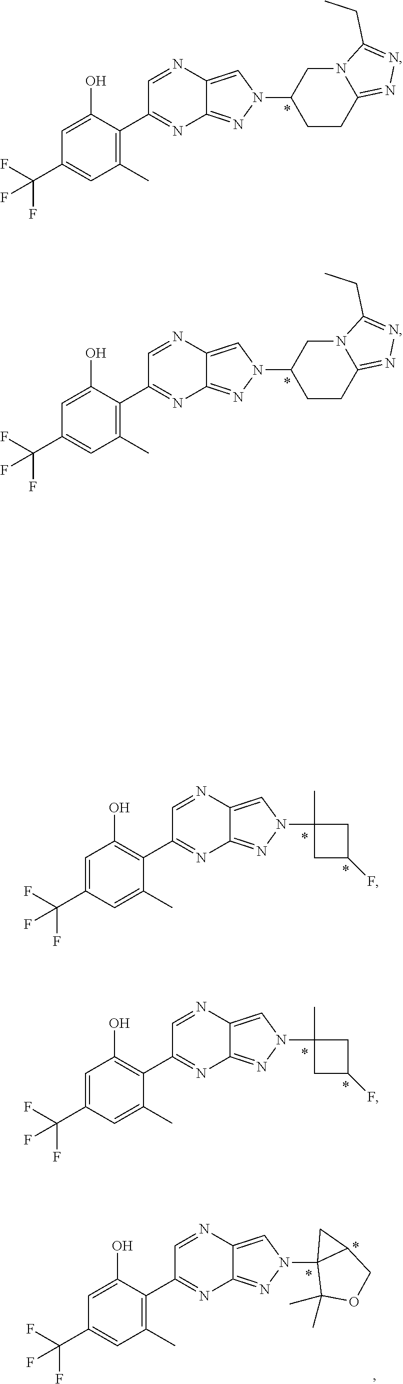 [SMILES: CCc1nnc2n1CC(n1cc3ncc(-c4c(C)cc(C(F)(F)F)cc4O)nc3n1)CC2 ; CCc1nnc2n1CC(n1cc3ncc(-c4c(C)cc(C(F)(F)F)cc4O)nc3n1)CC2 ; Cc1cc(C(F)(F)F)cc(O)c1-c1cnc2cn(C3(C)CC(F)C3)nc2n1 ; Cc1cc(C(F)(F)F)cc(O)c1-c1cnc2cn(C3(C)CC(F)C3)nc2n1 ; Cc1cc(C(F)(F)F)cc(O)c1-c1cnc2cn(C34CC3COC4(C)C)nc2n1]

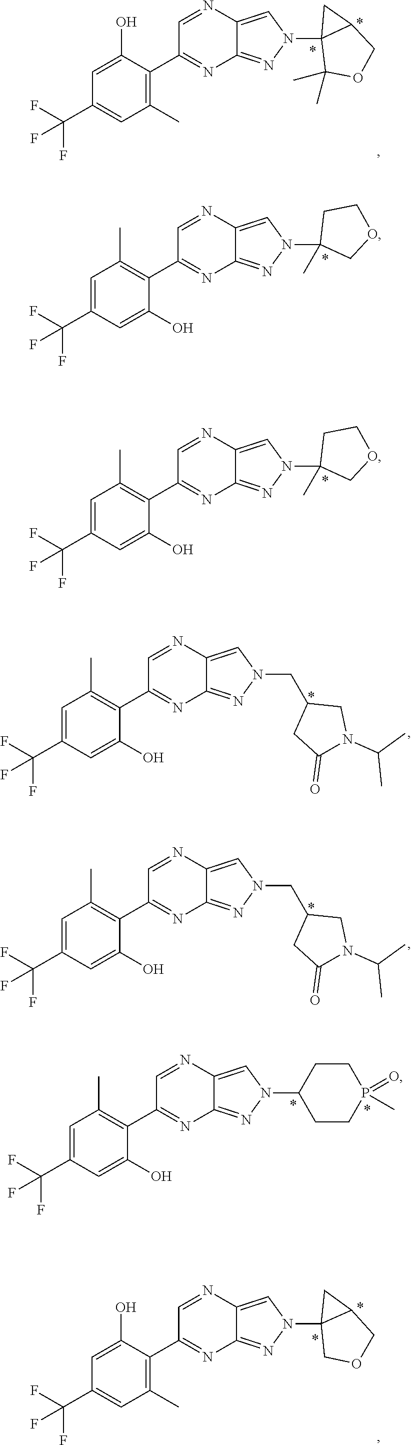

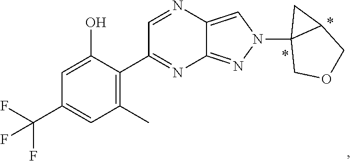 [SMILES: Cc1cc(C(F)(F)F)cc(O)c1-c1cnc2cn(C34COCC3C4)nc2n1]

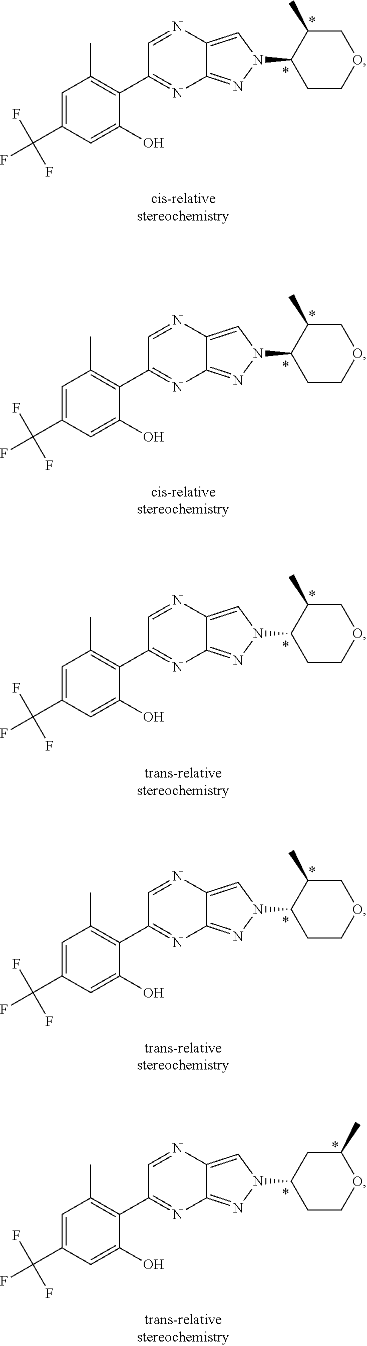 [SMILES: Cc1cc(C(F)(F)F)cc(O)c1-c1cnc2cn([C@@H]3CCOC[C@@H]3C)nc2n1 ; Cc1cc(C(F)(F)F)cc(O)c1-c1cnc2cn([C@@H]3CCOC[C@@H]3C)nc2n1 ; Cc1cc(C(F)(F)F)cc(O)c1-c1cnc2cn([C@H]3CCOC[C@@H]3C)nc2n1 ; Cc1cc(C(F)(F)F)cc(O)c1-c1cnc2cn([C@H]3CCOC[C@@H]3C)nc2n1 ; Cc1cc(C(F)(F)F)cc(O)c1-c1cnc2cn([C@H]3CCO[C@H](C)C3)nc2n1]

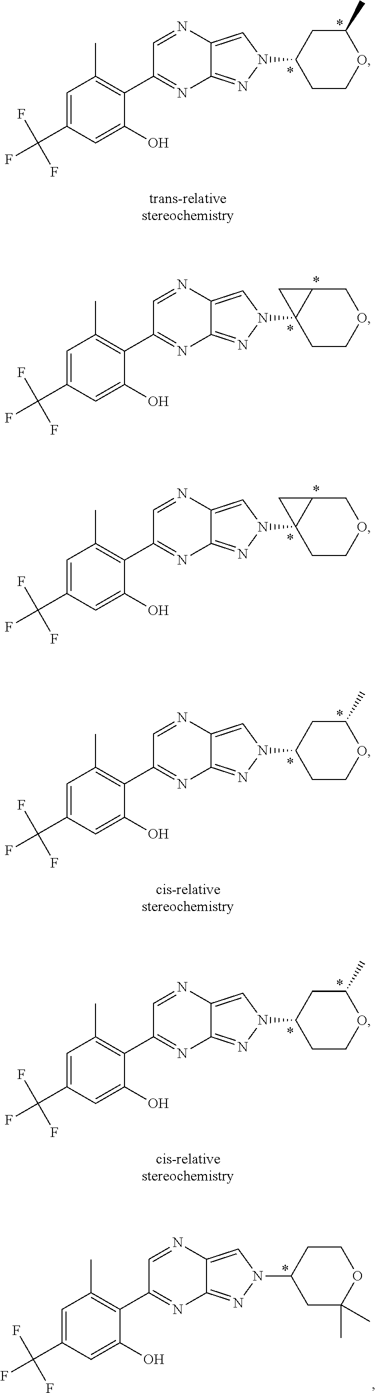 [SMILES: Cc1cc(C(F)(F)F)cc(O)c1-c1cnc2cn([C@H]3CCO[C@H](C)C3)nc2n1 ; Cc1cc(C(F)(F)F)cc(O)c1-c1cnc2cn([C@@]34CCOCC3C4)nc2n1 ; Cc1cc(C(F)(F)F)cc(O)c1-c1cnc2cn([C@@]34CCOCC3C4)nc2n1 ; Cc1cc(C(F)(F)F)cc(O)c1-c1cnc2cn([C@H]3CCO[C@@H](C)C3)nc2n1 ; Cc1cc(C(F)(F)F)cc(O)c1-c1cnc2cn([C@H]3CCO[C@@H](C)C3)nc2n1 ; Cc1cc(C(F)(F)F)cc(O)c1-c1cnc2cn(C3CCOC(C)(C)C3)nc2n1]

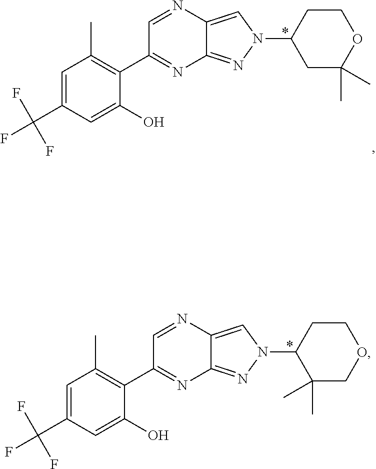 [SMILES: Cc1cc(C(F)(F)F)cc(O)c1-c1cnc2cn(C3CCOC(C)(C)C3)nc2n1 ; Cc1cc(C(F)(F)F)cc(O)c1-c1cnc2cn(C3CCOCC3(C)C)nc2n1]

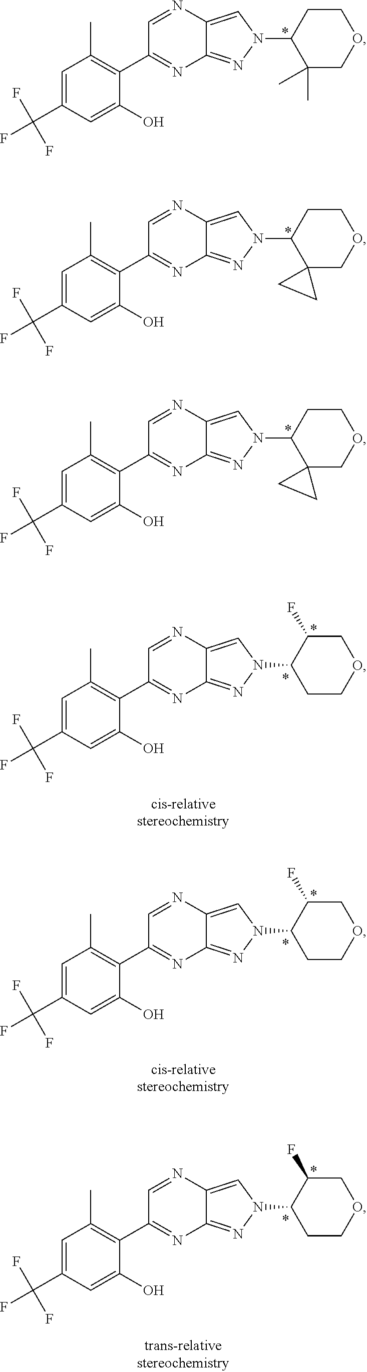 [SMILES: Cc1cc(C(F)(F)F)cc(O)c1-c1cnc2cn(C3CCOCC3(C)C)nc2n1 ; Cc1cc(C(F)(F)F)cc(O)c1-c1cnc2cn(C3CCOCC34CC4)nc2n1 ; Cc1cc(C(F)(F)F)cc(O)c1-c1cnc2cn(C3CCOCC34CC4)nc2n1 ; Cc1cc(C(F)(F)F)cc(O)c1-c1cnc2cn([C@H]3CCOC[C@H]3F)nc2n1 ; Cc1cc(C(F)(F)F)cc(O)c1-c1cnc2cn([C@H]3CCOC[C@H]3F)nc2n1 ; Cc1cc(C(F)(F)F)cc(O)c1-c1cnc2cn([C@H]3CCOC[C@@H]3F)nc2n1]

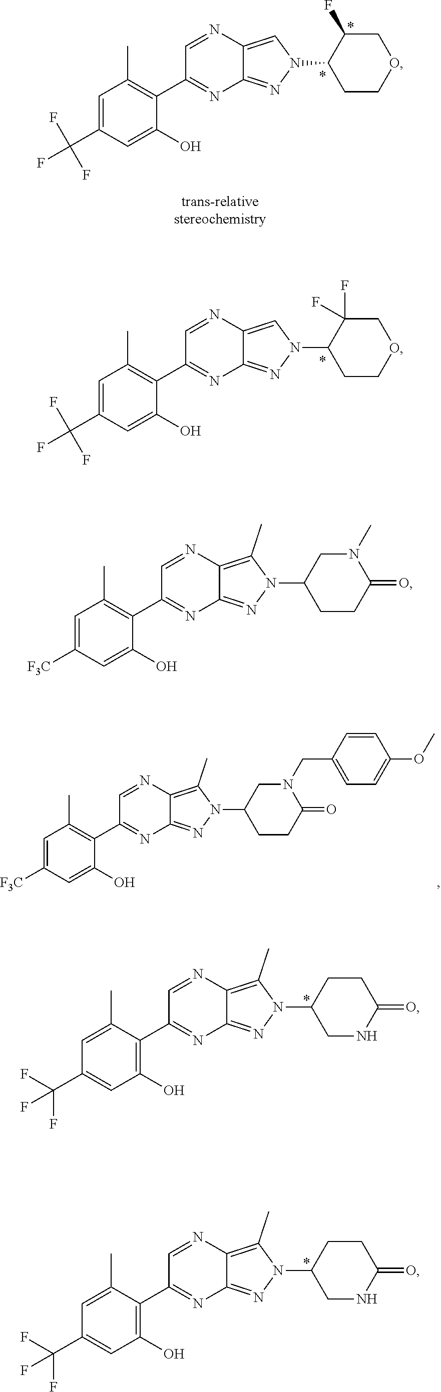 [SMILES: Cc1cc(C(F)(F)F)cc(O)c1-c1cnc2cn([C@H]3CCOC[C@@H]3F)nc2n1 ; Cc1cc(C(F)(F)F)cc(O)c1-c1cnc2cn(C3CCOCC3(F)F)nc2n1 ; Cc1cc(C(F)(F)F)cc(O)c1-c1cnc2c(C)n(C3CCC(=O)N(C)C3)nc2n1 ; COc1ccc(CN2CC(n3nc4nc(-c5c(C)cc(C(F)(F)F)cc5O)cnc4c3C)CCC2=O)cc1 ; Cc1cc(C(F)(F)F)cc(O)c1-c1cnc2c(C)n(C3CCC(=O)NC3)nc2n1 ; Cc1cc(C(F)(F)F)cc(O)c1-c1cnc2c(C)n(C3CCC(=O)NC3)nc2n1]

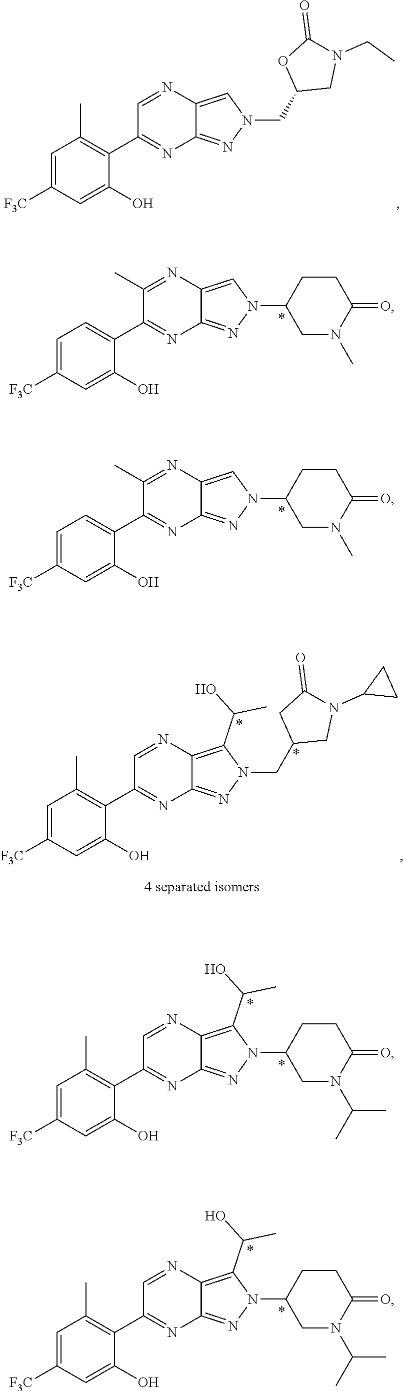 [SMILES: CCN1C[C@H](Cn2cc3ncc(-c4c(C)cc(C(F)(F)F)cc4O)nc3n2)OC1=O ; Cc1nc2cn(C3CCC(=O)N(C)C3)nc2nc1-c1ccc(C(F)(F)F)cc1O ; Cc1nc2cn(C3CCC(=O)N(C)C3)nc2nc1-c1ccc(C(F)(F)F)cc1O ; Cc1cc(C(F)(F)F)cc(O)c1-c1cnc2c(C(C)O)n(CC3CC(=O)N(C4CC4)C3)nc2n1 ; Cc1cc(C(F)(F)F)cc(O)c1-c1cnc2c(C(C)O)n(C3CCC(=O)N(C(C)C)C3)nc2n1 ; Cc1cc(C(F)(F)F)cc(O)c1-c1cnc2c(C(C)O)n(C3CCC(=O)N(C(C)C)C3)nc2n1]

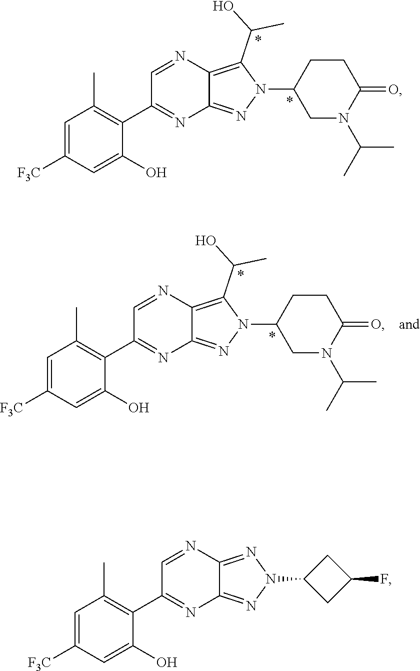 [SMILES: Cc1cc(C(F)(F)F)cc(O)c1-c1cnc2c(C(C)O)n(C3CCC(=O)N(C(C)C)C3)nc2n1 ; Cc1cc(C(F)(F)F)cc(O)c1-c1cnc2c(C(C)O)n(C3CCC(=O)N(C(C)C)C3)nc2n1 ; "and" ; Cc1cc(C(F)(F)F)cc(O)c1-c1cnc2nn([C@H]3C[C@H](F)C3)nc2n1]

and pharmaceutically acceptable salts thereof.

Particular examples of compounds of embodiments include:

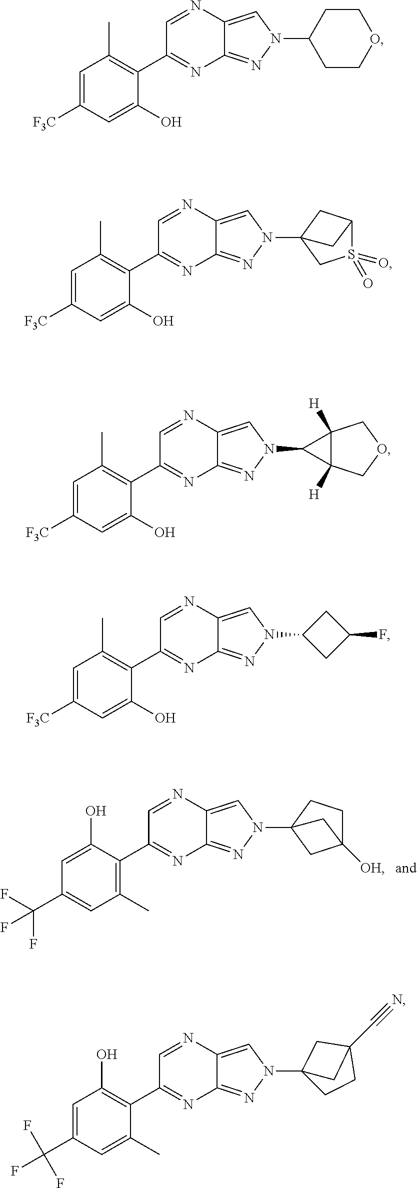

and pharmaceutically acceptable salts thereof.

In particular, exemplary compounds of an embodiment are

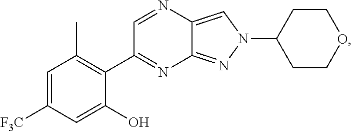 [SMILES: Cc1cc(C(F)(F)F)cc(O)c1-c1cnc2cn(C3CCOCC3)nc2n1]

and pharmaceutically acceptable salts thereof.

In particular, exemplary compounds of an embodiment are

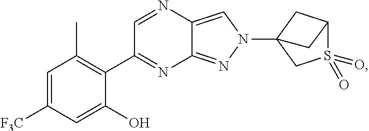 [SMILES: Cc1cc(C(F)(F)F)cc(O)c1-c1cnc2cn(C34CC(C3)S(=O)(=O)C4)nc2n1]

and pharmaceutically acceptable salts thereof.

In particular, exemplary compounds of an embodiment are

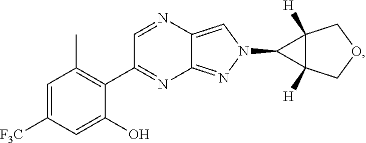 [SMILES: Cc1cc(C(F)(F)F)cc(O)c1-c1cnc2cn([C@H]3[C@@H]4COC[C@@H]43)nc2n1]

and pharmaceutically acceptable salts thereof.

In particular, exemplary compounds of an embodiment are

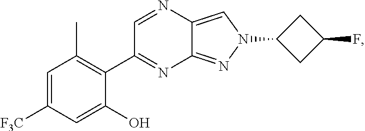 [SMILES: Cc1cc(C(F)(F)F)cc(O)c1-c1cnc2cn([C@H]3C[C@H](F)C3)nc2n1]

and pharmaceutically acceptable salts thereof.

In particular, exemplary compounds of an embodiment are

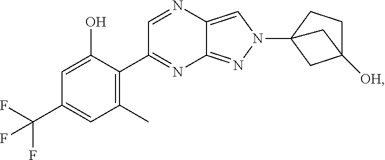 [SMILES: Cc1cc(C(F)(F)F)cc(O)c1-c1cnc2cn(C34CCC(O)(C3)C4)nc2n1]

and pharmaceutically acceptable salts thereof.

In particular, exemplary compounds of an embodiment are

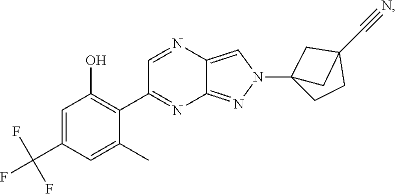 [SMILES: Cc1cc(C(F)(F)F)cc(O)c1-c1cnc2cn(C34CCC(C#N)(C3)C4)nc2n1]

and pharmaceutically acceptable salts thereof.

Although the specific stereochemistries described above are preferred, other stereoisomers, including diastereoisomers, enantiomers, epimers, and mixtures of these may also have utility in treating NLRP3 mediated diseases.

Synthetic methods for making the compounds are disclosed in the Examples shown below. Where synthetic details are not provided in the examples, the compounds are readily made by a person of ordinary skill in the art of medicinal chemistry or synthetic organic chemistry by applying the synthetic information provided herein. Where a stereochemical center is not defined, the structure represents a mixture of stereoisomers at that center. For such compounds, the individual stereoisomers, including enantiomers, diastereoisomers, and mixtures of these are also compounds of the invention.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

“Alkyl” means monovalent, saturated carbon chains, which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Non-limiting examples of alkyl groups include methyl, ethyl, propyl, isopropyl, butyl, sec- and tert-butyl, pentyl, hexyl, heptyl, octyl, nonyl, and the like.

“Alkylene” means bivalent saturated carbon chains, which may be linear, branched, or combinations thereof.

“Cycloalkyl” means a saturated monocyclic, bicyclic, or tricyclic carbocyclic ring, having a specified number of carbon atoms. Non-limiting examples of monocyclic cycloalkyl include cyclopropyl, cyclobutyl, cyclopentyl, cyclohexyl, cycloheptyl, and the like. Bicyclic carbocyclic rings, which feature two joined rings, may be fused (in which adjacent rings share at least two atoms and bridgehead carbons are directly connected, such as

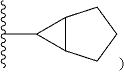 [SMILES: *C1C2CCCC12]

or bridged (in which one or more atoms spans or bridges another ring of atoms, such as

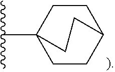 [SMILES: *C12CCC(CC1)CC2]

Tricyclic carbocyclic rings similarly feature three fused or bridged rings. Spirocyclic rings, in which a single carbon atom is shared by either two rings (such as

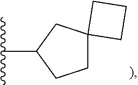 [SMILES: *C1CCC2(CCC2)C1]

are also contemplated herein and may be formed by two substituents attached to the same carbon atom.

“Heterocycloalkyl” means monocyclic, bicyclic, or tricyclic ring or ring system having 3 to 14 ring atoms and containing at least one ring heteroatom selected from N (including NH and NR*, where R* is a substituent such as an alkyl group), S (including SO and SO₂), O, and P (including PO, PO₂, and POR*, where R* is a substituent such as an alkyl group). The heterocycloalkyl ring may be substituted on the ring carbons and/or the ring nitrogen, sulfur, or phosphorus. Heterocycloalkyl rings may be non-aromatic or partially aromatic, in the case of bicyclic or tricyclic ring systems. Bicyclic heterocycloalkyls, which feature two joined rings, may be fused (in which adjacent rings share at least two atoms and bridgehead atoms are directly connected, such as

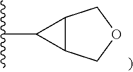 [SMILES: *C1C2COCC12]

or bridged (in which one or more atoms spans or bridges another ring of atoms, such as

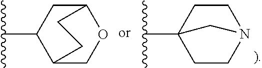

Tricyclic heterocycloalkyls similarly feature three fused or bridged rings. Spirocyclic rings, in which a single atom is shared by either two rings (such as

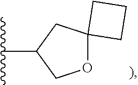 [SMILES: *C1COC2(CCC2)C1]

pre also contemplated herein and may be formed by two substituents attached to the same atom. Non-limiting examples of heterocycloalkyl groups include tetrahydrofuranyl, pyrrolidinyl, tetrahydrothiophenyl, azetidinyl, piperazinyl, piperidinyl, morpholinyl, oxetanyl, tetrahydropyranyl, thiomorpholine, tetrahydropyran, octahydro-1H-pyrrolo[2,3-c]pyridine, 5,6,7,8-tetrahydro-[1,2,4]triazolo[4,3-a]pyridine, 4,5,6,7-tetrahydro-1H-benzo[d][1,2,3]triazole, 3-azabicyclo[3.1.0]hexane, 5-azaspiro[2.4]heptane, 1-oxa-7-azaspiro[4.4]nonane, 1-oxa-8-azaspiro[4.5]decane, 3-oxa-1,8-diazaspiro[4.5]decane, 2,8-diazaspiro[4.5]decane, 1-oxa-3,8-diazaspiro[4.5]decane, 2-oxa-8-azaspiro[4.5]decane, 1,8-diazaspiro[4.5]decane, and 1-oxa-4,9-diazaspiro[5.5]undecane.

“Aryl” means a monocyclic, bicyclic, or tricyclic carbocyclic aromatic ring or ring system containing 6 to 14 carbon atoms, wherein at least one of the rings is aromatic. Non-limiting examples of aryl include phenyl and naphthyl.

“Heteroaryl” means a monocyclic, bicyclic, or tricyclic ring or ring system containing 5 to 14 ring atoms and containing at least one ring heteroatom selected from N (including NH and NR*, where R* is a substituent such as an alkyl group), S (including SO and SO₂), O, and P (including PO, PO₂, and POR*, where R* is a substituent such as an alkyl group), wherein at least one of the heteroatom containing rings is aromatic. In embodiments, a heteroaryl group is monocyclic and has 5 or 6 ring atoms (“5- or 6-membered monocyclic heteroaryl”). In other embodiments, a heteroaryl group is bicyclic and has 8 to 10 ring atoms (“8- to 10-membered bicyclic heteroaryl”). In other embodiments, a heteroaryl group is bicyclic and has 9 to 11 ring atoms (“9- to 11-membered bicyclic heteroaryl”). Non-limiting examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl, quinolyl, indolyl, isoquinolyl, quinazolinyl, dibenzofuranyl, and the like.

“Halogen” or “halo” includes fluorine, chlorine, bromine, and iodine. In one embodiment, halogen is fluorine, chorine, or bromine. In another embodiment, halogen is fluorine or chlorine. In another embodiment, halogen is chlorine or bromine. In another embodiment, halogen is fluorine or bromine. In another embodiment, halogen is fluorine. In another embodiment, halogen is chlorine. In another embodiment, halogen is bromine.

“Saturated” means containing only single bonds.

“Unsaturated” means containing at least one double or triple bond. In one embodiment, unsaturated means containing at least one double bond. In another embodiment, unsaturated means containing at least one triple bond.

When any variable (e.g., R^{a} etc.) occurs more than one time in any constituent or in Formula (I), its definition on each occurrence is independent of its definition at every other occurrence. Also, combinations of substituents and/or variables are permissible only if such combinations result in stable compounds. A squiggly or wavy line at the end of or across a bond in a substituent variable (such as or

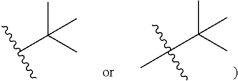

represents the point of attachment.

Under nomenclature used throughout this disclosure, the point of attachment is described first, followed by the terminal portion of the designated side chain. For example, a C₁₋₅ alkylcarbonylamino C₁₋₆ alkyl substituent is equivalent to:

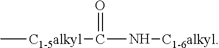

In choosing compounds of the present invention, one of ordinary skill in the art will recognize that the various substituents are to be chosen in conformity with well-known principles of chemical structure connectivity and stability.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

Compounds of Formula (I) may contain one or more asymmetric centers and can thus occur as racemates and racemic mixtures, single enantiomers, diastereomeric mixtures, and individual diastereomers. The present invention is meant to encompass all such isomeric forms of the compounds of Formula (I).

The independent syntheses of optical isomers and diastereoisomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration or sufficient heavy atoms to make an absolute assignment.

If desired, racemic mixtures of the compounds may be separated so that the individual enantiomers are isolated. The separation can be carried out by methods well-known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diastereoisomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula (I).

In the compounds of general Formula (I), the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural Formula (I). For example, different isotopic forms of hydrogen (H) include protium (¹H), deuterium (²H), and tritium (³H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Tritium is radioactive and may therefore provide for a radiolabeled compound, useful as a tracer in metabolic or kinetic studies. Isotopically-enriched compounds within structural Formula (I), can be prepared without undue experimentation by conventional techniques well known to those skilled in the art or by processes analogous to those described in the Schemes and Examples herein using appropriate isotopically-enriched reagents and/or intermediates.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

### Salts

It will be understood that, as used herein, references to the compounds of the present invention are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, formic, fumarate, gluceptate, gluconate, glutamate, glycollylars-anilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide, trifluoroacetate, and valerate. Where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

The term “prodrug” means compounds that are rapidly transformed, for example, by hydrolysis in blood, in vivo to the parent compound, e.g., conversion of a prodrug of Formula (I) to a compound of Formula (I), or to a salt thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. This invention includes prodrugs of the novel compounds of this disclosure. Solvates, and in particular, the hydrates of the compounds of the present disclosure are included in the present invention as well.

### Utilities

The compounds of the present invention are potent inhibitors of NOD-Like Receptor Protein 3 (NLPR3). The compounds, and pharmaceutically acceptable salts thereof, may be efficacious in the treatment of diseases, disorders, and conditions that are mediated by the inhibition of NOD-Like Receptor Protein 3 (NLPR3).

The present invention relates to the treatment or prevention of a disease, disorder or condition mediated by NLRP3 such as inflammation, an auto-immune disease, a cancer, an infection, a disease or disorder of the central nervous system, a metabolic disease, a cardiovascular disease, a fibrotic disease or fibrosis, a respiratory disease, a kidney disease, a liver disease, an ophthalmic or ocular disease, a skin disease, a lymphatic disease, a rheumatic disease, graft versus host disease, allodynia, or an NLRP3-related disease in a subject that has been determined to carry a germline or somatic non-silent mutation in NLRP3.

The disease, disorder, or condition mediated by NLRP3 includes but is not limited to: obesity, gout, pseudogout, osteoarthritis, familial cold autoinflammatory syndrome, Muckle-Wells syndrome, neonatal onset multisystem inflammatory disease, diabetes, non-alcoholic steatohepatitis (NASH), metabolic dysfunction-associated steatohepatitis (MASH), sepsis, age related macular degeneration, diabetic retinopathy, liver fibrosis, kidney fibrosis, atherosclerosis, heart failure, peripheral artery disease, myeloproliferative neoplasm, leukemia, myelodysplastic syndrome, myelofibrosis, lung cancer, colon cancer, Parkinson's disease, Alzheimer's disease, dementia with Lewy bodies (DLB), traumatic brain injury, spinal cord injury, amyotrophic lateral sclerosis, multiple sclerosis, atopic dermatitis, hidradenitis suppurativa, pericarditis, myocarditis, preeclampsia, dermatomyositis, Still's disease, juvenile idiopathic arthritis, age related macular degeneration, diabetic retinopathy, acute kidney disease, a chronic kidney disease, or a rare kidney disease. Diseases, disorders, or conditions mediated by NOD-Like Receptor Protein 3 (NLPR3)), also include, but are not limited to, obesity, gout, pseudogout, CAPS, NASH, MASH, fibrosis, osteoarthritis, atherosclerosis, heart failure, idiopathic pericarditis, myocarditis, atopic dermatitis, hidradenitis suppurativa, inflammatory bowel disease, cancer, Alzheimer's Disease, Parkinson's Disease, dementia with Lewy bodies (DLB), and traumatic brain injury.

In one embodiment of the present invention, the condition, disease, or disorder is obesity.

In another embodiment of the present invention, the condition, disease, or disorder is an inflammatory joint disease such as gout, pseudogout, or osteoarthritis.

In another embodiment, the cryopyrin-associated autoinflammatory syndrome is familial cold autoinflammatory syndrome, Muckle-Wells syndrome, or neonatal onset multisystem inflammatory disease.

In another embodiment, the metabolic disease is diabetes.

In another embodiment, the liver disease is NASH.

In another embodiment, the liver disease is MASH.

In another embodiment, the infection is sepsis.

In another embodiment, the ophthalmic or ocular disease is age related macular degeneration or diabetic retinopathy.

In another embodiment, the fibrotic disease is liver fibrosis or kidney fibrosis.

In some embodiments, the cardiovascular disease is atherosclerosis, heart failure or peripheral artery disease.

In another embodiment, the cancer is myeloproliferative neoplasm, leukemia, myelodysplastic syndrome, myelofibrosis, lung cancer, or colon cancer.

In another embodiment of the present invention, the condition, disease or disorder of the central nervous system is Parkinson's disease, Alzheimer's disease, dementia with Lewy bodies (DLB), traumatic brain injury, spinal cord injury, amyotrophic lateral sclerosis, or multiple sclerosis.

In another embodiment, the skin disease is atopic dermatitis or hidradenitis suppurativa (HS).

In another embodiment, the inflammatory disease is pericarditis or myocarditis.

In another embodiment, the inflammatory disease is preeclampsia.

In another embodiment, the rheumatic disease is dermatomyositis, Still's disease, or juvenile idiopathic arthritis.

In another embodiment, the ocular disease is age related macular degeneration or diabetic retinopathy.

In another embodiment, the kidney disease is an acute kidney disease, a chronic kidney disease, or a rare kidney disease.

One or more of these conditions or diseases may be treated, managed, prevented, reduced, alleviated, ameliorated, or controlled by the administration of a therapeutically effective amount of a compound of the present invention, or a pharmaceutically acceptable salt thereof, to a patient in need of treatment.

The compounds of the present invention may also be used for the manufacture of a medicament which may be useful for treating, preventing, managing, alleviating, ameliorating or controlling one or more of these conditions, diseases or disorders, including but not limited to: gout, pseudogout, osteoarthritis, familial cold autoinflammatory syndrome, Muckle-Wells syndrome, neonatal onset multisystem inflammatory disease, diabetes, NASH, MASH, sepsis, age related macular degeneration, diabetic retinopathy, liver fibrosis, kidney fibrosis, atherosclerosis, heart failure, peripheral artery disease, myeloproliferative neoplasm, leukemia, myelodysplastic syndrome, myelofibrosis, lung cancer, colon cancer, Parkinson's disease, Alzheimer's disease, dementia with Lewy bodies (DLB), traumatic brain injury, spinal cord injury, amyotrophic lateral sclerosis, multiple sclerosis, atopic dermatitis, hidradenitis suppurativa, pericarditis, myocarditis, preeclampsia, dermatomyositis, Still's disease, juvenile idiopathic arthritis, age related macular degeneration, diabetic retinopathy, acute kidney disease, a chronic kidney disease, or a rare kidney disease. The compounds of the present invention may also be used for the manufacture of a medicament which may be useful for treating, preventing, managing, alleviating, ameliorating or controlling one or more of these conditions, diseases or disorders, including but not limited to: gout, pseudogout, CAPS, NASH, MASH, fibrosis, osteoarthritis, atherosclerosis, heart failure, idiophathic pericarditis, myocarditis, atopic dermatitis, hidradenitis suppurativa, inflammatory bowel disease, cancer, Alzheimer's Disease, Parkinson's Disease, dementia with Lewy bodies (DLB), and traumatic brain injury.

Preferred uses of the compounds may be for the treatment of one or more of the following diseases by administering a therapeutically effective amount to a patient in need of treatment. The compounds may be used for manufacturing a medicament for the treatment of one or more of these diseases:
1) obesity,
2) gout,
3) pseudogout,
4) cryopyrin-associated periodic syndromes,
5) non-alcoholic steatohepatitis,
6) metabolic dysfunction-associated steatohepatitis,
7) fibrosis,
8) osteoarthritis,
9) atherosclerosis,
10) atopic dermatitis,
11) hidradenitis suppurativa,
12) Alzheimer's Disease,
13) Parkinson's Disease, and
14) dementia with Lewy bodies (DLB).

Treatment of a disease, disorder or condition mediated by NLPR3 or the NLPR3 inflammasome pathway refers to the administration of the compounds of the present invention to a subject with the disease, disorder, or condition.

One outcome of treatment may be reducing the disease, disorder, or condition mediated by NLPR3 or the NLPR3 inflammasome pathway. Another outcome of treatment may be alleviating the disease, disorder, or condition mediated by NLPR3 or the NLPR3 inflammasome pathway. Another outcome of treatment may be ameliorating the disease, disorder, or condition mediated by NLPR3 or the NLPR3 inflammasome pathway. Another outcome of treatment may be suppressing the disease, disorder, or condition mediated by mediated by NLPR3 or the NLPR3 inflammasome pathway. Another outcome of treatment may be managing the disease, disorder, or condition mediated by NLPR3 or the NLPR3 inflammasome pathway. Another outcome of treatment may be preventing the disease, disorder, or condition mediated by NLPR3 or the NLPR3 inflammasome pathway.

Prevention of the disease, disorder, or condition mediated by NLPR3 or the NLPR3 inflammasome pathway refers to the administration of the compounds of the present invention to a subject at risk of the disease, disorder, or condition. One outcome of prevention may be reducing the disease, disorder or condition mediated by NLPR3 or the NLPR3 inflammasome pathway in a subject at risk of the disease, disorder, or condition. Another outcome of prevention may be suppressing the disease, disorder, or condition mediated by NLPR3 or the NLPR3 inflammasome pathway in a subject at risk of the disease, disorder, or condition. Another outcome of prevention may be ameliorating the disease, disorder or condition mediated by NLPR3 or the NLPR3 inflammasome pathway in a subject at risk of the disease, disorder, or condition. Another outcome of prevention may be alleviating the disease, disorder, or condition mediated by NLPR3 or the NLPR3 inflammasome pathway in a subject at risk of the disease, disorder, or condition. Another outcome of prevention may be managing the disease, disorder, or condition mediated by NLPR3 or the NLPR3 inflammasome pathway in a subject at risk of the disease, disorder, or condition.

The terms “administration of” and or “administering a” compound should be understood to mean providing a compound of the invention or a prodrug of a compound of the invention to the individual or mammal in need of treatment.

The administration of the compound of structural Formula (I) in order to practice the present methods of therapy is carried out by administering an effective amount of the compound of structural Formula (I) to the mammal in need of such treatment or prophylaxis. The need for a prophylactic administration according to the methods of the present invention is determined via the use of well-known risk factors. The effective amount of an individual compound is determined, in the final analysis, by the physician or veterinarian in charge of the case but depends on factors such as the exact disease to be treated, the severity of the disease and other diseases or conditions from which the patient suffers, the chosen route of administration other drugs and treatments which the patient may concomitantly require, and other factors in the physician's judgment.

The usefulness of the present compounds in these diseases or disorders may be demonstrated in animal disease models that have been reported in the literature.

### Administration and Dose Ranges

Any suitable route of administration may be employed for providing a mammal, especially a human, with an effective dose of a compound of the present invention. For example, oral, intravenous, infusion, subcutaneous, transcutaneous, intramuscular, intradermal, transmucosal, intramucosal, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

In the treatment or prevention of disorders, diseases and/or conditions which require inhibition of NLRP3 a suitable dosage level will generally be about 0.0001 to about 500 mg per kg patient body weight per day which can be administered in single or multiple doses. In one embodiment, a suitable dosage level may be about 0.001 to about 500 mg per kg patient body weight per day. In another embodiment, a suitable dosage level may be about 0.001 to about 250 mg/kg per day. In another embodiment, a suitable dosage level may be about 0.01 to about 250 mg/kg per day. In another embodiment, a suitable dosage level may be about 0.1 to about 100 mg/kg per day. In another embodiment, a suitable dosage level may be about 0.05 to about 100 mg/kg per day. In another embodiment, a suitable dosage level may be about 0.1 to about 50 mg/kg per day. In another embodiment, a suitable dosage level may be about 0.05 to about 0.5 mg/kg per day. In another embodiment, a suitable dosage level may be about 0.5 to about 5 mg/kg per day. In another embodiment, a suitable dosage level may be about 5 to about 50 mg/kg per day. For oral administration, the compositions are preferably provided in the form of tablets containing about 0.01 to about 1000 mg of the active ingredient, particularly about 0.01, about 0.025, about 0.05, about 0.075, about 0.1, about 0.25, about 0.5, about 0.75, about 1.0, about 2.5, about 5.0, about 7.5, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1000.0 mg of the active ingredient for the symptomatic adjustment of the dosage to the patient to be treated. The compounds may be administered on a regimen of 1 to 8 times per day; preferably, 1 to 4 times a day; more preferably once or twice per day, even more preferably once a day. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode, and time of administration, rate of excretion, drug combination, the severity of the particular condition, and the host undergoing therapy.

The compounds of this invention may be used in pharmaceutical compositions comprising (a) the compound(s) or pharmaceutically acceptable salts thereof, and (b) a pharmaceutically acceptable carrier. The compounds of this invention may be used in pharmaceutical compositions in which the compound of the present invention or a pharmaceutically acceptable salt thereof is the only active ingredient. The compounds of this invention may also be used in pharmaceutical compositions that include one or more other active pharmaceutical ingredients.

The term “composition,” as in pharmaceutical composition, is intended to encompass a product comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

Compounds of the present invention may be used in combination with other drugs that may also be useful in the treatment or amelioration of the diseases or conditions for which compounds of the present invention are useful. Such other drugs may be administered, by a route and in an amount commonly used therefor, contemporaneously or sequentially with a compound of the present invention. In the treatment of patients who suffer from chronic inflammatory conditions, more than one drug may be administered. The compounds of this invention may generally be administered to a patient who is already taking one or more other drugs for these conditions. Often the compounds will be administered to a patient who is already being treated with one or more anti-pain compounds when the patient's pain is not adequately responding to treatment.

The combination therapy also includes therapies in which the compound of the present invention and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of the present invention.

Examples of other active ingredients that may be administered in combination with a compound of the present invention, and either administered separately or in the same pharmaceutical composition, include but are not limited to:
1. anti-steatotic agents;
2. anti-inflammatory agents;
3. immunooncology agent;
4. lipid-lowering agents;
5. cholesterol lowering agents;
6. glucose-lowering agents, including SGLT2 inhibitors;
7. anti-neovascular agents;
8. nonsteroidal anti-inflammatory drugs (“NSAIDs”);
9. acetyl-salicylic acid drugs (ASA) including aspirin;
10. paracetamol;
11. regenerative therapy treatments;
12. checkpoint inhibitors including anti-PD1 and anti-PDL1 inhibitors;
13. chemotherapy procedures;
14. radiation therapy;
15. surgical procedures;
16. urate-lowering therapy;
17. anabolics and cartilage regenerative therapy;
18. anti-fibrotics;
19. JAK inhibitors;
20. TNF-alpha inhibitors;
21. anti-hypertensive agents; and
22. STING/cGAS antagonists, and
   pharmaceutically acceptable salts thereof.

In another embodiment, the pharmaceutical composition comprises:
1. a compound as disclosed herein, or a pharmaceutically acceptable salt thereof;
2. one or more compounds, or pharmaceutically acceptable salts thereof, selected from the group:
   a. anti-steatotic agents;
   b. anti-inflammatory agents;
   c. immunooncology agent;
   d. lipid-lowering agents;
   e. cholesterol lowering agents;
   f. glucose-lowering agents, including SGLT2 inhibitors;
   g. anti-neovascular agents;
   h. nonsteroidal anti-inflammatory drugs (“NSAIDs”);
   i. acetyl-salicylic acid drugs (ASA) including aspirin;
   j. paracetamol;
   k. regenerative therapy treatments;
   l. checkpoint inhibitors including anti-PD1 and anti-PDL1 inhibitors;
   m. chemotherapy procedures;
   n. radiation therapy;
   o. surgical procedures;
   p. urate-lowering therapy;
   q. anabolics and cartilage regenerative therapy;
   r. anti-fibrotics;
   s. JAK inhibitors;
   t. TNF-alpha inhibitors;
   u. anti-hypertensive agents; and
   v. STING/cGAS antagonists; and
   w. pharmaceutically acceptable salts thereof; and
3. a pharmaceutically acceptable carrier.

Specific compounds of use in combination with a compound of the present invention include: anti-steatotic agents, including but not limited to, DGAT2 inhibitors.

Suitable anti-inflammatory agents include, but are not limited to, TNFα inhibitors, JAK inhibitors and NSAIDs.

Suitable lipid-lowering agents include, but are not limited to, statins and PCSK9.

Suitable immune-oncology agents include, but are not limited to, PD-L1 inhibitors and PD-1 inhibitors, and STING antagonists.

Suitable glucose-lowering agents include, but are not limited to, insulin, SGLT2 inhibitors, metformin, and GLP1-agonists.

Suitable anti-neovascular agents include, but are not limited to, anti-VEG-F treatment.

Suitable NSAIDs or non-steroidal anti-inflammatory drugs include, but are not limited to, aspirin, diclofenac, diflunisal, etodolac, fenoprofin, flurbiprofen, ibuprofen, indomethacin, ketoprofen, meclofenamic acid, mefenamic acid, meloxicam, naproxen, naproxen sodium, oxaprozin, piroxicam, sulindac, and tolmetin.

Suitable analgesics include, but are not limited to, acetaminophen and duloxetine.

The above combinations include combinations of a compound of the present invention not only with one other active compound, but also with two or more other active compounds. Non-limiting examples include combinations of compounds with two or more active compounds selected from: anti-steatotic agents, anti-inflammatory agents, lipid-lowering agents, anti-fibrosis, immunooncology agents, glucose-lowering agents and anti-neovascular agents, NSAIDs (non-steroidal anti-inflammatory drugs), and analgesics.

The present invention also provides a method for the treatment or prevention of a NLRP3 mediated disease, disorder or condition, which method comprises administration to a patient in need of such treatment or at risk of developing a NLRP3 mediated disease with a therapeutically effective amount of a NLRP3 inhibitor and an amount of one or more active ingredients, such that together they give effective relief.

In a further aspect of the present invention, there is provided a pharmaceutical composition comprising a NLRP3 inhibitor and one or more active ingredients, together with at least one pharmaceutically acceptable carrier or excipient.

Thus, according to a further aspect of the present invention there is provided the use of a NLRP3 inhibitor and one or more active ingredients for the manufacture of a medicament for the treatment or prevention of an NLRP3-mediated disease, disorder, or condition. In a further or alternative aspect of the present invention, there is therefore provided a product comprising a NLRP3 inhibitor and one or more active ingredients as a combined preparation for simultaneous, separate, or sequential use in the treatment or prevention of an NLRP3-mediated disease, disorder, or condition. Such a combined preparation may be, for example, in the form of a twin pack.

It will be appreciated that for the treatment or prevention of cardiometabolic disease, neurodegenerative disease, inflammatory joint diseases, fibrosis, or cancer, a compound of the present invention may be used in conjunction with another pharmaceutical agent effective to treat that disease, disorder, or condition.

The present invention also provides a method for the treatment or prevention of chronic inflammatory conditions, which method comprises administration to a patient in need of such treatment an amount of a compound of the present invention and an amount of another pharmaceutical agent effective to threat that disorder, disease, or condition, such that together they give effective relief.

The present invention also provides a method for the treatment or prevention of chronic inflammatory conditions, which method comprises administration to a patient in need of such treatment an amount of a compound of the present invention and an amount of another pharmaceutical agent useful in treating that particular condition, disorder, or disease, such that together they give effective relief.

The term “therapeutically effective amount” means the amount the compound of structural Formula (I) that will elicit the biological or medical response of a cell, tissue, system, animal, or human that is being sought by the researcher, veterinarian, medical doctor, or other clinician, which includes alleviation of the symptoms of the disorder being treated. The novel methods of treatment of this invention are for disorders known to those skilled in the art. The term “mammal” includes humans, and companion animals such as dogs and cats.

The weight ratio of the compound of the Formula (I) to the second active ingredient may be varied and will depend upon the effective dose of each ingredient. Generally, an effective dose of each will be used. Thus, for example, when a compound of the Formula (I) is combined with an anti-steatotic agent, the weight ratio of the compound of the Formula (I) generally range from about 1000:1 to about 1:1000, preferably about 200:1 to about 1:200. Combinations of a compound of the Formula (I) and other active ingredients will generally also be within the aforementioned range, but in each case, an effective dose of each active ingredient should be used.

### Exemplary Embodiments

Embodiments of the disclosure include but are not limited to the following exemplary embodiments.

A first exemplary embodiment is directed to a compound of Formula (I):

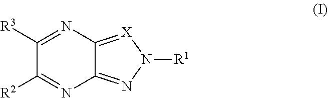

or a pharmaceutically acceptable salt thereof, wherein
X is selected from:
1) CR⁵, and
2) N;
R¹ is selected from:
1) C₁₋₆ alkyl,
2) C₀₋₃ alkylene-(monocyclic C₃₋₈ cycloalkyl),
3) C₀₋₃ alkylene-(bicyclic C₄₋₈ cycloalkyl),
4) C₀₋₃ alkylene-(C₅₋₁₀ spiro-cycloalkyl),
5) C₀₋₃ alkylene-(tricyclic C₆₋₉ cycloalkyl),
6) C₀₋₃ alkylene-(cubyl),
7) C₀₋₃ alkylene-(3- to 9-membered monocyclic heterocycloalkyl containing 1, 2, or 3 ring heteroatoms selected from N, S, O, and P),
8) C₀₋₃ alkylene-(4- to 9-membered bicyclic heterocycloalkyl containing 1, 2, or 3 ring heteroatoms selected from N, S, O, and P),
9) C₀₋₃ alkylene-(5- to 9-membered spirocyclic heterocycloalkyl containing 1, 2, or 3 ring heteroatoms selected from N, S, O, and P),
10) C₀₋₃ alkylene-(5- to 10-membered heteroaryl containing 1, 2, or 3 ring heteroatoms selected from N, S, O, and P),
11) NH—C₁₋₆ alkyl,
12) NH—C₀₋₃ alkylene-(monocyclic C₃₋₈ cycloalkyl),
13) NH—C₀₋₃ alkylene-(bicyclic C₄₋₈ cycloalkyl),
14) NH—C₀₋₃ alkylene-(C₅₋₁₀ spiro-cycloalkyl), and
15) NH—C₀₋₃ alkylene-(tricyclic C₆₋₉ cycloalkyl),
wherein said R¹ group is unsubstituted or substituted with 1 to 5 substituents independently selected from R⁶;
   R² is phenyl substituted by from 0 to 3 substituents independently selected from:
1) OH,
2) halo,
3) C₁₋₆ alkyl, and
4) C₁₋₆ haloalkyl containing from 1 to 3 independently selected halogens;
   R³ is selected from:
1) H,
2) halo, and
3) C₁₋₆ alkyl;
   R⁵ is selected from:
1) H,
2) OH,
3) CN,
4) C₁₋₆ alkyl,
5) C₁₋₆ alkoxy, and
6) C₃₋₇ cycloalkyl,
wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
   1) OH,
   2) halo,
   3) CN,
   4) NR^{a}₂,
   wherein each R^{a} is independently selected from H and C₁₋₆ alkyl; and each R⁶ is independently selected from:
      1) OH,
      2) halo,
      3) CN,
      4) oxo,

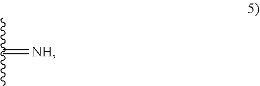 [SMILES: *=N]

6) C₁₋₆ alkyl, which may be straight or branched and which is unsubstituted or substituted with 1 to 4 substituents selected from OH, halo, CN, oxo, and C₁₋₆ alkoxy,
7) C₁₋₆ alkoxy, which may be straight or branched and which is unsubstituted or substituted with 1 to 4 substituents selected from OH, halo, CN, oxo, and C₁₋₆ alkoxy,
8) C₃₋₇ cycloalkyl, which is unsubstituted or substituted with 1 to 4 substituents selected from OH, halo, CN, oxo, C₁₋₃ alkyl, C₁₋₃ haloalkyl, and C₁₋₆ alkoxy,
9) 3- to 9-membered monocyclic heterocycloalkyl containing 1, 2, or
3 ring heteroatoms selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy,
10) C₅₋₇ aryl, which is unsubstituted or substituted with 1 to 4 substituents selected from OH, halo, CN, oxo, and C₁₋₆ alkoxy,
11) methylene-C₅₋₇ aryl, which is unsubstituted or substituted with 1 to 4 substituents selected from OH, halo, CN, oxo, and C₁₋₆ alkoxy, and
12) 5- to 10-membered heteroaryl containing at least one ring heteroatom selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy.

A second exemplary embodiment is directed to a compound of Formula (I):

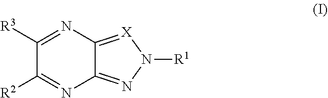

or a pharmaceutically acceptable salt thereof, wherein
X is selected from:
1) CR⁵, and
2) N;
   R¹ is selected from:
1) C₁₋₆ alkyl,
2) C₀₋₃ alkylene-(monocyclic C₃₋₈ cycloalkyl),
3) C₀₋₃ alkylene-(bicyclic C₄₋₈ cycloalkyl),
4) C₀₋₃ alkylene-(C₅₋₁₀ spiro-cycloalkyl),
5) C₀₋₃ alkylene-(tricyclic C₆₋₉ cycloalkyl),
6) C₀₋₃ alkylene-(3- to 9-membered monocyclic heterocycloalkyl containing 1, 2, or 3 ring heteroatoms selected from N, S, O, and P),
7) C₀₋₃ alkylene-(4- to 9-membered bicyclic heterocycloalkyl containing 1, 2, or 3 ring heteroatoms selected from N, S, O, and P),
8) C₀₋₃ alkylene-(5- to 9-membered spirocyclic heterocycloalkyl containing 1, 2, or 3 ring heteroatoms selected from N, S, O, and P), and
9) C₀₋₃ alkylene-(5- to 10-membered heteroaryl containing 1, 2, or 3 ring heteroatoms selected from N, S, O, and P),
wherein said R¹ group is unsubstituted or substituted with 1 to 5 substituents independently selected from R⁶;
   R² is phenyl substituted by from 0 to 3 substituents independently selected from:
1) OH,
2) halo,
3) C₁₋₆ alkyl, and
4) C₁₋₆ haloalkyl containing from 1 to 3 independently selected halogens;
   R³ is selected from:
1) H,
2) halo, and
3) C₁₋₆ alkyl;
   R⁵ is selected from:
1) H,
2) OH,
3) CN,
4) C₁₋₆ alkyl,
5) C₁₋₆ alkoxy, and
6) C₃₋₇ cycloalkyl,
wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
   1) OH,
   2) halo,
   3) CN,
   4) NR^{a}₂,
   wherein each R^{a} is independently selected from H and C₁₋₆ alkyl; and each R⁶ is independently selected from:
1) OH,
2) halo,
3) CN,
4) oxo,

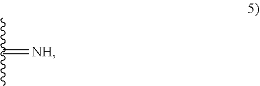 [SMILES: *=N]

6) C₁₋₆ alkyl, which may be straight or branched and which is unsubstituted or substituted with 1 to 4 substituents selected from OH, halo, CN, oxo, and C₁₋₆ alkoxy,
7) C₁₋₆ alkoxy, which may be straight or branched and which is unsubstituted or substituted with 1 to 4 substituents selected from OH, halo, CN, oxo, and C₁₋₆ alkoxy,
8) C₃₋₇ cycloalkyl, which is unsubstituted or substituted with 1 to 4 substituents selected from OH, halo, CN, oxo, C₁₋₃ alkyl, C₁₋₃ haloalkyl, and C₁₋₆ alkoxy,
9) 3- to 9-membered monocyclic heterocycloalkyl containing 1, 2, or 3 ring heteroatoms selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy,
10) C₅₋₇ aryl, which is unsubstituted or substituted with 1 to 4 substituents selected from OH, halo, CN, oxo, and C₁₋₆ alkoxy,
11) methylene-C₅₋₇ aryl, which is unsubstituted or substituted with 1 to 4 substituents selected from OH, halo, CN, oxo, and C₁₋₆ alkoxy, and
12) 5- to 10-membered heteroaryl containing at least one ring heteroatom selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy.

A third exemplary embodiment is directed to a compound, or a pharmaceutically acceptable salt thereof, according to any prior embodiment, wherein X is N.

A fourth exemplary embodiment is directed to a compound, or a pharmaceutically acceptable salt thereof, according to any prior embodiment, wherein X is CR⁵.

A fifth exemplary embodiment is directed to a compound, or a pharmaceutically acceptable salt thereof, according to any prior embodiment, wherein R⁵ is selected from:
1) H,
2) OH,
3) CN,
4) C₁₋₃ alkyl,
5) C₁₋₃ alkoxy, and
6) C₃₋₄ cycloalkyl,
wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
   1) OH,
   2) C₁,
   3) F,
   4) CN,
   5) NH₂, and
   6) CH₃.

A sixth exemplary embodiment is directed to a compound, or a pharmaceutically acceptable salt thereof, according to any prior embodiment, wherein R⁵ is selected from the group:
1) H,
2) CH₃, and
3) CH(OH)(CH₃).

A sixth exemplary embodiment is directed to a compound, or a pharmaceutically acceptable salt thereof, according to any prior embodiment, wherein R⁵ is H.

A seventh exemplary embodiment is directed to a compound, or a pharmaceutically acceptable salt thereof, according to any prior embodiment, wherein R¹ is selected from:
1) C₁₋₆ alkyl,
2) C₀₋₃ alkylene-(monocyclic C₃₋₈ cycloalkyl),
3) C₀₋₃ alkylene-(bicyclic C₄₋₈ cycloalkyl),
4) C₀₋₃ alkylene-(C₅₋₁₀ spiro-cycloalkyl),
5) C₀₋₃ alkylene-(tricyclic C₆₋₉ cycloalkyl),
6) C₀₋₃ alkylene-(3- to 9-membered monocyclic heterocycloalkyl containing 1, 2, or 3 ring heteroatoms selected from N, S, O, and P),
7) C₀₋₃ alkylene-(4- to 9-membered bicyclic heterocycloalkyl containing 1, 2, or 3 ring heteroatoms selected from N, S, O, and P),
8) C₀₋₃ alkylene-(5- to 9-membered spirocyclic heterocycloalkyl containing 1, 2, or 3 ring heteroatoms selected from N, S, O, and P), and
9) C₀₋₃ alkylene-(5- to 10-membered heteroaryl containing 1, 2, or 3 ring heteroatoms selected from N, S, O, and P), and
wherein said R¹ group is unsubstituted or substituted with 1 to 5 substituents independently selected from R⁶, and
   each R⁶ is independently selected from:
1) OH,
2) F,
3) CN,
4) oxo,
5) C₁₋₃ alkyl, which may be straight or branched and which is unsubstituted or substituted with 1 to 4 substituents selected from OH, F, CN, oxo, and OCH₃,
6) C₃ cycloalkyl, which is unsubstituted, and
7) methylene-C₅₋₇ aryl, which is unsubstituted or substituted with 1 to 4 substituents selected from OH, F, CN, oxo, and OCH₃.

An eighth exemplary embodiment is directed to a compound, or a pharmaceutically acceptable salt thereof, according to any prior embodiment, wherein R¹ is selected from:
1) C₁₋₄ alkyl,
2) monocyclic C₃₋₆ cycloalkyl,
3) methylene-(monocyclic C₃₋₆ cycloalkyl),
4) bicyclic C₅₋₈ cycloalkyl,
5) 5- to 6-membered monocyclic heterocycloalkyl containing 1 or 2 ring heteroatoms selected from N, S, and O,
6) methylene-(5- to 6-membered monocyclic heterocycloalkyl containing 1 or 2 ring heteroatoms selected from N, S, and O),
7) 6- to 9-membered bicyclic heterocycloalkyl containing 1, 2, or 3 ring heteroatoms selected from N, S, and O,
8) methylene-(4- to 9-membered bicyclic heterocycloalkyl containing 1, 2, or 3 ring heteroatoms selected from N, S, and O), and
wherein said R¹ group is unsubstituted or substituted with 1 to 5 substituents independently selected from R⁶, and
   each R⁶ is independently selected from:
1) OH,
2) F,
3) CN,
4) oxo,
5) C₁₋₃ alkyl, which may be straight or branched and which is unsubstituted or substituted with 1 to 4 substituents selected from OH, F, CN, oxo, and OCH₃,
6) C₃ cycloalkyl, which is unsubstituted, and
7) methylene-C₅₋₇ aryl, which is unsubstituted or substituted with 1 to 4 substituents selected from OH, F, CN, oxo, and OCH₃.

A ninth exemplary embodiment is directed to a compound, or a pharmaceutically acceptable salt thereof, according to any prior embodiment, wherein R¹ is selected from:

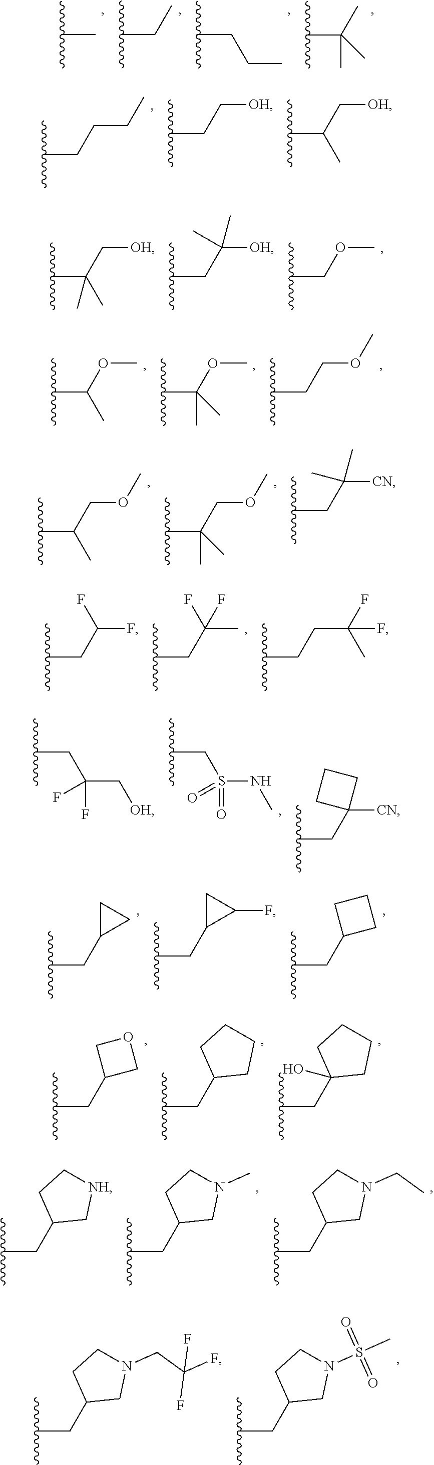

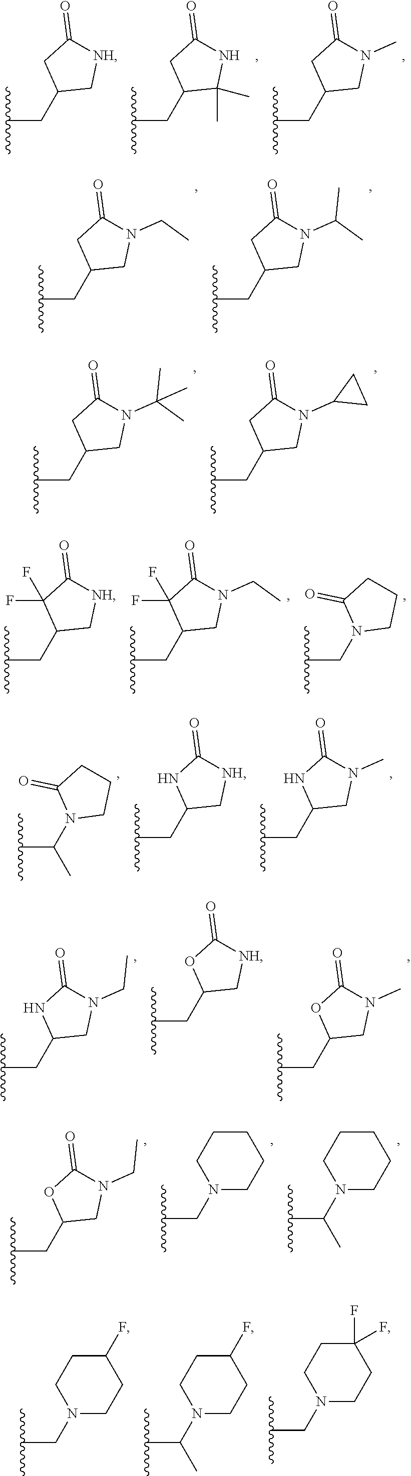

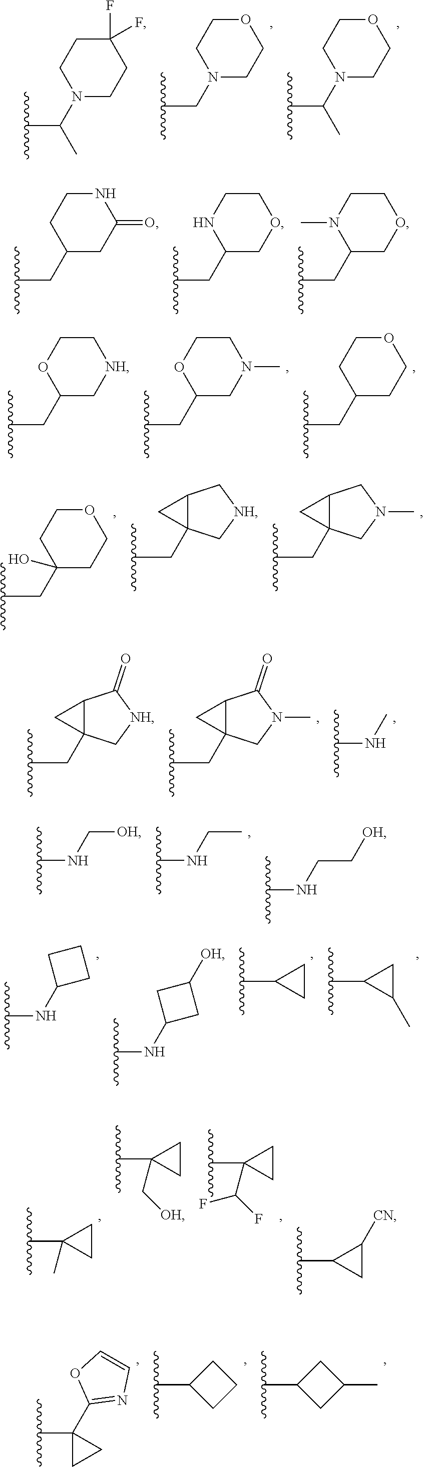

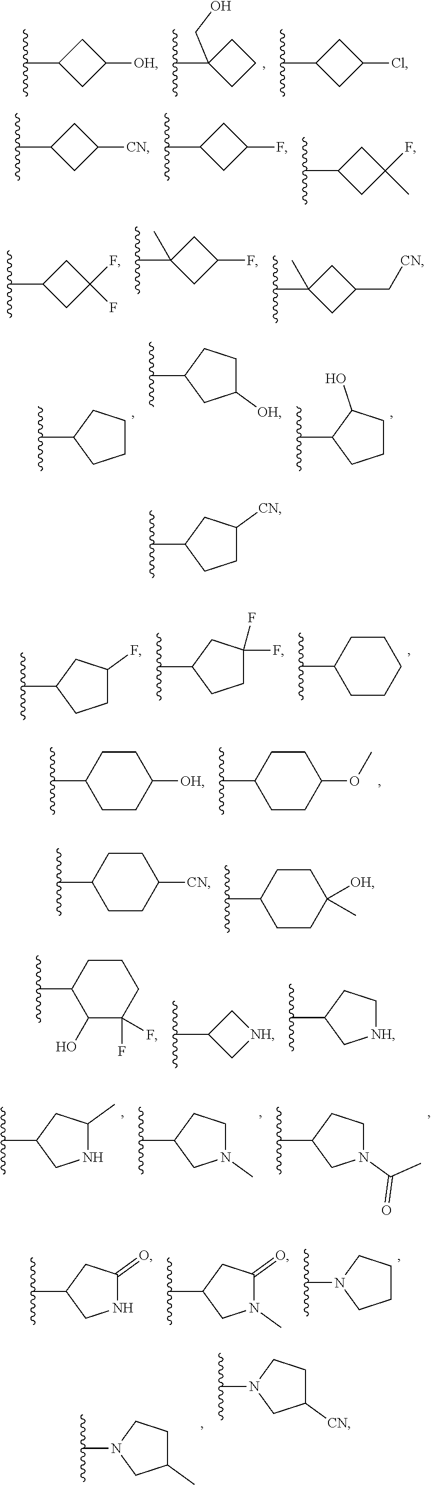

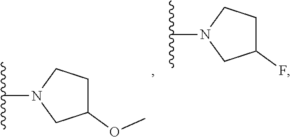

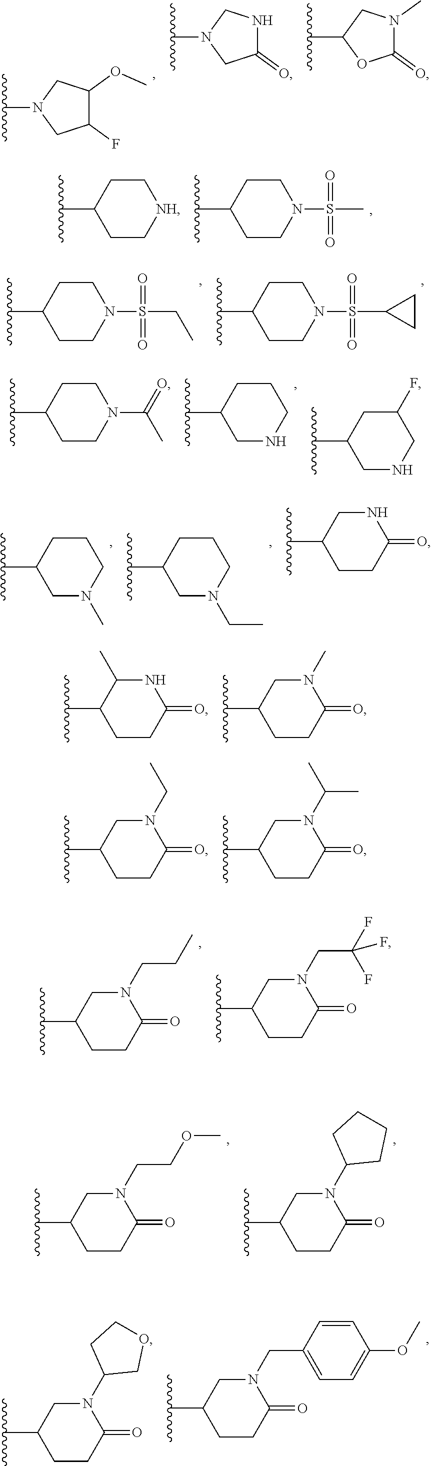

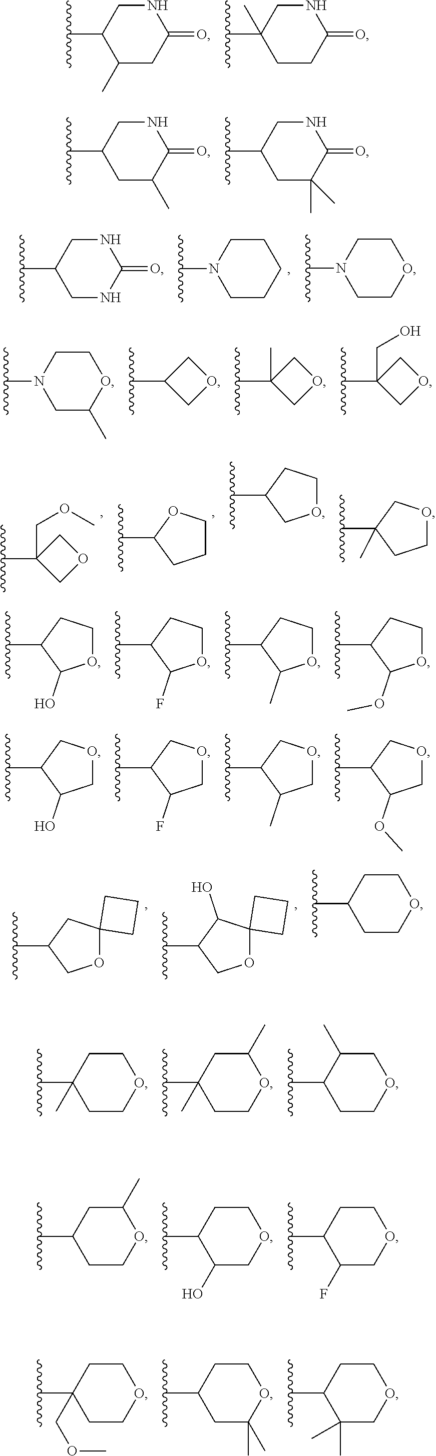

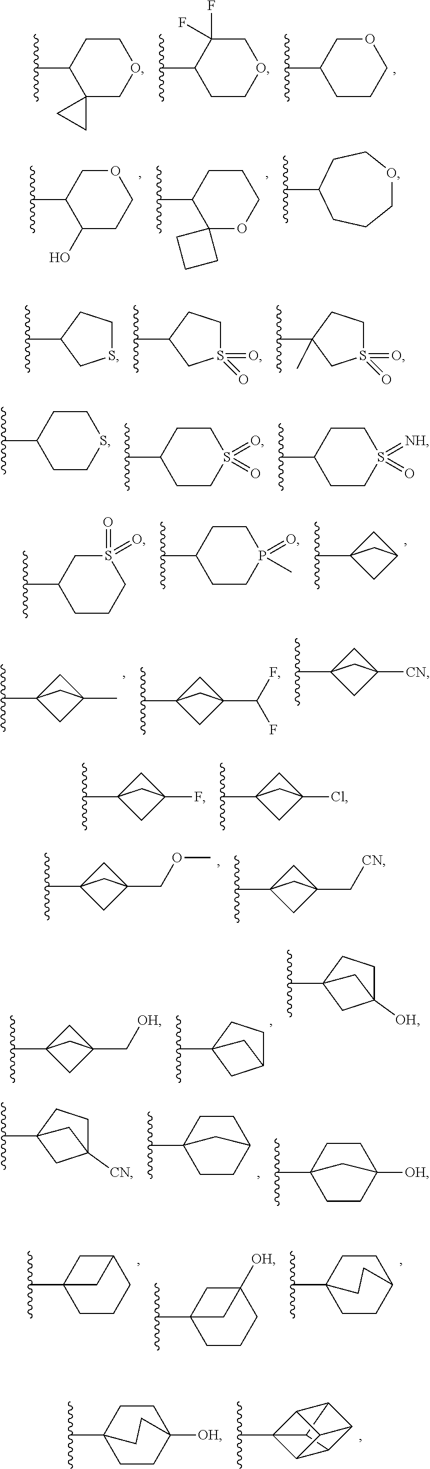

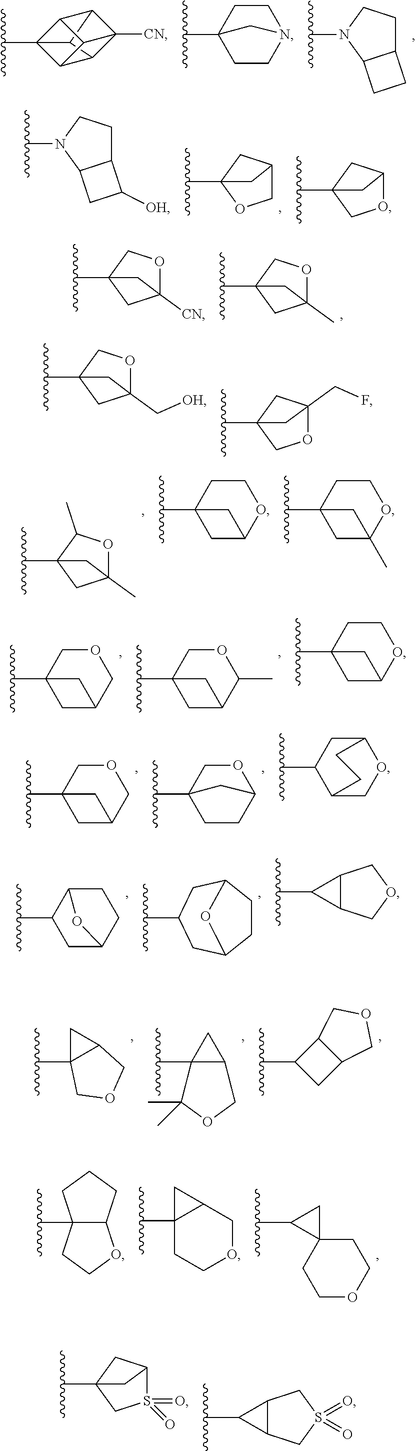

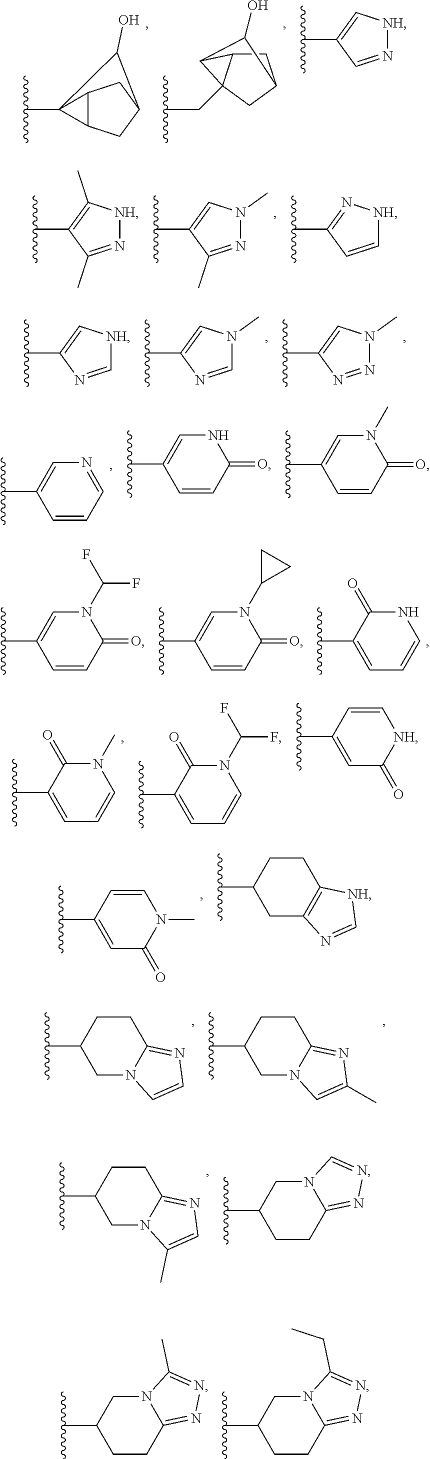

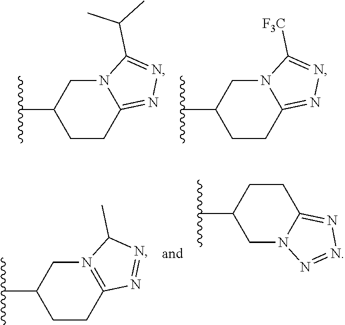

A tenth exemplary embodiment is directed to a compound, or a pharmaceutically acceptable salt thereof, according to any prior embodiment, wherein R¹ is selected from:

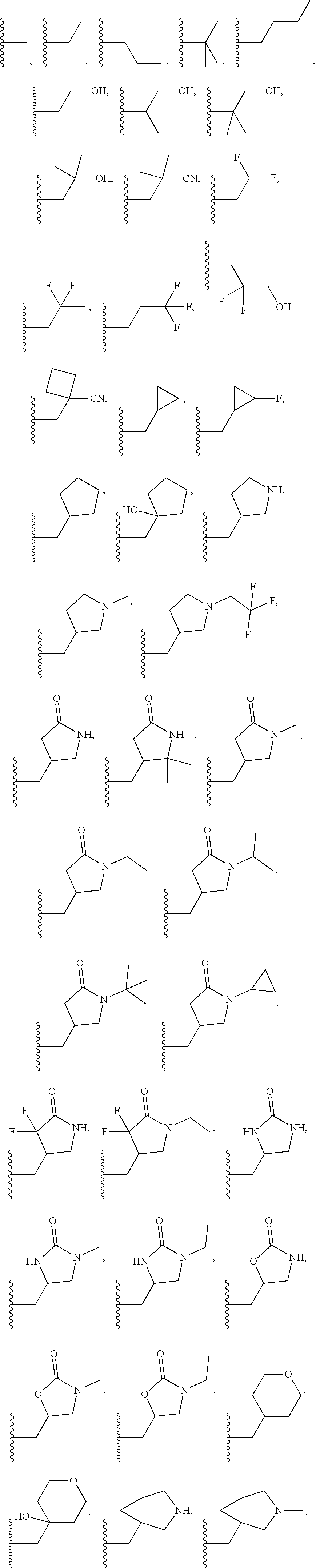

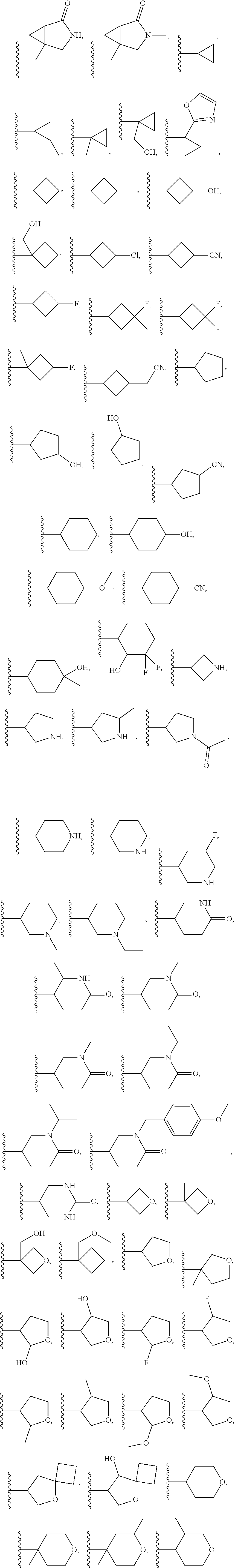

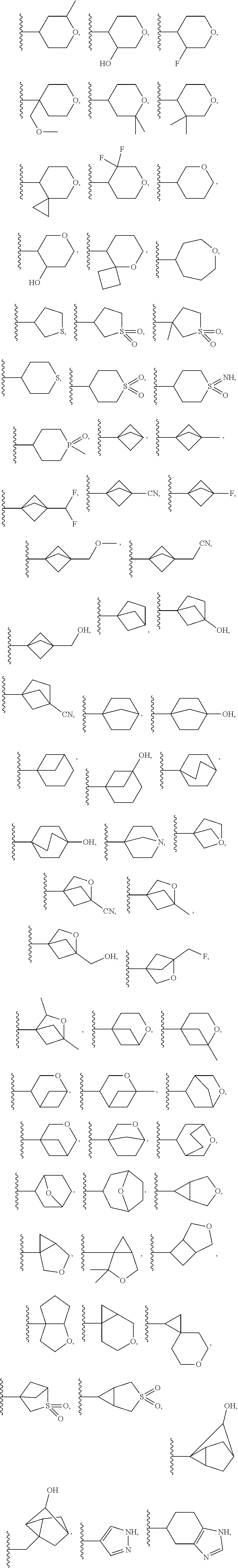

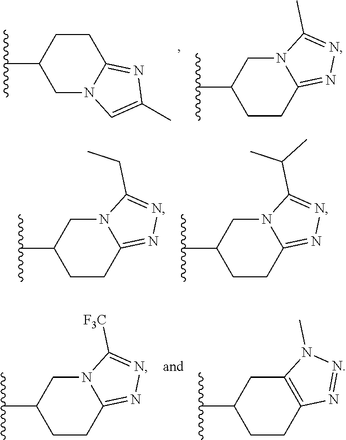

An eleventh exemplary embodiment is directed to a compound, or a pharmaceutically acceptable salt thereof, according to any prior embodiment, wherein R¹ is selected from:

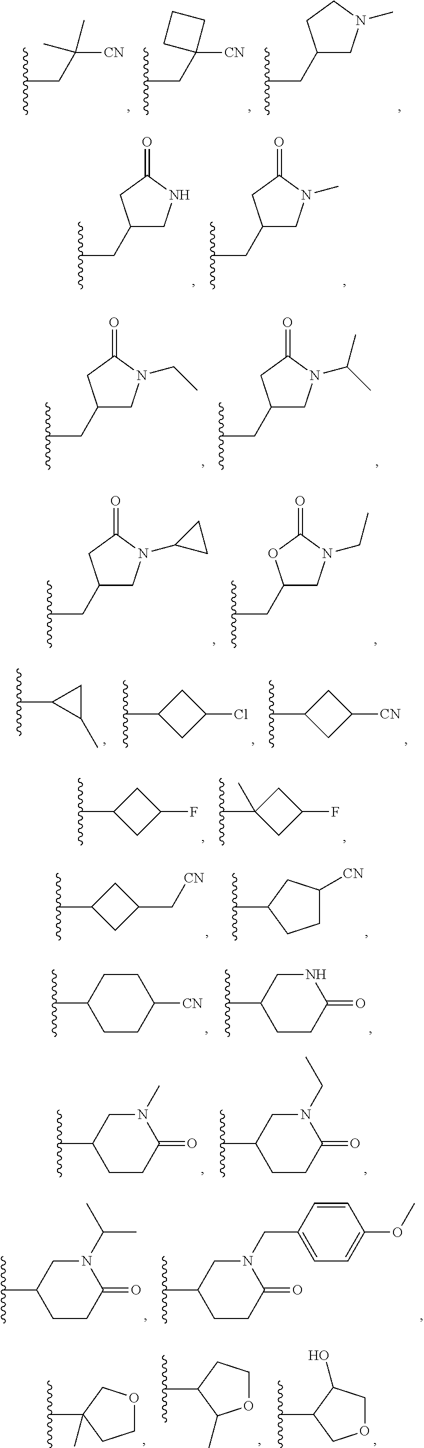

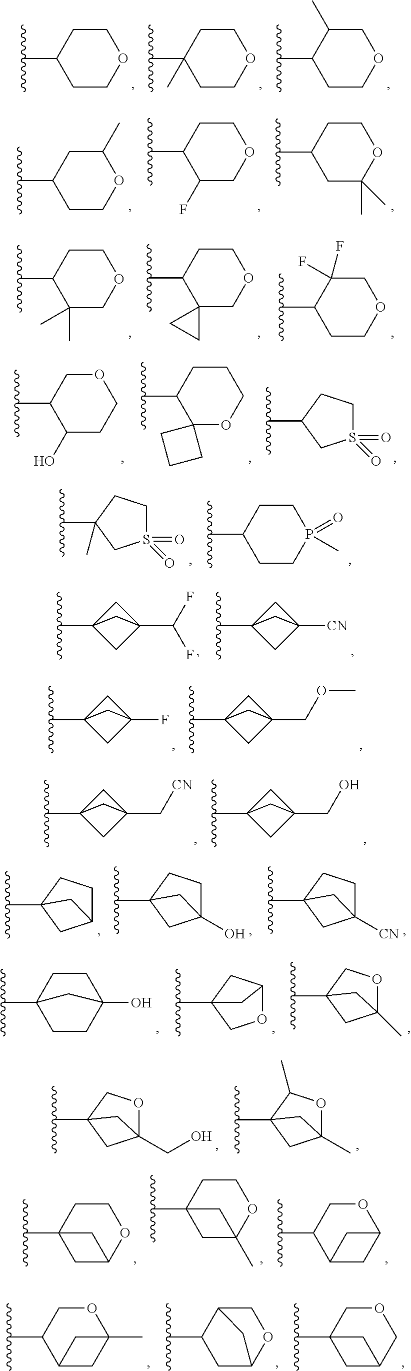

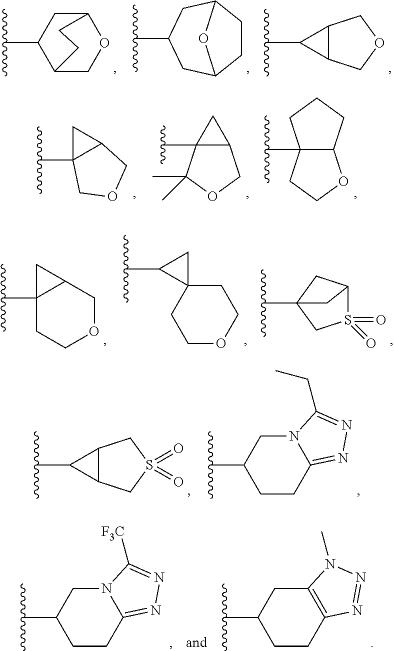

A twelfth exemplary embodiment is directed to a compound, or a pharmaceutically acceptable salt thereof, according to any prior embodiment, wherein R¹ is selected from:

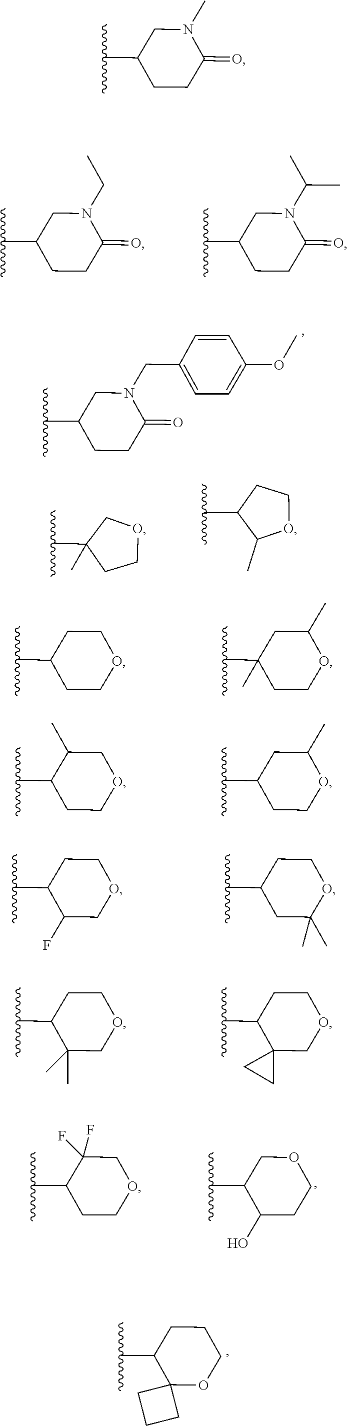

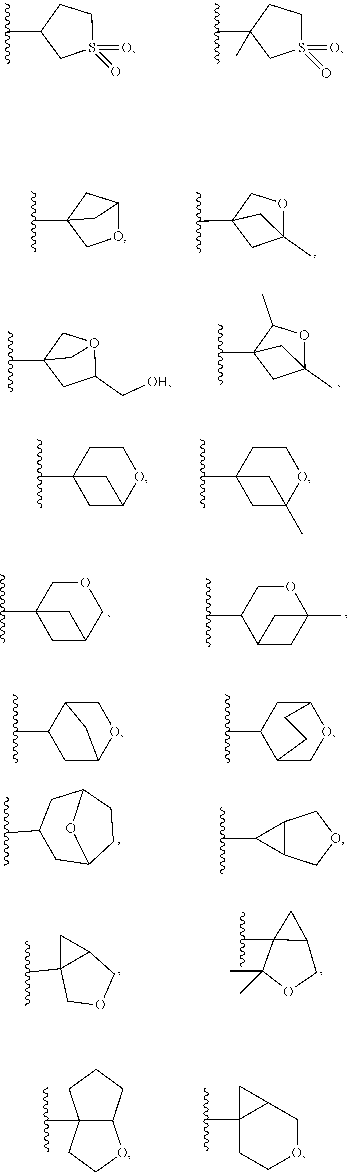

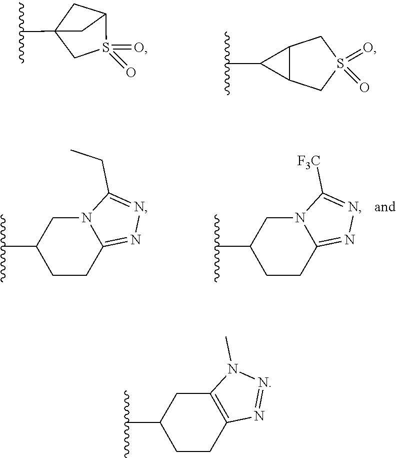 [SMILES: *C12CC(C1)S(=O)(=O)C2 ; *C1C2CS(=O)(=O)CC12 ; *C1CCc2nnc(CC)n2C1 ; *C1CCc2nnc(C(F)(F)F)n2C1 ; *C1CCc2nnn(C)c2C1]

A thirteenth exemplary embodiment is directed to a compound, or a pharmaceutically acceptable salt thereof, according to any prior embodiment, wherein R¹ is selected from:

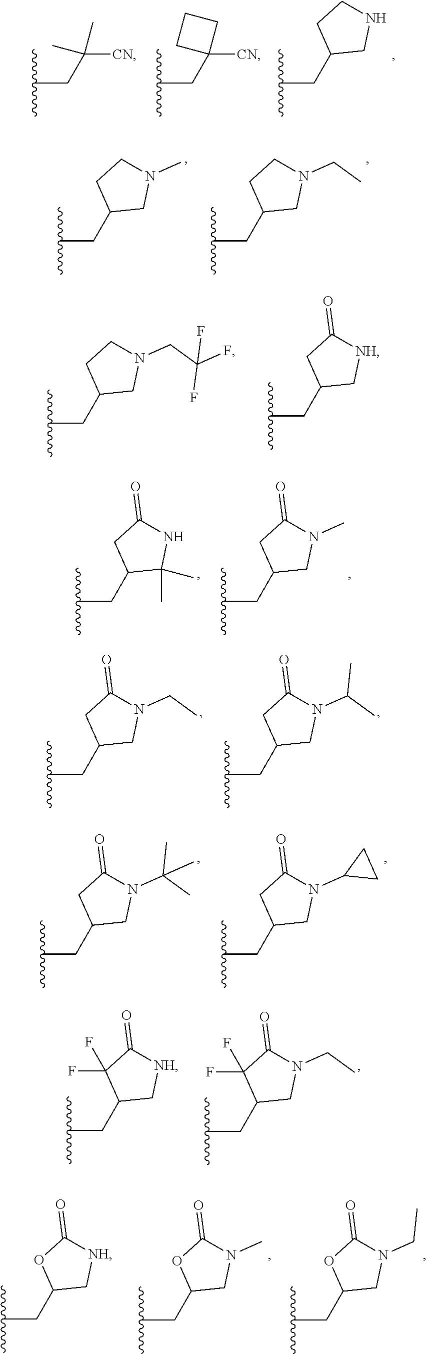

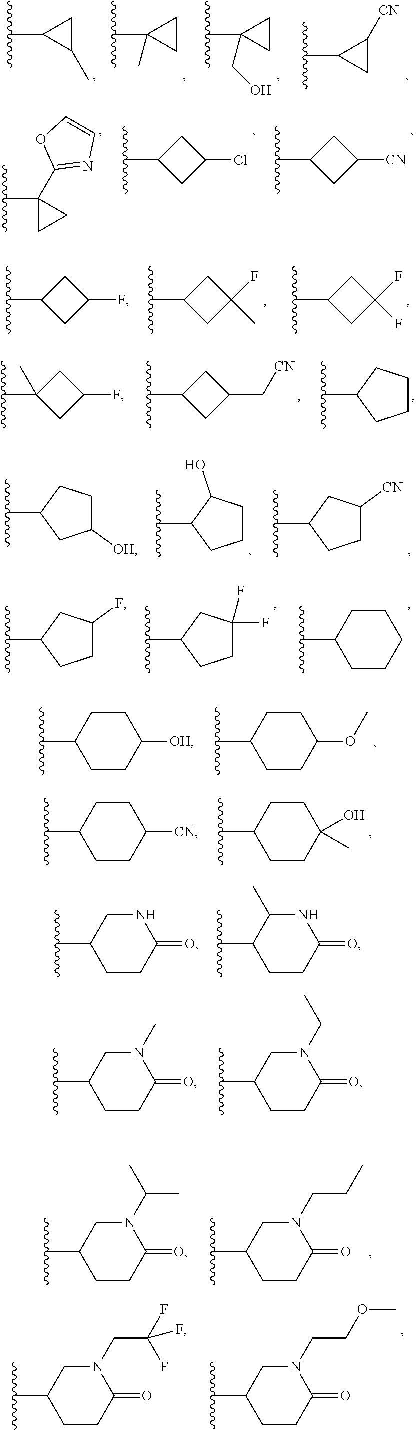

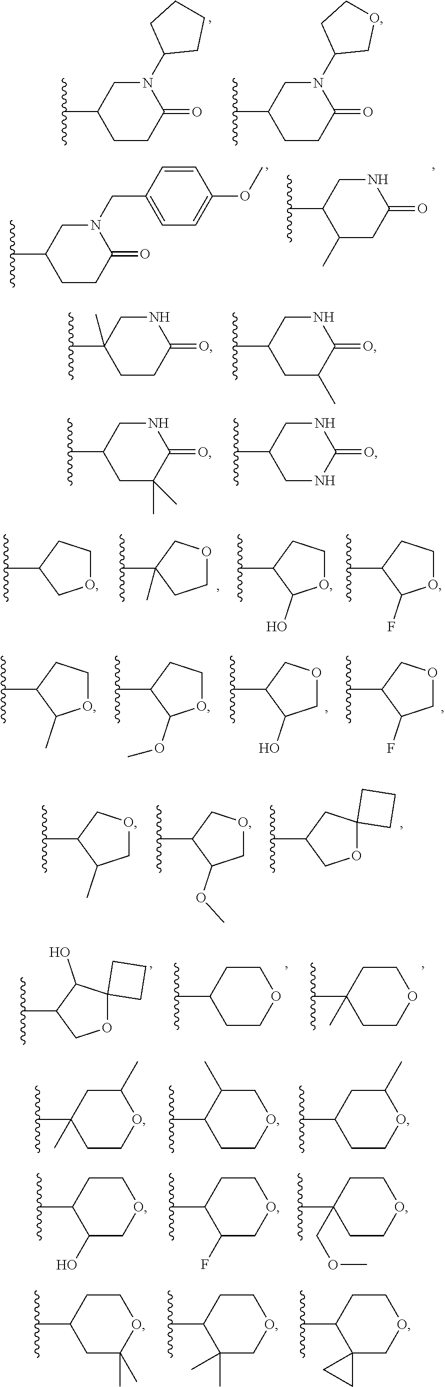

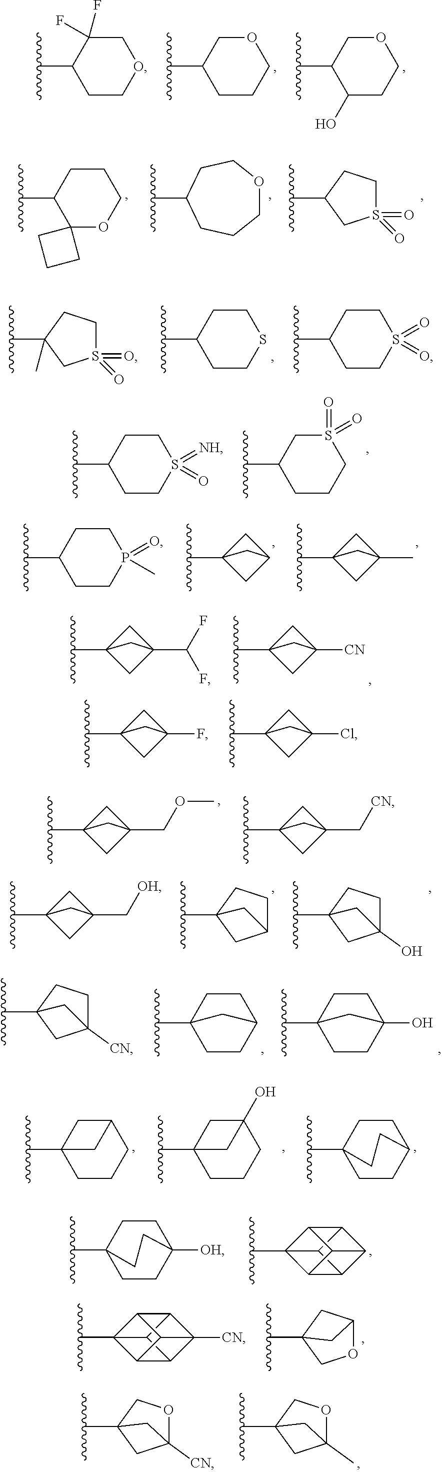

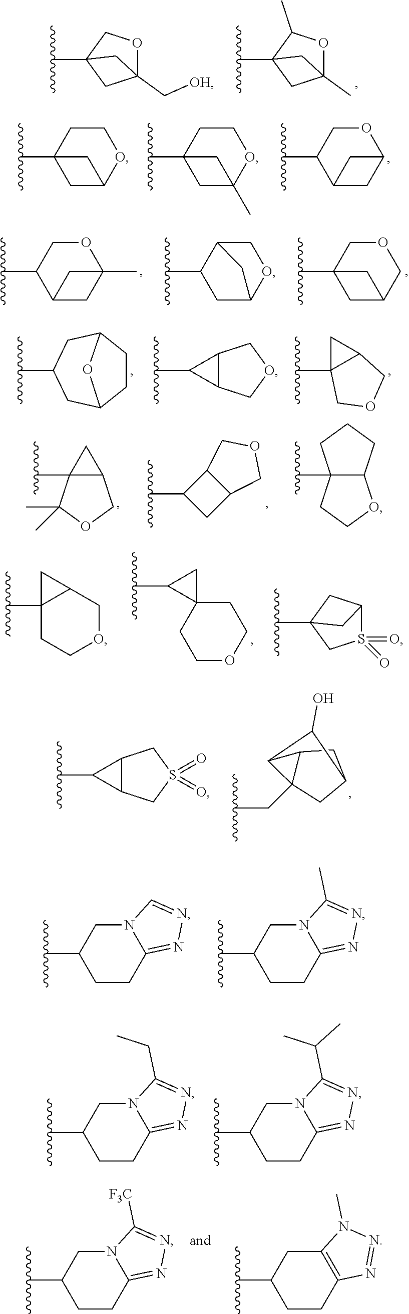

A fourteenth exemplary embodiment is directed to a compound, or a pharmaceutically acceptable salt thereof, according to any prior embodiment, wherein R¹ is selected from:

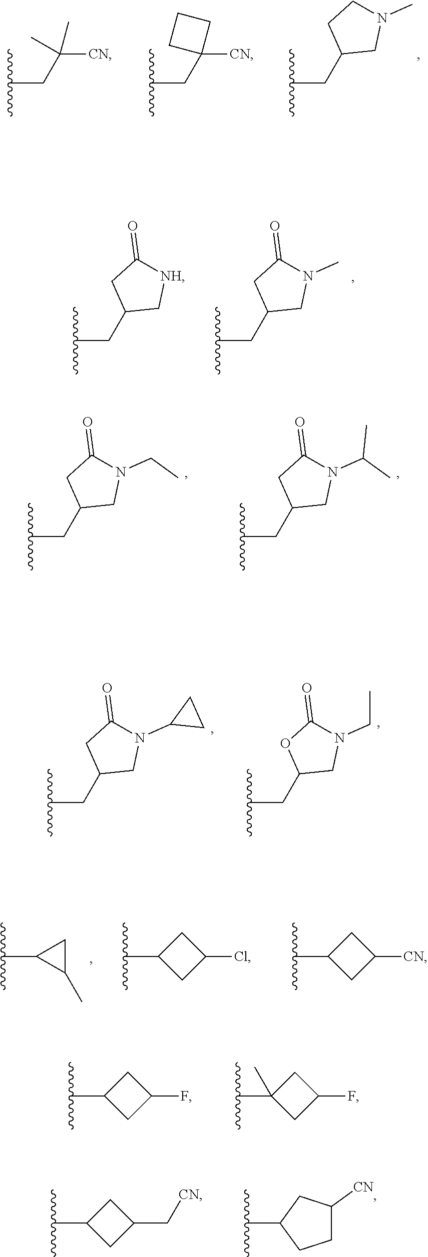

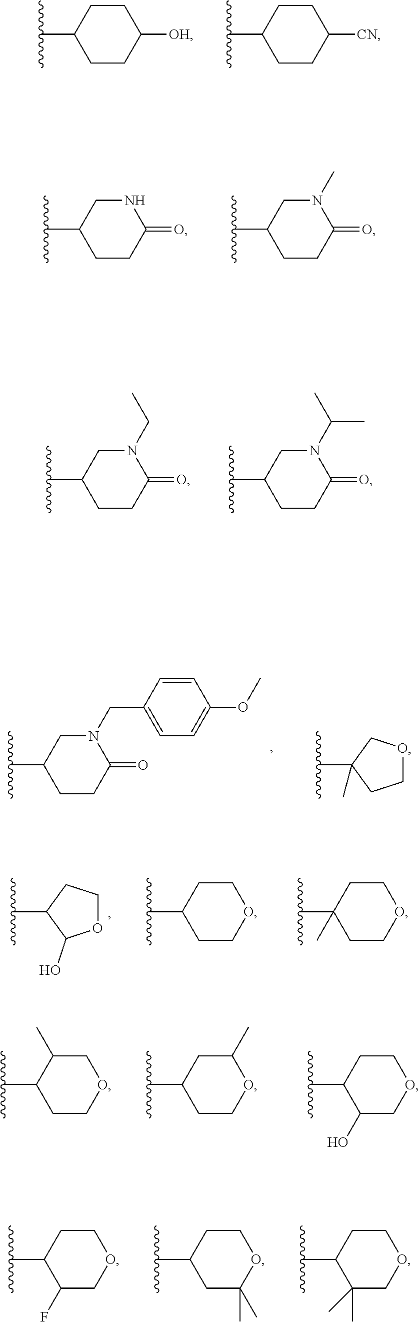

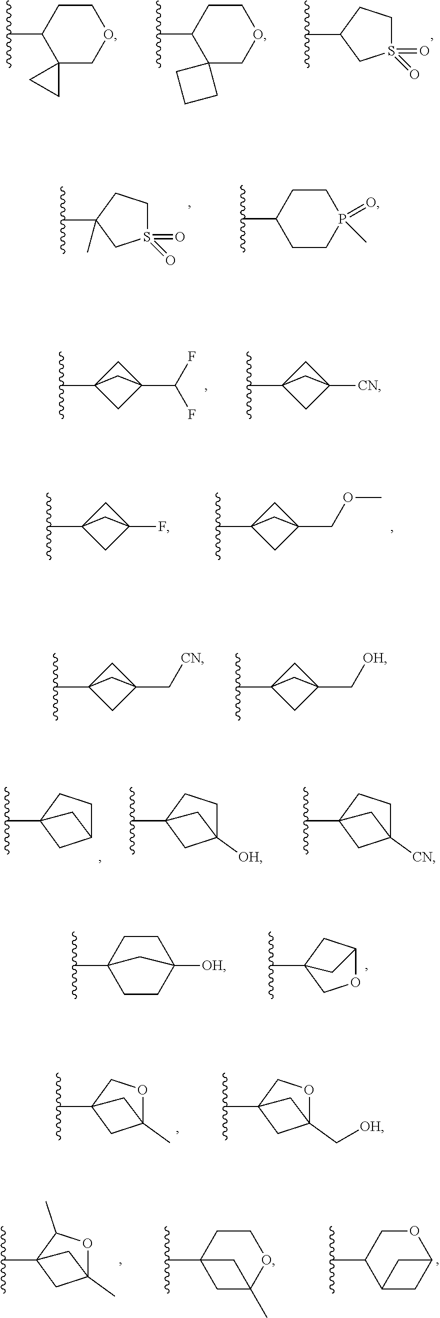

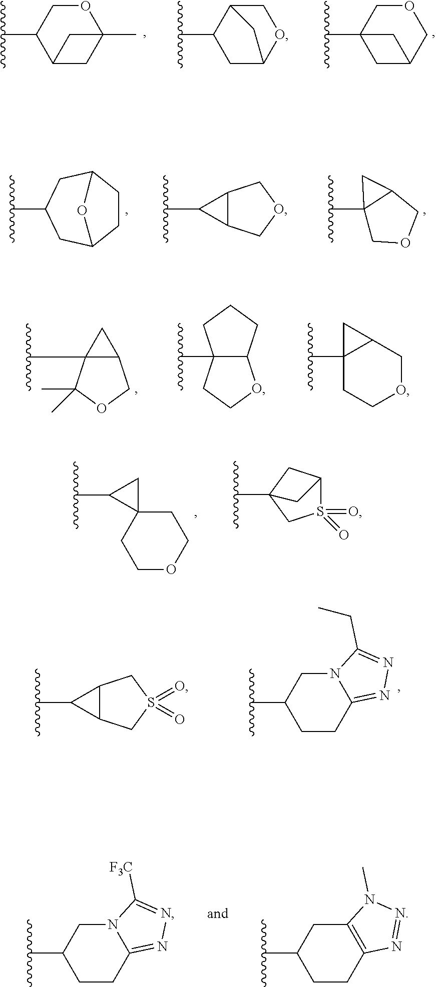

A fifteenth exemplary embodiment is directed to a compound, or a pharmaceutically acceptable salt thereof, according to any prior embodiment, wherein R¹ is selected from:

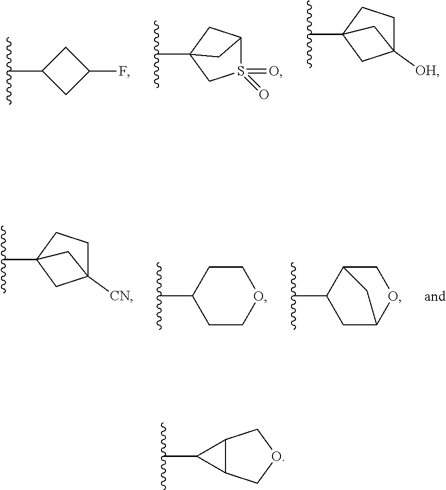

A sixteenth exemplary embodiment is directed to a compound, or a pharmaceutically acceptable salt thereof, according to any prior embodiment, wherein R¹ is selected from:

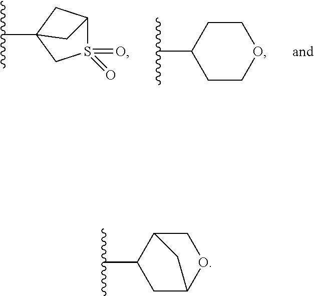

A seventeenth exemplary embodiment is directed to a compound, or a pharmaceutically acceptable salt thereof, according to any prior embodiment, wherein R² is phenyl substituted by 2 or 3 substituents selected independently from:
1) OH,
2) halo,
3) C₁₋₂ alkyl, and
4) C₁₋₂ haloalkyl containing from 1 to 3 independently selected halogens.

An eighteenth exemplary embodiment is directed to a compound, or a pharmaceutically acceptable salt thereof, according to any prior embodiment, wherein R² is phenyl substituted by 2 or 3 substituents selected independently from:
1) OH,
2) C₁,
3) CH₃, and
4) CF₃.

A nineteenth exemplary embodiment is directed to a compound, or a pharmaceutically acceptable salt thereof, according to any prior embodiment, wherein R² is phenyl substituted by OH and CF₃.

A twentieth exemplary embodiment is directed to a compound, or a pharmaceutically acceptable salt thereof, according to any prior embodiment, wherein R² is phenyl substituted by OH, CH₃, and CF₃.

A twenty-first exemplary embodiment is directed to a compound, or a pharmaceutically acceptable salt thereof, according to any prior embodiment, wherein R² is phenyl substituted by OH, CH₃, and Cl.

A twenty-second exemplary embodiment is directed to a compound, or a pharmaceutically acceptable salt thereof, according to any prior embodiment, wherein R² is selected from

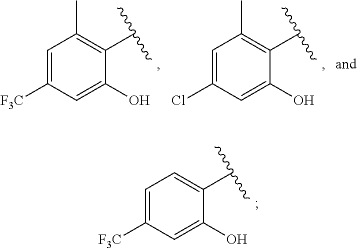

A twenty-third exemplary embodiment is directed to a compound, or a pharmaceutically acceptable salt thereof, according to any prior embodiment, wherein R² is

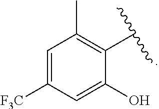 [SMILES: *c1c(C)cc(C(F)(F)F)cc1O]

A twenty-fourth exemplary embodiment is directed to a compound, or a pharmaceutically acceptable salt thereof, according to any prior embodiment, wherein R³ is H.

A twenty-fifth exemplary embodiment is directed to such a compound, or a pharmaceutically acceptable salt thereof, wherein R³ is selected from C₁₋₆ alkyl.

A twenty-sixth exemplary embodiment is directed to a compound, or a pharmaceutically acceptable salt thereof, according to any prior embodiment, wherein R³ is selected from CH₃.

A twenty-seventh exemplary embodiment is directed to a compound, or a pharmaceutically acceptable salt thereof, according to any prior embodiment, wherein the compound is selected from the group consisting of:
1) 3-methyl-2-(2-(tetrahydro-2H-pyran-4-yl)-2H-pyrazolo[3,4-b]pyrazin-6-yl)-5-(trifluoromethyl)phenol,
2) 4-(6-(2-hydroxy-6-methyl-4-(trifluoromethyl)phenyl)-2H-pyrazolo[3,4-b]pyrazin-2-yl)-2-thiabicyclo[2.1.1]hexane 2,2-dioxide,
3) 2-(2-((1S,4R,5R)-2-oxabicyclo[2.2.1]heptan-5-yl)-2H-pyrazolo[3,4-b]pyrazin-6-yl)-3-methyl-5-(trifluoromethyl)phenol,
4) 2-(2-((1R,4S,5S)-2-oxabicyclo[2.2.1]heptan-5-yl)-2H-pyrazolo[3,4-b]pyrazin-6-yl)-3-methyl-5-(trifluoromethyl)phenol,
5) (5S)-5-[6-[2-hydroxy-6-methyl-4-(trifluoromethyl)phenyl]pyrazolo[3,4-b]pyrazin-2-yl]-1-methyl-piperidin-2-one,
6) (R)-3-methyl-2-(2-((1-methylpyrrolidin-3-yl)methyl)-2H-pyrazolo[3,4-b]pyrazin-6-yl)-5-(trifluoromethyl)phenol,
7) (R)-4-((6-(2-hydroxy-6-methyl-4-(trifluoromethyl)phenyl)-2H-pyrazolo[3,4-b]pyrazin-2-yl)methyl)pyrrolidin-2-one,
8) 3-(6-(2-hydroxy-6-methyl-4-(trifluoromethyl)phenyl)-2H-pyrazolo[3,4-b]pyrazin-2-yl)bicyclo[1.1.1]pentane-1-carbonitrile,
9) 2-(2-((1R,5S,6S)-3-oxabicyclo[3.1.0]hexan-6-yl)-2H-pyrazolo[3,4-b]pyrazin-6-yl)-3-methyl-5-(trifluoromethyl)phenol,
10) 2-(2-((1R,5S,6R)-3-oxabicyclo[3.1.0]hexan-6-yl)-2H-pyrazolo[3,4-b]pyrazin-6-yl)-3-methyl-5-(trifluoromethyl)phenol,
11) (1S,4S)-4-(6-(2-hydroxy-6-methyl-4-(trifluoromethyl)phenyl)-2H-pyrazolo[3,4-b]pyrazin-2-yl)cyclohexane-1-carbonitrile,
12) 3-(6-(2-hydroxy-6-methyl-4-(trifluoromethyl)phenyl)-2H-pyrazolo[3,4-b]pyrazin-2-yl)-2,2-dimethylpropanenitrile,
13) 1-((6-(2-hydroxy-6-methyl-4-(trifluoromethyl)phenyl)-2H-pyrazolo[3,4-b]pyrazin-2-yl)methyl)cyclobutane-1-carbonitrile,
14) 2-(2-(3-oxabicyclo[3.1.1]heptan-1-yl)-2H-pyrazolo[3,4-b]pyrazin-6-yl)-3-methyl-5-(trifluoromethyl)phenol,
15) 3-methyl-2-(2-(1-methyl-2-oxabicyclo[3.1.1]heptan-5-yl)-2H-pyrazolo[3,4-b]pyrazin-6-yl)-5-(trifluoromethyl)phenol,
16) 2-(2-((1R,3R)-3-fluorocyclobutyl)-2H-pyrazolo[3,4-b]pyrazin-6-yl)-3-methyl-5-(trifluoromethyl)phenol,
17) 2-(2-(2-oxabicyclo[2.1.1]hexan-4-yl)-2H-pyrazolo[3,4-b]pyrazin-6-yl)-3-methyl-5-(trifluoromethyl)phenol,
18) 2-((1s,3s)-3-(6-(2-hydroxy-6-methyl-4-(trifluoromethyl)phenyl)-2H-pyrazolo[3,4-b]pyrazin-2-yl)cyclobutyl)acetonitrile,
19) (R and S)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
20) (R)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
21) (S)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
22) (1S,3R and 1R,3S)-3-(6-(2-hydroxy-6-methyl-4-(trifluoromethyl)phenyl)-2H-pyrazolo[3,4-b]pyrazin-2-yl)cyclopentane-1-carbonitrile,
23) (1S,3R)-3-(6-(2-hydroxy-6-methyl-4-(trifluoromethyl)phenyl)-2H-pyrazolo[3,4-b]pyrazin-2-yl)cyclopentane-1-carbonitrile,
24) (1R,3S)-3-(6-(2-hydroxy-6-methyl-4-(trifluoromethyl)phenyl)-2H-pyrazolo[3,4-b]pyrazin-2-yl)cyclopentane-1-carbonitrile,
25) 2-(2-((3aS,6aR and 3aR,6aS)-hexahydro-3aH-cyclopenta[b]furan-3a-yl)-2H-pyrazolo[3,4-b]pyrazin-6-yl)-3-methyl-5-(trifluoromethyl)phenol,
26) 2-(2-((3aS,6aR)-hexahydro-3aH-cyclopenta[b]furan-3a-yl)-2H-pyrazolo[3,4-b]pyrazin-6-yl)-3-methyl-5-(trifluoromethyl)phenol,
27) 2-(2-((3aR,6aS)-hexahydro-3aH-cyclopenta[b]furan-3a-yl)-2H-pyrazolo[3,4-b]pyrazin-6-yl)-3-methyl-5-(trifluoromethyl)phenol,
28) (S and R)-2-(2-(5-oxaspiro[3.5]nonan-9-yl)-2H-pyrazolo[3,4-b]pyrazin-6-yl)-3-methyl-5-(trifluoromethyl)phenol,
29) (S)-2-(2-(5-oxaspiro[3.5]nonan-9-yl)-2H-pyrazolo[3,4-b]pyrazin-6-yl)-3-methyl-5-(trifluoromethyl)phenol,
30) (R)-2-(2-(5-oxaspiro[3.5]nonan-9-yl)-2H-pyrazolo[3,4-b]pyrazin-6-yl)-3-methyl-5-(trifluoromethyl)phenol,
31) (S and R)-2-(2-(6-oxaspiro[2.5]octan-1-yl)-2H-pyrazolo[3,4-b]pyrazin-6-yl)-3-methyl-5-(trifluoromethyl)phenol,
32) (S)-2-(2-(6-oxaspiro[2.5]octan-1-yl)-2H-pyrazolo[3,4-b]pyrazin-6-yl)-3-methyl-5-(trifluoromethyl)phenol,
33) (R)-2-(2-(6-oxaspiro[2.5]octan-1-yl)-2H-pyrazolo[3,4-b]pyrazin-6-yl)-3-methyl-5-(trifluoromethyl)phenol,
34) (S and R)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
35) (S)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
36) (R)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
37) (S and R)-2-(2-(2-oxabicyclo[3.1.1]heptan-4-yl)-2H-pyrazolo[3,4-b]pyrazin-6-yl)-3-methyl-5-(trifluoromethyl)phenol,
38) (S)-2-(2-(2-oxabicyclo[3.1.1]heptan-4-yl)-2H-pyrazolo[3,4-b]pyrazin-6-yl)-3-methyl-5-(trifluoromethyl)phenol,
39) (R)-2-(2-(2-oxabicyclo[3.1.1]heptan-4-yl)-2H-pyrazolo[3,4-b]pyrazin-6-yl)-3-methyl-5-(trifluoromethyl)phenol,
40) 4-[6-[2-hydroxy-6-methyl-4-(trifluoromethyl)phenyl]pyrazolo[3,4-b]pyrazin-2-yl]bicyclo[2.1.1]hexan-1-ol,
41) 3-methyl-2-[2-(1-methyl-2-oxabicyclo[2.1.1]hexan-4-yl)pyrazolo[3,4-b]pyrazin-6-yl]-5-(trifluoromethyl)phenol,
42) 4-[6-[2-hydroxy-6-methyl-4-(trifluoromethyl)phenyl]pyrazolo[3,4-b]pyrazin-2-yl]norboman-1-ol,
43) 3-methyl-2-[2-[(3R)-3-methyl-1,1-dioxo-thiolan-3-yl]pyrazolo[3,4-b]pyrazin-6-yl]-5-(trifluoromethyl)phenol,
44) 2-[2-[(3R)-1,1-dioxothiolan-3-yl]pyrazolo[3,4-b]pyrazin-6-yl]-3-methyl-5-(trifluoromethyl)phenol,
45) 2-[2-[(3S)-1,1-dioxothiolan-3-yl]pyrazolo[3,4-b]pyrazin-6-yl]-3-methyl-5-(trifluoromethyl)phenol,
46) (3R,4S)-4-[6-[2-hydroxy-6-methyl-4-(trifluoromethyl)phenyl]pyrazolo[3,4-b]pyrazin-2-yl]tetrahydrofuran-3-ol,
47) (3S,4R)-4-[6-[2-hydroxy-6-methyl-4-(trifluoromethyl)phenyl]pyrazolo[3,4-b]pyrazin-2-yl]tetrahydrofuran-3-ol,
48) (3R,4R)-3-[6-[2-hydroxy-6-methyl-4-(trifluoromethyl)phenyl]pyrazolo[3,4-b]pyrazin-2-yl]tetrahydropyran-4-ol,
49) (3S,4S)-3-[6-[2-hydroxy-6-methyl-4-(trifluoromethyl)phenyl]pyrazolo[3,4-b]pyrazin-2-yl]tetrahydropyran-4-ol,
50) (1R,3R)-3-(6-(2-hydroxy-6-methyl-4-(trifluoromethyl)phenyl)-2H-pyrazolo[3,4-b]pyrazin-2-yl)cyclobutane-1-carbonitrile,
51) 2-[2-[3-(methoxymethyl)-1-bicyclo[1.1.1]pentanyl]pyrazolo[3,4-b]pyrazin-6-yl]-3-methyl-5-(trifluoromethyl)phenol,
52) 2-[2-(3-methoxy-1-bicyclo[1.1.1]pentanyl)pyrazolo[3,4-b]pyrazin-6-yl]-3-methyl-5-(trifluoromethyl)phenol,
53) 2-[2-[3-(difluoromethyl)-1-bicyclo[1.1.1]pentanyl]pyrazolo[3,4-b]pyrazin-6-yl]-3-methyl-5-(trifluoromethyl)phenol,
54) (1R,4R)-4-(6-(2-hydroxy-6-methyl-4-(trifluoromethyl)phenyl)-2H-pyrazolo[3,4-b]pyrazin-2-yl)cyclohexane-1-carbonitrile,
55) 2-[2-[1-(hydroxymethyl)-2-oxabicyclo[2.1.1]hexan-4-yl]pyrazolo[3,4-b]pyrazin-6-yl]-3-methyl-5-(trifluoromethyl)phenol,
56) 2-[2-(1-bicyclo[2.1.1]hexanyl)pyrazolo[3,4-b]pyrazin-6-yl]-3-methyl-5-(trifluoromethyl)phenol,
57) 2-[2-[3-(hydroxymethyl)-1-bicyclo[1.1.1]pentanyl]pyrazolo[3,4-b]pyrazin-6-yl]-3-methyl-5-(trifluoromethyl)phenol,
58) 2-(2-((1S,4S)-4-hydroxycyclohexyl)-2H-pyrazolo[3,4-b]pyrazin-6-yl)-3-methyl-5-(trifluoromethyl)phenol,
59) 3-methyl-2-[2-(8-oxabicyclo[3.2.1]octan-3-yl)pyrazolo[3,4-b]pyrazin-6-yl]-5-(trifluoromethyl)phenol,
60) 2-[3-[6-[2-hydroxy-6-methyl-4-(trifluoromethyl)phenyl]pyrazolo[3,4-b]pyrazin-2-yl]-1-bicyclo[1.1.1]pentanyl]acetonitrile,
61) 4-[6-[2-hydroxy-6-methyl-4-(trifluoromethyl)phenyl]pyrazolo[3,4-b]pyrazin-2-yl]bicyclo[2.1.1]hexane-1-carbonitrile,
62) 2-[2-(3-fluoro-1-bicyclo[1.1.1]pentanyl)pyrazolo[3,4-b]pyrazin-6-yl]-3-methyl-5-(trifluoromethyl)phenol,
63) 2-(2-((1R,3R)-3-chlorocyclobutyl)-2H-pyrazolo[3,4-b]pyrazin-6-yl)-3-methyl-5-(trifluoromethyl)phenol,
64) 3-methyl-2-[2-[(1R,2R)-2-methylcyclopropyl]pyrazolo[3,4-b]pyrazin-6-yl]-5-(trifluoromethyl)phenol,
65) 3-methyl-2-[2-(4-methyltetrahydropyran-4-yl)pyrazolo[3,4-b]pyrazin-6-yl]-5-(trifluoromethyl)phenol,
66) 3-methyl-2-[2-[(1S,2S)-2-methylcyclopropyl]pyrazolo[3,4-b]pyrazin-6-yl]-5-(trifluoromethyl)phenol,
67) (1R,5S,6S)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
68) (1R,5S,6R)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
69) 5-chloro-2-(2-((1R,3R)-3-fluorocyclobutyl)-2H-pyrazolo[3,4-b]pyrazin-6-yl)-3-methylphenol,
70) 2-(2-((1R,5S,6S)-3-oxabicyclo[3.1.0]hexan-6-yl)-2H-pyrazolo[3,4-b]pyrazin-6-yl)-5-chloro-3-methylphenol,
71) 2-(2-((1R,5S,6R)-3-oxabicyclo[3.1.0]hexan-6-yl)-2H-pyrazolo[3,4-b]pyrazin-6-yl)-5-chloro-3-methylphenol,
72) (R)-4-((6-(2-hydroxy-6-methyl-4-(trifluoromethyl)phenyl)-2H-pyrazolo[3,4-b]pyrazin-2-yl)methyl)-1-methylpyrrolidin-2-one,
73) (S)-4-((6-(2-hydroxy-6-methyl-4-(trifluoromethyl)phenyl)-2H-pyrazolo[3,4-b]pyrazin-2-yl)methyl)-1-methylpyrrolidin-2-one,
74) (R)-5-(6-(2-hydroxy-6-methyl-4-(trifluoromethyl)phenyl)-2H-pyrazolo[3,4-b]pyrazin-2-yl)-1-isopropylpiperidin-2-one,
75) (S)-5-(6-(2-hydroxy-6-methyl-4-(trifluoromethyl)phenyl)-2H-pyrazolo[3,4-b]pyrazin-2-yl)-1-isopropylpiperidin-2-one,
76) (R)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
77) (S)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
78) (R)-1-ethyl-5-(6-(2-hydroxy-6-methyl-4-(trifluoromethyl)phenyl)-2H-pyrazolo[3,4-b]pyrazin-2-yl)piperidin-2-one,
79) (S)-1-ethyl-5-(6-(2-hydroxy-6-methyl-4-(trifluoromethyl)phenyl)-2H-pyrazolo[3,4-b]pyrazin-2-yl)piperidin-2-one,
80) (R)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
81) (S)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
82) (R)-3-methyl-2-(2-(1-methyl-4,5,6,7-tetrahydro-TH-benzo[d][1,2,3]triazol-6-yl)-2H-pyrazolo[3,4-b]pyrazin-6-yl)-5-(trifluoromethyl)phenol,
83) (S)-3-methyl-2-(2-(1-methyl-4,5,6,7-tetrahydro-TH-benzo[d][1,2,3]triazol-6-yl)-2H-pyrazolo[3,4-b]pyrazin-6-yl)-5-(trifluoromethyl)phenol,
84) (R)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
85) (S)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
86) (R)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
87) (S)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
88) 2-(2-((1R,3R)-3-fluoro-1-methylcyclobutyl)-2H-pyrazolo[3,4-b]pyrazin-6-yl)-3-methyl-5-(trifluoromethyl)phenol,
89) 2-(2-((1S,3S)-3-fluoro-1-methylcyclobutyl)-2H-pyrazolo[3,4-b]pyrazin-6-yl)-3-methyl-5-(trifluoromethyl)phenol,
90) 2-(2-((1R,5R)-2,2-dimethyl-3-oxabicyclo[3.1.0]hexan-1-yl)-2H-pyrazolo[3,4-b]pyrazin-6-yl)-3-methyl-5-(trifluoromethyl)phenol,
91) 2-(2-((1S,5S)-2,2-dimethyl-3-oxabicyclo[3.1.0]hexan-1-yl)-2H-pyrazolo[3,4-b]pyrazin-6-yl)-3-methyl-5-(trifluoromethyl)phenol,
92) (S)-3-methyl-2-(2-(3-methyltetrahydrofuran-3-yl)-2H-pyrazolo[3,4-b]pyrazin-6-yl)-5-(trifluoromethyl)phenol,
93) (R)-3-methyl-2-(2-(3-methyltetrahydrofuran-3-yl)-2H-pyrazolo[3,4-b]pyrazin-6-yl)-5-(trifluoromethyl)phenol,
94) (R)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
95) (S)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
96) (1R,4R)-4-(6-(2-hydroxy-6-methyl-4-(trifluoromethyl)phenyl)-2H-pyrazolo[3,4-b]pyrazin-2-yl)-1-methylphosphinane 1-oxide,
97) (1S,4S)-4-(6-(2-hydroxy-6-methyl-4-(trifluoromethyl)phenyl)-2H-pyrazolo[3,4-b]pyrazin-2-yl)-1-methylphosphinane 1-oxide,
98) 2-(2-((1R,5R)-3-oxabicyclo[3.1.0]hexan-1-yl)-2H-pyrazolo[3,4-b]pyrazin-6-yl)-3-methyl-5-(trifluoromethyl)phenol,
99) 2-(2-((1S,5S)-3-oxabicyclo[3.1.0]hexan-1-yl)-2H-pyrazolo[3,4-b]pyrazin-6-yl)-3-methyl-5-(trifluoromethyl)phenol,
100) 3-methyl-2-(2-((3R,4R)-3-methyltetrahydro-2H-pyran-4-yl)-2H-pyrazolo[3,4-b]pyrazin-6-yl)-5-(trifluoromethyl)phenol,
101) 3-methyl-2-(2-((3S,4S)-3-methyltetrahydro-2H-pyran-4-yl)-2H-pyrazolo[3,4-b]pyrazin-6-yl)-5-(trifluoromethyl)phenol,
102) 3-methyl-2-(2-((3R,4S)-3-methyltetrahydro-2H-pyran-4-yl)-2H-pyrazolo[3,4-b]pyrazin-6-yl)-5-(trifluoromethyl)phenol,
103) 3-methyl-2-(2-((3S,4R)-3-methyltetrahydro-2H-pyran-4-yl)-2H-pyrazolo[3,4-b]pyrazin-6-yl)-5-(trifluoromethyl)phenol,
104) 3-methyl-2-(2-((2R,4S)-2-methyltetrahydro-2H-pyran-4-yl)-2H-pyrazolo[3,4-b]pyrazin-6-yl)-5-(trifluoromethyl)phenol,
105) 3-methyl-2-(2-((2S,4R)-2-methyltetrahydro-2H-pyran-4-yl)-2H-pyrazolo[3,4-b]pyrazin-6-yl)-5-(trifluoromethyl)phenol,
106) 2-(2-((1R,6S)-3-oxabicyclo[4.1.0]heptan-6-yl)-2H-pyrazolo[3,4-b]pyrazin-6-yl)-3-methyl-5-(trifluoromethyl)phenol,
107) 2-(2-((1S,6R)-3-oxabicyclo[4.1.0]heptan-6-yl)-2H-pyrazolo[3,4-b]pyrazin-6-yl)-3-methyl-5-(trifluoromethyl)phenol,
108) 3-methyl-2-(2-((2S,4S)-2-methyltetrahydro-2H-pyran-4-yl)-2H-pyrazolo[3,4-b]pyrazin-6-yl)-5-(trifluoromethyl)phenol,
109) 3-methyl-2-(2-((2R,4R)-2-methyltetrahydro-2H-pyran-4-yl)-2H-pyrazolo[3,4-b]pyrazin-6-yl)-5-(trifluoromethyl)phenol,
110) (R)-2-(2-(2,2-dimethyltetrahydro-2H-pyran-4-yl)-2H-pyrazolo[3,4-b]pyrazin-6-yl)-3-methyl-5-(trifluoromethyl)phenol,
111) (S)-2-(2-(2,2-dimethyltetrahydro-2H-pyran-4-yl)-2H-pyrazolo[3,4-b]pyrazin-6-yl)-3-methyl-5-(trifluoromethyl)phenol,
112) (R)-2-(2-(3,3-dimethyltetrahydro-2H-pyran-4-yl)-2H-pyrazolo[3,4-b]pyrazin-6-yl)-3-methyl-5-(trifluoromethyl)phenol,
113) (S)-2-(2-(3,3-dimethyltetrahydro-2H-pyran-4-yl)-2H-pyrazolo[3,4-b]pyrazin-6-yl)-3-methyl-5-(trifluoromethyl)phenol,
114) (R)-2-(2-(5-oxaspiro[2.5]octan-8-yl)-2H-pyrazolo[3,4-b]pyrazin-6-yl)-3-methyl-5-(trifluoromethyl)phenol,
115) (S)-2-(2-(5-oxaspiro[2.5]octan-8-yl)-2H-pyrazolo[3,4-b]pyrazin-6-yl)-3-methyl-5-(trifluoromethyl)phenol,
116) 2-(2-((3S,4S)-3-fluorotetrahydro-2H-pyran-4-yl)-2H-pyrazolo[3,4-b]pyrazin-6-yl)-3-methyl-5-(trifluoromethyl)phenol,
117) 2-(2-((3R,4R)-3-fluorotetrahydro-2H-pyran-4-yl)-2H-pyrazolo[3,4-b]pyrazin-6-yl)-3-methyl-5-(trifluoromethyl)phenol,
118) 2-(2-((3R,4S)-3-fluorotetrahydro-2H-pyran-4-yl)-2H-pyrazolo[3,4-b]pyrazin-6-yl)-3-methyl-5-(trifluoromethyl)phenol,
119) 2-(2-((3S,4R)-3-fluorotetrahydro-2H-pyran-4-yl)-2H-pyrazolo[3,4-b]pyrazin-6-yl)-3-methyl-5-(trifluoromethyl)phenol,
120) (R)-2-(2-(3,3-difluorotetrahydro-2H-pyran-4-yl)-2H-pyrazolo[3,4-b]pyrazin-6-yl)-3-methyl-5-(trifluoromethyl)phenol,
121) (S)-5-(6-(2-hydroxy-6-methyl-4-(trifluoromethyl)phenyl)-3-methyl-2H-pyrazolo[3,4-b]pyrazin-2-yl)-1-methylpiperidin-2-one,
122) (R and S)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
123) (R)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
124) (R)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
125) (R)-5-(6-(2-hydroxy-6-methyl-4-(trifluoromethyl)phenyl)-3-methyl-2H-pyrazolo[3,4-b]pyrazin-2-yl)piperidin-2-one,
126) (S)-5-(6-(2-hydroxy-6-methyl-4-(trifluoromethyl)phenyl)-3-methyl-2H-pyrazolo[3,4-b]pyrazin-2-yl)piperidin-2-one,
127) (R)-3-ethyl-5-((6-(2-hydroxy-6-methyl-4-(trifluoromethyl)phenyl)-2H-pyrazolo[3,4-b]pyrazin-2-yl)methyl)oxazolidin-2-one,
128) (R)-5-(6-(2-hydroxy-4-(trifluoromethyl)phenyl)-5-methyl-2H-pyrazolo[3,4-b]pyrazin-2-yl)-1-methylpiperidin-2-one,
129) (S)-5-(6-(2-hydroxy-4-(trifluoromethyl)phenyl)-5-methyl-2H-pyrazolo[3,4-b]pyrazin-2-yl)-1-methylpiperidin-2-one,
130) (S)-1-cyclopropyl-4-((6-(2-(ethoxymethoxy)-6-methyl-4-(trifluoromethyl)phenyl)-3-((S)-1-hydroxyethyl)-2H-pyrazolo[3,4-b]pyrazin-2-yl)methyl)pyrrolidin-2-one,
131) (S)-1-cyclopropyl-4-((6-(2-(ethoxymethoxy)-6-methyl-4-(trifluoromethyl)phenyl)-3-((R)-1-hydroxyethyl)-2H-pyrazolo[3,4-b]pyrazin-2-yl)methyl)pyrrolidin-2-one,
132) (R)-1-cyclopropyl-4-((6-(2-(ethoxymethoxy)-6-methyl-4-(trifluoromethyl)phenyl)-3-((R)-1-hydroxyethyl)-2H-pyrazolo[3,4-b]pyrazin-2-yl)methyl)pyrrolidin-2-one,
133) (R)-1-cyclopropyl-4-((6-(2-(ethoxymethoxy)-6-methyl-4-(trifluoromethyl)phenyl)-3-((S)-1-hydroxyethyl)-2H-pyrazolo[3,4-b]pyrazin-2-yl)methyl)pyrrolidin-2-one,
134) (S)-5-(6-(2-hydroxy-6-methyl-4-(trifluoromethyl)phenyl)-3-((S)-1-hydroxyethyl)-2H-pyrazolo[3,4-b]pyrazin-2-yl)-1-isopropylpiperidin-2-one,
135) (R)-5-(6-(2-hydroxy-6-methyl-4-(trifluoromethyl)phenyl)-3-((S)-1-hydroxyethyl)-2H-pyrazolo[3,4-b]pyrazin-2-yl)-1-isopropylpiperidin-2-one,
136) (R)-5-(6-(2-hydroxy-6-methyl-4-(trifluoromethyl)phenyl)-3-((R)-1-hydroxyethyl)-2H-pyrazolo[3,4-b]pyrazin-2-yl)-1-isopropylpiperidin-2-one,
137) (S)-5-(6-(2-hydroxy-6-methyl-4-(trifluoromethyl)phenyl)-3-((R)-1-hydroxyethyl)-2H-pyrazolo[3,4-b]pyrazin-2-yl)-1-isopropylpiperidin-2-one, and
138) 2-(2-((1R,3R)-3-fluorocyclobutyl)-2H-[1,2,3]triazolo[4,5-b]pyrazin-5-yl)-3-methyl-5-(trifluoromethyl)phenol.

A twenty-eighth exemplary embodiment is directed to a compound, or a pharmaceutically acceptable salt thereof, according to any prior embodiment, wherein the compound is selected from the group consisting of
1) 3-methyl-2-(2-(tetrahydro-2H-pyran-4-yl)-2H-pyrazolo[3,4-b]pyrazin-6-yl)-5-(trifluoromethyl)phenol,
2) 4-(6-(2-hydroxy-6-methyl-4-(trifluoromethyl)phenyl)-2H-pyrazolo[3,4-b]pyrazin-2-yl)-2-thiabicyclo[2.1.1]hexane 2,2-dioxide,
3) 2-(2-((1S,4R,5R or 1R,4S,5S)-2-oxabicyclo[2.2.1]heptan-5-yl)-2H-pyrazolo[3,4-b]pyrazin-6-yl)-3-methyl-5-(trifluoromethyl)phenol,
4) 2-(2-((1R,4S,5S or 1S,4R,5R)-2-oxabicyclo[2.2.1]heptan-5-yl)-2H-pyrazolo[3,4-b]pyrazin-6-yl)-3-methyl-5-(trifluoromethyl)phenol,
5) (5S)-5-[6-[2-hydroxy-6-methyl-4-(trifluoromethyl)phenyl]pyrazolo[3,4-b]pyrazin-2-yl]-1-methyl-piperidin-2-one,
6) (R)-3-methyl-2-(2-((1-methylpyrrolidin-3-yl)methyl)-2H-pyrazolo[3,4-b]pyrazin-6-yl)-5-(trifluoromethyl)phenol,
7) (R)-4-((6-(2-hydroxy-6-methyl-4-(trifluoromethyl)phenyl)-2H-pyrazolo[3,4-b]pyrazin-2-yl)methyl)pyrrolidin-2-one,
8) 3-(6-(2-hydroxy-6-methyl-4-(trifluoromethyl)phenyl)-2H-pyrazolo[3,4-b]pyrazin-2-yl)bicyclo[1.1.1]pentane-1-carbonitrile,
9) 2-(2-((1R,5S,6S)-3-oxabicyclo[3.1.0]hexan-6-yl)-2H-pyrazolo[3,4-b]pyrazin-6-yl)-3-methyl-5-(trifluoromethyl)phenol,
10) 2-(2-((1R,5S,6R)-3-oxabicyclo[3.1.0]hexan-6-yl)-2H-pyrazolo[3,4-b]pyrazin-6-yl)-3-methyl-5-(trifluoromethyl)phenol,
11) (1S,4S)-4-(6-(2-hydroxy-6-methyl-4-(trifluoromethyl)phenyl)-2H-pyrazolo[3,4-b]pyrazin-2-yl)cyclohexane-1-carbonitrile,
12) 3-(6-(2-hydroxy-6-methyl-4-(trifluoromethyl)phenyl)-2H-pyrazolo[3,4-b]pyrazin-2-yl)-2,2-dimethylpropanenitrile,
13) 1-((6-(2-hydroxy-6-methyl-4-(trifluoromethyl)phenyl)-2H-pyrazolo[3,4-b]pyrazin-2-yl)methyl)cyclobutane-1-carbonitrile,
14) 2-(2-(3-oxabicyclo[3.1.1]heptan-1-yl)-2H-pyrazolo[3,4-b]pyrazin-6-yl)-3-methyl-5-(trifluoromethyl)phenol,
15) 3-methyl-2-(2-(1-methyl-2-oxabicyclo[3.1.1]heptan-5-yl)-2H-pyrazolo[3,4-b]pyrazin-6-yl)-5-(trifluoromethyl)phenol,
16) 2-(2-((1R,3R)-3-fluorocyclobutyl)-2H-pyrazolo[3,4-b]pyrazin-6-yl)-3-methyl-5-(trifluoromethyl)phenol,
17) 2-(2-(2-oxabicyclo[2.1.1]hexan-4-yl)-2H-pyrazolo[3,4-b]pyrazin-6-yl)-3-methyl-5-(trifluoromethyl)phenol,
18) 2-((1s,3s)-3-(6-(2-hydroxy-6-methyl-4-(trifluoromethyl)phenyl)-2H-pyrazolo[3,4-b]pyrazin-2-yl)cyclobutyl)acetonitrile,
19) (R and S)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
20) (1S,3R and 1R,3S)-3-(6-(2-hydroxy-6-methyl-4-(trifluoromethyl)phenyl)-2H-pyrazolo[3,4-b]pyrazin-2-yl)cyclopentane-1-carbonitrile,
21) 2-(2-((3aS,6aR and 3aR,6aS)-hexahydro-3aH-cyclopenta[b]furan-3a-yl)-2H-pyrazolo[3,4-b]pyrazin-6-yl)-3-methyl-5-(trifluoromethyl)phenol,
22) (S and R)-2-(2-(5-oxaspiro[3.5]nonan-9-yl)-2H-pyrazolo[3,4-b]pyrazin-6-yl)-3-methyl-5-(trifluoromethyl)phenol,
23) (S and R)-2-(2-(6-oxaspiro[2.5]octan-1-yl)-2H-pyrazolo[3,4-b]pyrazin-6-yl)-3-methyl-5-(trifluoromethyl)phenol,
24) (S and R)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
25) (S and R)-2-(2-(2-oxabicyclo[3.1.1]heptan-4-yl)-2H-pyrazolo[3,4-b]pyrazin-6-yl)-3-methyl-5-(trifluoromethyl)phenol,
26) 4-[6-[2-hydroxy-6-methyl-4-(trifluoromethyl)phenyl]pyrazolo[3,4-b]pyrazin-2-yl]bicyclo[2.1.1]hexan-1-ol,
27) 3-methyl-2-[2-(1-methyl-2-oxabicyclo[2.1.1]hexan-4-yl)pyrazolo[3,4-b]pyrazin-6-yl]-5-(trifluoromethyl)phenol,
28) 4-[6-[2-hydroxy-6-methyl-4-(trifluoromethyl)phenyl]pyrazolo[3,4-b]pyrazin-2-yl]norboman-1-ol,
29) 3-methyl-2-[2-[(3R)-3-methyl-1,1-dioxo-thiolan-3-yl]pyrazolo[3,4-b]pyrazin-6-yl]-5-(trifluoromethyl)phenol,
30) 2-[2-[(3R)-1,1-dioxothiolan-3-yl]pyrazolo[3,4-b]pyrazin-6-yl]-3-methyl-5-(trifluoromethyl)phenol,
31) 2-[2-[(3S)-1,1-dioxothiolan-3-yl]pyrazolo[3,4-b]pyrazin-6-yl]-3-methyl-5-(trifluoromethyl)phenol,
32) (3R,4S)-4-[6-[2-hydroxy-6-methyl-4-(trifluoromethyl)phenyl]pyrazolo[3,4-b]pyrazin-2-yl]tetrahydrofuran-3-ol,
33) (3S,4R)-4-[6-[2-hydroxy-6-methyl-4-(trifluoromethyl)phenyl]pyrazolo[3,4-b]pyrazin-2-yl]tetrahydrofuran-3-ol,
34) (3R,4R)-3-[6-[2-hydroxy-6-methyl-4-(trifluoromethyl)phenyl]pyrazolo[3,4-b]pyrazin-2-yl]tetrahydropyran-4-ol,
35) (3S,4S)-3-[6-[2-hydroxy-6-methyl-4-(trifluoromethyl)phenyl]pyrazolo[3,4-b]pyrazin-2-yl]tetrahydropyran-4-ol,
36) (1R,3R)-3-(6-(2-hydroxy-6-methyl-4-(trifluoromethyl)phenyl)-2H-pyrazolo[3,4-b]pyrazin-2-yl)cyclobutane-1-carbonitrile,
37) 2-[2-[3-(methoxymethyl)-1-bicyclo[1.1.1]pentanyl]pyrazolo[3,4-b]pyrazin-6-yl]-3-methyl-5-(trifluoromethyl)phenol,
38) 2-[2-(3-methoxy-1-bicyclo[1.1.1]pentanyl)pyrazolo[3,4-b]pyrazin-6-yl]-3-methyl-5-(trifluoromethyl)phenol,
39) 2-[2-[3-(difluoromethyl)-1-bicyclo[1.1.1]pentanyl]pyrazolo[3,4-b]pyrazin-6-yl]-3-methyl-5-(trifluoromethyl)phenol,
40) (1R,4R)-4-(6-(2-hydroxy-6-methyl-4-(trifluoromethyl)phenyl)-2H-pyrazolo[3,4-b]pyrazin-2-yl)cyclohexane-1-carbonitrile,
41) 2-[2-[1-(hydroxymethyl)-2-oxabicyclo[2.1.1]hexan-4-yl]pyrazolo[3,4-b]pyrazin-6-yl]-3-methyl-5-(trifluoromethyl)phenol,
42) 2-[2-(1-bicyclo[2.1.1]hexanyl)pyrazolo[3,4-b]pyrazin-6-yl]-3-methyl-5-(trifluoromethyl)phenol,
43) 2-[2-[3-(hydroxymethyl)-1-bicyclo[1.1.1]pentanyl]pyrazolo[3,4-b]pyrazin-6-yl]-3-methyl-5-(trifluoromethyl)phenol,
44) 2-(2-((1S,4S)-4-hydroxycyclohexyl)-2H-pyrazolo[3,4-b]pyrazin-6-yl)-3-methyl-5-(trifluoromethyl)phenol,
45) 3-methyl-2-[2-(8-oxabicyclo[3.2.1]octan-3-yl)pyrazolo[3,4-b]pyrazin-6-yl]-5-(trifluoromethyl)phenol,
46) 2-[3-[6-[2-hydroxy-6-methyl-4-(trifluoromethyl)phenyl]pyrazolo[3,4-b]pyrazin-2-yl]-1-bicyclo[1.1.1]pentanyl]acetonitrile,
47) 4-[6-[2-hydroxy-6-methyl-4-(trifluoromethyl)phenyl]pyrazolo[3,4-b]pyrazin-2-yl]bicyclo[2.1.1]hexane-1-carbonitrile,
48) 2-[2-(3-fluoro-1-bicyclo[1.1.1]pentanyl)pyrazolo[3,4-b]pyrazin-6-yl]-3-methyl-5-(trifluoromethyl)phenol,
49) 2-(2-((1R,3R)-3-chlorocyclobutyl)-2H-pyrazolo[3,4-b]pyrazin-6-yl)-3-methyl-5-(trifluoromethyl)phenol,
50) 3-methyl-2-[2-[(1R,2R)-2-methylcyclopropyl]pyrazolo[3,4-b]pyrazin-6-yl]-5-(trifluoromethyl)phenol,
51) 3-methyl-2-[2-(4-methyltetrahydropyran-4-yl)pyrazolo[3,4-b]pyrazin-6-yl]-5-(trifluoromethyl)phenol,
52) 3-methyl-2-[2-[(1S,2S)-2-methylcyclopropyl]pyrazolo[3,4-b]pyrazin-6-yl]-5-(trifluoromethyl)phenol,
53) (1R,5S,6S or 1R,5S,6R)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
54) (1R,5S,6R or 1R,5S,6S)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
55) 5-chloro-2-(2-((1R,3R)-3-fluorocyclobutyl)-2H-pyrazolo[3,4-b]pyrazin-6-yl)-3-methylphenol,
56) 2-(2-((1R,5S,6S)-3-oxabicyclo[3.1.0]hexan-6-yl)-2H-pyrazolo[3,4-b]pyrazin-6-yl)-5-chloro-3-methylphenol,
57) 2-(2-((1R,5S,6R)-3-oxabicyclo[3.1.0]hexan-6-yl)-2H-pyrazolo[3,4-b]pyrazin-6-yl)-5-chloro-3-methylphenol,
58) (R or S)-4-((6-(2-hydroxy-6-methyl-4-(trifluoromethyl)phenyl)-2H-pyrazolo[3,4-b]pyrazin-2-yl)methyl)-1-methylpyrrolidin-2-one,
59) (S or R)-4-((6-(2-hydroxy-6-methyl-4-(trifluoromethyl)phenyl)-2H-pyrazolo[3,4-b]pyrazin-2-yl)methyl)-1-methylpyrrolidin-2-one,
60) (R or S)-5-(6-(2-hydroxy-6-methyl-4-(trifluoromethyl)phenyl)-2H-pyrazolo[3,4-b]pyrazin-2-yl)-1-isopropylpiperidin-2-one,
61) (S or R)-5-(6-(2-hydroxy-6-methyl-4-(trifluoromethyl)phenyl)-2H-pyrazolo[3,4-b]pyrazin-2-yl)-1-isopropylpiperidin-2-one,
62) (R or S)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
63) (S or R)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
64) (R or S)-1-ethyl-5-(6-(2-hydroxy-6-methyl-4-(trifluoromethyl)phenyl)-2H-pyrazolo[3,4-b]pyrazin-2-yl)piperidin-2-one,
65) (S or R)-1-ethyl-5-(6-(2-hydroxy-6-methyl-4-(trifluoromethyl)phenyl)-2H-pyrazolo[3,4-b]pyrazin-2-yl)piperidin-2-one,
66) (R or S)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
67) (S or R)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
68) (R or S)-3-methyl-2-(2-(1-methyl-4,5,6,7-tetrahydro-1H-benzo[d][1,2,3]triazol-6-yl)-2H-pyrazolo[3,4-b]pyrazin-6-yl)-5-(trifluoromethyl)phenol,
69) (S or R)-3-methyl-2-(2-(1-methyl-4,5,6,7-tetrahydro-1H-benzo[d][1,2,3]triazol-6-yl)-2H-pyrazolo[3,4-b]pyrazin-6-yl)-5-(trifluoromethyl)phenol,
70) (R or S)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
71) (S or R)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
72) (R or S)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
73) (S or R)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
74) 2-(2-((1R,3R or 1S,3S)-3-fluoro-1-methylcyclobutyl)-2H-pyrazolo[3,4-b]pyrazin-6-yl)-3-methyl-5-(trifluoromethyl)phenol,
75) 2-(2-((1S,3S or 1R,3R)-3-fluoro-1-methylcyclobutyl)-2H-pyrazolo[3,4-b]pyrazin-6-yl)-3-methyl-5-(trifluoromethyl)phenol,
76) 2-(2-((1R,5R or 1S,5S)-2,2-dimethyl-3-oxabicyclo[3.1.0]hexan-1-yl)-2H-pyrazolo[3,4-b]pyrazin-6-yl)-3-methyl-5-(trifluoromethyl)phenol,
77) 2-(2-((1S,5S or 1R,5R)-2,2-dimethyl-3-oxabicyclo[3.1.0]hexan-1-yl)-2H-pyrazolo[3,4-b]pyrazin-6-yl)-3-methyl-5-(trifluoromethyl)phenol,
78) (S or R)-3-methyl-2-(2-(3-methyltetrahydrofuran-3-yl)-2H-pyrazolo[3,4-b]pyrazin-6-yl)-5-(trifluoromethyl)phenol,
79) (R or S)-3-methyl-2-(2-(3-methyltetrahydrofuran-3-yl)-2H-pyrazolo[3,4-b]pyrazin-6-yl)-5-(trifluoromethyl)phenol,
80) (R or S)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
81) (S or R)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
82) (1R,4R or 1S,4S)-4-(6-(2-hydroxy-6-methyl-4-(trifluoromethyl)phenyl)-2H-pyrazolo[3,4-b]pyrazin-2-yl)-1-methylphosphinane 1-oxide,
83) 2-(2-((1R,5R or 1S,5S)-3-oxabicyclo[3.1.0]hexan-1-yl)-2H-pyrazolo[3,4-b]pyrazin-6-yl)-3-methyl-5-(trifluoromethyl)phenol,
84) 2-(2-((1S,5S or 1R,5R)-3-oxabicyclo[3.1.0]hexan-1-yl)-2H-pyrazolo[3,4-b]pyrazin-6-yl)-3-methyl-5-(trifluoromethyl)phenol,
85) 3-methyl-2-(2-((3R,4R or 3S,4S)-3-methyltetrahydro-2H-pyran-4-yl)-2H-pyrazolo[3,4-b]pyrazin-6-yl)-5-(trifluoromethyl)phenol,
86) 3-methyl-2-(2-((3S,4S or 3R,4R)-3-methyltetrahydro-2H-pyran-4-yl)-2H-pyrazolo[3,4-b]pyrazin-6-yl)-5-(trifluoromethyl)phenol,
87) 3-methyl-2-(2-((3R,4S or 3S,4R)-3-methyltetrahydro-2H-pyran-4-yl)-2H-pyrazolo[3,4-b]pyrazin-6-yl)-5-(trifluoromethyl)phenol,
88) 3-methyl-2-(2-((3S,4R or 3R,4S)-3-methyltetrahydro-2H-pyran-4-yl)-2H-pyrazolo[3,4-b]pyrazin-6-yl)-5-(trifluoromethyl)phenol,
89) 3-methyl-2-(2-((2R,4S or 2S,4R)-2-methyltetrahydro-2H-pyran-4-yl)-2H-pyrazolo[3,4-b]pyrazin-6-yl)-5-(trifluoromethyl)phenol,
90) 3-methyl-2-(2-((2S,4R or 2R,4S)-2-methyltetrahydro-2H-pyran-4-yl)-2H-pyrazolo[3,4-b]pyrazin-6-yl)-5-(trifluoromethyl)phenol,
91) 2-(2-((1R,6S or 1S,6R)-3-oxabicyclo[4.1.0]heptan-6-yl)-2H-pyrazolo[3,4-b]pyrazin-6-yl)-3-methyl-5-(trifluoromethyl)phenol,
92) 2-(2-((1S,6R or 1R,6S)-3-oxabicyclo[4.1.0]heptan-6-yl)-2H-pyrazolo[3,4-b]pyrazin-6-yl)-3-methyl-5-(trifluoromethyl)phenol,
93) 3-methyl-2-(2-((2S,4S or 2R,4R)-2-methyltetrahydro-2H-pyran-4-yl)-2H-pyrazolo[3,4-b]pyrazin-6-yl)-5-(trifluoromethyl)phenol,
94) 3-methyl-2-(2-((2R,4R or 2S,4S)-2-methyltetrahydro-2H-pyran-4-yl)-2H-pyrazolo[3,4-b]pyrazin-6-yl)-5-(trifluoromethyl)phenol,
95) (R or S)-2-(2-(2,2-dimethyltetrahydro-2H-pyran-4-yl)-2H-pyrazolo[3,4-b]pyrazin-6-yl)-3-methyl-5-(trifluoromethyl)phenol,
96) (S or R)-2-(2-(2,2-dimethyltetrahydro-2H-pyran-4-yl)-2H-pyrazolo[3,4-b]pyrazin-6-yl)-3-methyl-5-(trifluoromethyl)phenol,
97) (R or S)-2-(2-(3,3-dimethyltetrahydro-2H-pyran-4-yl)-2H-pyrazolo[3,4-b]pyrazin-6-yl)-3-methyl-5-(trifluoromethyl)phenol,
98) (S or R)-2-(2-(3,3-dimethyltetrahydro-2H-pyran-4-yl)-2H-pyrazolo[3,4-b]pyrazin-6-yl)-3-methyl-5-(trifluoromethyl)phenol,
99) (R or S)-2-(2-(5-oxaspiro[2.5]octan-8-yl)-2H-pyrazolo[3,4-b]pyrazin-6-yl)-3-methyl-5-(trifluoromethyl)phenol,
100) (S or R)-2-(2-(5-oxaspiro[2.5]octan-8-yl)-2H-pyrazolo[3,4-b]pyrazin-6-yl)-3-methyl-5-(trifluoromethyl)phenol,
101) 2-(2-((3S,4S or 3R,4R)-3-fluorotetrahydro-2H-pyran-4-yl)-2H-pyrazolo[3,4-b]pyrazin-6-yl)-3-methyl-5-(trifluoromethyl)phenol,
102) 2-(2-((3R,4R or 3S,4S)-3-fluorotetrahydro-2H-pyran-4-yl)-2H-pyrazolo[3,4-b]pyrazin-6-yl)-3-methyl-5-(trifluoromethyl)phenol,
103) 2-(2-((3R,4S or 3S,4R)-3-fluorotetrahydro-2H-pyran-4-yl)-2H-pyrazolo[3,4-b]pyrazin-6-yl)-3-methyl-5-(trifluoromethyl)phenol,
104) 2-(2-((3S,4R or 3R,4S)-3-fluorotetrahydro-2H-pyran-4-yl)-2H-pyrazolo[3,4-b]pyrazin-6-yl)-3-methyl-5-(trifluoromethyl)phenol,
105) (R or S)-2-(2-(3,3-difluorotetrahydro-2H-pyran-4-yl)-2H-pyrazolo[3,4-b]pyrazin-6-yl)-3-methyl-5-(trifluoromethyl)phenol,
106) (S or R)-5-(6-(2-hydroxy-6-methyl-4-(trifluoromethyl)phenyl)-3-methyl-2H-pyrazolo[3,4-b]pyrazin-2-yl)-1-methylpiperidin-2-one,
107) (R and S)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
108) (R or S)-5-(6-(2-hydroxy-6-methyl-4-(trifluoromethyl)phenyl)-3-methyl-2H-pyrazolo[3,4-b]pyrazin-2-yl)piperidin-2-one,
109) (S or R)-5-(6-(2-hydroxy-6-methyl-4-(trifluoromethyl)phenyl)-3-methyl-2H-pyrazolo[3,4-b]pyrazin-2-yl)piperidin-2-one,
110) (R)-3-ethyl-5-((6-(2-hydroxy-6-methyl-4-(trifluoromethyl)phenyl)-2H-pyrazolo[3,4-b]pyrazin-2-yl)methyl)oxazolidin-2-one,
111) (R or S)-5-(6-(2-hydroxy-4-(trifluoromethyl)phenyl)-5-methyl-2H-pyrazolo[3,4-b]pyrazin-2-yl)-1-methylpiperidin-2-one,
112) (S or R)-5-(6-(2-hydroxy-4-(trifluoromethyl)phenyl)-5-methyl-2H-pyrazolo[3,4-b]pyrazin-2-yl)-1-methylpiperidin-2-one,
113) (S or R)-1-cyclopropyl-4-((6-(2-(ethoxymethoxy)-6-methyl-4-(trifluoromethyl)phenyl)-3-((S or R)-1-hydroxyethyl)-2H-pyrazolo[3,4-b]pyrazin-2-yl)methyl)pyrrolidin-2-one,
114) (S or R)-1-cyclopropyl-4-((6-(2-(ethoxymethoxy)-6-methyl-4-(trifluoromethyl)phenyl)-3-((R or S)-1-hydroxyethyl)-2H-pyrazolo[3,4-b]pyrazin-2-yl)methyl)pyrrolidin-2-one,
115) (R or S)-1-cyclopropyl-4-((6-(2-(ethoxymethoxy)-6-methyl-4-(trifluoromethyl)phenyl)-3-((R or S)-1-hydroxyethyl)-2H-pyrazolo[3,4-b]pyrazin-2-yl)methyl)pyrrolidin-2-one,
116) (R or S)-1-cyclopropyl-4-((6-(2-(ethoxymethoxy)-6-methyl-4-(trifluoromethyl)phenyl)-3-((S or R)-1-hydroxyethyl)-2H-pyrazolo[3,4-b]pyrazin-2-yl)methyl)pyrrolidin-2-one,
117) (S or R)-5-(6-(2-hydroxy-6-methyl-4-(trifluoromethyl)phenyl)-3-((S or R)-1-hydroxyethyl)-2H-pyrazolo[3,4-b]pyrazin-2-yl)-1-isopropylpiperidin-2-one,
118) (R or S)-5-(6-(2-hydroxy-6-methyl-4-(trifluoromethyl)phenyl)-3-((S or R)-1-hydroxyethyl)-2H-pyrazolo[3,4-b]pyrazin-2-yl)-1-isopropylpiperidin-2-one,
119) (R or S)-5-(6-(2-hydroxy-6-methyl-4-(trifluoromethyl)phenyl)-3-((R or S)-1-hydroxyethyl)-2H-pyrazolo[3,4-b]pyrazin-2-yl)-1-isopropylpiperidin-2-one,
120) (S or R)-5-(6-(2-hydroxy-6-methyl-4-(trifluoromethyl)phenyl)-3-((R or S)-1-hydroxyethyl)-2H-pyrazolo[3,4-b]pyrazin-2-yl)-1-isopropylpiperidin-2-one, and
121) 2-(2-((1R,3R)-3-fluorocyclobutyl)-2H-[1,2,3]triazolo[4,5-b]pyrazin-5-yl)-3-methyl-5-(trifluoromethyl)phenol.

A twenty-nineth exemplary embodiment is directed to a compound, or a pharmaceutically acceptable salt thereof, according to any prior embodiment, wherein the compound is selected:

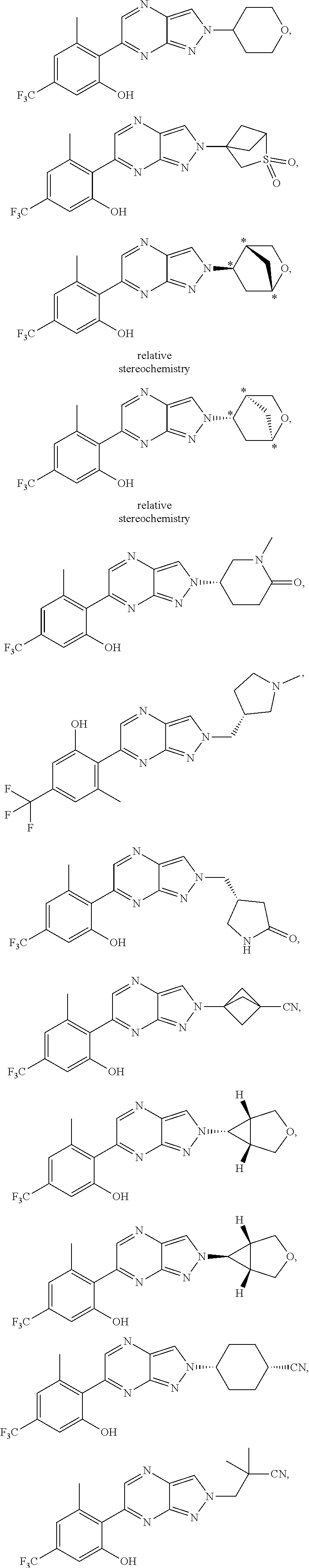

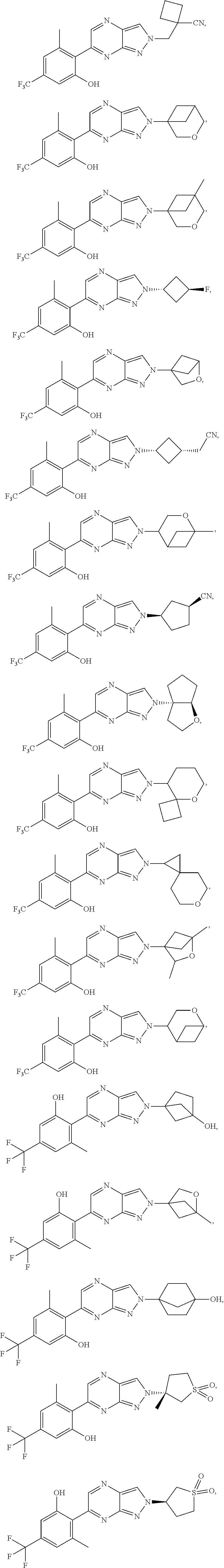

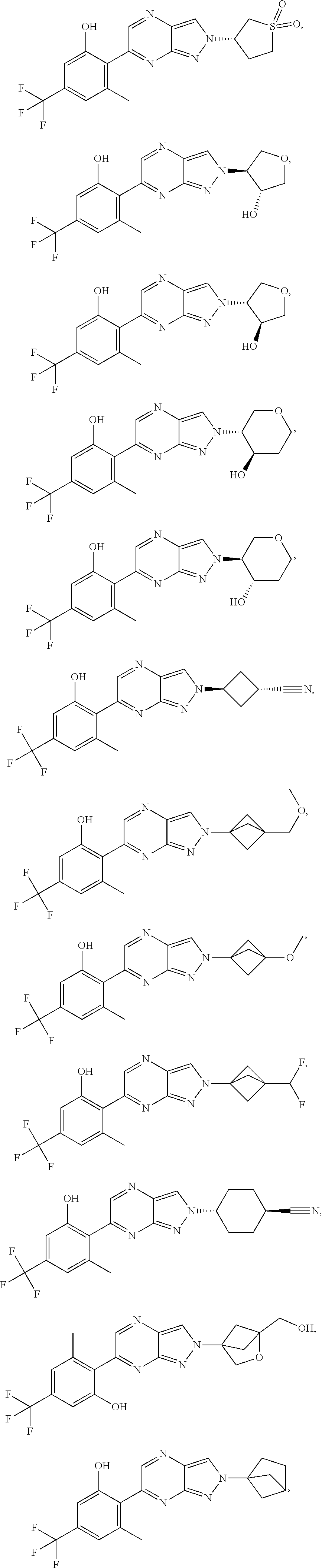

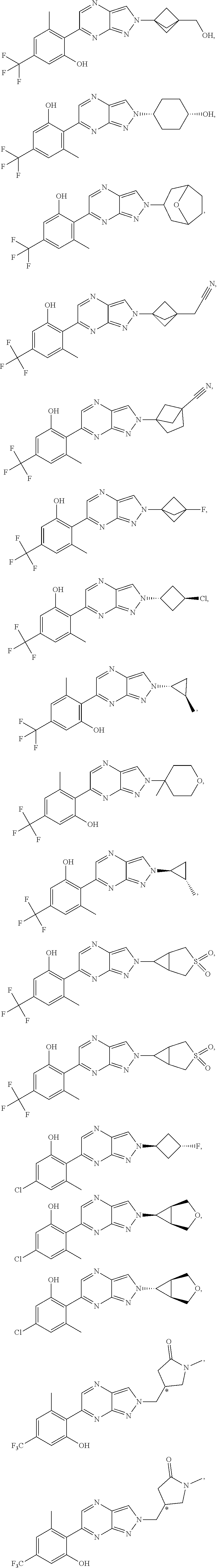

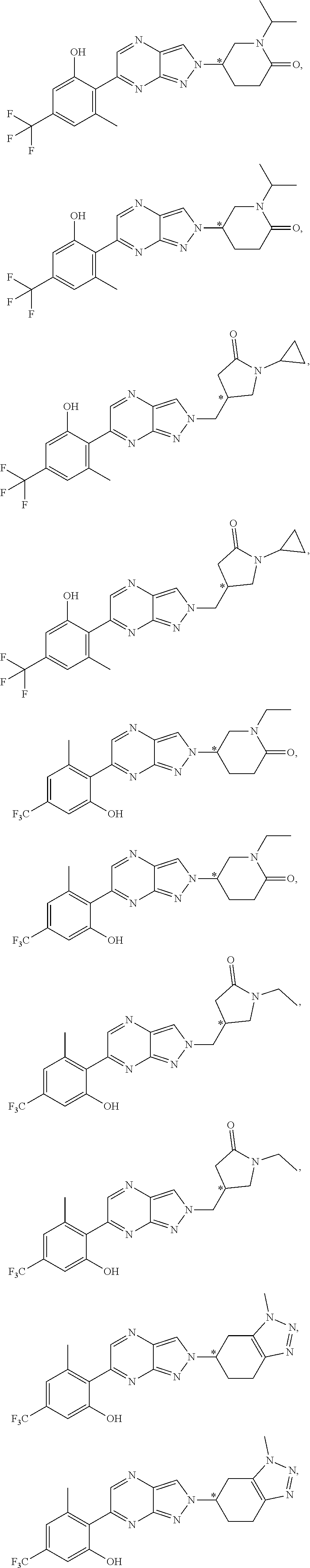

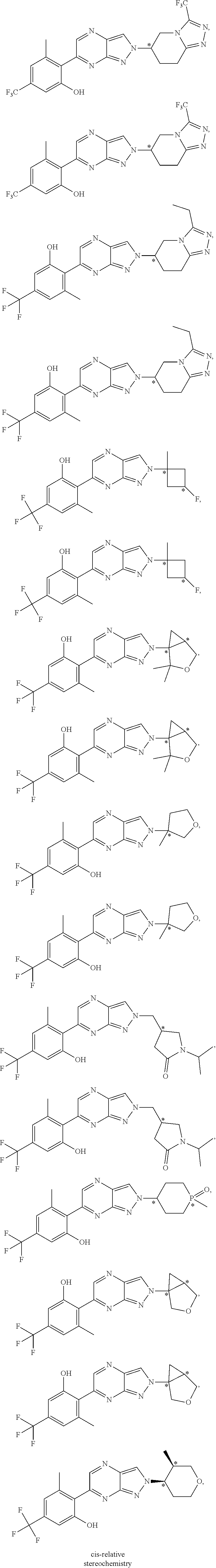

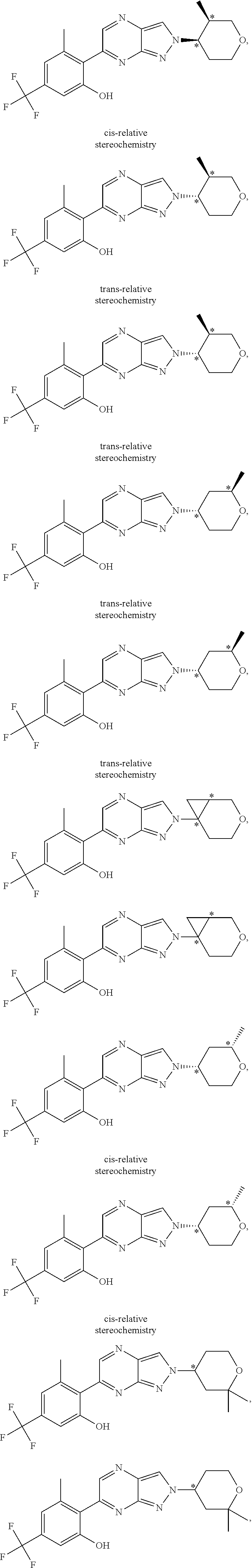

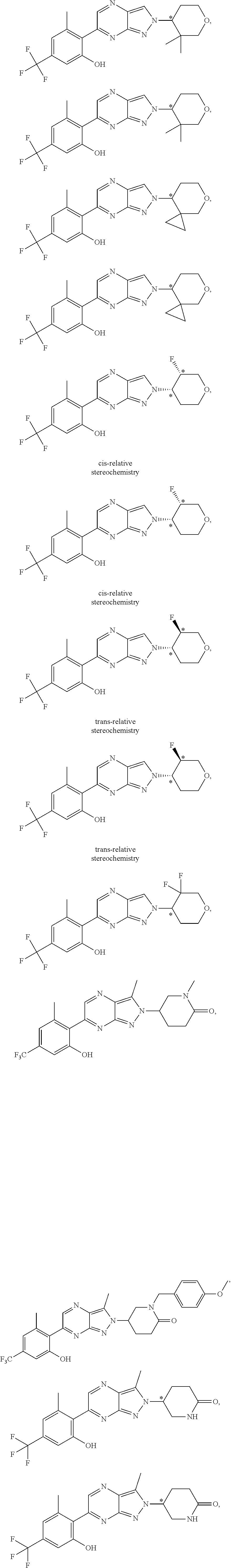

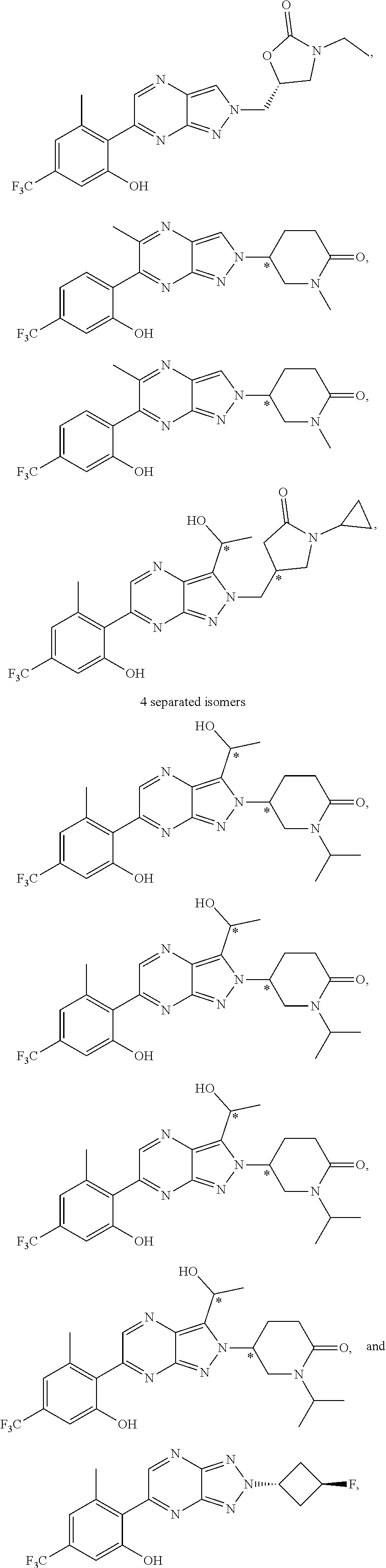

and pharmaceutically acceptable salts thereof.

A thirtieth exemplary embodiment is directed to a pharmaceutical composition comprising a compound, or a pharmaceutically acceptable salt thereof, according to any prior embodiment, and a pharmaceutically acceptable carrier.

A thirty-first exemplary embodiment is directed to a use of a compound, or a pharmaceutically acceptable salt thereof, according to any prior embodiment, for the preparation of a medicament useful for the treatment of a disorder, condition, or disease that is responsive to the inhibition of NLRP3 in a mammal in need thereof.

A thirty-second exemplary embodiment is directed to a use of a compound, or a pharmaceutically acceptable salt thereof, according to any prior embodiment, for the manufacture of a medicament for the treatment, prevention or control of an inflammatory disorder, a fibrotic disorder, a cardiovascular disorder, a metabolic disorder, and a neurodegenerative disorder.

A thirty-third exemplary embodiment is directed to a use according to any prior embodiment, wherein the disorder is an inflammatory disorder.

A thirty-fourth exemplary embodiment is directed to a use according to any prior embodiment, wherein the inflammatory disorder is selected from: an auto-immune disorder, an auto-inflammatory disorder, an inflammatory joint disorder, an inflammatory skin disorder, and a neuroinflammatory disorder.

A thirty-fifth exemplary embodiment is directed to a use according to any prior embodiment, wherein the disorder is selected from: atherosclerosis, non-alcoholic steatohepatitis, Alzheimer's disease, Parkinson's disease, and dementia with Lewy bodies.

A thirty-sixth exemplary embodiment is directed to a use according to any prior embodiment, wherein the disorder is selected from: atherosclerosis, non-alcoholic steatohepatitis, Alzheimer's disease, and Parkinson's disease.

A thirty-sixth exemplary embodiment is directed to a compound, or a pharmaceutically acceptable salt thereof, for use in therapy.

A thirty-seventh exemplary embodiment is directed to a method of treating or preventing a disorder, condition or disease that is responsive to the inhibition of NLRP3 in a patient in need thereof comprising administration to said patient of a therapeutically effective amount of such a compound, or a pharmaceutically acceptable salt thereof.

A thirty-eighth exemplary embodiment is directed to a method according to any prior embodiment, wherein the disorder is selected from: an inflammatory disorder, a fibrotic disorder, a cardiovascular disorder, a metabolic disorder, or a neurodegenerative disorder.

A thirty-ninth exemplary embodiment is directed to a method according to any prior embodiment, wherein the disorder is an inflammatory disorder.

A fortieth exemplary embodiment is directed to a method according to any prior embodiment, wherein the inflammatory disorder is selected from: an auto-immune disorder, an auto-inflammatory disorder, an inflammatory joint disorder, an inflammatory skin disorder, and a neuroinflammatory disorder.

A forty-first exemplary embodiment is directed to a method according to any prior embodiment, wherein the disorder is selected from: atherosclerosis, non-alcoholic steatohepatitis, Alzheimer's disease, Parkinson's disease, and dementia with Lewy bodies.

A forty-second exemplary embodiment is directed to a method according to any prior embodiment, wherein the disorder is selected from: atherosclerosis, non-alcoholic steatohepatitis, Alzheimer's disease, and Parkinson's disease.

### Methods of Synthesis

The following reaction schemes and Examples illustrate methods which may be employed for the synthesis of the compounds of Formula (I) described in this invention. These reaction schemes and Examples are provided to illustrate the invention and are not to be construed as limiting the invention in any manner. All substituents are as defined above unless indicated otherwise. Several strategies based upon synthetic transformations known in the literature of organic synthesis may be employed for the preparation of the compounds of Formula (I). The scope of the invention is defined by the appended claims. Compound names were generated in Chemdraw Version 21.0.0.28.

### EXAMPLES

The following examples are meant to be illustrative and should not be construed as further limiting. The contents of the figures and all references, patents, and published patent applications cited throughout this application are expressly incorporated herein by reference.

Abbreviations used herein are set forth in Table 1.

TABLE 1
Abbreviations

| | |
|---|---|
| * | designates a stereocenter |
| [M + H]⁺ | mass of protonated molecular ion |
| [M − H]⁻ | mass of deprotonated molecular ion |
| [Rh(COD)Cl]₂ | chloro(1,5-cyclooctadiene)rhodium(I) dimer |
| ° C. | degrees Celsius |
| ¹H NMR | proton nuclear magnetic resonance spectroscopy |
| Ac | acetyl |
| AcOH | acetic acid |
| AMD | age-related macular degeneration |
| aq. or aq | aqueous |
| ASA | acetyl-salicyclic acid drugs (including aspirin) |
| ASC | apoptosis-associated speck-like protein containing a CARD |
| ASC SPECK | (defined in text) |
| ATCC | American type culture collection |
| B₂pin₂ | bis(pinacolato)diboron |
| Boc or boc | tert-butoxycarbonyl |
| BPin ester | boronic acid pinacol ester |
| br | broad |
| BSA | bovine serum albumin |
| BTFFH | bis(tetramethylene)fluoroformamidinium hexafluorophosphate |
| Bu or nBu | n-butyl |
| Bz | benzoyl |
| Calc'd or calcd. | calculated |
| CAPS | cryopyrin-associated periodic syndromes |
| CDI | 1,1′-carbonyl-diimidazole |
| cGAS | Cyclic GMP-AMP synthase |
| conc. | concentrated |
| ^{c}Pr | cyclopropyl |
| d | doublet |
| DAMPS | danger-associated molecular patterns |
| DAST | diethylaminosulfur trifluoride |
| DCE | dichloroethane |
| DCM | dichloromethane |
| dd | doublet of doublets |
| DDQ | 2,3-dichloro-5,6-dicyano-1,4-benzoquinone |
| DEA | diethanolamine |
| Dess-Martin Periodinane | 1,1,1-tris(acetyloxy)-1,1-dihydro-1,2-benziodoxol-3-(1H)-one |
| DFMS | bis(((difluoromethyl)sulfinyl)oxy)zinc |
| DIAD | diisopropyl azodicarboxylate |
| DIC | N,N′-diisopropyl-carbodiimide |
| DIPEA | N,N-diisopropylethylamine |
| DMA | dimethylacetamide |
| DME | dimethoxyethane |
| DMF | dimethylformamide |
| DMSO | dimethylsulfoxide |
| DMSO-d₆ | hexadeuterodimethyl sulfoxide |
| dppf | 1,1′-bis(diphenylphosphino)-ferrocene |
| dqd | doublet of a quarter of doublets |
| DRAQ5 ™ | DRAQ5 ™ Fluorescent Probe Solution (5 mM) (Thermo |
| | Scientific ™ Catalog No. 62251 |
| dtbbpy | 4,4′-di-tert-butyl-2,2′-dipyridyl |
| EC₅₀ | half maximal effective concentration |
| EDC | 1-ethyl-3-(3-dimethylaminopropyl)-carbodiimide |
| ee | enantiomeric excess |
| equiv | equivalent(s) |
| ESI | electrospray ionization |
| Et | ethyl, CH₂CH₃ |
| Et₃N | triethylamine |
| Et₂O | diethyl ether |
| EtOAc | ethyl acetate |
| EtOH | ethanol |
| FA (see example 3) | formic acid |
| g | grams |
| GLP1 | glucagon-like peptide 1 |
| Gsdmd | gasdermin D |
| h or hr(s) | hour(s) |
| HOAT | 1-Hydroxy-7-azabenzotriazole |
| HPLC | high-performance liquid chromatography |
| Hz | Hertz |
| IL-18 | interleukin-18 |
| IL-lβ | interleukin-Iβ |
| ⁱPr | isopropyl, CH₂(CH₃) |
| iPrMgCl | isopropylmagnesium chloride |
| ⁱPrOH or IPA | isopropyl alcohol |
| J | coupling constant, in MHz |
| JAK | Janus kinase |
| KF | potassium fluoride |
| kg | kilogram |
| KHMDS | potassium bis(trimethylsilyl)amide |
| L | liter |
| LAH | lithium aluminum hydride |
| LC | liquid chromatography |
| LCMS | liquid chromatography/mass spectrometry |
| LRMS | low resolution mass spectrometry |
| LRR | leucine-rich repeat |
| m | multiplet |
| M | Molar |
| MASH | metabolic dysfunction-associated steatohepatitis |
| Me | methyl, CH₃ |
| MeCN | acetonitrile |
| MeMgBr | Methylmagnesium bromide |
| MeOH | methanol |
| mg | milligram(s) |
| MHz | megahertz |
| min | minute(s) |
| mL | milliliter(s) |
| mM | millimolar |
| mm | millimeter |
| mmol | millimole(s) |
| mol | mole(s) |
| MPLC | medium pressure liquid chromatography |
| MS | mass spectroscopy |
| NaHMDS | Sodium bis(trimethylsilyl)amide |
| NaN₃ | sodium azide |
| NaSEt | sodium ethanethiolate |
| NASH | non-alcoholic steatohepatitis |
| NBD | nucleotide-binding site domain |
| NBS | N-bromosuccinimide |
| NF-KB | nuclear factor kappa B |
| NH₄OAc | ammonium acetate |
| NHPI | N-hydroxyphthalimide |
| NLR | NOD-like receptors |
| NLRP3 | NOD-like receptor protein 3 |
| nM | nanomolar |
| NMO | 4-Methylmorpholine N-oxide |
| NMP | N-methylpyrrolidine |
| NOD | nucleotide-binding oligomerization domain |
| NSAIDs | nonsteroidal anti-inflammatory drugs |
| OAc | acetate |
| oxo | 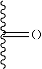 [SMILES: *=O] |
| PAMP | pathogen-associated molecular patterns |
| PBS | phosphate-buffered saline |
| PCC | pyridinium chlorochromate |
| PCSK9 | Proprotein convertase subtilisin/kexin type 9 |
| Pd(dppf)Cl₂ | [1,1′-bis-(diphenylphosphino)-ferrocene]dichloropalladium(II) |
| Pd(OAc)₂ | Palladium(II) acetate |
| Pd(PPh₃)₄ | tetrakis(triphenylphosphine)palladium(0) |
| Pd(tBu₃P)₂ | bis(tri-tert-butylphosphine)-palladium(0) |
| Pd/C | palladium on carbon |
| PD1 | Programmed cell death protein 1 |
| PdCl₂(PPh₃)₂ | Dichlorobis(triphenylphosphine)palladium(II) |
| PDL1 | Programmed death-ligand 1 |
| PE | petroleum ether |
| PG | protecting group |
| Ph or ph | phenyl |
| PMA | phorbol 12-myristate 13-acetate |
| ppm | parts per million |
| pr | propyl |
| prep | preparative |
| prolL-1β | pro-interleukin 1 beta |
| PRR | pattern recognition receptor |
| q | quartet |
| rac | racemic |
| RPMI | Roswell Park Memorial Institute |
| rt or RT | room temperature, approximately 25° C. |
| s | singlet |
| SARS | severe acute respiratory syndrome |
| sat. or satd | saturated |
| SEMCl | chloromethyl 2-trimethylsilylethyl ether |
| SFC | Supercritical Fluid Chromatography |
| SGLT2 | Sodium-glucose cotransporter-2 |
| S_{N}Ar | nucleophilic aromatic substitution |
| STING | Stimulator of Interferon Genes |
| t | triplet |
| t-AmOH | 2-methylbutan-2-ol |
| TBAF | tetrabutylammonium fluoride |
| TBHP | tert-butyl hydroperoxide |
| ^{t}Bu | tert-butyl |
| ^{t}BuOH | tert-butyl alcohol |
| td | triplet of doublet |
| TEA or Et₃N | triethylamine |
| tert | tertiary |
| TFA | trifluoroacetic acid |
| TfOH | triflic acid |
| THF | tetrahydrofuran |
| THP-1 | Tohoku Hospital Pediatrics-1 |
| Ti(OEt)₄ | titanium (IV) ethoxide |
| Ti(OiPr)₄ | titanium (IV) isopropoxide |
| TLC | thin layer chromatography |
| TMHD | 2,2,6,6-tetramethyl-3,5-heptanedione |
| TMS-Diazomethane | trimethylsilyl-diazomethane |
| TNF-alpha or TNFα | Tumour necrosis factor α |
| Ts | tosylate |
| TsCl | 4-toluenesulfonyl chloride |
| tt | triplet of triplets |
| tween-20 | polyethylene glycol sorbitan monolaurate |
| UV | ultraviolet |
| v/v | volume/volume |
| VEG-F | Vascular Endothelial Growth Factor |
| wt % | Percentage by weight |
| XPhos Pd G3 | (2-Dicyclohexylphosphino-2′,4′,6′-triisopropyl-1,1′-biphenyl)[2- |
| | (2′-amino-1,1′-biphenyl)]palladium(II) methanesulfonate |
| δ | chemical shift |
| μL | microliter |

### Instrumentation and Chiral Separation Methods

Reverse phase chromatography was carried out on a Waters 150 equipped with a column selected from the following: Phenomenex Synergi C18 (250 mm×30 mm×4 micron), Phenomenex Luna C18 (250 mm×21 mm×5 micron), Agilent Zorbax Bonus-RP (150 mm×21 mm×5 micron), Waters X-Select CSH C18 (150 mm×19 mm×5 micron). Conditions included either high pH (0-100% acetonitrile/water eluent comprising 0.1% v/v NH₄OH) or low pH (0-100% acetonitrile/water eluent comprising 0.1% v/v TFA or Formic Acid) and are noted for some examples.

SFC chiral resolution was carried out on Waters Thar 80 SFC or Berger MG II preparative SFC systems. The general preparative conditions of separating diastereomeric or enantiomeric mixtures of compounds using chiral SFC are as set forth in Table 2.

TABLE 2
General Preparative Conditions

| Chiral | | |
|---|---|---|
| Column | Mobile Phase | Method |
| IC | 35% MeOH (0.1% | A |
| | NH₄OH)/CO₂ | |
| IG | 30% MeOH (0.1% | B |
| | NH₄OH)/CO₂ | |
| IC | 30% MeOH (0.1% | C |
| | NH₄OH)/CO₂ | |
| A2S-5 | 40% EtOH/CO₂ | D |
| IC | 30% MeOH/CO₂ | E |
| IC | 25% MeOH (0.1% | F |
| | NH₄OH)/CO₂ | |
| AD-H | 40% MeOH/CO₂ | G |
| A6 | 30% MeOH (0.1% | H |
| | DIPEA)/CO₂ | |
| IG | 50% EtOH (0.1% | I |
| | NH₄OH)/CO₂ | |
| IG | 50% EtOH (0.05% | J |
| | DIPEA)/CO₂ | |
| IC | 40% MeOH (0.1% | K |
| | Et₂NH)/CO₂ | |
| IG | 20% MeOH (0.1% | L |
| | NH₄OH)/CO₂ | |
| IG | 15% MeOH (0.1% | M |
| | NH₄OH)/CO₂ | |
| ID | 20% MeOH (0.1% | N |
| | NH₄OH)/CO₂ | |
| IH | 20% MeOH (0.1% | O |
| | NH₄OH)/CO₂ | |
| ID | 25% MeOH (0.1% | P |
| | NH₄OH)/CO₂ | |
| IC | 20% MeOH (0.1% | Q |
| | NH₄OH)/CO₂ | |
| AD-H | 30% EtOH/CO₂ | R |
| IG | 35% MeOH/CO₂ | S |
| AD | 25% iPrOH (0.1% | T |
| | NH₄OH)/CO₂ | |
| AD | 35% iPrOH (0.1% | U |
| | NH₄OH)/CO₂ | |

Proton or ¹H NMR was acquired using a Bruker 500 MHz NEO NMR spectrometer equipped with a 5 mm iProbe in accordance with standard analytical techniques, unless specified otherwise, and results of spectral analysis are reported. Chemical shift (δ) values are reported in delta (δ) units, parts per million (ppm). Chemical shifts for ¹H NMR spectra are given relative to signals for residual non-deuterated solvent (CDCl₃ referenced at δ 7.26 ppm; DMSO-d₆ referenced at δ 2.50 ppm; CD₃OD referenced at δ 3.31 ppm).

### General Schemes

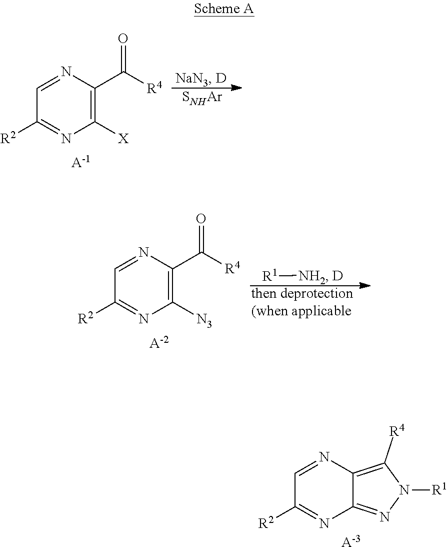

Scheme A illustrates the synthetic sequence for preparation of pyrazolopyrazine derivatives such as A-3. First, nucleophilic aromatic substitution (S_{N}Ar) reaction of NaN₃ gives the azido-carbonyl derivatives such as A-2. Subsequently, reaction of the azido-carbonyl A-2 with primary amines under thermal conditions affords pyrazolopyrazine products that are deprotected in situ (when applicable) to afford compounds of the formula A-3.

### Intermediate 1: 2-(2-(ethoxymethoxy)-6-methyl-4-(trifluoromethyl)phenyl)-4,4,5,5-tetramethyl-1,3,2-dioxaborolane

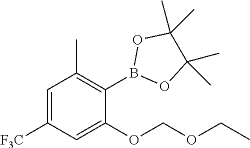 [SMILES: CCOCOc1cc(C(F)(F)F)cc(C)c1B1OC(C)(C)C(C)(C)O1]

Step 1: 3-Methyl-5-(trifluoromethyl)phenol: To a solution of 3-bromo-5-(trifluoromethyl)phenol (500 g, 2.07 mol), K₂CO₃ (858.9 g, 6.22 mol) and Pd(dppf)Cl₂ (75.8 g, 103.7 mmol) in 1,4-dioxane (7.5 L) under N2 atmosphere was added portion-wise trimethyl-1,3,5,2,4,6-trioxa-triborinane (1.04 kg, 4.15 mol, 50 wt % in THF). The resulting mixture was stirred for 12 h at 100° C., followed by cooling to rt. The reaction was quenched with ice water at 0° C., then diluted with EtOAc. The organic layer was separated, washed with brine, dried over anhydrous Na₂SO₄ and concentrated under reduced pressure. The resulting crude residue was purified by silica gel chromatography (EtOAc:PE) to afford the title compound. LCMS [M−H]⁻=175.1 (calcd. 175.0).

Step 2: 2-Iodo-3-methyl-5-(trifluoromethyl)phenol: NaH (128.5 g, 3.21 mol, 60 wt %) was added at 0° C. to a stirring solution of 3-methyl-5-(trifluoromethyl)phenol (283 g, 1.61 mol) in toluene (1.42 L) under N2 atmosphere. The resulting mixture was stirred at 0° C. for 30 min, followed by the portion-wise addition of a solution of 12 (306.1 g, 1.21 mmol) in toluene (5.66 L). The resulting mixture was stirred at 20° C. for 3 h, then quenched by pouring onto a water/ice bath. The mixture was diluted with EtOAc, and the layers were separated. The organic layer was washed with brine, dried over anhydrous Na₂SO₄, and concentrated under reduced pressure to give the title compound, which was used in the next step without further purification.

Step 3: 1-(Ethoxymethoxy)-2-iodo-3-methyl-5-(trifluoromethyl)benzene: Chloromethyl ethyl ether (289.9 g, 3.07 mol) was added at 0° C. to a stirring solution of 2-iodo-3-methyl-5-(trifluoromethyl)phenol (463 g, 1.53 mol) and Cs₂CO₃ (998.9 g, 3.07 mmol) in DMF (4.6 L) under N2 atmosphere. The resulting mixture was stirred for 8 h at rt, then cooled to 0° C. and quenched with addition of ice water. The resulting mixture was diluted with EtOAc, and the layers separated. The organic layer was washed with brine, dried over anhydrous Na₂SO₄, and concentrated under reduced pressure. The resulting crude residue was purified by silica gel chromatography (EtOAc:PE) to afford the title compound. ¹H NMR (300 MHz, DMSO-d₆) δ 7.55 (s, 1H), 7.18 (s, 1H), 5.42 (s, 2H), 3.75-3.65 (m, 2H), 2.50 (s, 3H), 1.21-1.10 (m, 3H).

Step 4: 2-(2-(Ethoxymethoxy)-6-methyl-4-(trifluoromethyl)phenyl)-4,4,5,5-tetramethyl-1,3,2-dioxaborolane: A mixture containing 1-(ethoxymethoxy)-2-iodo-3-methyl-5-(trifluoromethyl)benzene (330 g, 916.4 mmol), B₂pin₂ (469.1 g, 3.67 mol), Et₃N (556.4 g, 5.50 mol), Pd(OAc)₂ (10.3 g, 45.8 mmol), and (2-biphenyl)dicyclohexylphosphine (32.1 g, 91.6 mmol) in 1,4-dioxane (3.3 L) was placed under N2 atmosphere. The reaction mixture was stirred for 6 h at 100° C., then cooled to 25° C. and quenched with ice water. The resulting mixture was filtered, and the residue was washed with EtOAc. The filtrate layers were separated. The organic layer was washed with brine, dried over anhydrous Na₂SO₄, and concentrated under reduced pressure. The resulting crude residue was purified by silica gel chromatography (EtOAc:PE), and the solvent was removed under vacuum. The resulting residue was dissolved in hexanes and stirred for 5 min at −30° C. Then the precipitate was collected by filtration to afford the title compound. ¹H NMR (300 MHz, CDCl₃) δ 7.13-7.03 (m, 2H), 5.23 (s, 2H), 3.74 (q, J=7.1 Hz, 2H), 2.41 (s, 3H), 1.41 (s, 12H), 1.24 (t, J=7.1 Hz, 3H).

### Intermediate 2: 3-azido-5-(2-(ethoxymethoxy)-6-methyl-4-(trifluoromethyl)phenyl)pyrazine-2-carbaldehyde

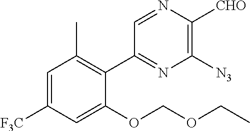 [SMILES: CCOCOc1cc(C(F)(F)F)cc(C)c1-c1cnc(C=O)c(N=[N+]=[N-])n1]

Step 1: methyl 3-chloro-5-(2-(ethoxymethoxy)-6-methyl-4-(trifluoromethyl) phenyl)pyrazine-2-carboxylate: 1,4-Dioxane (25 mL) and H₂O (5 mL) were added to a vial containing methyl 3,5-dichloropyrazine-2-carboxylate (Combi-Blocks, 1.2 g, 5.8 mmol), 2-(2-(ethoxymethoxy)-6-methyl-4-(trifluoromethyl)phenyl)-4,4,5,5-tetramethyl-1,3,2-dioxaborolane (Intermediate 1, 2.7 g, 7.5 mmol), K₂CO₃ (4.0 g, 29 mmol), and Pd(dppf)Cl₂ (473 mg, 0.58 mmol). The mixture was degassed with N2 for 15 min, then heated to 100° C. and stirred for 12 h. Then the reaction mixture was cooled to rt, diluted with H₂O and EtOAc, and filtered over a pad of diatomaceous earth (CELITE® 545). Then the layers were separated, and the aq. layer was extracted with EtOAc (×3). The combined organic layers were dried over MgSO₄, filtered, and the solvent was removed under reduced pressure. The resulting crude mixture was purified by silica gel chromatography (EtOAc:hexanes) to afford the title compound. LCMS [M+H]⁺=405.2 (calcd. 405.1).

Step 2: (3-chloro-5-(2-(ethoxymethoxy)-6-methyl-4-(trifluoromethyl)phenyl)pyrazin-2-yl)methanol: A solution of methyl 3-chloro-5-(2-(ethoxymethoxy)-6-methyl-4-(trifluoromethyl) phenyl)pyrazine-2-carboxylate (823 mg, 2.0 mmol) in THF (18 mL) and MeOH (2 mL) was cooled to 0° C. A solution of LiBH₄ (2.0 mL, 4.0 mmol, 2M in THF) was added dropwise, and the mixture was stirred at 0° C. for 10 min. Then the reaction mixture was quenched with MeOH (21 mL) and the mixture stirred at rt for an additional 10 min. Then the reaction mixture was diluted with EtOAc and 1M HCl aq. solution, the layers were separated, and the aqueous layer was extracted with EtOAc (×3). The combined organic layers were dried over MgSO₄, filtered, and the solvent was removed under reduced pressure. The resulting residue containing the title compound was used in the next step without further purification. LCMS [M+H]⁺=377.2 (calcd. 377.1).

Step 3: 3-chloro-5-(2-(ethoxymethoxy)-6-methyl-4-(trifluoromethyl)phenyl)pyrazine-2-carbaldehyde: To a solution of (3-chloro-5-(2-(ethoxymethoxy)-6-methyl-4-(trifluoromethyl) phenyl)pyrazin-2-yl)methanol (766 mg, 2.0 mmol) in CH₂Cl₂ (10 mL), Dess-Martin Periodinane (905 mg, 2.1 mmol) was added at 0° C. Then the mixture was stirred at rt for 12 h. The resulting precipitate was filtered, and the solvent removed under reduced pressure. The resulting crude mixture was purified by reverse phase HPLC (C18 stationary phase, MeCN/H₂O+0.1% TFA) to afford the title compound. LCMS [M+H]+=375.2 (calcd. 375.1).

Step 4: 3-azido-5-(2-(ethoxymethoxy)-6-methyl-4-(trifluoromethyl)phenyl)pyrazine-2-carbaldehyde: NaN₃ (48 mg, 0.73 mmol) was added portion-wise to a solution of 3-chloro-5-(2-(ethoxymethoxy)-6-methyl-4-(trifluoromethyl)phenyl)pyrazine-2-carbaldehyde (250 mg, 0.67 mmol) in DMF (1.3 mL) at rt. The mixture was then stirred at rt for 12 h. Then the reaction mixture was diluted with H₂O and EtOAc, the layers were separated, and the aq. layer was extracted with EtOAc (×3). The combined organic layers were dried over MgSO₄, filtered, and the solvent was removed under reduced pressure. The resulting crude residue was purified by silica gel chromatography (EtOAc:hexanes) to afford the title compound. LCMS [M+H]⁺=382.2 (calcd. 382.1).

The compound listed in Table 3 was prepared using procedures similar to the procedure described for Intermediate 2 using the appropriate starting materials.

TABLE 3
Intermediate 3

| Intermediate | Structure | Name | LCMS [M + H]⁺ |
|---|---|---|---|
| 3 | 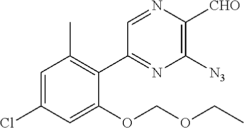 [SMILES: CCOCOc1cc(Cl)cc(C)c1-c1cnc(C=O)c(N=[N+]=[N-])n1] | 3-azido-5-(4-chloro-2- (ethoxymethoxy)-6- methylphenyl)pyrazine- 2-carbaldehyde | Calcd. 348.1, found 348.0 |

### Intermediate 4: 1-(3-azido-5-(2-(ethoxymethoxy)-6-methyl-4-(trifluoromethyl)phenyl) pyrazin-2-yl)ethan-1-one

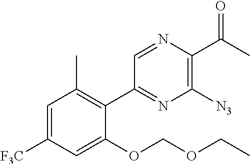 [SMILES: CCOCOc1cc(C(F)(F)F)cc(C)c1-c1cnc(C(C)=O)c(N=[N+]=[N-])n1]

Step 1: 1-(3,5-dichloropyrazin-2-yl)ethan-1-one: MeMgBr (2.1 mL, 6.32 mmol, 3M in Et₂₀) was added dropwise to a solution of 3,5-dichloropyrazine-2-carbonitrile (1 g, 5.75 mmol) in Et₂₀ (10 mL) at 78° C. The reaction mixture was warmed to rt and stirred for 4 h. Then the reaction mixture was cooled to 0° C. and quenched with 1M HCl solution. Then the mixture was diluted with H₂O and EtOAc, the layers were separated, and the aq. layer was extracted with EtOAc (×3). The combined organic layers were dried over MgSO₄ and filtered, and the solvent was removed under reduced pressure. The resulting residue containing the title compound was used in the next step without further purification. LCMS [M+H]⁺=191.1 (calcd. 191.0).

Step 2: 1-(3-chloro-5-(2-(ethoxymethoxy)-6-methyl-4-(trifluoromethyl)phenyl)pyrazin-2-yl)ethan-1-one: 1,4-Dioxane (20 mL) and H₂O (2 mL) were added to a vial containing 1-(3,5-dichloropyrazin-2-yl)ethan-1-one (1.1 g, 5.8 mmol), 2-(2-(ethoxymethoxy)-6-methyl-4-(trifluoromethyl)phenyl)-4,4,5,5-tetramethyl-1,3,2-dioxaborolane (Intermediate 1, 1.35 g, 3.7 mmol), Cs₂CO₃ (5.6 g, 17.2 mmol), and Pd(dppf)Cl₂ (337 mg, 0.46 mmol). The mixture was degassed with N2 for 15 min, then heated to 100° C. and stirred for 12 h. Then the reaction mixture was cooled to rt and diluted with H₂O and EtOAc. Then the layers were separated, and the aq. layer was extracted with EtOAc (×3). The combined organic layers were dried over MgSO₄ and filtered, and the solvent was removed under reduced pressure. The resulting crude mixture was purified by silica gel chromatography (EtOAc:PE) to afford the title compound. LCMS [M+H]⁺=389.0 (calcd. 389.1).

Step 3: 1-(3-azido-5-(2-(ethoxymethoxy)-6-methyl-4-(trifluoromethyl)phenyl) pyrazin-2-yl)ethan-1-one: NaN₃ (33 mg, 0.51 mmol) was added to a solution of 1-(3-chloro-5-(2-(ethoxymethoxy)-6-methyl-4-(trifluoromethyl)phenyl)pyrazin-2-yl)ethan-1-one (100 mg, 0.26 mmol) in DMF (1.5 mL) at rt. The reaction mixture was then heated to 60° C. and stirred for 2 h. Then the reaction mixture was cooled to rt, diluted with H₂O and EtOAc, the layers were separated, and the aq. layer was extracted with EtOAc (×3). The combined organic layers were dried over MgSO₄ and filtered, and the solvent was removed under reduced pressure. The resulting crude mixture was purified by reverse phase HPLC (C18 stationary phase, MeCN/H₂O+0.1% TFA) to afford the title compound. LCMS [M+H]⁺=396.1 (calcd. 396.1).

### Intermediate 5: 5-hydroxy-1-isopropylpiperidin-2-one

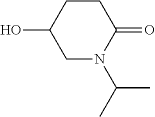

Step 1: 5-((tert-butyldiphenylsilyl)oxy)-1-isopropylpiperidin-2-one: K₂CO₃ (2.74 g, 19.8 mmol) and KOH (1.11 g, 19.80 mmol) were added to a mixture of 5-((tert-butyldiphenylsilyl) oxy)piperidin-2-one (3.5 g, 9.90 mmol), 2-iodopropane (16.8 g, 99 mmol), 2-bromopropane (12.2 g, 99 mmol) and tetrabutylammonium bromide (3.19 g, 9.9 mmol) in toluene (40 mL) at rt. The reaction mixture was then heated to 50° C. and stirred for 16 h. Then the reaction mixture was cooled to rt, triturated with EtOAc, filtered, and the solvents were removed under reduced pressure. The crude residue was diluted with H₂O and EtOAc, the layers were separated, and the aq. layer was extracted with EtOAc (×3). The combined organic layers were dried over MgSO₄, filtered, and the solvent was removed under reduced pressure. The resulting crude mixture was purified by reverse phase HPLC (C18 stationary phase, MeCN/H₂O+0.5% TFA) to afford the title compound. LCMS [M+H]⁺=396.2 (calcd. 396.2).

Step 2: 5-hydroxy-1-isopropylpiperidin-2-one: KF (4.41 g, 76 mmol) was added to a solution of 5-((tert-butyldiphenylsilyl)oxy)-1-isopropylpiperidin-2-one (1.5 g, 3.79 mmol) in MeOH (15 mL) at rt, and then the reaction mixture was heated to 80° C. and stirred for 12 h. Then the reaction mixture was cooled to rt, and the solvents were removed under reduced pressure. The resulting crude residue was triturated with DCM, filtered, and the solvents were removed under reduced pressure to afford the title compound. LCMS [M+H]⁺=158.1 (calcd. 158.1).

### Intermediate 6: (1-cyclopropyl-5-oxopyrrolidin-3-yl)methyl 4-methylbenzenesulfonate

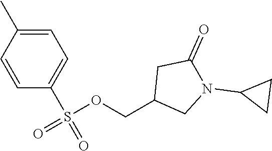 [SMILES: Cc1ccc(S(=O)(=O)OCC2CC(=O)N(C3CC3)C2)cc1]

To a mixture of 1-cyclopropyl-4-(hydroxymethyl)pyrrolidin-2-one (1.2 g, 7.73 mmol) and TsCl (2.2 g, 11.6 mmol) in DCM (10 mL) at 0° C., Et₃N (3.2 mL, 23.2 mmol) was added. Then the reaction mixture was stirred at rt for 18 h. Then the reaction mixture was diluted with H₂O and EtOAc, the layers were separated, and the aq. layer was extracted with EtOAc (×3). The combined organic layers were dried over MgSO₄, filtered, and the solvent was removed under reduced pressure. The resulting crude mixture was purified by silica gel chromatography (EtOAc:PE) to afford the title compound. LCMS [M+H]⁺=310.1 (calcd. 310.1).

The compound listed in Table 4 was prepared using procedures similar to the procedure described for Intermediate 6 using the appropriate starting materials.

TABLE 4
Intermediate 7

| Intermediate | Structure | Name | LCMS [M + H]⁺ |
|---|---|---|---|
| 7 | 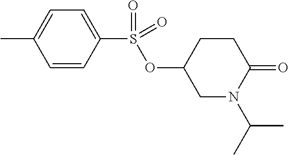 [SMILES: Cc1ccc(S(=O)(=O)OC2CCC(=O)N(C(C)C)C2)cc1] | 1-isopropyl-6- oxopiperidin-3-yl 4- methylbenzenesulfonate | Calcd. 312.1, found 312.1 |

### Intermediate 8: 1-(6-(2-(ethoxymethoxy)-6-methyl-4-(trifluoromethyl)phenyl)-2H-pyrazolo[3,4-b]pyrazin-3-yl)ethan-1-one

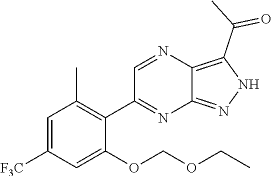 [SMILES: CCOCOc1cc(C(F)(F)F)cc(C)c1-c1cnc2c(C(C)=O)[nH]nc2n1]

Step 1: 6-chloro-2-((2-(trimethylsilyl)ethoxy)methyl)-2H-pyrazolo[3,4-b]pyrazine or 6-chloro-1-((2-(trimethylsilyl)ethoxy)methyl)-1H-pyrazolo[3,4-b]pyrazine: NaH (780 mg, 19.4 mmol, 60 wt % in mineral oil) was added to a solution of 6-chloro-2H-pyrazolo[3,4-b]pyrazine (2.0 g, 12.9 mmol) in DMF (30 mL) at 0° C., and the reaction mixture was then stirred for 20 min. SEMCl (2.8 mL, 15.5 mmol) was then added to the reaction mixture at 0° C., and the resulting solution was warmed to rt and stirred for 2 h. Then the reaction mixture was diluted with H₂O and EtOAc, the layers were separated, and the aq. layer was extracted with EtOAc (×3). The combined organic layers were dried over MgSO₄, filtered, and the solvent was removed under reduced pressure. The resulting crude mixture was purified by silica gel chromatography (EtOAc:PE) to afford the title compound (the position of SEM group was not determined). LCMS [M+H]⁺=285.0 (calcd. 285.1).

Step 2: 6-(2-(ethoxymethoxy)-6-methyl-4-(trifluoromethyl)phenyl)-2-((2-(trimethylsilyl)ethoxy)methyl)-2H-pyrazolo[3,4-b]pyrazine: MeCN (25 mL) and H₂O (5 mL) were added to a vial containing 6-chloro-2-((2-(trimethylsilyl)ethoxy)methyl)-2H-pyrazolo[3,4-b]pyrazine (2.5 g, 8.78 mmol), 2-(2-(ethoxymethoxy)-6-methyl-4-(trifluoromethyl)phenyl)-4,4,5,5-tetramethyl-1,3,2-dioxaborolane or 6-chloro-1-((2-(trimethylsilyl)ethoxy)methyl)-1H-pyrazolo[3,4-b]pyrazine (Intermediate 1, 4.74 g, 13.17 mmol), Na₂CO₃ (2.79 g, 26.3 mmol), and Pd(dppf)Cl₂ (642 mg, 0.88 mmol). The mixture was degassed with N2 for 15 min, then heated to 100° C. and stirred for 12 h. Then the reaction mixture was cooled to rt and diluted with H₂O and EtOAc. Then the layers were separated, and the aq. layer was extracted with EtOAc (×3). The combined organic layers were dried over MgSO₄, filtered, and the solvent was removed under reduced pressure. The resulting crude mixture was purified by silica gel chromatography (EtOAc:PE) to afford the title compound. LCMS [M+H]⁺=483.2 (calcd. 483.2).

Step 3: 6-(2-(ethoxymethoxy)-6-methyl-4-(trifluoromethyl)phenyl)-2H-pyrazolo[3,4-b]pyrazine: TBAF (21.8 mL, 21.76 mmol) was added to a solution of 6-(2-(ethoxymethoxy)-6-methyl-4-(trifluoromethyl)phenyl)-2-((2-(trimethylsilyl)ethoxy)methyl)-2H-pyrazolo[3,4-b]pyrazine (3.5 g, 7.25 mmol) in THF (30 mL) under N2 atmosphere at rt. The reaction mixture was then heated to 70° C. and stirred for 5 h. The resulting reaction mixture was then cooled to rt, diluted with H₂O and EtOAc, the layers were separated, and the aq. layer was extracted with EtOAc (×3). The combined organic layers were dried over MgSO₄, filtered, and the solvent was removed under reduced pressure. The resulting crude mixture was purified by silica gel chromatography (EtOAc:PE) to afford the title compound. LCMS [M+H]⁺=353.0 (calcd. 353.1).

Step 4: 3-bromo-6-(2-(ethoxymethoxy)-6-methyl-4-(trifluoromethyl)phenyl)-2H-pyrazolo[3,4-b]pyrazine: NBS (1.0 g, 5.53 mmol) was added to a solution of 6-(2-(ethoxymethoxy)-6-methyl-4-(trifluoromethyl)phenyl)-2H-pyrazolo[3,4-b]pyrazine (1.3 g, 3.69 mmol) in DMF (10 mL) at rt, and the reaction mixture was stirred for 2 h. The resulting reaction mixture was diluted with H₂O and EtOAc, the layers were separated, and the aq. layer was extracted with EtOAc (×3). The combined organic layers were dried over MgSO₄, filtered, and the solvent was removed under reduced pressure The resulting crude mixture was purified by silica gel chromatography (EtOAc:PE) to afford the title compound. LCMS [M+H]⁺=431.0 (calcd. 431.0).

Step 5: 1-(6-(2-(ethoxymethoxy)-6-methyl-4-(trifluoromethyl)phenyl)-2H-pyrazolo[3,4-b]pyrazin-3-yl)ethan-1-one: Tributyl(1-ethoxyvinyl)stannane (1.5 mL, 4.52 mmol) was added to a solution of 3-bromo-6-(2-(ethoxymethoxy)-6-methyl-4-(trifluoromethyl)phenyl)-2H-pyrazolo[3,4-b]pyrazine (1.3 g, 3.01 mmol) and PdCl₂(PPh₃)₂ (127 mg, 0.18 mmol) in 1,4-dioxane (15 mL) at rt. The mixture was degassed with N2 for 15 min, then heated to 80° C. and stirred for 5 h. Then the reaction mixture was cooled to rt, HCl (0.75 M in H₂O, 3 mL) was added, and stirred for 2 h. The reaction mixture was then quenched with aq. KF solution (60 mL), stirred for 30 min, and filtered. The filtrate was extracted with EtOAc (3×), the combined organic layers were dried over MgSO₄, filtered, and the solvent was removed under reduced pressure. The resulting crude mixture was purified by silica gel chromatography (EtOAc:PE) to afford the title compound. LCMS [M+H]⁺=395.0 (calcd. 395.1).

### Example 1: 3-methyl-2-(2-(tetrahydro-2H-pyran-4-yl)-2H-pyrazolo[3,4-b]pyrazin-6-yl)-5-(trifluoromethyl)phenol

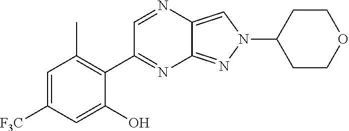 [SMILES: Cc1cc(C(F)(F)F)cc(O)c1-c1cnc2cn(C3CCOCC3)nc2n1]

Tetrahydro-2H-pyran-4-amine (Combi-Blocks, 146 mg, 1.4 mmol) was added to a microwave vial containing a solution of 3-azido-5-(2-(ethoxymethoxy)-6-methyl-4-(trifluoromethyl)phenyl)pyrazine-2-carbaldehyde (Intermediate 2, 50 mg, 0.13 mmol) in MeCN (4.8 mL) at rt. Then the microwave vial was sealed, and the reaction mixture was heated to 80° C. and stirred for 16 h. Then the reaction mixture was cooled to 40° C. followed by dropwise addition of HCl (0.75 mL, 3.0 mmol, 4 M in 1,4-dioxane). The reaction mixture was then stirred at 40° C. for 2 h. Then the reaction mixture was cooled to rt, quenched with sat. NaHCO₃ solution and diluted with H₂O and EtOAc. Then the layers were separated, and the aq. layer was extracted with EtOAc (×3). The combined organic layers were dried over MgSO₄, filtered, and the solvent was removed under reduced pressure. The resulting crude mixture was purified by silica gel chromatography (EtOAc:hexanes) to afford the title compound. LCMS [M+H]⁺=379.2 (calcd. 379.1). ¹H NMR (500 MHz, DMSO-d₆) δ 10.37 (s, 1H), 8.95 (s, 1H), 8.55 (s, 1H), 7.18 (s, 1H), 7.11 (s, 1H), 4.86 (tt, J=10.6, 5.0 Hz, 1H), 4.15-3.98 (m, 2H), 3.56 (td, J=11.5, 2.9 Hz, 2H), 2.24-2.12 (m, 4H), 2.16 (s, 3H).

### Example 2: 4-(6-(2-hydroxy-6-methyl-4-(trifluoromethyl)phenyl)-2H-pyrazolo[3,4-b]pyrazin-2-yl)-2-thiabicyclo[2.1.1]hexane 2,2-dioxide

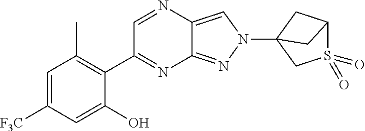 [SMILES: Cc1cc(C(F)(F)F)cc(O)c1-c1cnc2cn(C34CC(C3)S(=O)(=O)C4)nc2n1]

DIPEA (0.16 mL, 0.0.9 mmol) was added to a microwave vial containing 4-amino-2-thiabicyclo[2.1.1]hexane 2,2-dioxide hydrochloride (Enamine, 55 mg, 0.30 mmol) in MeCN (1.2 mL) at rt. The mixture was stirred at rt for 45 min followed by addition of a solution of 3-azido-5-(2-(ethoxymethoxy)-6-methyl-4-(trifluoromethyl)phenyl)pyrazine-2-carbaldehyde (Intermediate 2, 110 mg, 0.30 mmol) in MeCN (1.5 mL). Then the microwave vial was sealed, and the reaction mixture was heated to 80° C. and stirred for 16 h. Then the reaction mixture was cooled to 40° C. followed by dropwise addition of HCl (0.25 mL, 0.9 mmol, 4 M in 1,4-dioxane). The reaction mixture was then stirred at 40° C. for 2 h. Then the reaction mixture was cooled to rt, quenched with sat. NaHCO₃ solution and diluted with H₂O and EtOAc. Then the layers were separated, and the aq. layer was extracted with EtOAc (×3). The combined organic layers were dried over MgSO₄, filtered, and the solvent was removed under reduced pressure. The resulting crude mixture was purified by silica gel chromatography (EtOAc:hexanes) to afford the title compound. LCMS [M+H]⁺=425.1 (calcd. 425.1). ¹H NMR (500 MHz, DMSO-d₆) δ 10.47 (s, 1H), 9.06 (d, J=1.5 Hz, 1H), 8.61 (s, 1H), 7.19 (s, 1H), 7.11 (s, 1H), 4.17 (s, 1H), 3.89 (s, 2H), 3.29-3.25 (m, 2H), 2.73 (dd, J=6.4, 2.3 Hz, 2H), 2.16 (s, 3H).

Examples 3 and 4: 2-(2-((1S,4R,5R or 1R,4S,5S)-2-oxabicyclo[2.2.1]heptan-5-yl)-2H-pyrazolo[3,4-b]pyrazin-6-yl)-3-methyl-5-(trifluoromethyl)phenol (Example 3) and 2-(2-((1R,4S,5S or 1S,4R,5R)-2-oxabicyclo[2.2.1]heptan-5-yl)-2H-pyrazolo[3,4-b]pyrazin-6-yl)-3-methyl-5-(trifluoromethyl)phenol (Example 4)

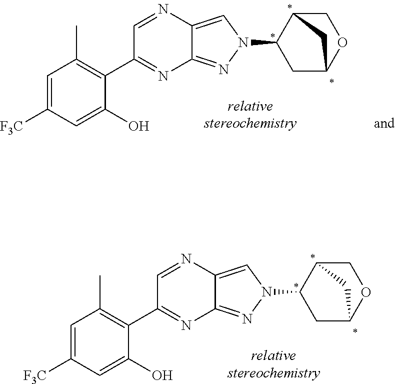 [SMILES: Cc1cc(C(F)(F)F)cc(O)c1-c1cnc2cn([C@@H]3C[C@@H]4C[C@H]3CO4)nc2n1 ; Cc1cc(C(F)(F)F)cc(O)c1-c1cnc2cn([C@H]3C[C@H]4C[C@@H]3CO4)nc2n1]

DIPEA (50 μL, 0.3 mmol) was added to a microwave vial containing (1S,4R,5R and 1R,4S,5S)-2-oxabicyclo[2.2.1]heptan-5-amine (Enamine, 18 mg, 0.12 mmol) in MeCN (0.5 mL) at rt. The mixture was stirred at rt for 45 min followed by addition of a solution of 3-azido-5-(2-(ethoxymethoxy)-6-methyl-4-(trifluoromethyl)phenyl)pyrazine-2-carbaldehyde (Intermediate 2, 38 mg, 0.1 mmol) in MeCN (0.5 mL). Then the microwave vial was sealed, and the reaction mixture was heated to 80° C. and stirred for 12 h. Then the reaction mixture was cooled to 40° C. followed by dropwise addition of HCl (0.15 mL, 0.6 mmol, 4 M in 1,4-dioxane). The reaction mixture was then stirred at 40° C. for 3 h. Then the reaction mixture was cooled to rt, and the solvents were removed under reduced pressure. The resulting crude mixture was purified by reverse phase HPLC (C18 stationary phase, MeCN/H₂O+0.1% FA) to afford a racemic mixture of the title compounds. The title compounds were resolved by preparative chiral SFC with Method F. The faster eluting isomer of the title compound was obtained (Example 3, 100% ee). ¹H NMR (500 MHz, DMSO-d₆) δ 10.38 (s, 1H), 8.99 (s, 1H), 8.56 (s, 1H), 7.18 (s, 1H), 7.11 (s, 1H), 5.04 (dd, J=8.2, 3.8 Hz, 1H), 4.49 (s, 1H), 3.68-3.61 (m, 2H), 2.99 (s, 1H), 2.41 (d, J=13.5 Hz, 1H), 2.33-2.25 (m, 1H), 2.18 (s, 1H), 2.16 (s, 3H), 1.67 (d, J=10.3 Hz, 1H). LCMS [M+H]⁺=391.2 (calcd. 391.1). The slower eluting isomer of the title compound was obtained (Example 4, 10000 ee). ¹H NMR (500 MHI-z, DMSO-d₆) δ 10.38 (s, TH), 8.99 (s, TH), 8.56 (s, TH), 7.18 (s, TH), 7.11 (s, TH), 5.04 (dd, J=8.2, 3.8 Hz, TH), 4.49 (s, TH), 3.68-3.61 (m, 2H), 2.99 (s, TH), 2.41 (d, J=13.5 Hz, TH), 2.33-2.25 (in, TH), 2.18 (s, TH), 2.16 (s, 3H), 1.67 (d, J=10.3 Hz, TH). LCMS [M+H]⁺=391.2 (calcd. 391.1).

The compounds listed in Table 5 were prepared using procedures similar to those described for Examples 1-4 using the appropriate starting materials. Addition of DIPEA was particularly important when the starting material amine was an acid salt.

TABLE 5
Examples 5-57

| | | | LCMS |
|---|---|---|---|
| Example | Structure | Name | [M + H]⁺ |
| 5 | 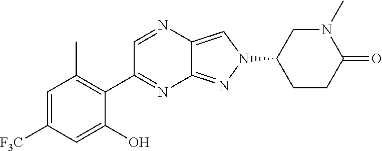 [SMILES: Cc1cc(C(F)(F)F)cc(O)c1-c1cnc2cn([C@H]3CCC(=O)N(C)C3)nc2n1] | (5S)-5-[6-[2-hydroxy-6-methyl-4- (trifluoromethyl)phenyl]pyrazolo[3,4- b]pyridazin-2-yl]-1-methyl-piperidin- 2-one | Calcd. 406.2, found 406.4 |
| 6 | 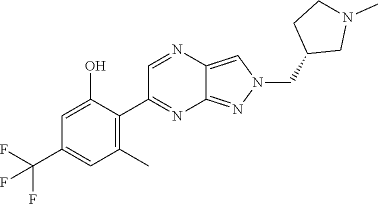 [SMILES: Cc1cc(C(F)(F)F)cc(O)c1-c1cnc2cn(C[C@@H]3CCN(C)C3)nc2n1] | (R)-3-methyl-2-(2-((1- methylpyrrolidin-3-yl)methyl)-2H- pyrazolo[3,4-b]pyrazin-6-yl)-5- (trifluoromethyl)phenol | Calcd. 392.2, found 392.4 |
| 7 | 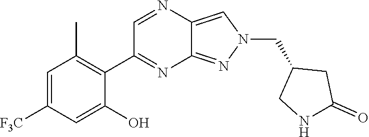 [SMILES: Cc1cc(C(F)(F)F)cc(O)c1-c1cnc2cn(C[C@H]3CNC(=O)C3)nc2n1] | (R)-4-((6-(2-hydroxy-6-methyl-4- (trifluoromethyl)phenyl)-2H- pyrazolo[3,4-b]pyrazin-2- yl)methyl)pyrrolidin-2-one | Calcd. 392.1, found 392.3 |
| 8 | 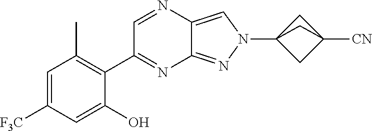 [SMILES: Cc1cc(C(F)(F)F)cc(O)c1-c1cnc2cn(C34CC(C#N)(C3)C4)nc2n1] | 3-(6-(2-hydroxy-6-methyl-4- (trifluoromethyl)phenyl)-2H- pyrazolo[3,4-b]pyrazin-2- yl)bicyclo[1.1.1]pentane-1- carbonitrile | Calcd. 386.1, found 386.4 |
| 9 | 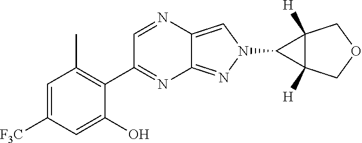 [SMILES: Cc1cc(C(F)(F)F)cc(O)c1-c1cnc2cn([C@@H]3[C@@H]4COC[C@@H]43)nc2n1] | 2-(2-((1R,5S,6s)-3- oxabicyclo[3.1.0]hexan-6-yl)-2H- pyrazolo[3,4-b]pyrazin-6-yl)-3- methyl-5-(trifluoromethyl)phenol | Calcd. 377.1, found 377.3 |
| 10 | 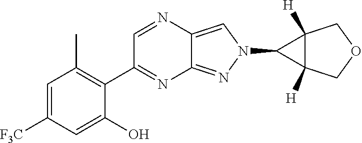 [SMILES: Cc1cc(C(F)(F)F)cc(O)c1-c1cnc2cn([C@H]3[C@@H]4COC[C@@H]43)nc2n1] | 2-(2-((1R,5S,6r)-3- oxabicyclo[3.1.0]hexan-6-yl)-2H- pyrazolo[3,4-b]pyrazin-6-yl)-3- methyl-5-(trifluoromethyl)phenol | Calcd. 377.1, found 377.3 |
| 11 | 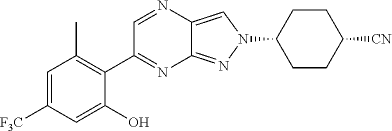 [SMILES: Cc1cc(C(F)(F)F)cc(O)c1-c1cnc2cn([C@H]3CC[C@@H](C#N)CC3)nc2n1] | (1s,4s)-4-(6-(2-hydroxy-6-methyl-4- (trifluoromethyl)phenyl)-2H- pyrazolo[3,4-b]pyrazin-2- yl)cyclohexane-1-carbonitrile | Calcd. 402.2, found 402.0 |
| 12 | 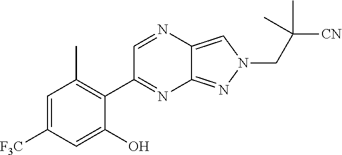 [SMILES: Cc1cc(C(F)(F)F)cc(O)c1-c1cnc2cn(CC(C)(C)C#N)nc2n1] | 3-(6-(2-hydroxy-6-methyl-4- (trifluoromethyl)phenyl)-2H- pyrazolo[3,4-b]pyrazin-2-yl)-2,2- dimethylpropanenitrile | Calcd. 376.1, found 376.0 |
| 13 | 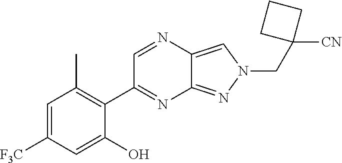 [SMILES: Cc1cc(C(F)(F)F)cc(O)c1-c1cnc2cn(CC3(C#N)CCC3)nc2n1] | 1-((6-(2-hydroxy-6-methyl-4- (trifluoromethyl)phenyl)-2H- pyrazolo[3,4-b]pyrazin-2- yl)methyl)cyclobutane-1-carbonitrile | Calcd. 388.1, found 388.0 |
| 14 | 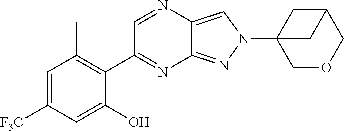 [SMILES: Cc1cc(C(F)(F)F)cc(O)c1-c1cnc2cn(C34COCC(C3)C4)nc2n1] | 2-(2-(3-oxabicyclo[3.1.1]heptan-1- yl)-2H-pyrazolo[3,4-b]pyrazin-6-yl)- 3-methyl-5-(trifluoromethyl)phenol | Calcd. 391.1, found 391.5 |
| 15 | 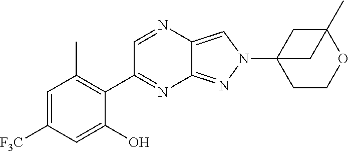 [SMILES: Cc1cc(C(F)(F)F)cc(O)c1-c1cnc2cn(C34CCOC(C)(C3)C4)nc2n1] | 3-methyl-2-(2-(1-methyl-2- oxabicyclo[3.1.1]heptan-5-yl)-2H- pyrazolo[3,4-b]pyrazin-6-yl)-5- (trifluoromethyl)phenol | Calcd. 405.2, found 405.6 |
| 16 | 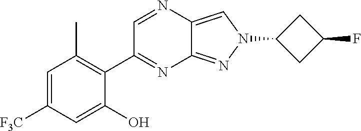 [SMILES: Cc1cc(C(F)(F)F)cc(O)c1-c1cnc2cn([C@H]3C[C@H](F)C3)nc2n1] | 2-(2-((1r,3r)-3-fluorocyclobutyl)-2H- pyrazolo[3,4-b]pyrazin-6-yl)-3- methyl-5-(trifluoromethyl)phenol | Calcd. 367.1, found 367.5 |
| 17 | 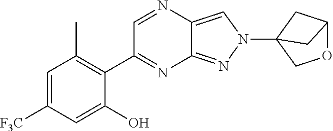 [SMILES: Cc1cc(C(F)(F)F)cc(O)c1-c1cnc2cn(C34COC(C3)C4)nc2n1] | 2-(2-(2-oxabicyclo[2.1.1]hexan-4-yl)- 2H-pyrrolo[3,4-b]pyrazin-6-yl)-3- methyl-5-(trifluoromethyl)phenol | Calcd. 377.1, found 377.0 |
| 18 | 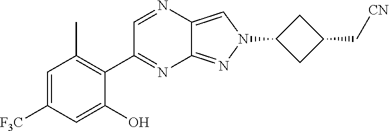 [SMILES: Cc1cc(C(F)(F)F)cc(O)c1-c1cnc2cn([C@H]3C[C@@H](CC#N)C3)nc2n1] | 2-((1r,3s)-3-(6-(2-hydroxy-6-methyl- 4-(trifluoromethyl)phenyl)-2H- pyrazolo[3,4-b]pyrazin-2- yl)cyclobutyl)acetonitrile | Calcd. 388.1, found 388.0 |
| 19 | 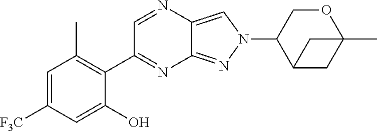 [SMILES: Cc1cc(C(F)(F)F)cc(O)c1-c1cnc2cn(C3COC4(C)CC3C4)nc2n1] | (R and S)-3-methyl-2-(2-(1-methyl-2- oxabicyclo[3.1.1]heptan-4-yl)-2H- pyrazolo[3,4-b]pyrazin-6-yl)-5- (trifluoromethyl)phenol | Calcd. 405.2, found 405.3 |
| 20 | 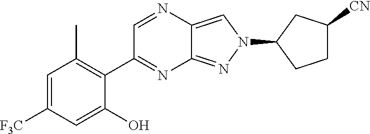 [SMILES: Cc1cc(C(F)(F)F)cc(O)c1-c1cnc2cn([C@@H]3CC[C@H](C#N)C3)nc2n1] | (1S,3R and 1R,3S)-3-(6-(2-hydroxy- 6-methyl-4-(trifluoromethyl)phenyl)- 2H-pyrazolo[3,4-b]pyrazin-2- yl)cyclopentane-1-carbonitrile | Calcd. 388.1, found 388.0 |
| 21 | 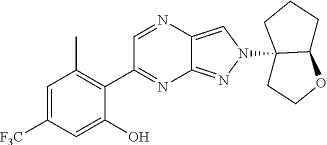 [SMILES: Cc1cc(C(F)(F)F)cc(O)c1-c1cnc2cn([C@]34CCC[C@H]3OCC4)nc2n1] | 2-(2-((3aS,6aR and 3aR,6aS)- hexahydro-3aH-cyclopenta[b]furan- 3a-yl)-2H-pyrazolo[3,4-b]pyrazin-6- yl)-3-methyl-5- (trifluoromethyl)phenol | Calcd. 405.2, found 405.3 |
| 22 | 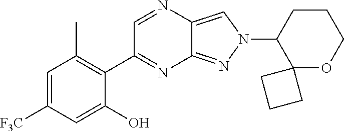 [SMILES: Cc1cc(C(F)(F)F)cc(O)c1-c1cnc2cn(C3CCCOC34CCC4)nc2n1] | (S and R)-2-(2-(5- oxaspiro[3.5]nonan-9-yl)-2H- pyrazolo[3,4-b]pyrazin-6-yl)-3- methyl-5-(trifluoromethyl)phenol | Calcd. 419.2, found 419.3 |
| 23 | 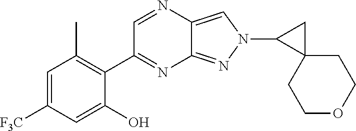 [SMILES: Cc1cc(C(F)(F)F)cc(O)c1-c1cnc2cn(C3CC34CCOCC4)nc2n1] | (S and R)-2-(2-(6-oxaspiro[2.5]octan- 1-yl)-2H-pyrazolo[3,4-b]pyrazin-6- yl)-3-methyl-5- (trifluoromethyl)phenol | Calcd. 405.2, found 405.3 |
| 24 | 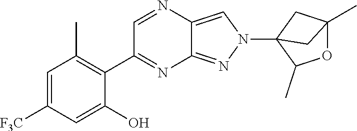 [SMILES: Cc1cc(C(F)(F)F)cc(O)c1-c1cnc2cn(C34CC(C)(C3)OC4C)nc2n1] | (S and R)-2-(2-(1,3-dimethyl-2- oxabicyclo[2.1.1]hexan-4-yl)-2H- pyrazolo[3,4-b]pyrazin-6-yl)-3- methyl-5-(trifluoromethyl)phenol | Calcd. 405.2, found 405.4 |
| 25 | 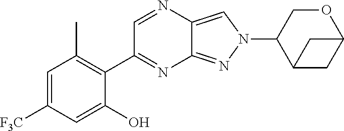 [SMILES: Cc1cc(C(F)(F)F)cc(O)c1-c1cnc2cn(C3COC4CC3C4)nc2n1] | (S and R)-2-(2-(2- oxabicyclo[3.1.1]heptan-4-yl)-2H- pyrazolo[3,4-b]pyrazin-6-yl)-3- methyl-5-(trifluoromethyl)phenol | Calcd. 391.1, found 391.4 |
| 26 | 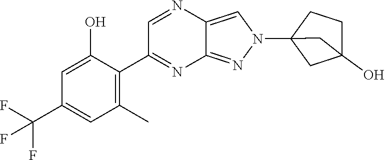 [SMILES: Cc1cc(C(F)(F)F)cc(O)c1-c1cnc2cn(C34CCC(O)(C3)C4)nc2n1] | 4-[6-[2-hydroxy-6-methyl-4- (trifluoromethyl)phenyl]pyrazolo[3,4- b]pyrazin-2-yl]bicyclo[2.1.1]hexan- 1-ol | Calcd. 391.1, found 391.4 |
| 27 | 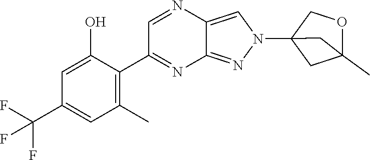 [SMILES: Cc1cc(C(F)(F)F)cc(O)c1-c1cnc2cn(C34COC(C)(C3)C4)nc2n1] | 3-methyl-2-[2-(1-methyl-2- oxabicyclo[2.1.1]hexan-4- yl)pyrazolo[3,4-b]pyrazin-6-yl]-5- (trifluoromethyl)phenol | Calcd. 391.1, found 391.5 |
| 28 | 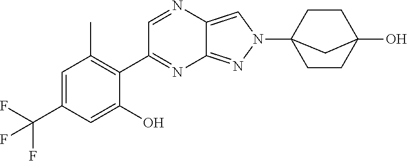 [SMILES: Cc1cc(C(F)(F)F)cc(O)c1-c1cnc2cn(C34CCC(O)(CC3)C4)nc2n1] | 4-[6-[2-hydroxy-6-methyl-4- (trifluoromethyl)phenyl]pyrazolo[3,4- b]pyrazin-2-yl]norbornan-1-ol | Calcd. 405.2, found 405.4 |
| 29 | 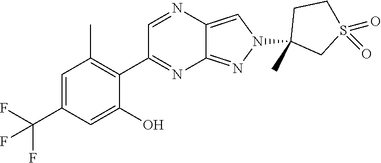 [SMILES: Cc1cc(C(F)(F)F)cc(O)c1-c1cnc2cn([C@]3(C)CCS(=O)(=O)C3)nc2n1] | 3-methyl-2-[2-[(3R)-3-methyl-1,1- dioxo-thiolan-3-yl]pyrazolo[3,4- b]pyrazin-6-yl]-5- (trifluoromethyl)phenol | Calcd. 427.1, found 427.4 |
| 30 | 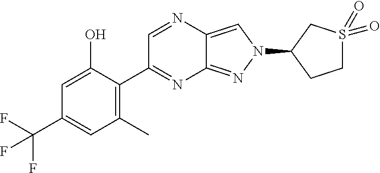 [SMILES: Cc1cc(C(F)(F)F)cc(O)c1-c1cnc2cn([C@@H]3CCS(=O)(=O)C3)nc2n1] | 2-[2-[(3R)-1,1-dioxothiolan-3- yl]pyrazolo[3,4-b]pyrazin-6-yl]-3- methyl-5-(trifluoromethyl)phenol | Calcd. 413.1, found 413.3 |
| 31 | 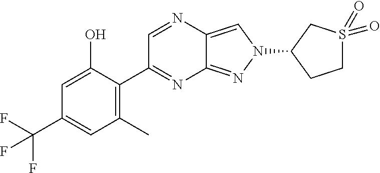 [SMILES: Cc1cc(C(F)(F)F)cc(O)c1-c1cnc2cn([C@H]3CCS(=O)(=O)C3)nc2n1] | 2-[2-[(3S)-1,1-dioxothiolan-3- yl]pyrazolo[3,4-b]pyrazin-6-yl]-3- methyl-5-(trifluoromethyl)phenol | Calcd. 413.1, found 413.3 |
| 32 | 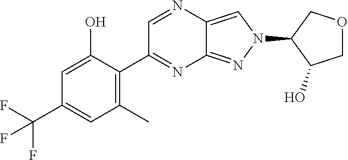 [SMILES: Cc1cc(C(F)(F)F)cc(O)c1-c1cnc2cn([C@H]3COC[C@@H]3O)nc2n1] | (3R,4S)-4-[6-[2-hydroxy-6-methyl-4- (trifluoromethyl)phenyl]pyrazolo[3,4- b]pyrazin-2-yl]tetrahydrofuran-3-ol | Calcd. 381.1, found 381.2 |
| 33 | 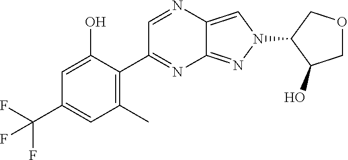 [SMILES: Cc1cc(C(F)(F)F)cc(O)c1-c1cnc2cn([C@@H]3COC[C@H]3O)nc2n1] | (3S,4R)-4-[6-[2-hydroxy-6-methyl-4- (trifluoromethyl)phenyl]pyrazolo[3,4- b]pyrazin-2-yl]tetrahydrofuran-3-ol | Calcd. 381.1, found 381.1 |
| 34 | 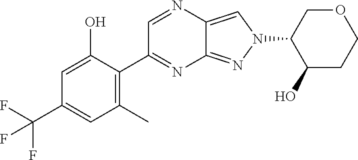 [SMILES: Cc1cc(C(F)(F)F)cc(O)c1-c1cnc2cn([C@@H]3COCC[C@H]3O)nc2n1] | (3R,4R)-3-[6-[2-hydroxy-6-methyl-4- (trifluoromethyl)phenyl]pyrazolo[3,4- b]pyrazin-2-yl]tetrahydropyran-4-ol | Calcd. 395.1, found 395.2 |
| 35 | 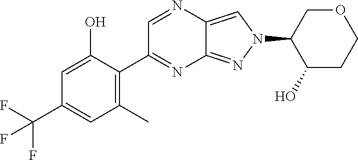 [SMILES: Cc1cc(C(F)(F)F)cc(O)c1-c1cnc2cn([C@H]3COCC[C@@H]3O)nc2n1] | (3S,4S)-3-[6-[2-hydroxy-6-methyl-4- (trifluoromethyl)phenyl]pyrazolo[3,4- b]pyrazin-2-yl]tetrahydropyran-4-ol | Calcd. 395.1, found 395.2 |
| 36 | 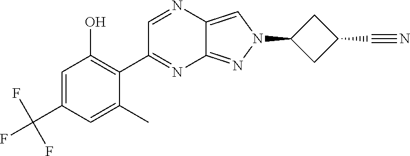 [SMILES: Cc1cc(C(F)(F)F)cc(O)c1-c1cnc2cn([C@H]3C[C@H](C#N)C3)nc2n1] | (1r,3r)-3-(6-(2-hydroxy-6-methyl-4- (trifluoromethyl)phenyl)-2H- pyrazolo[3,4-b]pyrazin-2- yl)cyclobutane-1-carbonitrile | Calcd. 374.1, found 374.3 |
| 37 | 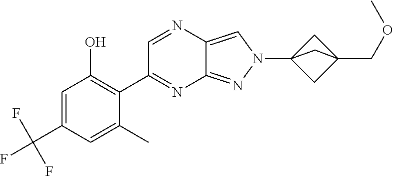 [SMILES: COCC12CC(n3cc4ncc(-c5c(C)cc(C(F)(F)F)cc5O)nc4n3)(C1)C2] | 2-[2-[3-(methoxymethyl)-1- bicyclo[1.1.1]pentanyl]pyrazolo[3,4- b]pyrazin-6-yl]-3-methyl-5- (trifluoromethyl)phenol | Calcd. 405.2, found 405.3 |
| 38 | 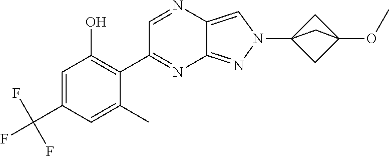 [SMILES: COC12CC(n3cc4ncc(-c5c(C)cc(C(F)(F)F)cc5O)nc4n3)(C1)C2] | 2-[2-(3-methoxy-1- bicyclo[1.1.1]pentanyl)pyrazolo[3,4- b]pyrazin-6-yl]-3-methyl-5- (trifluoromethyl)phenol | Calcd. 391.1, found 391.4 |
| 39 | 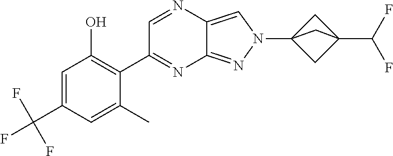 [SMILES: Cc1cc(C(F)(F)F)cc(O)c1-c1cnc2cn(C34CC(C(F)F)(C3)C4)nc2n1] | 2-[2-[3-(difluoromethyl)-1- bicyclo[1.1.1]pentanyl]pyrazolo[3,4- b]pyrazin-6-yl]-3-methyl-5- (trifluoromethyl)phenol | Calcd. 411.1, found 411.4 |
| 40 | 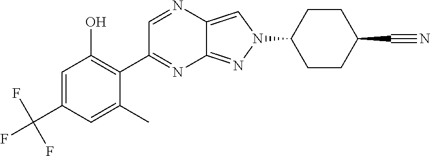 [SMILES: Cc1cc(C(F)(F)F)cc(O)c1-c1cnc2cn([C@H]3CC[C@H](C#N)CC3)nc2n1] | (1R,4R)-4-(6-(2-hydroxy-6-methyl-4- (trifluoromethyl)phenyl-2H- pyrazolo[3,4-b]pyrazin-2- yl)cyclohexane-1-carbonitrile | Calcd. 402.2, found 402.4 |
| 41 | 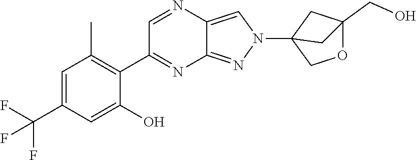 [SMILES: Cc1cc(C(F)(F)F)cc(O)c1-c1cnc2cn(C34COC(CO)(C3)C4)nc2n1] | 2-[2-[1-(hydroxymethyl)-2- oxabicyclo[2.1.1]hexan-4- yl]pyrazolo[3,4-b]pyrazin-6-yl]-3- methyl-5-(trifluoromethyl)phenol | Calcd. 407.1, found 407.3 |
| 42 | 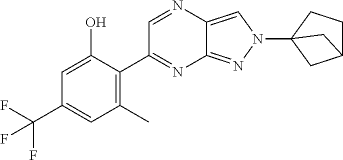 [SMILES: Cc1cc(C(F)(F)F)cc(O)c1-c1cnc2cn(C34CCC(C3)C4)nc2n1] | 2-[2-(1- bicyclo[2.1.1]hexanyl)pyrazolo[3,4- b]pyrazin-6-yl]-3-methyl-5- (trifluoromethyl)phenol | Calcd. 375.1, found 375.3 |
| 43 | 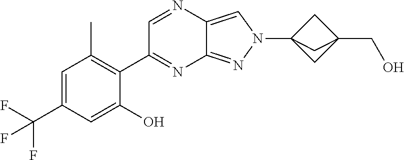 [SMILES: Cc1cc(C(F)(F)F)cc(O)c1-c1cnc2cn(C34CC(CO)(C3)C4)nc2n1] | 2-[2-[3-(hydroxymethyl)-1- bicyclo[1.1.1]pentanyl]pyrazolo[3,4- b]pyrazin-6-yl]-3-methyl-5- (trifluoromethyl)phenol | Calcd. 391.1, found 391.3 |
| 44 | 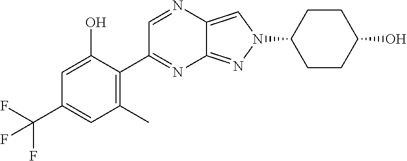 [SMILES: Cc1cc(C(F)(F)F)cc(O)c1-c1cnc2cn([C@H]3CC[C@@H](O)CC3)nc2n1] | 2-(2-((1s,4s)-4-hydroxycyclohexyl)- 2H-pyrazolo[3,4-b]pyrazin-6-yl)-3- methyl-4-(trifluoromethyl)phenol | Calcd. 393.2, found 393.3 |
| 45 | 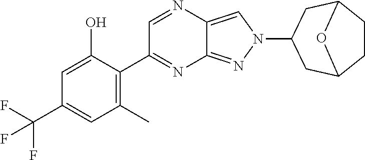 [SMILES: Cc1cc(C(F)(F)F)cc(O)c1-c1cnc2cn(C3CC4CCC(C3)O4)nc2n1] | 3-methyl-2-[2-(8- oxabicyclo[3.2.1]octan-3- yl)pyrazolo[3,4-b]pyrazin-6-yl]-5- (trifluoromethyl)phenol | Calcd. 405.2, found 405.3 |
| 46 | 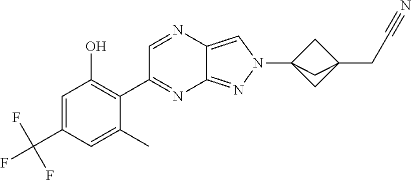 [SMILES: Cc1cc(C(F)(F)F)cc(O)c1-c1cnc2cn(C34CC(CC#N)(C3)C4)nc2n1] | 2-[3-[6-[2-hydroxy-6-methyl-4- (trifluoromethyl)phenyl]pyrazolo[3,4- b]pyrazin-2-yl]-1- bicyclo[1.1.1]pentanyl]acetonitrile | Calcd. 400.1, found 400.3 |
| 47 | 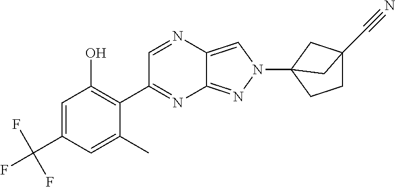 [SMILES: Cc1cc(C(F)(F)F)cc(O)c1-c1cnc2cn(C34CCC(C#N)(C3)C4)nc2n1] | 4-[6-[2-hydroxy-6-methyl-4- (trifluoromethyl)phenyl]pyrazolo[3,4- b]pyrazin-2-yl]bicyclo[2.1.1]hexane- 1-carbonitrile | Calcd. 400.1, found 400.3 |
| 48 | 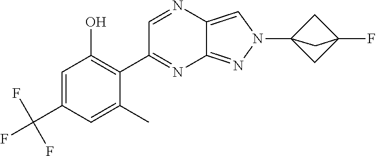 [SMILES: Cc1cc(C(F)(F)F)cc(O)c1-c1cnc2cn(C34CC(F)(C3)C4)nc2n1] | 2-[2-(3-fluoro-1- bicyclo[1.1.1]pentanyl)pyrazolo[3,4- b]pyrazin-6-yl]-3-methyl-5- (trifluoromethyl)phenol | Calcd. 379.1, found 379.3 |
| 49 | 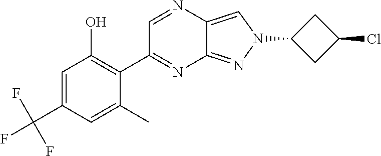 [SMILES: Cc1cc(C(F)(F)F)cc(O)c1-c1cnc2cn([C@H]3C[C@H](Cl)C3)nc2n1] | 2-(2-((1R,3R)-3-chlorocyclobutyl)- 2H-pyrazolo[3,4-b]pyrazin-6-yl)-3- methyl-5-(trifluoromethyl)phenol | Calcd. 383.1, found 383.3 |
| 50 | 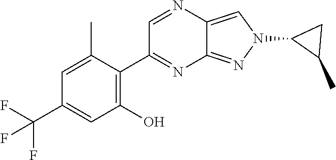 [SMILES: Cc1cc(C(F)(F)F)cc(O)c1-c1cnc2cn([C@@H]3C[C@H]3C)nc2n1] | 3-methyl-2-[2-[(1R,2R)-2- methylcyclopropyl]pyrazolo[3,4- b]pyrazin-6-yl]-5- (trifluoromethyl)phenol | Calcd. 349.1, found 349.1 |
| 51 | 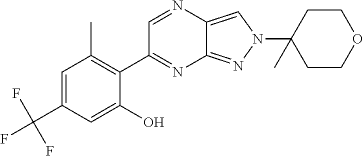 [SMILES: Cc1cc(C(F)(F)F)cc(O)c1-c1cnc2cn(C3(C)CCOCC3)nc2n1] | 3-methyl-2-[2-(4- methyltetrahydropyran-4- yl)pyrazolo[3,4-b]pyrazin-6-yl]-5- (trifluoromethyl)phenol | Calcd. 393.2, found 393.3 |
| 52 | 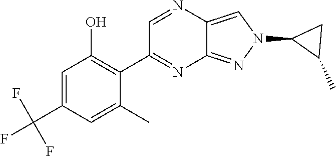 [SMILES: Cc1cc(C(F)(F)F)cc(O)c1-c1cnc2cn([C@H]3C[C@@H]3C)nc2n1] | 3-methyl-2-[2-[(1S,2S)-2- methylcyclopropyl]pyrazolo[3,4- b]pyrazin-6-yl]-5- (trifluoromethyl)phenol | Calcd. 349.1, found 349.2 |
| 53 | 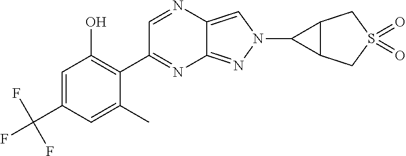 [SMILES: Cc1cc(C(F)(F)F)cc(O)c1-c1cnc2cn(C3C4CS(=O)(=O)CC43)nc2n1] | (1R,5S,6s or 1R,5S,6r)-6-(6-(2- hydroxy-6-methyl-4- (trifluoromethyl)phenyl)-2H- pyrazolo[3,4-b]pyrazin-2-yl)-3- thiabicyclo[3.1.0]hexane 3,3-dioxide | Calcd. 425.1, found 425.3 |
| 54 | 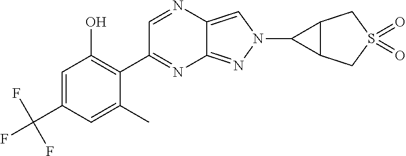 [SMILES: Cc1cc(C(F)(F)F)cc(O)c1-c1cnc2cn(C3C4CS(=O)(=O)CC43)nc2n1] | (1R,5S,6r or 1R,5S,6s)-6-(6-(2- hydroxy-6-methyl-4- (trifluoromethyl)phenyl)-2H- pyrazolo[3,4-b]pyrazin-2-yl)-3- thiabicyclo[3.1.0]hexane 3,3-dioxide | Calcd. 425.1, found 425.3 |
| 55 | 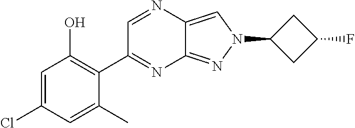 [SMILES: Cc1cc(Cl)cc(O)c1-c1cnc2cn([C@H]3C[C@H](F)C3)nc2n1] | 5-chloro-2-(2-((1r,3r)-3- fluorocyclobutyl)-2H-pyrazolo[3,4- b]pyrazin-6-yl)-3-methylphenol | Calcd. 333.1, found 333.1 |
| 56 | 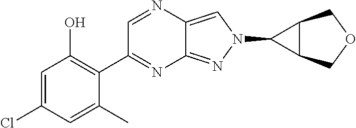 [SMILES: Cc1cc(Cl)cc(O)c1-c1cnc2cn([C@@H]3[C@@H]4COC[C@@H]43)nc2n1] | 2-(2-((1R,5S,6s)-3- oxabicyclo[3.1.0]hexan-6-yl)-2H- pyrazolo[3,4-b]pyrazin-6-yl)-5- chloro-3-methylphenol | Calcd. 343.1, found 343.1 |
| 57 | 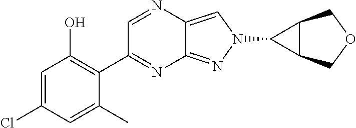 [SMILES: Cc1cc(Cl)cc(O)c1-c1cnc2cn([C@H]3[C@@H]4COC[C@@H]43)nc2n1] | 2-(2-((1R,5S,6r)-3- oxabicyclo[3.1.0]hexan-6-yl)-2H- pyrazolo[3,4-b]pyrazin-6-yl)-5- chloro-3-methylphenol | Calcd. 343.1, found 343.1 |

The compounds listed in Table 6 were prepared using procedures similar to those described for Examples 1-4 using the appropriate starting materials. Enantio- and diastereomeric products were separated using chiral SFC methods specified in the table. For those pairs of enantiomers, the fast-eluting isomer was listed first. Addition of DIPEA was particularly important when the starting material amine was an acid salt.

TABLE 6
Examples 58-105

| Example | Structure | Name | LCMS [M + H]⁺ | Chiral Method |
|---|---|---|---|---|
| 58 | 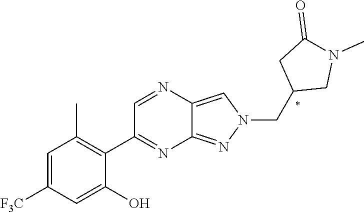 [SMILES: Cc1cc(C(F)(F)F)cc(O)c1-c1cnc2cn(CC3CC(=O)N(C)C3)nc2n1] | (R or S)-4-((6-(2-hydroxy-6- methyl-4-(trifluoromethyl) phenyl)-2H-pyrazolo[3,4- b]pyrazin-2-yl)methyl)-1- methylpyrrolidin-2-one | Calcd. 406.2, found 406.2 | A |
| 59 | 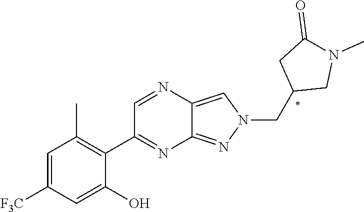 [SMILES: Cc1cc(C(F)(F)F)cc(O)c1-c1cnc2cn(CC3CC(=O)N(C)C3)nc2n1] | (S or R)-4-((6-(2-hydroxy-6- methyl-4-(trifluoromethyl) phenyl)-2H-pyrazolo[3,4- b]pyrazin-2-yl)methyl)-1- methylpyrrolidin-2-one | Calcd. 406.2, found 406.2 | A |
| 60 | 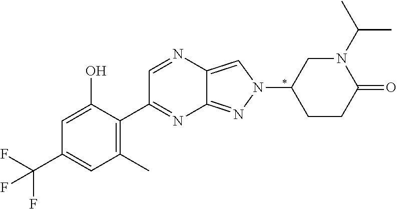 [SMILES: Cc1cc(C(F)(F)F)cc(O)c1-c1cnc2cn(C3CCC(=O)N(C(C)C)C3)nc2n1] | (R or S)-5-(6-(2-hydroxy-6- methyl-4-(trifluoromethyl) phenyl)-2H-pyrazolo[3,4- b]pyrazin-2-yl)-1- isopropylpiperidin-2-one | Calcd. 434.2, found 434.2 | B |
| 61 | 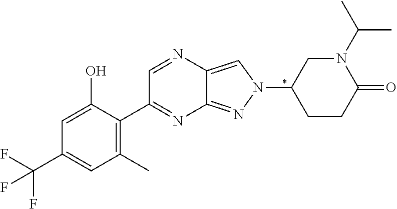 [SMILES: Cc1cc(C(F)(F)F)cc(O)c1-c1cnc2cn(C3CCC(=O)N(C(C)C)C3)nc2n1] | (S or R)-5-(6-(2-hydroxy-6- methyl-4-(trifluoromethyl) phenyl)-2H-pyrazolo[3,4- b]pyrazin-2-yl)-1- isopropylpiperidin-2-one | Calcd. 434.2, found 434.2 | B |
| 62 | 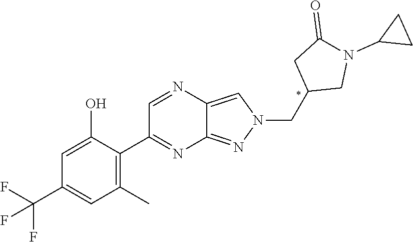 [SMILES: Cc1cc(C(F)(F)F)cc(O)c1-c1cnc2cn(CC3CC(=O)N(C4CC4)C3)nc2n1] | (R or S)-1-cyclopropyl-4- ((6-(2-hydroxy-6-methyl-4- (trifluoromethyl)phenyl)- 2H-pyrazolo[3,4-b]pyrazin- 2-yl)methyl)pyrrolidin-2- one | Calcd. 432.2, found 432.2 | C |
| 63 | 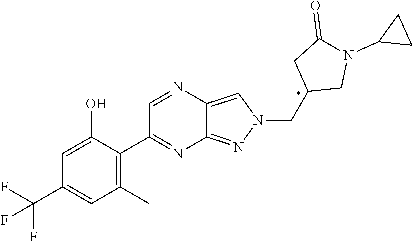 [SMILES: Cc1cc(C(F)(F)F)cc(O)c1-c1cnc2cn(CC3CC(=O)N(C4CC4)C3)nc2n1] | (S or R)-1-cyclopropyl-4- ((6-(2-hydroxy-6-methyl-4- (trifluoromethyl)phenyl)- 2H-pyrazolo[3,4-b]pyrazin- 2-yl)methyl)pyrrolidin-2- one | Calcd. 432.2, found 432.2 | C |
| 64 | 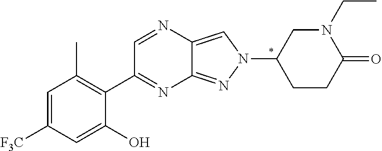 [SMILES: CCN1CC(n2cc3ncc(-c4c(C)cc(C(F)(F)F)cc4O)nc3n2)CCC1=O] | (R or S)-1-ethyl-5-(6-(2- hydroxy-6-methyl-4- (trifluoromethyl)phenyl)- 2H-pyrazolo[3,4-b]pyrazin- 2-yl)piperidin-2-one | Calcd. 420.2, found 420.3 | D |
| 65 | 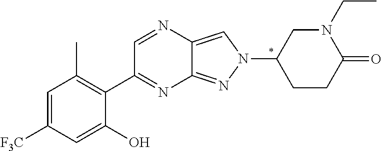 [SMILES: CCN1CC(n2cc3ncc(-c4c(C)cc(C(F)(F)F)cc4O)nc3n2)CCC1=O] | (S or R)-1-ethyl-5-(6-(2- hydroxy-6-methyl-4- (trifluoromethyl)phenyl)- 2H-pyrazolo[3,4-b]pyrazin- 2-yl)piperidin-2-one | Calcd. 420.2, found 420.3 | D |
| 66 | 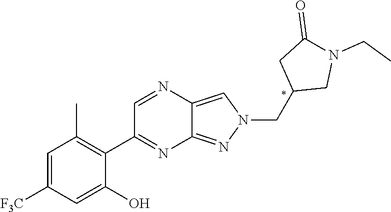 [SMILES: CCN1CC(Cn2cc3ncc(-c4c(C)cc(C(F)(F)F)cc4O)nc3n2)CC1=O] | (R or S)-1-ethyl-4-((6-(2- hydroxy-6-methyl-4- (trifluoromethyl)phenyl)- 2H-pyrazolo[3,4-b]pyrazin- 2-yl)methyl)pyrrolidin-2- one | Calcd. 420.2, found 420.3 | E |
| 67 | 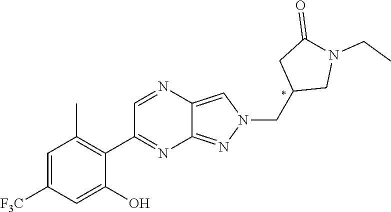 [SMILES: CCN1CC(Cn2cc3ncc(-c4c(C)cc(C(F)(F)F)cc4O)nc3n2)CC1=O] | (S or R)-1-ethyl-4-((6-(2- hydroxy-6-methyl-4- (trifluoromethyl)phenyl)- 2H-pyrazolo[3,4-b]pyrazin- 2-yl)methyl)pyrrolidin-2-one | Calcd. 420.2, found 420.3 | E |
| 68 | 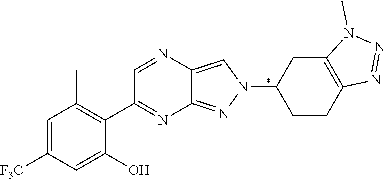 [SMILES: Cc1cc(C(F)(F)F)cc(O)c1-c1cnc2cn(C3CCc4nnn(C)c4C3)nc2n1] | (R or S)-3-methyl-2-(2-(1- methyl-4,5,6,7-tetrahydro- 1H-benzo[b][1,2,3]triazol- 6-yl)-2H-pyrazolo[3,4- b]pyrazin-6-yl)-5- (trifluoromethyl)phenol | Calcd. 430.2, found 430.2 | G |
| 69 | 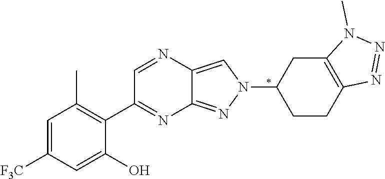 [SMILES: Cc1cc(C(F)(F)F)cc(O)c1-c1cnc2cn(C3CCc4nnn(C)c4C3)nc2n1] | (S or R)-3-methyl-2-(2-(1- methyl-4,5,6,7-tetrahydro- 1H-benzo[d][1,2,3]triazol- 6-yl)-2H-pyrazolo[3,4- b]pyrazin-6-yl)-5- (trifluoromethyl)phenol | Calcd. 430.2, found 430.2 | G |
| 70 | 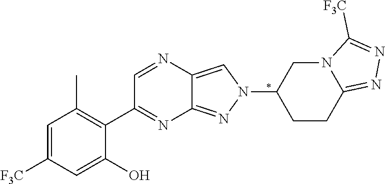 [SMILES: Cc1cc(C(F)(F)F)cc(O)c1-c1cnc2cn(C3CCc4nnc(C(F)(F)F)n4C3)nc2n1] | (R or S)-3-methyl-5- (trifluoromethyl)-2-(2-(3- (trifluoromethyl)-5,6,7,8- tetrahydro- [1,2,4]triazolo[4,3- a]pyridin-6-yl)-2H- pyrazolo[3,4-b]pyrazin-6- yl)phenol | Calcd. 484.1, found 484.2 | H |
| 71 | 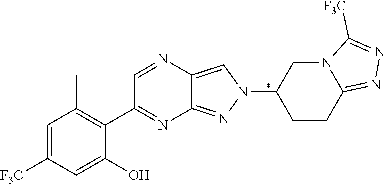 [SMILES: Cc1cc(C(F)(F)F)cc(O)c1-c1cnc2cn(C3CCc4nnc(C(F)(F)F)n4C3)nc2n1] | (S or R)-3-methyl-5- (trifluoromethyl)-2-(2-(3- (trifluoromethyl)-5,6,7,8- tetrahydro- [1,2,4]triazolo[4,3- a]pyridin-6-yl)-2H- pyrazolo[3,4-b]pyrazin-6- yl)phenol | Calcd. 484.1, found 484.2 | H |
| 72 | 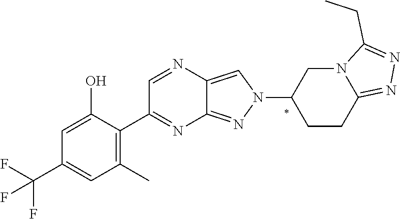 [SMILES: CCc1nnc2n1CC(n1cc3ncc(-c4c(C)cc(C(F)(F)F)cc4O)nc3n1)CC2] | (R or S)-2-(2-(3-ethyl- 5,6,7,8-tetrahydro- [1,2,4]triazolo[4,3- a]pyridin-6-yl)-2H- pyrazolo[3,4-b]pyrazin-6- yl)-3-methyl-5- (trifluoromethyl)phenol | Calcd. 444.2, found 444.2 | K |
| 73 | 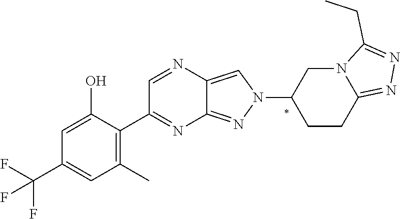 [SMILES: CCc1nnc2n1CC(n1cc3ncc(-c4c(C)cc(C(F)(F)F)cc4O)nc3n1)CC2] | (S or R)-2-(2-(3-ethyl- 5,6,7,8-tetrahydro- [1,2,4]triazolo[4,3- a]pyridin-6-yl)-2H- pyrazolo[3,4-b]pyrazin-6- yl)-3-methyl-5- (trifluoromethyl)phenol | Calcd. 444.2, found 444.2 | K |
| 74 | 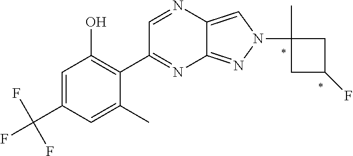 [SMILES: Cc1cc(C(F)(F)F)cc(O)c1-c1cnc2cn(C3(C)CC(F)C3)nc2n1] | 2-(2-((1R,3R or 1S,3S)-3- fluoro-1-methylcyclobutyl)- 2H-pyrazolo[3,4-b]pyrazin- 6-yl)-3-methyl-5- (trifluoromethyl)phenol | Calcd. 381.1, found 381.2 | L |
| 75 | 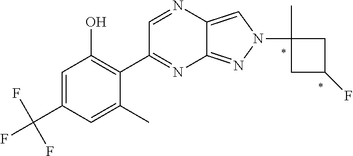 [SMILES: Cc1cc(C(F)(F)F)cc(O)c1-c1cnc2cn(C3(C)CC(F)C3)nc2n1] | 2-(2-((1S,3S or 1R,3R)-3- fluoro-1-methylcyclobutyl)- 2H-pyrazolo[3,4-b]pyrazin- 6-yl)-3-methyl-5- (trifluoromethyl)phenol | Calcd. 381.1, found 381.2 | L |
| 76 | 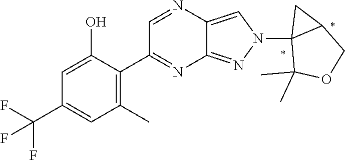 [SMILES: Cc1cc(C(F)(F)F)cc(O)c1-c1cnc2cn(C34CC3COC4(C)C)nc2n1] | 2-(2-((1R,5R or 1S,5S)-2,2- dimethyl-3- oxabicyclo[3.1.0]hexan-1- yl)-2H-pyrazolo[3,4- b]pyrazin-6-yl)-3-methyl-5- (trifluoromethyl)phenol | Calcd. 405.2, found 405.2 | M |
| 77 | 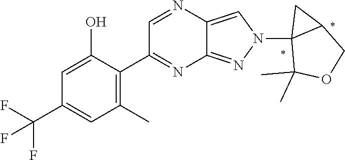 [SMILES: Cc1cc(C(F)(F)F)cc(O)c1-c1cnc2cn(C34CC3COC4(C)C)nc2n1] | 2-(2-((1S,5S or 1R,5R)-2,2- dimethyl-3- oxabicyclo[3.1.0]hexan-1- yl)-2H-pyrazolo[3,4- b]pyrazin-6-yl)-3-methyl-5- (trifluoromethyl)phenol | Calcd. 405.2, found 405.2 | M |
| 78 | 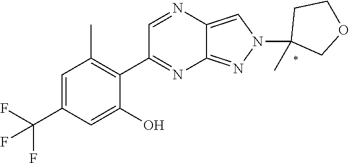 [SMILES: Cc1cc(C(F)(F)F)cc(O)c1-c1cnc2cn(C3(C)CCOC3)nc2n1] | (S or R)-3-methyl-2-(2-(3- methyltetrahydrofuran-3- yl)-2H-pyrazolo[3,4- b]pyrazin-6-yl)-5- (trifluoromethyl)phenol | Calcd. 379.1, found 379.2 | N |
| 79 | 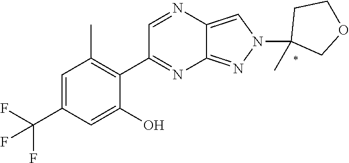 [SMILES: Cc1cc(C(F)(F)F)cc(O)c1-c1cnc2cn(C3(C)CCOC3)nc2n1] | (R or S)-3-methyl-2-(2-(3- methyltetrahydrofuran-3- yl)-2H-pyrazolo[3,4- b]pyrazin-6-yl)-5- (trifluoromethyl)phenol | Calcd. 379.1, found 379.2 | N |
| 80 | 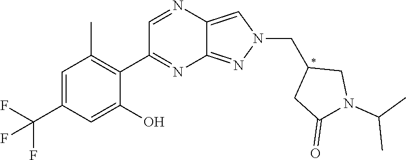 [SMILES: Cc1cc(C(F)(F)F)cc(O)c1-c1cnc2cn(CC3CC(=O)N(C(C)C)C3)nc2n1] | (R or S)-4-((6-(2-hydroxy-6- methyl-4- (trifluoromethyl)phenyl)- 2H-pyrazolo[3,4-b]pyrazin- 2-yl)methyl)-1- isopropylpyrrolidin-2-one | Calcd. 434.2, found 434.2 | L |
| 81 | 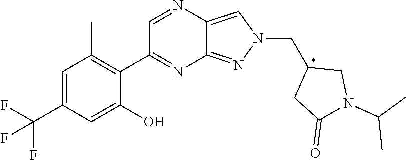 [SMILES: Cc1cc(C(F)(F)F)cc(O)c1-c1cnc2cn(CC3CC(=O)N(C(C)C)C3)nc2n1] | (S or R)-4-((6-(2-hydroxy-6- methyl-4- (trifluoromethyl)phenyl)- 2H-pyrazolo[3,4-b]pyrazin- 2-yl)methyl)-1- isopropylpyrrolidin-2-one | Calcd. 434.2, found 434.2 | L |
| 82 | 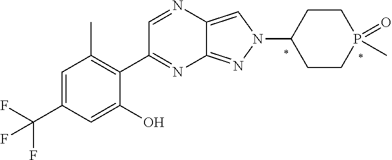 [SMILES: Cc1cc(C(F)(F)F)cc(O)c1-c1cnc2cn(C3CCP(C)(=O)CC3)nc2n1] | (1R,4R or 1S,4S)-4-(6-(2- hydroxy-6-methyl-4- (trifluoromethyl)phenyl)- 2H-pyrazolo[3,4-b]pyrazin- 2-yl)-1-methylphosphinane 1-oxide | Calcd. 425.1, found 425.2 | O (Peak 2) |
| 83 | 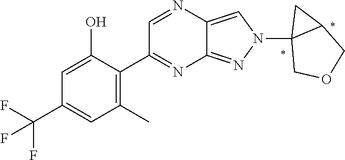 [SMILES: Cc1cc(C(F)(F)F)cc(O)c1-c1cnc2cn(C34COCC3C4)nc2n1] | 2-(2-((1R,5R or 1S,5S)-3- oxabicyclo[3.1.0]hexan-1- yl)-2H-pyrazolo[3,4- b]pyrazin-6-yl)-3-methyl-5- (trifluoromethyl)phenol | Calcd. 377.1, found 377.2 | L |
| 84 | 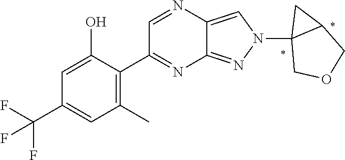 [SMILES: Cc1cc(C(F)(F)F)cc(O)c1-c1cnc2cn(C34COCC3C4)nc2n1] | 2-(2-((1S,5S or 1R,5R)-3- oxabicyclo[3.1.0]hexan-1- yl)-2H-pyrazolo[3,4- b]pyrazin-6-yl)-3-methyl-5- (trifluoromethyl)phenol | Calcd. 377.1, found 377.2 | L |
| 85 | 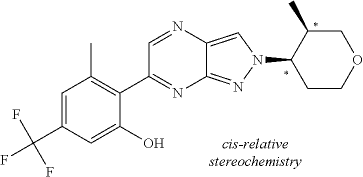 [SMILES: Cc1cc(C(F)(F)F)cc(O)c1-c1cnc2cn([C@@H]3CCOC[C@@H]3C)nc2n1] | 3-methyl-2-(2-((3R,4R or 3S,4S)-3-methyltetrahydro- 2H-pyran-4-yl)-2H- pyrazolo[3,4-b]pyrazin-6- yl)-5- (trifluoromethyl)phenol | Calcd. 393.2, found 393.2 | P |
| 86 | 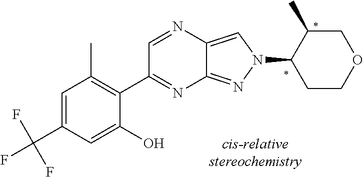 [SMILES: Cc1cc(C(F)(F)F)cc(O)c1-c1cnc2cn([C@@H]3CCOC[C@@H]3C)nc2n1] | 3-methyl-2-(2-((3S,4S or 3R,4R)-3-methyltetrahydro- 2H-pyran-4-yl)-2H- pyrazolo[3,4-b]pyrazin-6- yl)-5- (trifluoromethyl)phenol | Calcd. 393.2, found 393.2 | P |
| 87 | 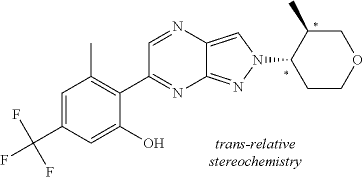 [SMILES: Cc1cc(C(F)(F)F)cc(O)c1-c1cnc2cn([C@H]3CCOC[C@@H]3C)nc2n1] | 3-methyl-2-(2-((3R,4S or 3S,4R)-3-methyltetrahydro- 2H-pyran-4-yl)-2H- pyrazolo[3,4-b]pyrazin-6- yl)-5- (trifluoromethyl)phenol | Calcd. 393.2, found 393.2 | P |
| 88 | 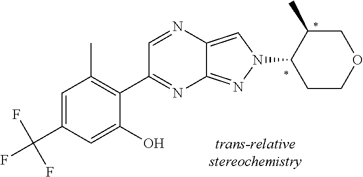 [SMILES: Cc1cc(C(F)(F)F)cc(O)c1-c1cnc2cn([C@H]3CCOC[C@@H]3C)nc2n1] | 3-methyl-2-(2-((3S,4R or 3R,4S)-3-methyltetrahydro- 2H-pyran-4-yl)-2H- pyrazolo[3,4-b]pyrazin-6- yl)-5- (trifluoromethyl)phenol | Calcd. 393.2, found 393.2 | P |
| 89 | 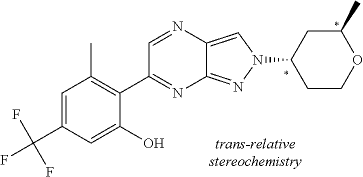 [SMILES: Cc1cc(C(F)(F)F)cc(O)c1-c1cnc2cn([C@H]3CCO[C@H](C)C3)nc2n1] | 3-methyl-2-(2-((2R,4S or 2S,4R)-2-methyltetrahydro- 2H-pyran-4-yl)-2H- pyrazolo[3,4-b]pyrazin-6- yl)-5- (trifluoromethyl)phenol | Calcd. 393.2, found 393.2 | N |
| 90 | 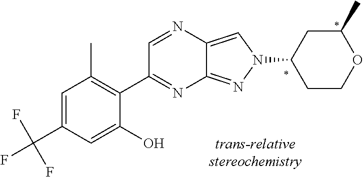 [SMILES: Cc1cc(C(F)(F)F)cc(O)c1-c1cnc2cn([C@H]3CCO[C@H](C)C3)nc2n1] | 3-methyl-2-(2-((2S,4R or 2R,4S)-2-methyltetrahydro- 2H-pyran-4-yl)-2H- pyrazolo[3,4-b]pyrazin-6- yl)-5- (trifluoromethyl)phenol | Calcd. 393.2, found 393.2 | N |
| 91 | 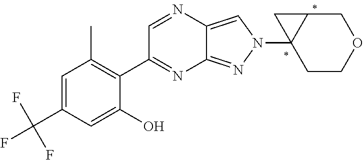 [SMILES: Cc1cc(C(F)(F)F)cc(O)c1-c1cnc2cn(C34CCOCC3C4)nc2n1] | 2-(2-((1R,6S or 1S,6R)-3- oxabicyclo[4.1.0]heptan-6- yl)-2H-pyrazolo[3,4- b]pyrazin-6-yl)-3-methyl-5- (trifluoromethyl)phenol | Calcd. 391.1, found 391.2 | P |
| 92 | 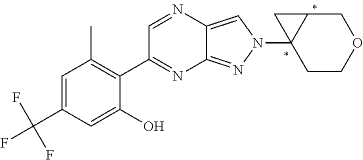 [SMILES: Cc1cc(C(F)(F)F)cc(O)c1-c1cnc2cn(C34CCOCC3C4)nc2n1] | 2-(2-((1S,6R or 1R,6S)-3- oxabicyclo[4.1.0]heptan-6- yl)-2H-pyrazolo[3,4-b] pyrazin-6-yl)-3-methyl-5- (trifluoromethyl)phenol | Calcd. 391.1, found 391.2 | P |
| 93 | 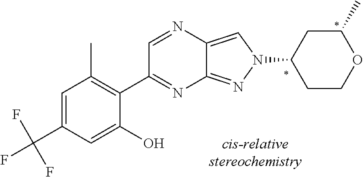 [SMILES: Cc1cc(C(F)(F)F)cc(O)c1-c1cnc2cn([C@H]3CCO[C@@H](C)C3)nc2n1] | 3-methyl-2-(2-((2S,4S or 2R,4R)-2-methyltetrahydro- 2H-pyran-4-yl)-2H- pyrazolo[3,4-b]pyrazin-6- yl)-5- (trifluoromethyl)phenol | Calcd. 393.2, found 393.2 | Q |
| 94 | 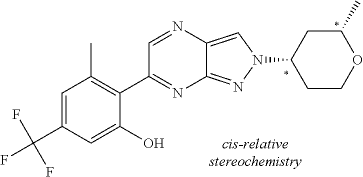 [SMILES: Cc1cc(C(F)(F)F)cc(O)c1-c1cnc2cn([C@H]3CCO[C@@H](C)C3)nc2n1] | 3-methyl-2-(2-((2R,4R or 2S,4S)-2-methyltetrahydro- 2H-pyran-4-yl)-2H- pyrazolo[3,4-b]pyrazin-6- yl)-5- (trifluoromethyl)phenol | Calcd. 393.2, found 393.2 | Q |
| 95 | 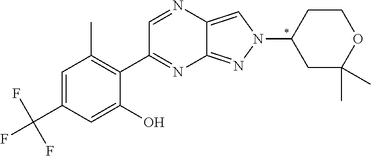 [SMILES: Cc1cc(C(F)(F)F)cc(O)c1-c1cnc2cn(C3CCOC(C)(C)C3)nc2n1] | (R or S)-2-(2-(2,2- dimethyltetrahydro-2H- pyran-4-yl)-2H- pyrazolo[3,4-b]pyrazin-6- yl)-3-methyl-5- (trifluoromethyl)phenol | Calcd. 407.2, found 407.2 | Q |
| 96 | 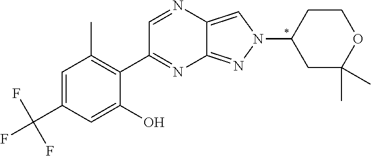 [SMILES: Cc1cc(C(F)(F)F)cc(O)c1-c1cnc2cn(C3CCOC(C)(C)C3)nc2n1] | (S or R)-2-(2-(2,2- dimethyltetrahydro-2H- pyran-4-yl)-2H- pyrazolo[3,4-b]pyrazin-6- yl)-3-methyl-5- (trifluoromethyl)phenol | Calcd. 407.2, found 407.2 | Q |
| 97 | 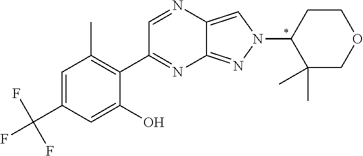 [SMILES: Cc1cc(C(F)(F)F)cc(O)c1-c1cnc2cn(C3CCOCC3(C)C)nc2n1] | (R or S)-2-(2-(3,3- dimethyltetrahydro-2H- pyran-4-yl)-2H- pyrazolo[3,4-b]pyrazin-6- yl)-3-methyl-5- (trifluoromethyl)phenol | Calcd. 407.2, found 407.3 | L |
| 98 | 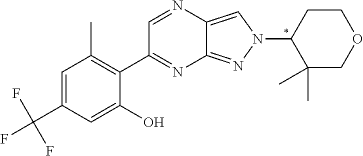 [SMILES: Cc1cc(C(F)(F)F)cc(O)c1-c1cnc2cn(C3CCOCC3(C)C)nc2n1] | (S or R)-2-(2-(3,3- dimethyltetrahydro-2H- pyran-4-yl)-2H- pyrazolo[3,4-b]pyrazin-6- yl)-3-methyl-5- (trifluoromethyl)phenol | Calcd. 407.2, found 407.3 | L |
| 99 | 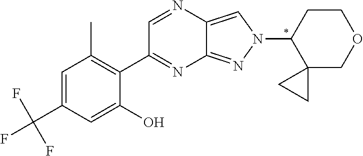 [SMILES: Cc1cc(C(F)(F)F)cc(O)c1-c1cnc2cn(C3CCOCC34CC4)nc2n1] | (R or S)-2-(2-(5- oxaspiro[2.5]octan-8-yl)- 2H-pyrazolo[3,4-b]pyrazin- 6-yl)-3-methyl-5- (trifluoromethyl)phenol | Calcd. 405.2, found 405.3 | Q |
| 100 | 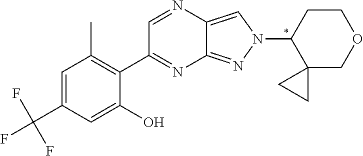 [SMILES: Cc1cc(C(F)(F)F)cc(O)c1-c1cnc2cn(C3CCOCC34CC4)nc2n1] | (S or R)-2-(2-(5- oxaspiro[2.5]octan-8-yl)- 2H-pyrazolo[3,4-b]pyrazin- 6-yl)-3-methyl-5- (trifluoromethyl)phenol | Calcd. 405.2, found 405.3 | Q |
| 101 | 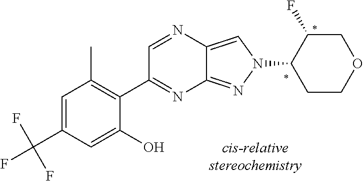 [SMILES: Cc1cc(C(F)(F)F)cc(O)c1-c1cnc2cn([C@H]3CCOC[C@H]3F)nc2n1] | 2-(2-((3S,4S or 3R,4R)-3- fluorotetrahydro-2H-pyran- 4-yl)-2H-pyrazolo[3,4- b]pyrazin-6-yl)-3-methyl-5- (trifluoromethyl)phenol | Calcd. 397.1, found 397.3 | R |
| 102 | 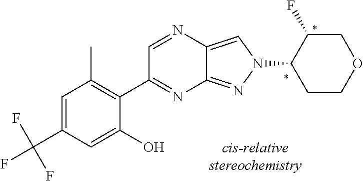 [SMILES: Cc1cc(C(F)(F)F)cc(O)c1-c1cnc2cn([C@H]3CCOC[C@H]3F)nc2n1] | 2-(2-((3R,4R or 3S,4S)-3- fluorotetrahydro-2H-pyran- 4-yl)-2H-pyrazolo[3,4- b]pyrazin-6-yl)-3-methyl-5- (trifluoromethyl)phenol | Calcd. 397.1, found 397.3 | R |
| 103 | 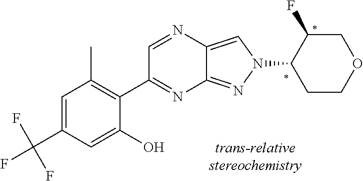 [SMILES: Cc1cc(C(F)(F)F)cc(O)c1-c1cnc2cn([C@H]3CCOC[C@@H]3F)nc2n1] | 2-(2-((3R,4S or 3S,4R)-3- fluorotetrahydro-2H-pyran- 4-yl)-2H-pyrazolo[3,4- b]pyrazin-6-yl)-3-methyl-5- (trifluoromethyl)phenol | Calcd. 397.1, found 397.3 | R |
| 104 | 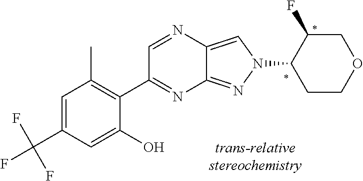 [SMILES: Cc1cc(C(F)(F)F)cc(O)c1-c1cnc2cn([C@H]3CCOC[C@@H]3F)nc2n1] | 2-(2-((3S,4R or 3R,4S)-3- fluorotetrahydro-2H-pyran- 4-yl)-2H-pyrazolo[3,4- b]pyrazin-6-yl)-3-methyl-5- (trifluoromethyl)phenol | Calcd. 397.1, found 397.3 | R |
| 105 | 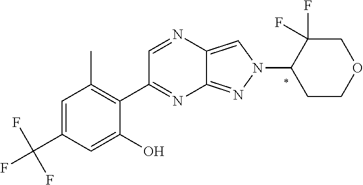 [SMILES: Cc1cc(C(F)(F)F)cc(O)c1-c1cnc2cn(C3CCOCC3(F)F)nc2n1] | (R or S)-2-(2-(3,3- difluorotetrahydro-2H- pyran-4-yl)-2H- pyrazolo[3,4-b]pyrazin-6- yl)-3-methyl-5- (trifluoromethyl)phenol | Calcd. 415.1, found 415.3 | S (Peak 2) |

### Example 106: (S or R)-5-(6-(2-hydroxy-6-methyl-4-(trifluoromethyl)phenyl)-3-methyl-2H-pyrazolo[3,4-b]pyrazin-2-yl)-1-methylpiperidin-2-one (Peak 2)

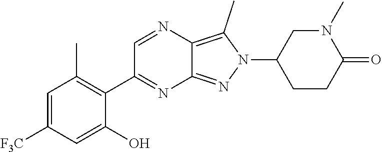 [SMILES: Cc1cc(C(F)(F)F)cc(O)c1-c1cnc2c(C)n(C3CCC(=O)N(C)C3)nc2n1]

DIPEA (0.03 mL, 0.15 mmol) was added to a mixture of (R and S)-5-amino-1-methylpiperidin-2-one (19.5 mg, 0.15 mmol) and 1-(3-azido-5-(2-(ethoxymethoxy)-6-methyl-4-(trifluoromethyl)phenyl)pyrazin-2-yl)ethan-1-one (Intermediate 4, 20 mg, 0.05 mmol) at rt. The neat reaction mixture was then heated to 90° C. and stirred for 15 min. Then the reaction mixture was cooled to rt, dissolved in DCM (1 mL), and TFA (0.04 mL, 0.5 mmol) was added. Then the reaction mixture was stirred at rt for 1 h. The solvents were then removed under reduced pressure, and the resulting crude mixture was purified by reverse phase HPLC (C18 stationary phase, MeCN/H₂O+0.100 TFA) to afford a racemic mixture of the title compounds. The title compounds were resolved by preparative chiral SFC with Method I. The slower eluting isomer of the title compound was obtained (Example 106, 100% ee). ¹H NMR (CD₃OD, 400 MHz) δ 8.52 (s, 1H), 7.12 (s, 1H), 7.05 (s, 1H), 5.29 (br d, J=5.6 Hz, 1H), 4.03 (dd, J=12.4, 8.1 Hz, 1H), 3.80 (dd, J=12.3, 5.5 Hz, 1H), 3.02 (s, 3H), 2.85 (s, 3H), 2.67-2.53 (m, 3H), 2.39-2.29 (m, 1H), 2.21 (s, 3H). LCMS [M+H]⁺=420.1 (calcd. 420.2).

The compound listed in Table 7 was prepared using procedures similar to those described for Example 106 using the appropriate starting materials. Addition of DIPEA was particularly important when the starting material amine was an acid salt.

TABLE 7
Example 107

| | | | LCMS |
|---|---|---|---|
| Example | Structure | Name | [M + H]⁺ |
| 107 | 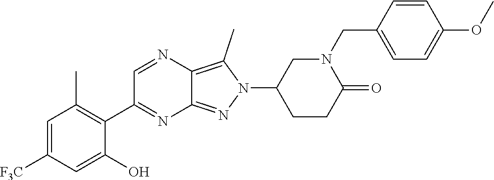 [SMILES: COc1ccc(CN2CC(n3nc4nc(-c5c(C)cc(C(F)(F)F)cc5O)cnc4c3C)CCC2=O)cc1] | (R and S)-5-(6-(2-hydroxy-6-methyl- 4-(trifluoromethyl)phenyl)-3-methyl-2H- pyrazolo[3,4-b]pyrazin-2-yl)-1- (4-methoxybenzyl)piperidin-2-one | Calcd. 526.2, found 526.2 |

The compounds listed in Table 8 were prepared using procedures similar to those described for Example 106 using the appropriate starting materials. Enantio- and diastereomeric products were separated using chiral SFC methods specified in the table. For those pairs of enantiomers, the fast-eluting isomer was listed first. Addition of DIPEA was particularly important when the starting material amine was an acid salt.

TABLE 8
Examples 108-109

| | | | LCMS | Chiral |
|---|---|---|---|---|
| Example | Structure | Name | [M + H]⁺ | Method |
| 108 | 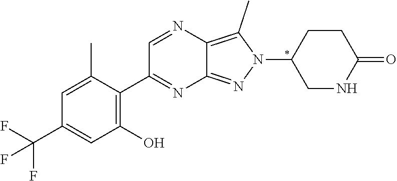 [SMILES: Cc1cc(C(F)(F)F)cc(O)c1-c1cnc2c(C)n(C3CCC(=O)NC3)nc2n1] | (R or S)-5-(6-(2-hydroxy-6- methyl-4-(trifluoromethyl) phenyl)-3-methyl-2H- pyrazolo[3,4-b]pyrazin-2- yl)piperidin-2-one | Calcd. 406.2, found 406.0 | J |
| 109 | 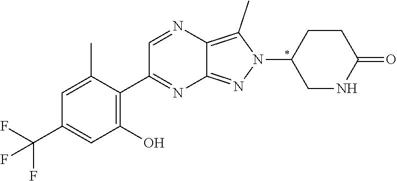 [SMILES: Cc1cc(C(F)(F)F)cc(O)c1-c1cnc2c(C)n(C3CCC(=O)NC3)nc2n1] | (S or R)-5-(6-(2-hydroxy-6- methyl-4-(trifluoromethyl) phenyl)-3-methyl-2H- pyrazolo[3,4-b]pyrazin-2- yl)piperidin-2-one | Calcd. 406.2, found 406.3 | J |

### Example 110: (R)-3-ethyl-5-((6-(2-hydroxy-6-methyl-4-(trifluoromethyl)phenyl)-2H-pyrazolo[3,4-b]pyrazin-2-yl)methyl)oxazolidin-2-one

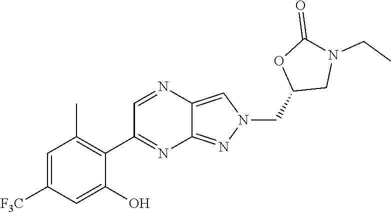 [SMILES: CCN1C[C@H](Cn2cc3ncc(-c4c(C)cc(C(F)(F)F)cc4O)nc3n2)OC1=O]

Step 1: (R)-5-((6-(2-(ethoxymethoxy)-6-methyl-4-(trifluoromethyl)phenyl)-2H-pyrazolo[3,4-b]pyrazin-2-yl)methyl)oxazolidin-2-one: DIPEA (0.14 mL, 0.79 mmol) was added to a microwave vial containing (S)-5-(aminomethyl)oxazolidin-2-one hydrochloride (Pharmablock, 44 mg, 0.29 mmol) in MeCN (1.3 mL) at rt. The mixture was stirred at rt for 45 min followed by addition of a solution of 3-azido-5-(2-(ethoxymethoxy)-6-methyl-4-(trifluoromethyl)phenyl) pyrazine-2-carbaldehyde (Intermediate 2, 100 mg, 0.26 mmol) in MeCN (1.3 mL). Then the microwave vial was sealed, and the reaction mixture was heated to 60° C. and stirred for 12 h. Then the reaction mixture was cooled to rt, and the solvents were removed under reduced pressure. The resulting crude mixture was purified by silica gel chromatography (DCM:MeOH) to afford the title compound. LCMS [M+H]⁺=452.2 (calcd. 452.2).

Step 2: (R)-5-((6-(2-(ethoxymethoxy)-6-methyl-4-(trifluoromethyl)phenyl)-2H-pyrazolo[3,4-b]pyrazin-2-yl)methyl)-3-ethyloxazolidin-2-one: KHMDS (0.51 mL, 0.05 mmol, 1 M in THF) was added at −78° C. to a solution of (R)-5-((6-(2-(ethoxymethoxy)-6-methyl-4-(trifluoromethyl)phenyl)-2H-pyrazolo[3,4-b]pyrazin-2-yl)methyl)oxazolidin-2-one (19 mg, 0.04 mmol) in THF (0.4 mL) under N2 atmosphere. The resulting mixture was stirred at −78° C. for 30 min, followed by the addition of iodoethane (13 μL, 0.17 mmol). The resulting mixture was warmed up to rt and stirred for 12 h. The reaction mixture was then quenched with saturated aq. solution of NH₄Cl (20 μL), and the resulting crude residue was purified by silica gel chromatography (EtOAc:EtOH) to afford the title compound. LCMS [M+H]⁺=480.3 (calcd. 480.2).

Step 3: (R)-3-ethyl-5-((6-(2-hydroxy-6-methyl-4-(trifluoromethyl)phenyl)-2H-pyrazolo[3,4-b]pyrazin-2-yl)methyl)oxazolidin-2-one: HCl (0.1 mL, 0.42 mmol, 4.0 M in 1,4-dioxane) was added to a vial containing (R)-5-((6-(2-(ethoxymethoxy)-6-methyl-4-(trifluoromethyl)phenyl)-2H-pyrazolo[3,4-b]pyrazin-2-yl)methyl)-3-ethyloxazolidin-2-one (20 mg, 0.04 mmol) in MeOH (0.4 mL) at 0° C. The reaction mixture was then warmed to rt and stirred for 12 h. Then, the reaction mixture was quenched with ammonia solution (1 mL, 7 M in MeOH), and the solvents were removed under reduced pressure. The resulting crude residue was purified by reverse phase HPLC (C18 stationary phase, MeCN/H₂O+0.1% TFA) to afford the title compound. ¹H NMR (500 MHz, DMSO-d₆) δ 10.48 (d, J=2.1 Hz, 1H), 8.85 (s, 1H), 8.58 (s, 1H), 7.12 (s, 1H), 7.12 (s, 1H), 5.11 (s, 1H), 4.91-4.78 (m, 2H), 3.74 (t, J=9.2 Hz, 1H), 3.56 (dd, J=8.9, 5.6 Hz, 1H), 3.15-3.03 (m, 2H), 2.16 (s, 3H), 0.91 (t, J=7.2 Hz, 3H). LCMS [M+H]⁺=422.4 (calcd. 422.1).

Examples 111 and 112: (R or S)-5-(6-(2-hydroxy-4-(trifluoromethyl)phenyl)-5-methyl-2H-pyrazolo[3,4-b]pyrazin-2-yl)-1-methylpiperidin-2-one (Example 111) and (S or R)-5-(6-(2-hydroxy-4-(trifluoromethyl)phenyl)-5-methyl-2H-pyrazolo[3,4-b]pyrazin-2-yl)-1-methylpiperidin-2-one (Example 112)

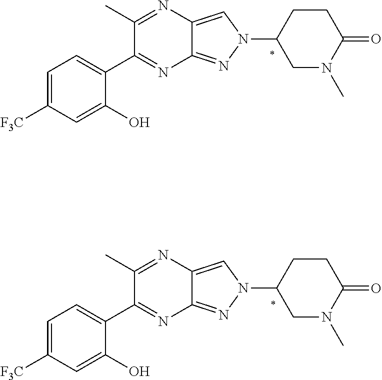 [SMILES: Cc1nc2cn(C3CCC(=O)N(C)C3)nc2nc1-c1ccc(C(F)(F)F)cc1O ; Cc1nc2cn(C3CCC(=O)N(C)C3)nc2nc1-c1ccc(C(F)(F)F)cc1O]

Step 1: (R and S)-5-(6-chloro-5-methyl-2H-pyrazolo[3,4-b]pyrazin-2-yl)-1-methylpiperidin-2-one: To a solution of 6-chloro-5-methyl-1H-pyrazolo[3,4-b]pyrazine (200 mg, 1.19 mmol) in DMF (3 mL) was added 1-methyl-6-oxopiperidin-3-yl 4-methylbenzenesulfonate (336 mg, 1.19 mmol) and cesium carbonate (773 mg, 2.37 mmol) at rt. The reaction mixture was then warmed to 100° C. and stirred for 4 h. Then the reaction mixture was cooled to rt, the solvents were removed under reduced pressure, and the resulting crude residue purified by reverse phase HPLC (C18 stationary phase, MeCN/H₂O+0.1% TFA) to afford the title compound. LCMS [M+H]⁺=279.9 (calcd. 280.1).

Step 2: (R or S)-5-(6-(2-hydroxy-4-(trifluoromethyl)phenyl)-5-methyl-2H-pyrazolo[3,4-b]pyrazin-2-yl)-1-methylpiperidin-2-one and (S or R)-5-(6-(2-hydroxy-4-(trifluoromethyl)phenyl)-5-methyl-2H-pyrazolo[3,4-b]pyrazin-2-yl)-1-methylpiperidin-2-one: To a solution of (R and S)-5-(6-chloro-5-methyl-2H-pyrazolo[3,4-b]pyrazin-2-yl)-1-methylpiperidin-2-one (23 mg, 0.08 mmol) and (2-hydroxy-4-(trifluoromethyl)phenyl)boronic acid (18.6 mg, 0.090 mmol) in t-AmOH (0.5 mL) and water (0.1 mL), cesium carbonate (80 mg, 0.247 mmol) and chloro[(di(1-adamantyl)-N-butylphosphine)-2-(2-aminobiphenyl)]palladium(II) (2.8 mg, 4 μmol) were added. The mixture was degassed with N2 for 15 min, then heated to 100° C. and stirred for 2 h. Then the reaction mixture was cooled to rt, and the solvents removed under reduced pressure. The resulting crude residue was purified by reverse phase HPLC (C18 stationary phase, MeCN/H₂O+0.10% TFA) to afford a racemic mixture of the title compounds. The title compounds were resolved by preparative chiral SFC with Method I. The faster eluting isomer of the title compound was obtained (Example 111, 100% ee). ¹H NMR (400 MHz, CD₃OD) δ 8.59 (s, 1H), 7.48 (d, J=7.9 Hz, 1H), 7.29 (d, J=8.1 Hz, 1H), 7.22 (s, 1H), 5.19 (br d, J=7.6 Hz, 1H), 4.10-3.96 (m, 1H), 3.93 (br d, J=5.2 Hz, 1H), 3.01 (s, 3H), 2.68-2.56 (m, 2H), 2.56-2.50 (m, 4H), 2.46 (br dd, J=11.3, 6.0 Hz, 1H). LCMS [M+H]⁺=406.1 (calcd. 406.1). The slower eluting isomer of the title compound was obtained (Example 112, 98% ee). ¹H NMR (400 MHz, CD₃OD) δ 8.59 (s, 1H), 7.48 (d, J=7.9 Hz, 1H), 7.29 (d, J=8.1 Hz, 1H), 7.22 (s, 1H), 5.19 (br d, J=7.6 Hz, 1H), 4.10-3.96 (m, 1H), 3.93 (br d, J=5.2 Hz, 1H), 3.01 (s, 3H), 2.68-2.56 (m, 2H), 2.56-2.50 (m, 4H), 2.46 (br dd, J=11.3, 6.0 Hz, 1H). LCMS [M+H]⁺=406.1 (calcd. 406.1).

Examples 113, 114, 115, and 116: (S or R)-1-cyclopropyl-4-((6-(2-(ethoxymethoxy)-6-methyl-4-(trifluoromethyl)phenyl)-3-((S or R)-1-hydroxyethyl)-2H-pyrazolo[3,4-b]pyrazin-2-yl)methyl)pyrrolidin-2-one (Example 113), (S or R)-1-cyclopropyl-4-((6-(2-(ethoxymethoxy)-6-methyl-4-(trifluoromethyl)phenyl)-3-((R or S)-1-hydroxyethyl)-2H-pyrazolo[3,4-b]pyrazin-2-yl)methyl)pyrrolidin-2-one (Example 114), (R or S)-1-cyclopropyl-4-((6-(2-(ethoxymethoxy)-6-methyl-4-(trifluoromethyl)phenyl)-3-((R or S)-1-hydroxyethyl)-2H-pyrazolo[3,4-b]pyrazin-2-yl)methyl)pyrrolidin-2-one (Example 115), and (R or S)-1-cyclopropyl-4-((6-(2-(ethoxymethoxy)-6-methyl-4-(trifluoromethyl)phenyl)-3-((S or R)-1-hydroxyethyl)-2H-pyrazolo[3,4-b]pyrazin-2-yl)methyl)pyrrolidin-2-one (Example 116)

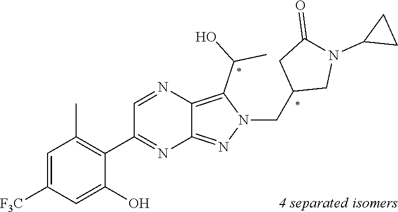 [SMILES: Cc1cc(C(F)(F)F)cc(O)c1-c1cnc2c(C(C)O)n(CC3CC(=O)N(C4CC4)C3)nc2n1]

Step 1: (R and S)-4-((3-acetyl-6-(2-(ethoxymethoxy)-6-methyl-4-(trifluoromethyl)phenyl)-2H-pyrazolo[3,4-b]pyrazin-2-yl)methyl)-1-cyclopropylpyrrolidin-2-one: Cs₂CO₃ (661 mg, 2.03 mmol) was added to solution of 1-(6-(2-(ethoxymethoxy)-6-methyl-4-(trifluoromethyl)phenyl)-2H-pyrazolo[3,4-b]pyrazin-3-yl)ethan-1-one (Intermediate 8, 400 mg, 1.01 mmol) and (1-cyclopropyl-5-oxopyrrolidin-3-yl)methyl 4-methylbenzenesulfonate (Intermediate 6, 314 mg, 1.01 mmol) in THF (5 mL) at rt. The reaction mixture was heated to 50° C. and stirred for 16 h. Then the reaction mixture was cooled to rt, filtered, and the solvents were removed under reduced pressure. The resulting crude residue was purified by reverse phase HPLC (C18 stationary phase, MeCN/H₂O+0.1% TFA) to afford the title compound. LCMS [M+H]⁺=532.3 (calcd. 532.2).

Step 2: (R and S)-1-cyclopropyl-4-((6-(2-(ethoxymethoxy)-6-methyl-4-(trifluoromethyl)phenyl)-3-((R and S)-1-hydroxyethyl)-2H-pyrazolo[3,4-b]pyrazin-2-yl)methyl)pyrrolidin-2-one: NaBH₄ (10 mg, 0.26 mmol) was added to a solution of (R and S)-4-((3-acetyl-6-(2-(ethoxymethoxy)-6-methyl-4-(trifluoromethyl)phenyl)-2H-pyrazolo[3,4-b]pyrazin-2-yl)methyl)-1-cyclopropylpyrrolidin-2-one (70 mg, 0.13 mmol) in THF (1 mL) and MeOH (0.1 mL) at 0° C. under N2 atmosphere. The reaction mixture was then warmed to rt and stirred for 2 h. Then the reaction mixture was diluted with H₂O and EtOAc, the layers were separated, and the aq. layer was extracted with EtOAc (×3). The combined organic layers were dried over MgSO₄, filtered, and the solvent was removed under reduced pressure. The resulting crude residue containing the title compound was carried forward in the next step without further purification. LCMS [M+H]⁺=534.2 (calcd. 534.2).

Step 3: (S or R)-1-cyclopropyl-4-((6-(2-(ethoxymethoxy)-6-methyl-4-(trifluoromethyl)phenyl)-3-((S or R)-1-hydroxyethyl)-2H-pyrazolo[3,4-b]pyrazin-2-yl)methyl)pyrrolidin-2-one, (S or R)-1-cyclopropyl-4-((6-(2-(ethoxymethoxy)-6-methyl-4-(trifluoromethyl)phenyl)-3-((R or S)-1-hydroxyethyl)-2H-pyrazolo[3,4-b]pyrazin-2-yl)methyl)pyrrolidin-2-one, (R or S)-1-cyclopropyl-4-((6-(2-(ethoxymethoxy)-6-methyl-4-(trifluoromethyl)phenyl)-3-((R or S)-1-hydroxyethyl)-2H-pyrazolo[3,4-b]pyrazin-2-yl)methyl)pyrrolidin-2-one, and (R or S)-1-cyclopropyl-4-((6-(2-(ethoxymethoxy)-6-methyl-4-(trifluoromethyl)phenyl)-3-((S or R)-1-hydroxyethyl)-2H-pyrazolo[3,4-b]pyrazin-2-yl)methyl)pyrrolidin-2-one: TFA (0.5 mL) was added to a solution of (R and S)-1-cyclopropyl-4-((6-(2-(ethoxymethoxy)-6-methyl-4-(trifluoromethyl)phenyl)-3-((R and S)-1-hydroxyethyl)-2H-pyrazolo[3,4-b]pyrazin-2-yl)methyl)pyrrolidin-2-one (70 mg, 0.13 mmol) in DCM (1.5 mL) at rt and stirred for 2 h. Then the solvents were removed under reduced pressure, and the resulting crude residue purified by reverse phase HPLC (C18 stationary phase, MeCN/H₂O+0.1% TFA) to afford enantio- and diastereomeric mixture of title compounds. The title compounds were resolved by preparative chiral SFC with Method T.

The fastest eluting isomer (Peak 1) of the title compound was obtained (Example 113, 98% ee). ¹H NMR (400 MHz, CDCl₃) δ 10.25 (s, 1H), 8.72 (s, 1H), 7.20 (s, 1H), 7.14 (s, 1H), 5.55 (q, J=6.6 Hz, 1H), 4.63 (d, J=6.9 Hz, 2H), 3.43-3.51 (m, 1H), 3.32-3.39 (m, 1H), 3.20-3.30 (m, 1H), 2.61-2.68 (m, 1H), 2.55-2.60 (m, 1H), 2.53 (s, 3H), 2.21-2.30 (m, 1H), 1.95 (d, J=6.7 Hz, 3H), 0.75-0.81 (m, 2H), 0.59-0.74 (m, 2H). LCMS [M+H]⁺=476.2 (calcd. 476.2).

The second eluting isomer (Peak 2) of the title compound was obtained (Example 114, 96% ee). ¹H NMR (400 MHz, CDCl₃) δ 10.22 (s, 1H), 8.68 (s, 1H), 7.19 (s, 1H), 7.11 (s, 1H), 5.63 (q, J=6.7 Hz, 1H), 4.80 (dd, J=7.3, 13.1 Hz, 1H), 4.51 (dd, J=7.0, 13.1 Hz, 1H), 3.47-3.57 (m, 1H), 3.30 (d, J=6.6 Hz, 2H), 2.61-2.70 (m, 1H), 2.47 (s, 3H), 2.41 (dd, J=8.5, 17.1 Hz, 1H), 2.15-2.24 (m, 1H), 1.85 (d, J=6.7 Hz, 3H), 0.79 (d, J=7.4 Hz, 2H), 0.71-0.77 (m, 2H). LCMS [M+H]⁺=476.2 (calcd. 476.2).

The third eluting isomer (Peak 3) of the title compound was obtained (Example 115, 97% ee). ¹H NMR (400 MHz, CDCl₃) δ 10.22 (s, 1H), 8.68 (s, 1H), 7.19 (s, 1H), 7.11 (s, 1H), 5.63 (q, J=6.7 Hz, 1H), 4.80 (dd, J=7.3, 13.1 Hz, 1H), 4.52 (dd, J=7.1, 13.1 Hz, 1H), 3.49-3.58 (m, 1H), 3.30 (d, J=6.6 Hz, 2H), 2.63-2.70 (m, 1H), 2.48 (s, 3H), 2.42 (dd, J=8.5, 17.0 Hz, 1H), 2.17-2.24 (m, 1H), 1.86 (d, J=6.7 Hz, 3H), 0.78-0.83 (m, 2H), 0.68-0.77 (m, 2H). LCMS [M+H]⁺=476.2 (calcd. 476.2).

The slowest eluting isomer (Peak 4) of the title compound was obtained (Example 116, 97% ee). ¹H NMR (400 MHz, CDCl₃) δ 10.19 (s, 1H), 8.71 (s, 1H), 7.20 (s, 1H), 7.14 (s, 1H), 5.55 (q, J=6.7 Hz, 1H), 4.63 (d, J=7.2 Hz, 2H), 3.42-3.51 (m, 1H), 3.31-3.38 (m, 1H), 3.20-3.30 (m, 1H), 2.60-2.66 (m, 1H), 2.53-2.59 (m, 1H), 2.52 (s, 3H), 2.22-2.30 (m, 1H), 1.95 (d, J=6.8 Hz, 3H), 0.73-0.81 (m, 2H), 0.63-0.74 (m, 2H). LCMS [M+H]⁺=476.2 (calcd. 476.2).

The compounds listed in Table 9 were prepared using procedures similar to those described for Examples 113-116 using the appropriate starting materials. Enantio- and diastereomeric products were separated using chiral SFC methods specified in the table. For those pairs of enantiomers, the fast-eluting isomer was listed first. Addition of DIPEA was particularly important when the starting material amine was an acid salt.

TABLE 9
Examples 117-120

| | | | LCMS | Chiral |
|---|---|---|---|---|
| Example | Structure | Name | [M + H]⁺ | Method |
| 117 | 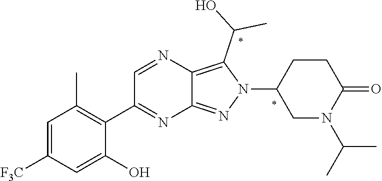 [SMILES: Cc1cc(C(F)(F)F)cc(O)c1-c1cnc2c(C(C)O)n(C3CCC(=O)N(C(C)C)C3)nc2n1] | (S or R)-5-(6-(2-hydroxy-6- methyl-4- (trifluoromethyl)phenyl)-3-((S or R)-1-hydroxyethyl)-2H- pyrazolo[3,4-b]pyrazin-2-yl)- 1- isopropylpiperidin-2-one | Calcd. 478.2, found 478.2 | U (Peak 1) |
| 118 | 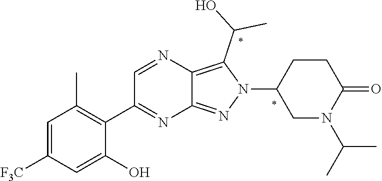 [SMILES: Cc1cc(C(F)(F)F)cc(O)c1-c1cnc2c(C(C)O)n(C3CCC(=O)N(C(C)C)C3)nc2n1] | (R or S)-5-(6-(2-hydroxy-6- methyl-4- (trifluoromethyl)phenyl)-3-((S or R)-1-hydroxyethyl)-2H- pyrazolo[3,4-b]pyrazin-2-yl)- 1-isopropylpiperidin-2-one | Calcd. 478.2, found 478.2 | U (Peak 2) |
| 119 | 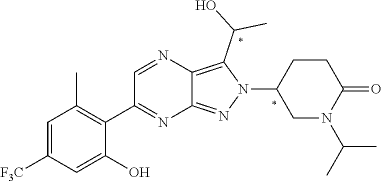 [SMILES: Cc1cc(C(F)(F)F)cc(O)c1-c1cnc2c(C(C)O)n(C3CCC(=O)N(C(C)C)C3)nc2n1] | (R or S)-5-(6-(2-hydroxy-6- methyl-4- (trifluoromethyl)phenyl)-3-((R or S)-1-hydroxyethyl)-2H- pyrazolo[3,4-b]pyrazin-2-yl)- 1-isopropylpiperidin-2-one | Calcd. 478.2, found 478.2 | U (Peak 3) |
| 120 | 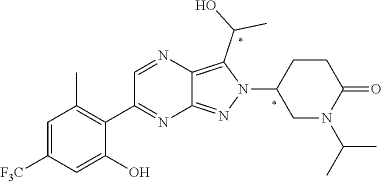 [SMILES: Cc1cc(C(F)(F)F)cc(O)c1-c1cnc2c(C(C)O)n(C3CCC(=O)N(C(C)C)C3)nc2n1] | (S or R)-5-(6-(2-hydroxy-6- methyl-4- (trifluoromethyl)phenyl)-3-((R or S)-1-hydroxyethyl)-2H- pyrazolo[3,4-b]pyrazin-2-yl)- 1-isopropylpiperidin-2-one | Calcd. 478.2, found 478.2 | U (Peak 4) |

### Example 121: 2-(2-((1r,3r)-3-fluorocyclobutyl)-2H-[1,2,3]triazolo[4,5-b]pyrazin-5-yl)-3-methyl-5-(trifluoromethyl)phenol

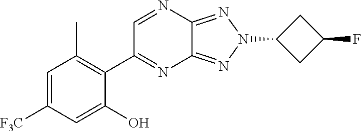 [SMILES: Cc1cc(C(F)(F)F)cc(O)c1-c1cnc2nn([C@H]3C[C@H](F)C3)nc2n1]

Step 1: 6-bromo-N2-(4-methoxybenzyl)pyrazine-2,3-diamine: (4-methoxyphenyl)methanamine (7.46 g, 54.4 mmol) and DIPEA (23 mL, 130 mmol) were added to a solution of 3,5-dibromopyrazin-2-amine (11.0 g, 43.5 mmol) in n-butanol (140 mL) at 0° C. under N2 atmosphere. The reaction mixture was heated to 125° C. and stirred for 12 h. Then the reaction mixture was cooled to rt, diluted with H₂O and EtOAc, the layers were separated, and the aq. layer was extracted with EtOAc (×3). The combined organic layers were dried over MgSO₄, filtered, and the solvent was removed under reduced pressure. The resulting crude residue containing the title compound was carried forward in the next step without further purification. LCMS [M+H]⁺=308.8 (calcd. 309.0).

Step 2: 6-bromo-1-(4-methoxybenzyl)-1H-[1,2,3]triazolo[4,5-b]pyrazine: A solution of NaNO₂ (8.70 g, 126 mmol) in H₂O (16 mL) was added to a solution of 6-bromo-N2-(4-methoxybenzyl)pyrazine-2,3-diamine (3.9 g, 12.6 mmol) in AcOH (22 mL) and H₂O (22 mL) at 0° C. The reaction mixture was then heated to 65° C. and stirred for 12 h. Then the reaction mixture was cooled to rt, and the majority of solvents were removed under reduced pressure. The resulting crude residue was diluted with H₂O and EtOAc, the layers were separated, and the aq. layer was extracted with EtOAc (×3). The combined organic layers were dried over MgSO₄, filtered, and the solvent was removed under reduced pressure. The crude mixture was purified by silica gel chromatography (EtOAc:PE) to afford the title compound. LCMS [M+H]⁺=319.8 (calcd. 320.0).

Step 3: 6-(2-methoxy-6-methyl-4-(trifluoromethyl)phenyl)-1-(4-methoxybenzyl)-1H-[1,2,3]triazolo[4,5-b]pyrazine: To a solution of 6-bromo-1-(4-methoxybenzyl)-1H-[1,2,3]triazolo[4,5-b]pyrazine (500 mg, 1.56 mmol) and 2-(2-methoxy-6-methyl-4-(trifluoromethyl)phenyl)-4,4,5,5-tetramethyl-1,3,2-dioxaborolane (494 mg, 1.56 mmol) in t-AmOH (10 mL) and water (2 mL), Cs₂CO₃ (1.53 g, 4.69 mmol) and chloro[(di(1-adamantyl)-N-butylphosphine)-2-(2-aminobiphenyl)]palladium(II) (104 mg, 0.16 mmol) was added. The mixture was degassed with N2 for 15 min, then heated to 100° C. and stirred for 12 h. Then the reaction mixture was cooled to rt, and the solvents were removed under reduced pressure. The resulting crude residue was purified by silica gel chromatography (EtOAc:PE) to afford the title compound. LCMS [M+H]⁺=430.1 (calcd. 430.1).

Step 4: 6-(2-methoxy-6-methyl-4-(trifluoromethyl)phenyl)-1H-[1,2,3]triazolo[4,5-b]pyrazine: TFA (3 mL) and TfOH (0.3 mL) were added to a solution of 6-(2-methoxy-6-methyl-4-(trifluoromethyl)phenyl)-1-(4-methoxybenzyl)-1H-[1,2,3]triazolo[4,5-b]pyrazine (1.4 g, 3.26 mmol) in DCM (15 mL) at rt, and the reaction mixture was stirred for 12 h. Then the solvents were removed under reduced pressure, and the resulting crude residue was purified by reverse phase MPLC (C18 stationary phase, MeCN/H₂O+0.5% TFA) to afford the title compound. LCMS [M+H]⁺=309.9 (calcd. 310.1).

Step 5: 2-((1R,3R)-3-fluorocyclobutyl)-5-(2-methoxy-6-methyl-4-(trifluoromethyl)phenyl)-2H-[1,2,3]triazolo[4,5-b]pyrazine: DIAD (0.24 mL, 1.21 mmol) was added to a solution of PPh₃ (424 mg, 1.62 mmol) and (cis)-3-fluorocyclobutan-1-ol (80 mg, 0.89 mmol) in THF (3 mL) at 0° C. The reaction mixture was then stirred at rt for 4 min, followed by addition of 6-(2-methoxy-6-methyl-4-(trifluoromethyl)phenyl)-1H-[1,2,3]triazolo[4,5-b]pyrazine (250 mg, 0.81 mmol). The resulting reaction mixture was stirred at rt for 12 h. Then, the reaction mixture was diluted with H₂O and EtOAc, the layers were separated, and the aq. layer was extracted with EtOAc (×3). The combined organic layers were dried over MgSO₄ and filtered, and the solvent was removed under reduced pressure. The crude mixture was purified by reverse phase HPLC (C18 stationary phase, MeCN/H₂O+0.1% TFA) to afford the title compound. LCMS [M+H]⁺=382.1 (calcd. 382.1).

Step 6: 2-(2-((1r,3r)-3-fluorocyclobutyl)-2H-[1,2,3]triazolo[4,5-b]pyrazin-5-yl)-3-methyl-5-(trifluoromethyl)phenol: NaSEt (353 mg, 4.20 mmol) was added to a solution of 2-((1R,3R)-3-fluorocyclobutyl)-5-(2-methoxy-6-methyl-4-(trifluoromethyl)phenyl)-2H-[1,2,3]triazolo[4,5-b]pyrazine (80 mg, 0.21 mmol) in DMF (2 mL) at rt under N2 atmosphere. The reaction mixture was heated to 100° C. and stirred for 3 h. Then the reaction mixture was cooled to rt and directly purified by reverse phase HPLC (C18 stationary phase, MeCN/H₂O+0.04% NH₄OH+10 mM NH₄HCO₃) to afford the title compound. LCMS [M+H]⁺=368.1 (calcd. 368.1). ¹H NMR (400 MHz, CDCl₃) δ 9.01 (s, 1H), 7.22 (s, 1H), 7.18 (s, 1H), 5.26-5.00 (m, 2H), 3.37-3.21 (m, 4H), 2.55 (s, 3H).

### Example 122: NLRP3 Inflammasome Activity

Activation of the canonical NLRP3 inflammasome requires two steps, priming and activation. A priming signal such as a pathogen activated molecular patterns (PAMPs) or danger-activated molecular patterns (DAMPs) are recognized by Toll-like receptors leads to nuclear factor kappa B (NF-KB)-mediated signaling. This in turn, up-regulates transcription of inflammasome-related components, including inactive NLRP3 and proIL-1β (Bauernfeind et al., J. Immunol. 2009, 183, 787-791; Franchi et al., Nat. Immunol. 2012, 13, 325-332; Franchi et al., J. Immunol. 2014, 193, 4214-4222). The second step is activation which induces oligomerization of NLRP3 and subsequent assembly of NLRP3, apoptosis-associated speck-like protein containing a CARD (ASC), and procaspase-1 into an inflammasome complex. This triggers the transformation of procaspase-1 to caspase-1, and the production and secretion of mature IL-1β and IL-18 (Kim et al., J. Inflamm. 2015, 12, 41; Ozaki et al., J. Inflamm. Res. 2015, 8, 15-27; Rabeony et al., Eur. J. Immunol. 2015, 45, 2847). During inflammasome complex assembly, the oligomerization of NLRP3 triggers the nucleation of ASC and an event commonly referred to as “ASC SPECK” formation, as it is identified in the cell as a discrete puncta within the cell after staining and visualization of ASC using common immunocytochemical methods.

The ability of compounds to inhibit NLRP3 inflammasome activation was determined in vitro by monitoring formation of the ASC-SPECK in human monocytic THP-1 cells after stimulation. THP-1 cells (ATCC catalog #TIB-202) were maintained in complete growth media containing Roswell Park Memorial Institute RPMI (ATCC catalog #30-2001), 10% heat inactivated fetal bovine serum, 1× penicillin/streptomycin and 0.05 mM 2-mercaptoethanol. At the start of the assay, undifferentiated THP-1 cells were plated at a density of 20,000 cells per well in a 384-well plate (Poly-D-lysine coated Cell Carrier Ultra microplate, Perkin Elmer catalog #6057500) in complete growth media supplemented with 10 ng/ml phorbol 12-myristate 13-acetate (PMA; Sigma catalog #P8139), and then incubated overnight. The next day, media was replaced with assay media (RPMI (Gibco catalog #11875-093), 0.01% bovine serum albumin (BSA)). Compounds were serially diluted in DMSO and then added to wells one hour prior to the addition of 12.5 μg/ml Gramicidin (Enzo Lifescience, catalog #ALX-350-233-M005). All incubations were carried out at 37° C. (5% CO₂/95% air). Following a 3-hour treatment with gramicidin, cells are fixed with 4% paraformaldehyde and stored at 4° C. until immunofluorescence staining.

Immunofluorescence staining: Anti-ASC antibody (MBL catalog #D086-3) was desalted and labeled with Alexa 488 antibody labeling kit (Thermo catalog #A20181) prior to use as described below. After fixation, the following steps were carried out at rt. Cells were first permeabilized with 0.3% polyethylene glycol tert-octylphenyl ether (Triton X-100) in phosphate-buffered saline (PBS) for 15 minutes and then incubated in blocking buffer containing 5% goat serum, 0.3% polyethylene glycol sorbitan monolaurate (Tween-20) and 0.03% sodium azide in PBS for 1 hour. Cells were stained with a mixture of ASC-Alexa 488 antibody (diluted 1:200 in blocking buffer) and nuclear stain DRAQ5 (1:5000 in blocking buffer, Thermo catalog #62251) in blocking buffer for 1 hour. Following a wash with 0.3% polyethylene glycol sorbitan monolaurate (Tween-20) in PBS, plates were imaged with an Opera Phenix High Content Screening System. The number of DRAQ5 positive cells containing ASC SPECKS were quantified in each well.

Data analysis: EC₅₀ values were calculated by standard curve-fitting analysis using an internally developed program in TIBCO Spotfire software.

The compounds of the present invention inhibit NLRP3 inflammasome activation in the above Biological Assay and have EC₅₀ values of less than 5 micromolar. Specific EC₅₀ values of the compounds of Examples 1-121 in the above Biological Assay are listed in Table 10.

TABLE 10

| | EC₅₀ |
|---|---|
| Ex. | (nM) |
| 1 | 71 |
| 2 | 50 |
| 3 | 35 |
| 4 | 34 |
| 5 | 13 |
| 6 | 35 |
| 7 | 46 |
| 8 | 38 |
| 9 | 59 |
| 10 | 34 |
| 11 | 1499 |
| 12 | 658 |
| 13 | 264 |
| 14 | 154 |
| 15 | 84 |
| 16 | 25 |
| 17 | 55 |
| 18 | 465 |
| 19 | 876 |
| 20 | 186 |
| 21 | 814 |
| 22 | 202 |
| 23 | 461 |
| 24 | 427 |
| 25 | 261 |
| 26 | 51 |
| 27 | 193 |
| 28 | 55 |
| 29 | 232 |
| 30 | 407 |
| 31 | 265 |
| 32 | 34 |
| 33 | 71 |
| 34 | 44 |
| 35 | 37 |
| 36 | 18 |
| 37 | 220 |
| 38 | 141 |
| 39 | 90 |
| 40 | 81 |
| 41 | 78 |
| 42 | 70 |
| 43 | 26 |
| 44 | 121 |
| 45 | 70 |
| 46 | 213 |
| 47 | 61 |
| 48 | 65 |
| 49 | 31 |
| 50 | 103 |
| 51 | 142 |
| 52 | 40 |
| 53 | 981 |
| 54 | 98 |
| 55 | 27 |
| 56 | 42 |
| 57 | 60 |
| 58 | 298 |
| 59 | 20 |
| 60 | 7 |
| 61 | 101 |
| 62 | 291 |
| 63 | 12 |
| 64 | 10 |
| 65 | 79 |
| 66 | 359 |
| 67 | 7 |
| 68 | 40 |
| 69 | 15 |
| 70 | 12 |
| 71 | 198 |
| 72 | 20 |
| 73 | 142 |
| 74 | 66 |
| 75 | 262 |
| 76 | 99 |
| 77 | 445 |
| 78 | 198 |
| 79 | 173 |
| 80 | 8 |
| 81 | 437 |
| 82 | 189 |
| 83 | 11 |
| 84 | 60 |
| 85 | 42 |
| 86 | 326 |
| 87 | 564 |
| 88 | 291 |
| 89 | 292 |
| 90 | 101 |
| 91 | 60 |
| 92 | 132 |
| 93 | 332 |
| 94 | 92 |
| 95 | 388 |
| 96 | 235 |
| 97 | 683 |
| 98 | 237 |
| 99 | 580 |
| 100 | 169 |
| 101 | 117 |
| 102 | 314 |
| 103 | 321 |
| 104 | 291 |
| 105 | 433 |
| 106 | 191 |
| 107 | 279 |
| 108 | 66 |
| 109 | 279 |
| 110 | 168 |
| 111 | 175 |
| 112 | 605 |
| 113 | 649 |
| 114 | 57 |
| 115 | 287 |
| 116 | 28 |
| 117 | 29 |
| 118 | 26 |
| 119 | 291 |
| 120 | 3881 |
| 121 | 411 |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

What is claimed is:

1. A compound of Formula (I): 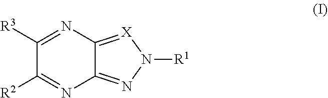
or a pharmaceutically acceptable salt thereof, wherein
X is selected from:
1) CR⁵, and
2) N;
R¹ is selected from:
1) C₁₋₆ alkyl,
2) C₀₋₃ alkylene-(monocyclic C₃₋₈ cycloalkyl),
3) C₀₋₃ alkylene-(bicyclic C₄₋₈ cycloalkyl),
4) C₀₋₃ alkylene-(C₅₋₁₀ spiro-cycloalkyl),
5) C₀₋₃ alkylene-(tricyclic C₆₋₉ cycloalkyl),
6) C₀₋₃ alkylene-(cubyl),
7) C₀₋₃ alkylene-(3- to 9-membered monocyclic heterocycloalkyl containing 1, 2, or 3 ring heteroatoms selected from N, S, O, and P),
8) C₀₋₃ alkylene-(4- to 9-membered bicyclic heterocycloalkyl containing 1, 2, or 3 ring heteroatoms selected from N, S, O, and P),
9) C₀₋₃ alkylene-(5- to 9-membered spirocyclic heterocycloalkyl containing 1, 2, or 3 ring heteroatoms selected from N, S, O, and P),
10) C₀₋₃ alkylene-(5- to 10-membered heteroaryl containing 1, 2, or 3 ring heteroatoms selected from N, S, O, and P),
11) NH—C₁₋₆ alkyl,
12) NH—C₀₋₃ alkylene-(monocyclic C₃₋₈ cycloalkyl),
13) NH—C₀₋₃ alkylene-(bicyclic C₄₋₈ cycloalkyl),
14) NH—C₀₋₃ alkylene-(C₅₋₁₀ spiro-cycloalkyl), and
15) NH—C₀₋₃ alkylene-(tricyclic C₆₋₉ cycloalkyl),
wherein said R¹ group is unsubstituted or substituted with 1 to 5 substituents independently selected from R⁶;
R² is phenyl substituted by from 0 to 3 substituents independently selected from:
1) OH,
2) halo,
3) C₁₋₆ alkyl, and
4) C₁₋₆ haloalkyl containing from 1 to 3 independently selected halogens;
R³ is selected from:
1) H,
2) halo, and
3) C₁₋₆ alkyl;
R⁵ is selected from:
1) H,
2) OH,
3) CN,
4) C₁₋₆ alkyl,
5) C₁₋₆ alkoxy, and
6) C₃₋₇ cycloalkyl,
wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
1) OH,
2) halo,
3) CN,
4) NR^{a}₂,
wherein each R^{a} is independently selected from H and C₁₋₆ alkyl; and
each R⁶ is independently selected from:
1) OH,
2) halo,
3) CN,
4) oxo, 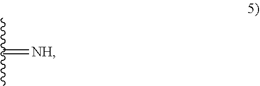 [SMILES: *=N]
6) C₁₋₆ alkyl, which may be straight or branched and which is unsubstituted or substituted with 1 to 4 substituents selected from OH, halo, CN, oxo, and C₁₋₆ alkoxy,
7) C₁₋₆ alkoxy, which may be straight or branched and which is unsubstituted or substituted with 1 to 4 substituents selected from OH, halo, CN, oxo, and C₁₋₆ alkoxy,
8) C₃₋₇ cycloalkyl, which is unsubstituted or substituted with 1 to 4 substituents selected from OH, halo, CN, oxo, C₁₋₃ alkyl, C₁₋₃ haloalkyl, and C₁₋₆ alkoxy,
9) 3- to 9-membered monocyclic heterocycloalkyl containing 1, 2, or 3 ring heteroatoms selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy,
10) C₅₋₇ aryl, which is unsubstituted or substituted with 1 to 4 substituents selected from OH, halo, CN, oxo, and C₁₋₆ alkoxy,
11) methylene-C₅₋₇ aryl, which is unsubstituted or substituted with 1 to 4 substituents selected from OH, halo, CN, oxo, and C₁₋₆ alkoxy, and
12) 5- to 10-membered heteroaryl containing at least one ring heteroatom selected from N, S, O, and P, and which is unsubstituted or substituted with 1 to 4 substituents selected from OH, halo, CN, oxo, C₁₋₃ alkyl, C₁₋₃ haloalkyl, and C₁₋₆ alkoxy.

2. The compound according to claim 1, or a pharmaceutically acceptable salt thereof, wherein X is N.

3. The compound according to claim 1, or a pharmaceutically acceptable salt thereof, wherein X is CR⁵.

4. The compound according to claim 3, or a pharmaceutically acceptable salt thereof, wherein R⁵ is selected from:
1) H,
2) OH,
3) CN,
4) C₁₋₃ alkyl,
5) C₁₋₃ alkoxy, and
6) C₃₋₄ cycloalkyl,
wherein said R⁵ C₁₋₆ alkyl, R⁵ C₁₋₆ alkoxy, or R⁵ C₃₋₇ cycloalkyl is unsubstituted or substituted with 1 to 5 substituents independently selected from:
1) OH,
2) C₁,
3) F,
4) CN,
5) NH₂, and
6) CH₃.

5. The compound according to claim 3, or a pharmaceutically acceptable salt thereof, wherein R⁵ is selected from the group:
1) H,
2) CH₃, and
3) CH(OH)(CH₃).

6. The compound according to claim 3, or a pharmaceutically acceptable salt thereof, wherein R⁵ is H.

7. The compound according to claim 1, or a pharmaceutically acceptable salt thereof, wherein R¹ is selected from:
1) C₁₋₆ alkyl,
2) C₀₋₃ alkylene-(monocyclic C₃₋₈ cycloalkyl),
3) C₀₋₃ alkylene-(bicyclic C₄₋₈ cycloalkyl),
4) C₀₋₃ alkylene-(C₅₋₁₀ spiro-cycloalkyl),
5) C₀₋₃ alkylene-(tricyclic C₆₋₉ cycloalkyl),
6) C₀₋₃ alkylene-(3- to 9-membered monocyclic heterocycloalkyl containing 1, 2, or
3 ring heteroatoms selected from N, S, O, and P),
7) C₀₋₃ alkylene-(4- to 9-membered bicyclic heterocycloalkyl containing 1, 2, or 3 ring heteroatoms selected from N, S, O, and P),
8) C₀₋₃ alkylene-(5- to 9-membered spirocyclic heterocycloalkyl containing 1, 2, or 3 ring heteroatoms selected from N, S, O, and P), and
9) C₀₋₃ alkylene-(5- to 10-membered heteroaryl containing 1, 2, or 3 ring heteroatoms selected from N, S, O, and P), and
wherein said R¹ group is unsubstituted or substituted with 1 to 5 substituents independently selected from R⁶, and
each R⁶ is independently selected from:
1) OH,
2) F,
3) CN,
4) oxo,
5) C₁₋₃ alkyl, which may be straight or branched and which is unsubstituted or substituted with 1 to 4 substituents selected from OH, F, CN, oxo, and OCH₃,
6) C₃ cycloalkyl, which is unsubstituted, and
7) methylene-C₅₋₇ aryl, which is unsubstituted or substituted with 1 to 4 substituents selected from OH, F, CN, oxo, and OCH₃.

8. The compound according to claim 1, or a pharmaceutically acceptable salt thereof, wherein R¹ is selected from:
1) C₁₋₄ alkyl,
2) monocyclic C₃₋₆ cycloalkyl,
3) methylene-(monocyclic C₃₋₆ cycloalkyl),
4) bicyclic C₅₋₈ cycloalkyl,
5) 5- to 6-membered monocyclic heterocycloalkyl containing 1 or 2 ring heteroatoms selected from N, S, and O,
6) methylene-(5- to 6-membered monocyclic heterocycloalkyl containing 1 or 2 ring heteroatoms selected from N, S, and O),
7) 6- to 9-membered bicyclic heterocycloalkyl containing 1, 2, or 3 ring heteroatoms selected from N, S, and O,
8) methylene-(4- to 9-membered bicyclic heterocycloalkyl containing 1, 2, or 3 ring heteroatoms selected from N, S, and O), and
wherein said R¹ group is unsubstituted or substituted with 1 to 5 substituents independently selected from R⁶, and
each R⁶ is independently selected from:
1) OH,
2) F,
3) CN,
4) oxo,
5) C₁₋₃ alkyl, which may be straight or branched and which is unsubstituted or substituted with 1 to 4 substituents selected from OH, F, CN, oxo, and OCH₃,
6) C₃ cycloalkyl, which is unsubstituted, and
7) methylene-C₅₋₇ aryl, which is unsubstituted or substituted with 1 to 4 substituents selected from OH, F, CN, oxo, and OCH₃.

9. The compound according to claim 1, or a pharmaceutically acceptable salt thereof, wherein R¹ is selected from:

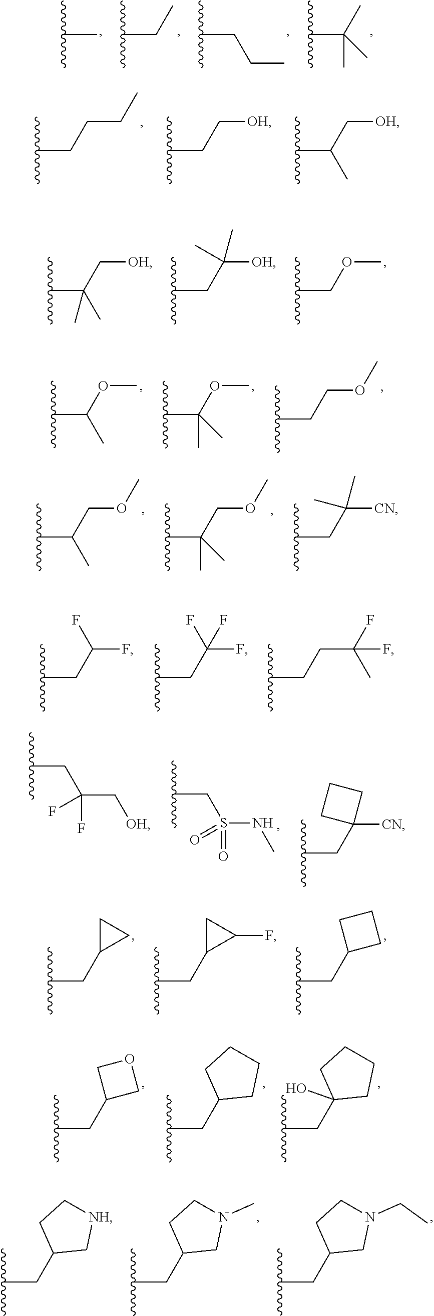

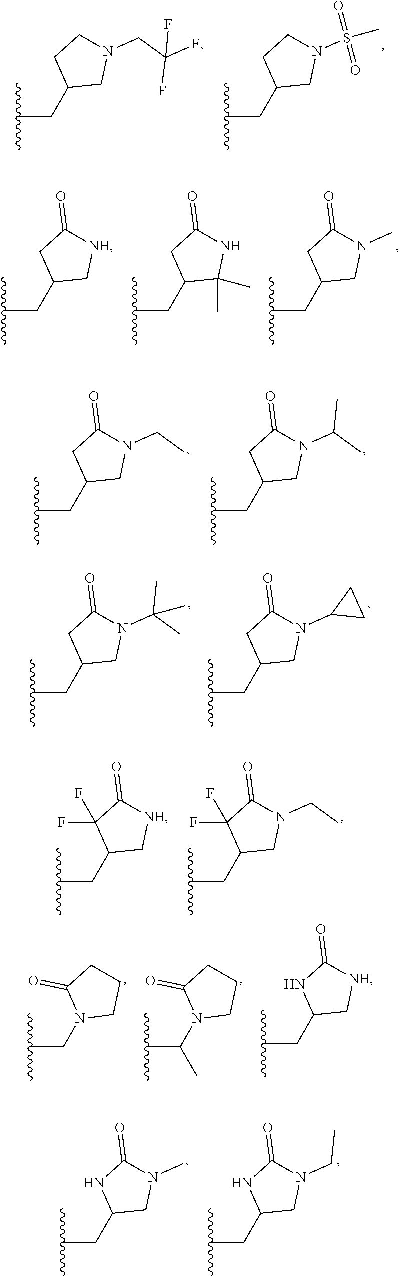

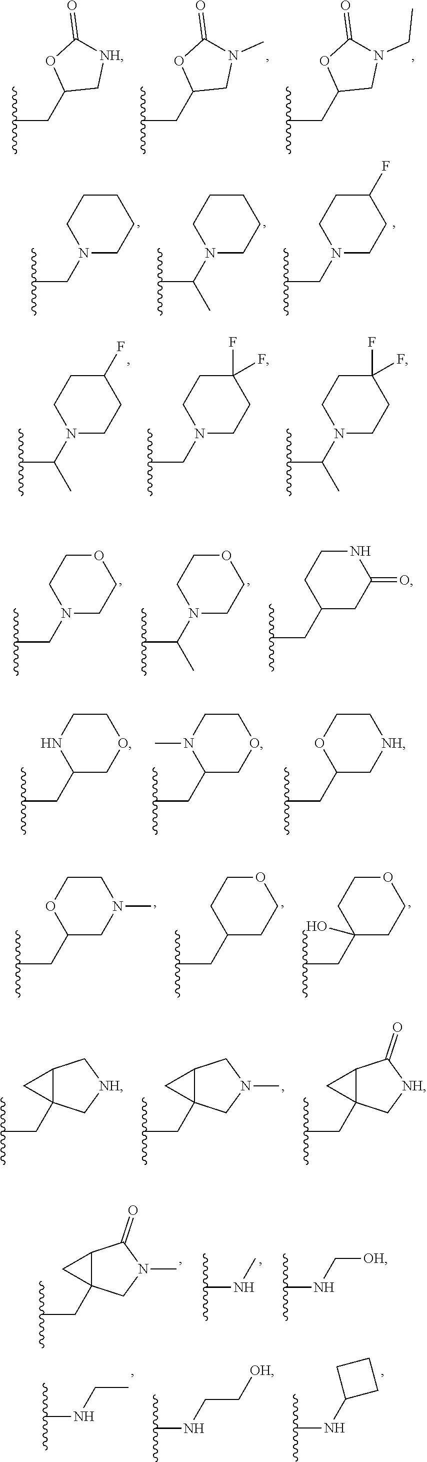

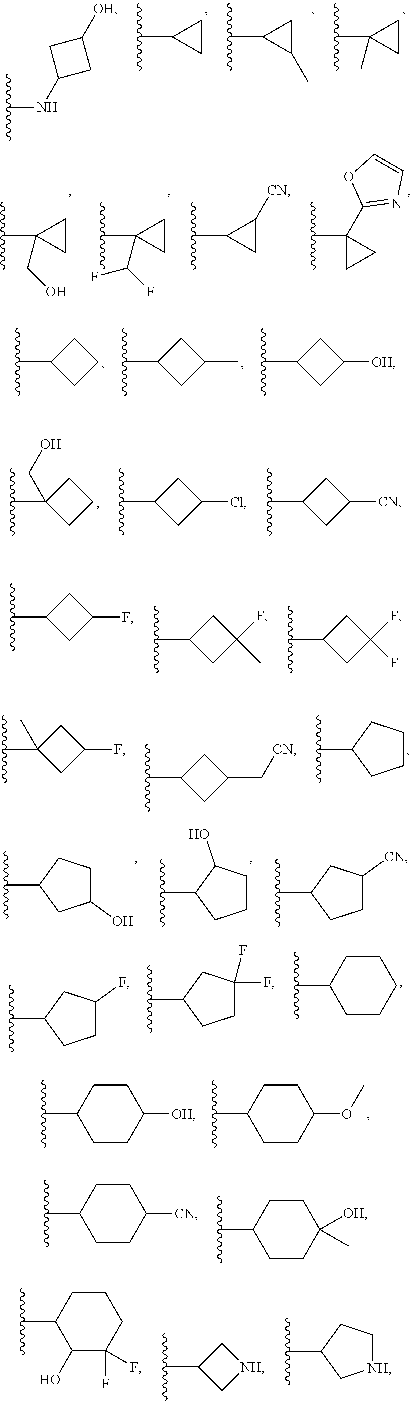

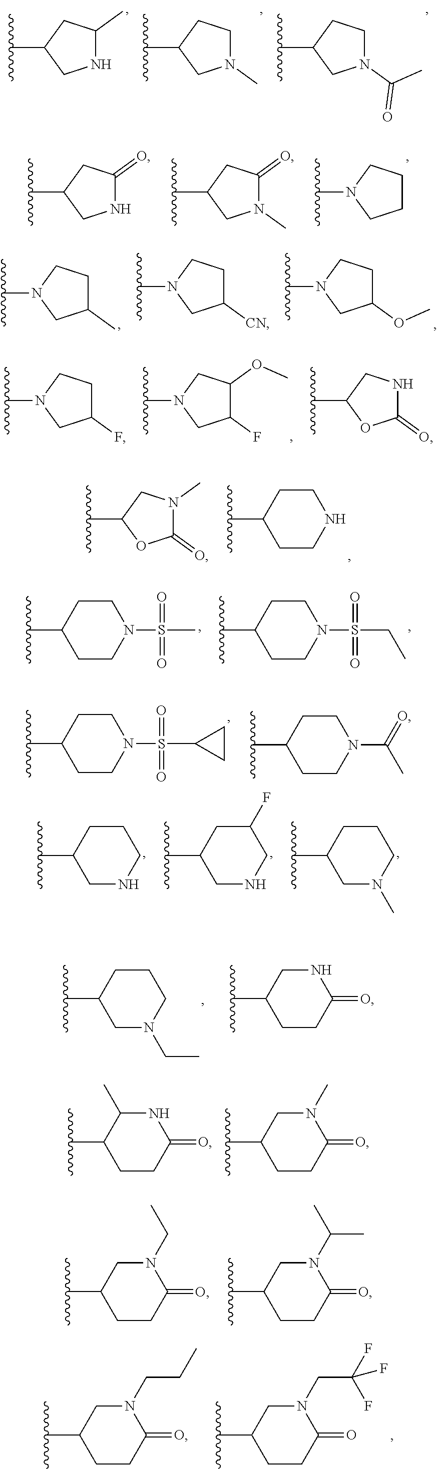

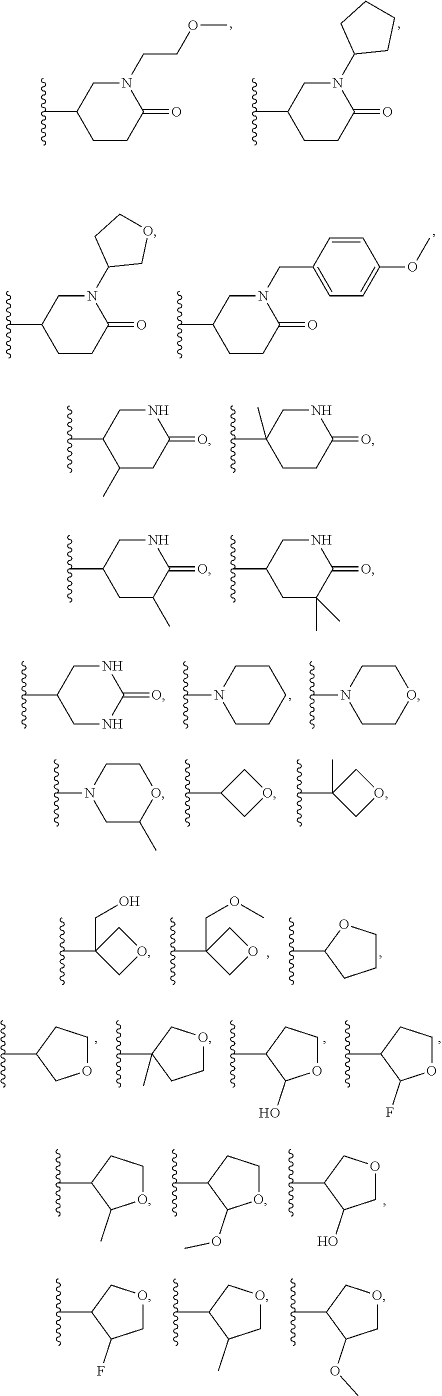

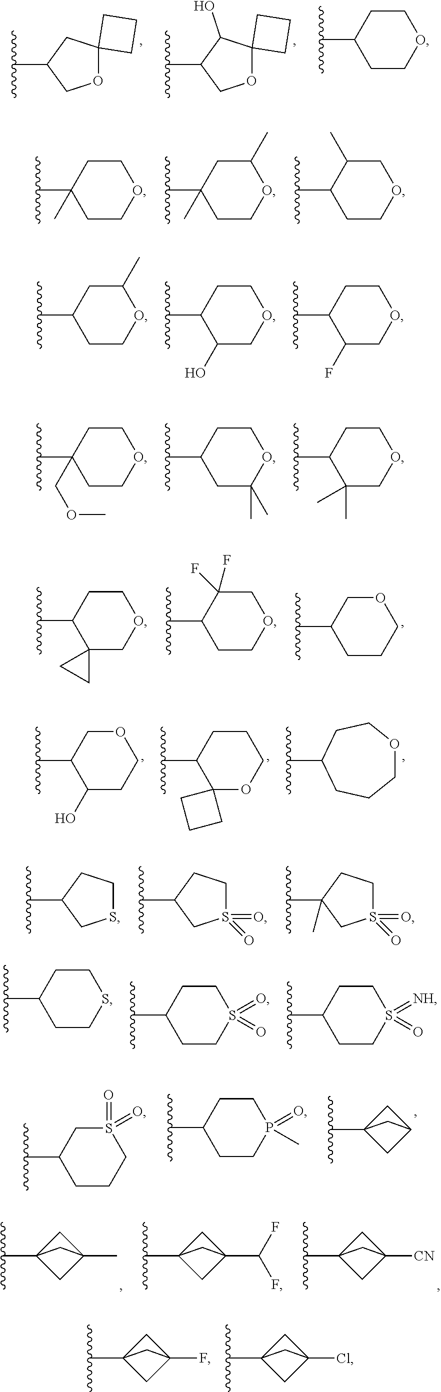

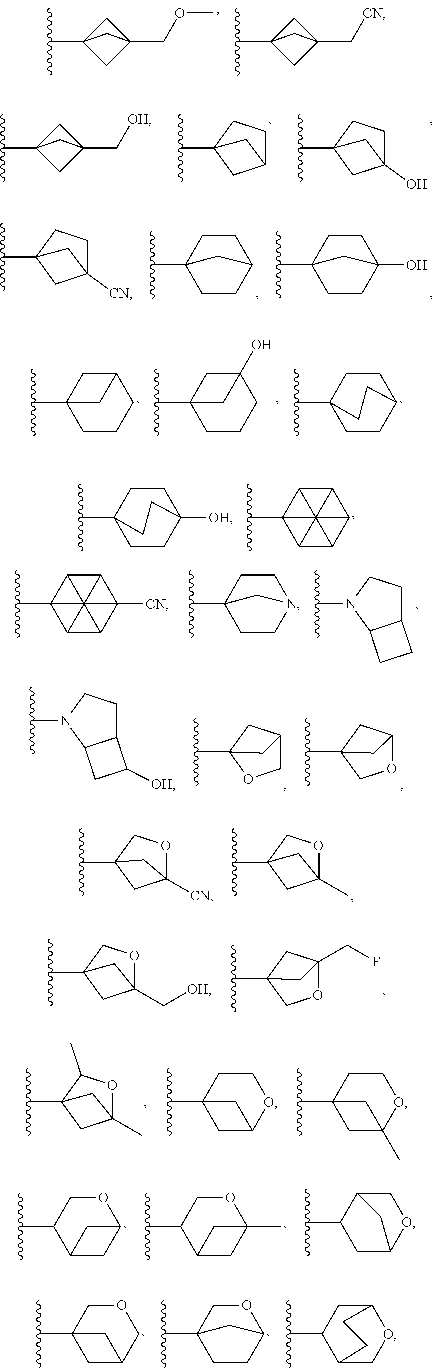

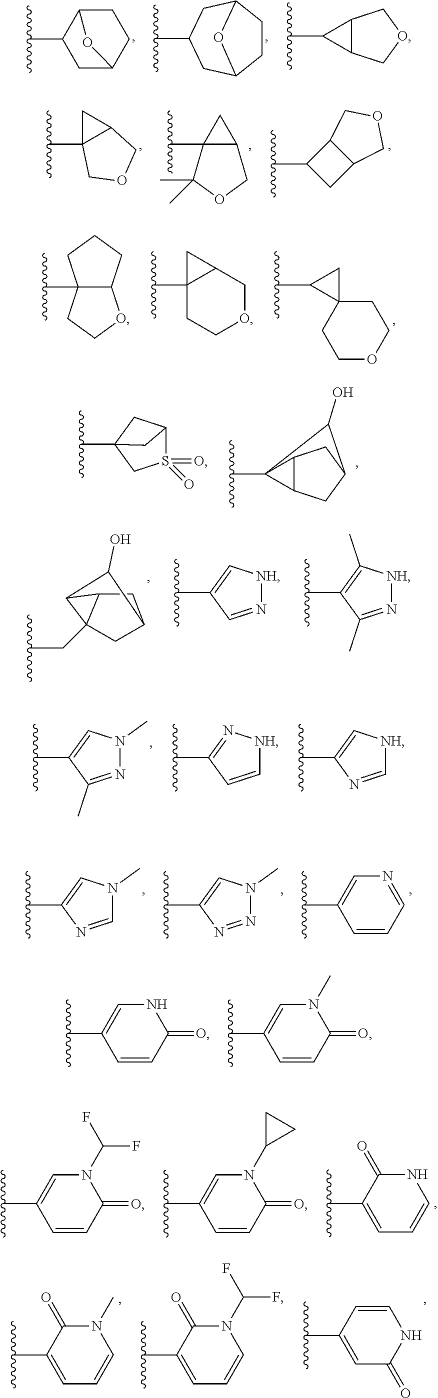

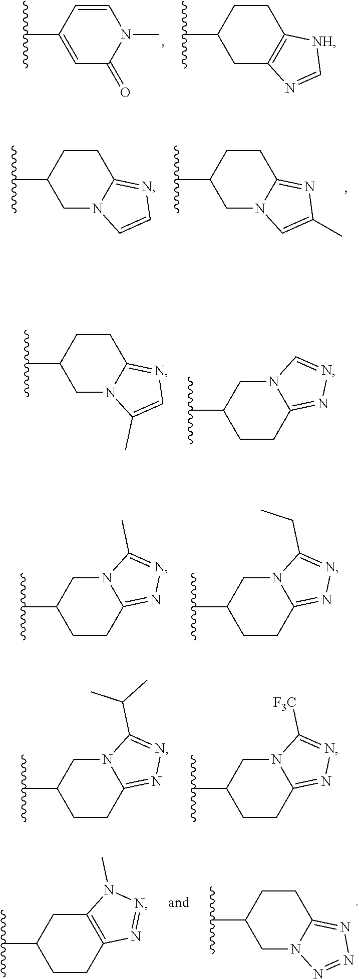

10. The compound according to claim 9, or a pharmaceutically acceptable salt thereof, wherein R¹ is selected from:

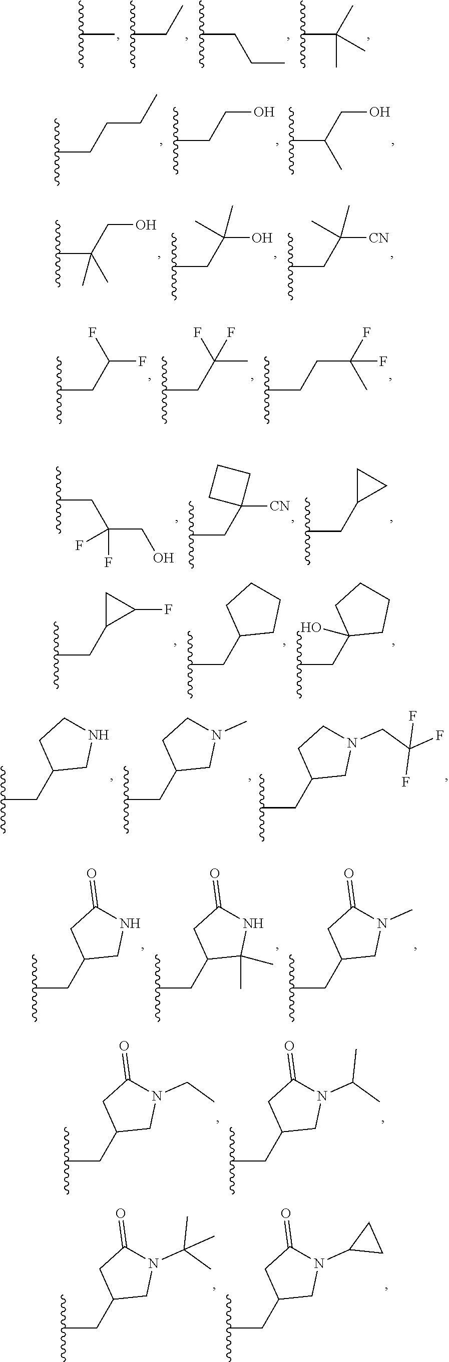

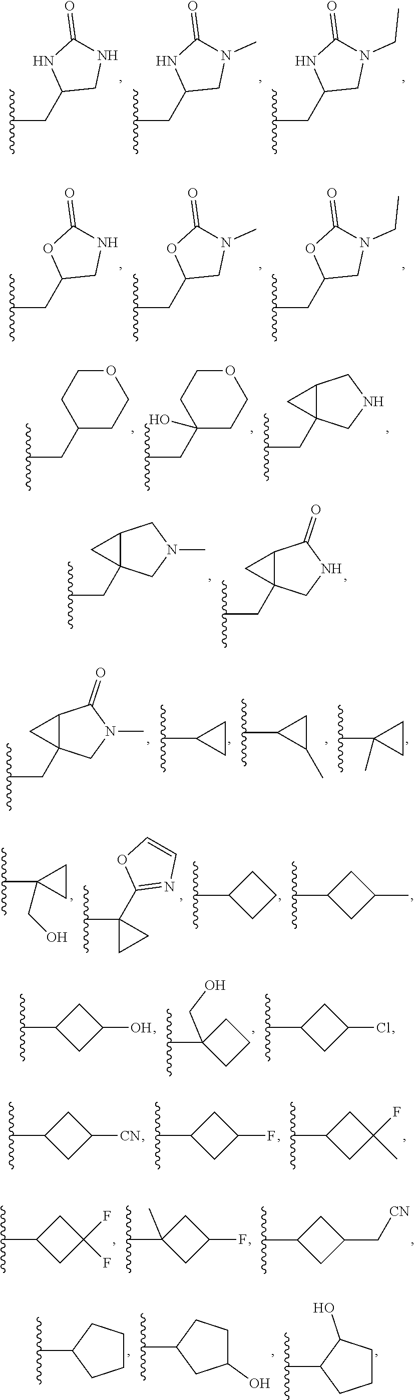

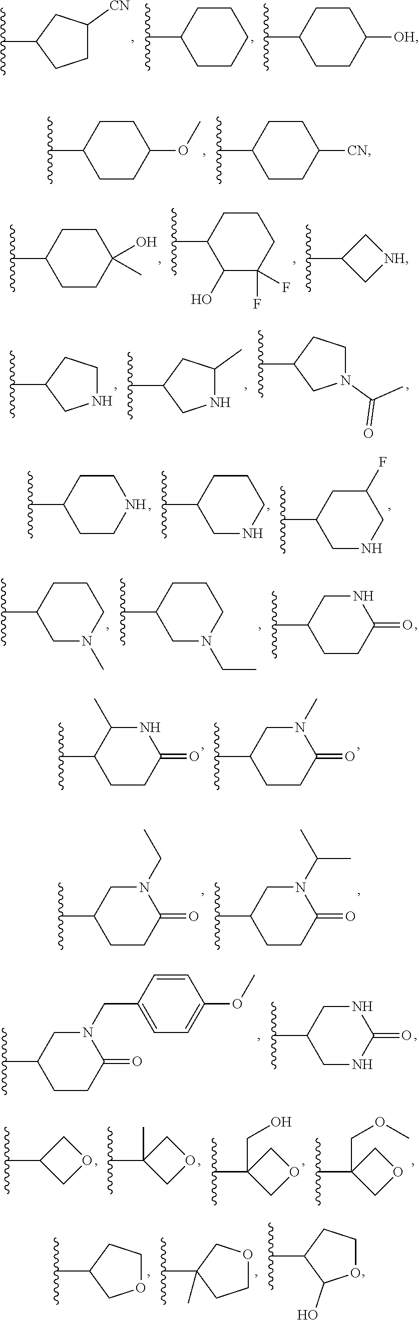

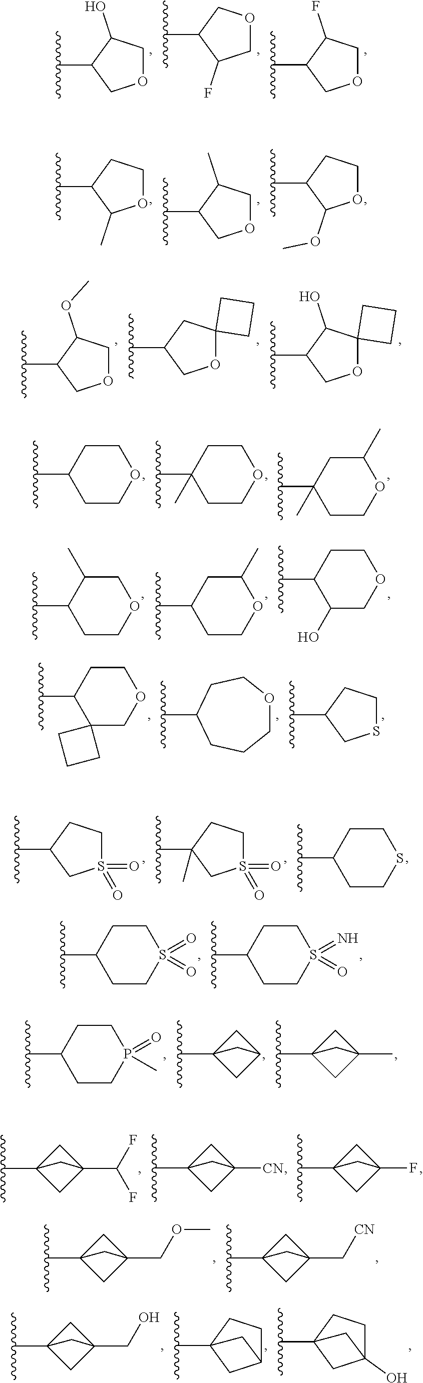

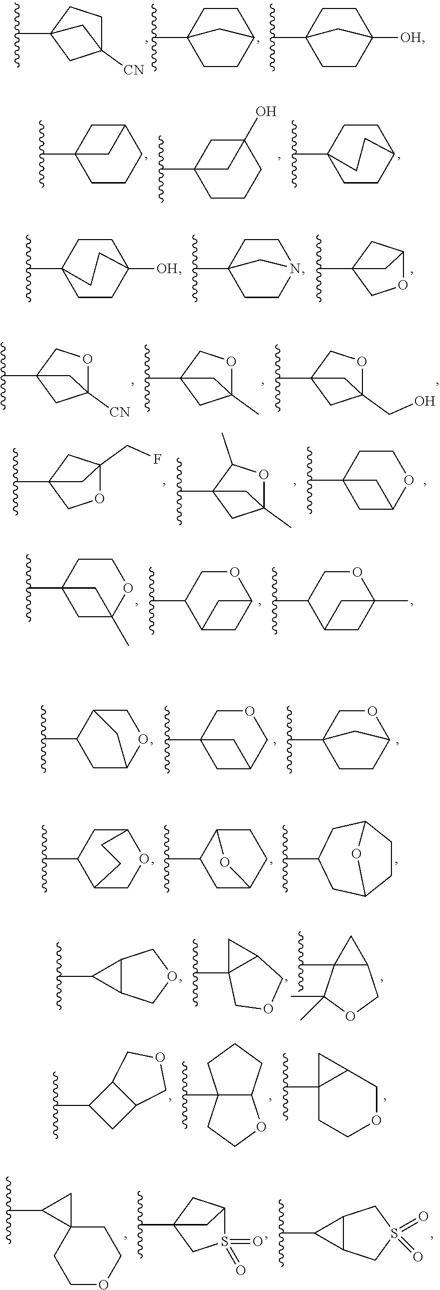

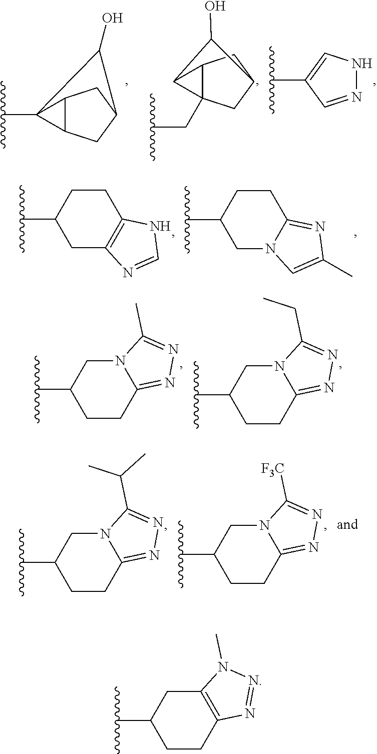

11. The compound according to claim 9, or a pharmaceutically acceptable salt thereof, wherein R¹ is selected from:

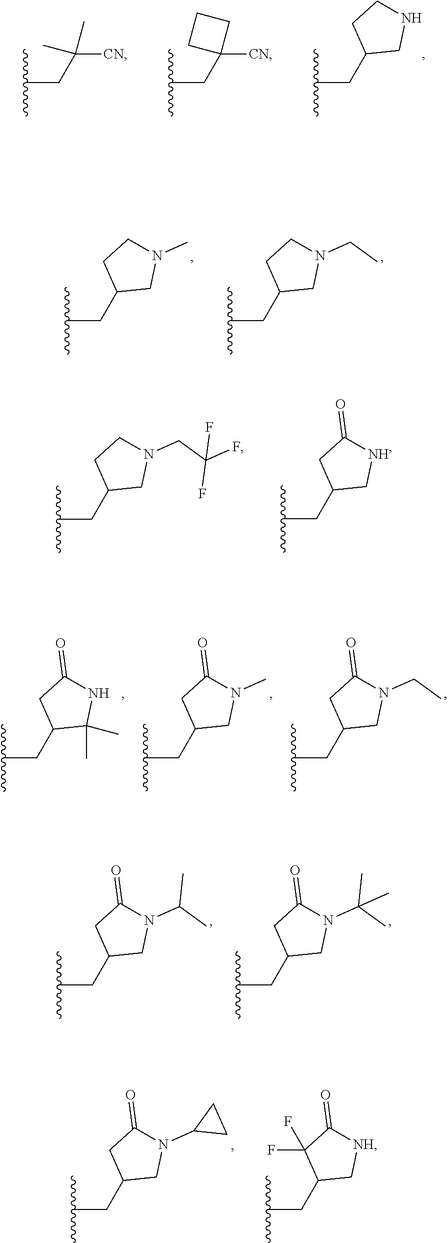

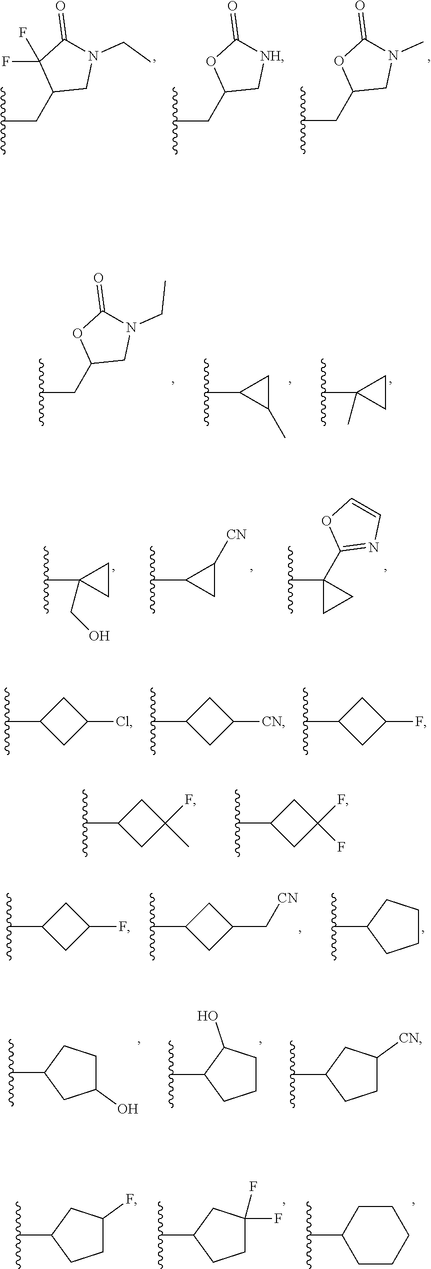

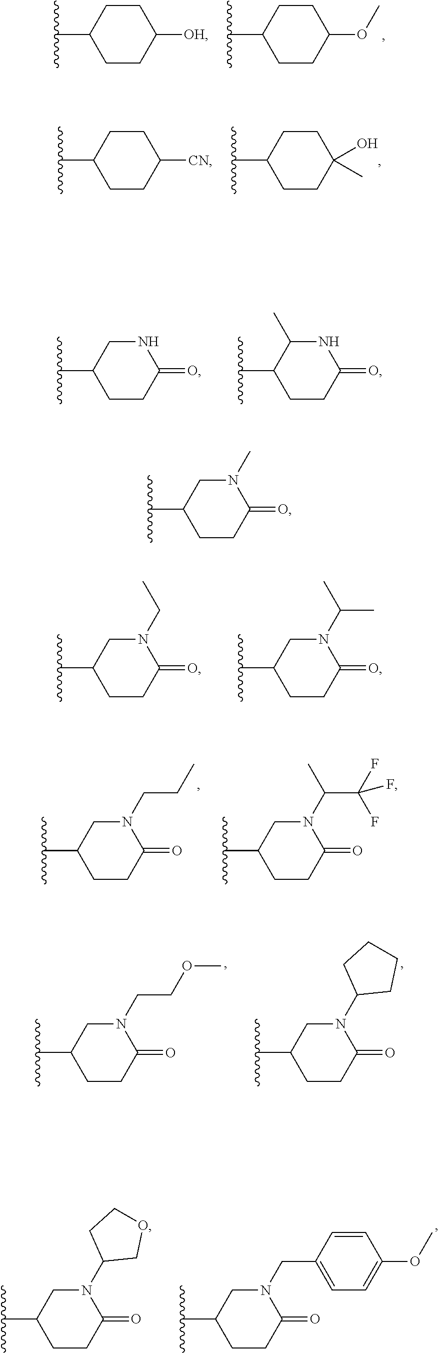

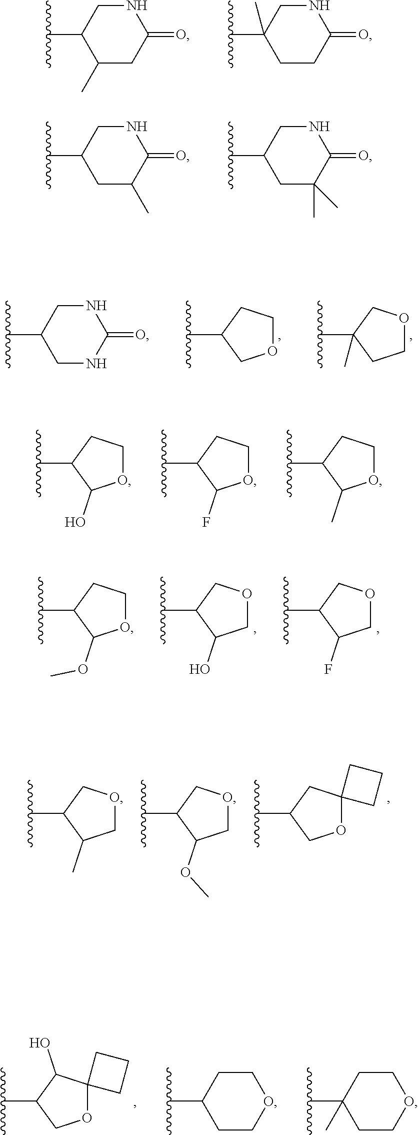

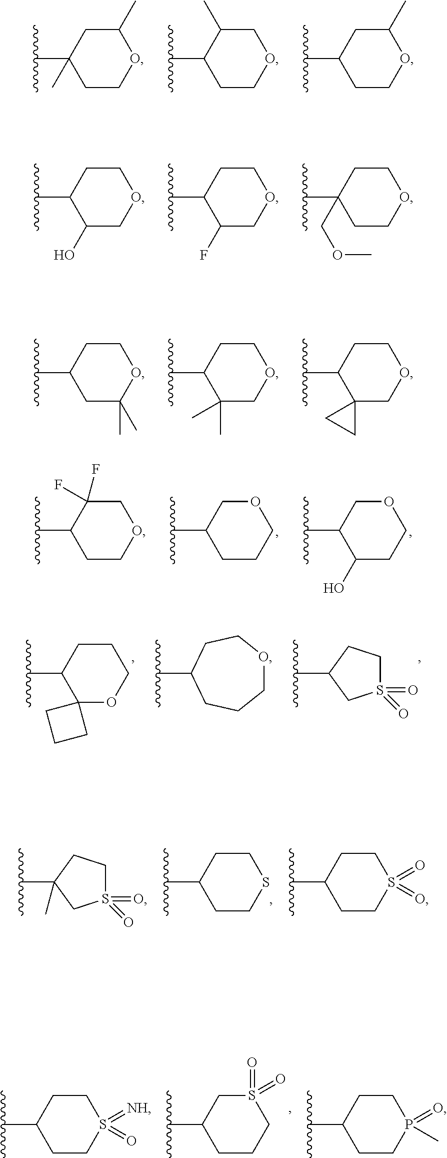

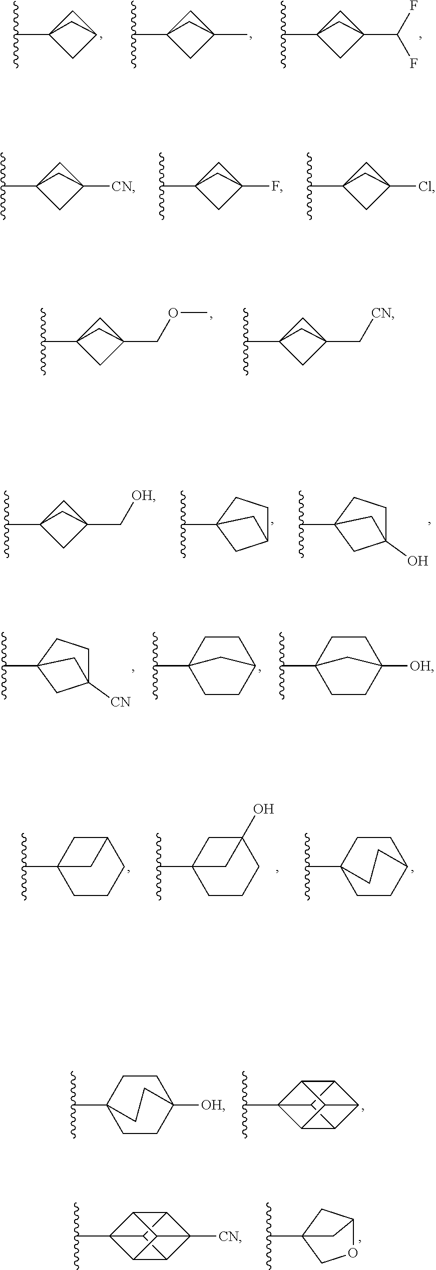

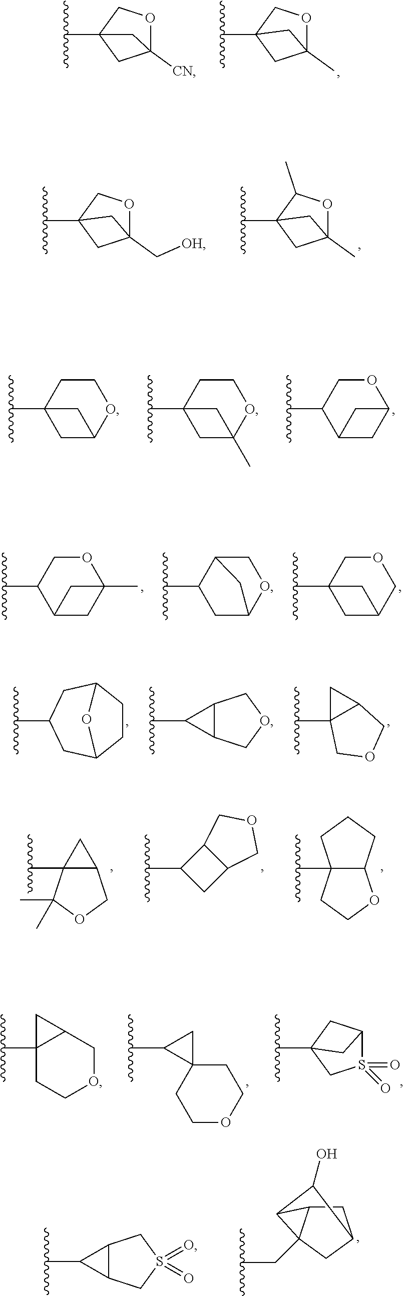

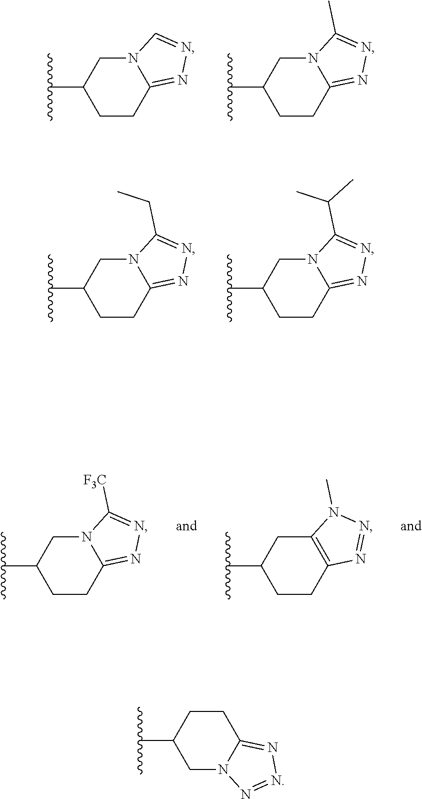

12. The compound according to claim 9, or a pharmaceutically acceptable salt thereof, wherein R¹ is selected from:

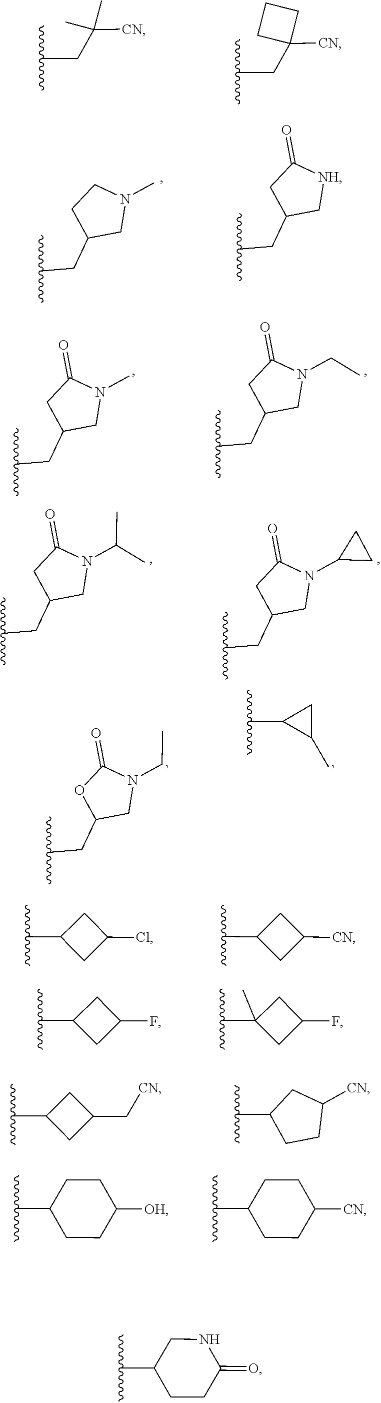

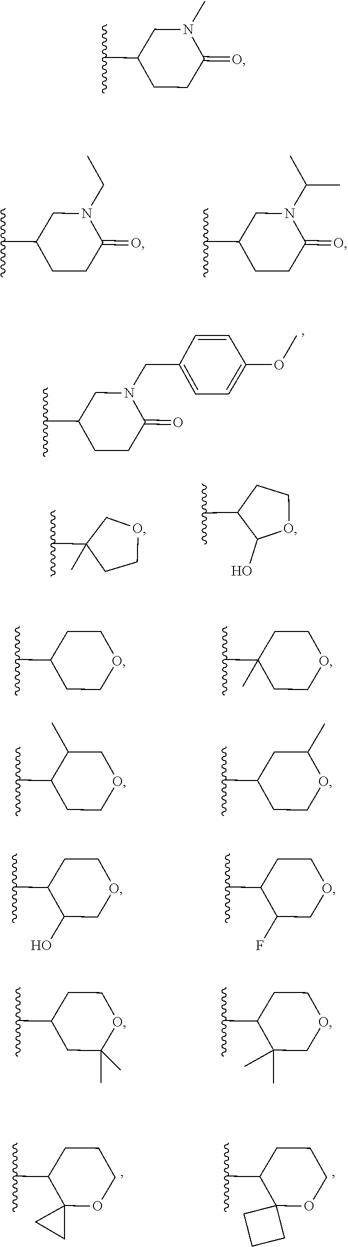

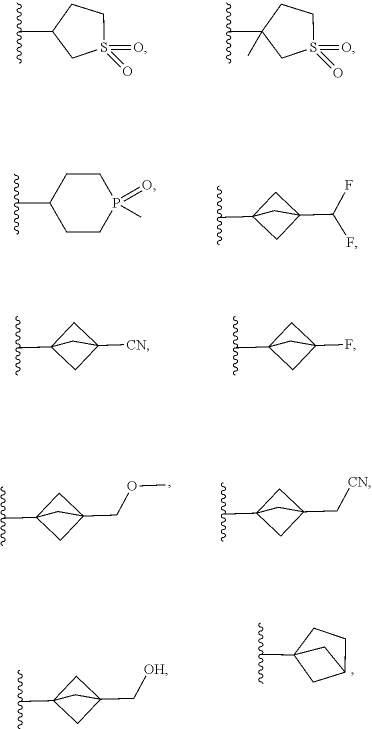

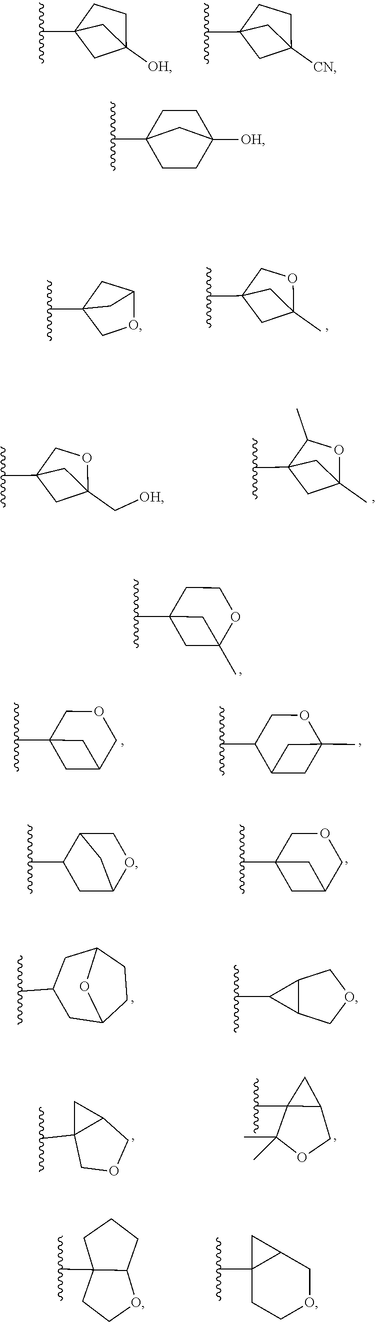

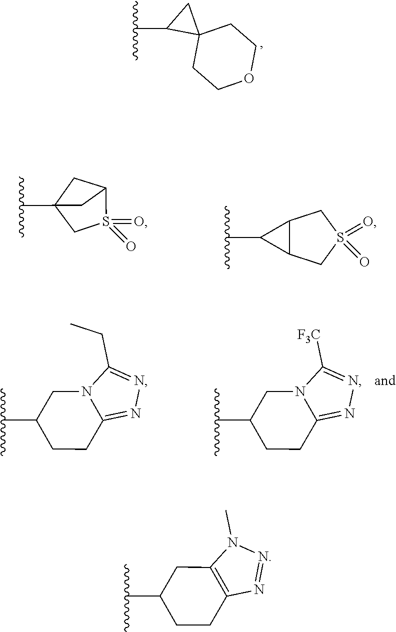 [SMILES: *C1CC12CCOCC2 ; *C12CC(C1)S(=O)(=O)C2 ; *C1C2CS(=O)(=O)CC12 ; *C1CCc2nnc(CC)n2C1 ; *C1CCc2nnc(C(F)(F)F)n2C1 ; *C1CCc2nnn(C)c2C1]

13. The compound according to claim 9, or a pharmaceutically acceptable salt thereof, wherein R¹ is selected from:

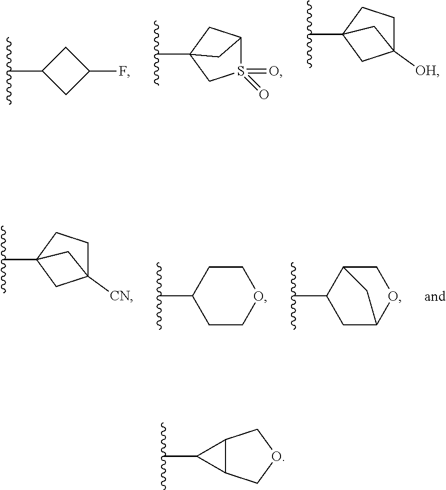

14. The compound according to claim 9, or a pharmaceutically acceptable salt thereof, wherein R¹ is selected from:

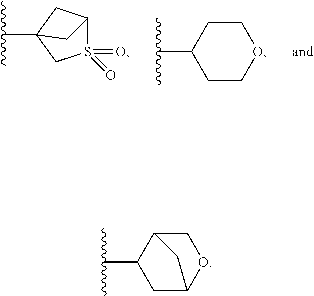

15. The compound according to claim 1, or a pharmaceutically acceptable salt thereof, wherein R² is phenyl substituted by 2 or 3 substituents selected independently from:
1) OH,
2) halo,
3) C₁₋₂ alkyl, and
4) C₁₋₂ haloalkyl containing from 1 to 3 independently selected halogens.

16. The compound according to claim 15, or a pharmaceutically acceptable salt thereof, wherein R² is phenyl substituted by 2 or 3 substituents selected independently from:
1) OH,
2) C₁,
3) CH₃, and
4) CF₃.

17. The compound according to claim 16, or a pharmaceutically acceptable salt thereof, wherein R² is phenyl substituted by OH and CF₃.

18. The compound according to claim 16, or a pharmaceutically acceptable salt thereof, wherein R² is phenyl substituted by OH, CH₃, and CF₃.

19. The compound according to claim 16, or a pharmaceutically acceptable salt thereof, wherein R² is phenyl substituted by OH, CH₃, and C₁.

20. The compound according to claim 16, or a pharmaceutically acceptable salt thereof, wherein R² is selected from

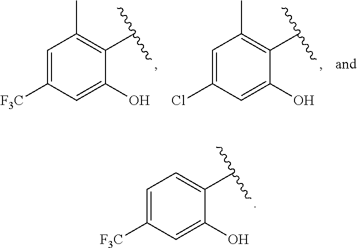

21. The compound according to claim 16, or a pharmaceutically acceptable salt thereof, wherein R² is

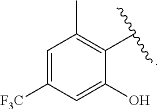 [SMILES: *c1c(C)cc(C(F)(F)F)cc1O]

22. The compound according to claim 1, or a pharmaceutically acceptable salt thereof, wherein R³ is H.

23. The compound according to claim 1, or a pharmaceutically acceptable salt thereof, wherein R³ is selected from C₁₋₆ alkyl.

24. The compound according to claim 23, or a pharmaceutically acceptable salt thereof, wherein R³ is CH₃.

25. The compound according to claim 1, or a pharmaceutically acceptable salt thereof, wherein the compound is selected from the group consisting of:
1) 3-methyl-2-(2-(tetrahydro-2H-pyran-4-yl)-2H-pyrazolo[3,4-b]pyrazin-6-yl)-5-(trifluoromethyl)phenol,
2) 4-(6-(2-hydroxy-6-methyl-4-(trifluoromethyl)phenyl)-2H-pyrazolo[3,4-b]pyrazin-2-yl)-2-thiabicyclo[2.1.1]hexane 2,2-dioxide,
3) 2-(2-((1S,4R,5R)-2-oxabicyclo[2.2.1]heptan-5-yl)-2H-pyrazolo[3,4-b]pyrazin-6-yl)-3-methyl-5-(trifluoromethyl)phenol,
4) 2-(2-((1R,4S,5S)-2-oxabicyclo[2.2.1]heptan-5-yl)-2H-pyrazolo[3,4-b]pyrazin-6-yl)-3-methyl-5-(trifluoromethyl)phenol,
5) (5S)-5-[6-[2-hydroxy-6-methyl-4-(trifluoromethyl)phenyl]pyrazolo[3,4-b]pyrazin-2-yl]-1-methyl-piperidin-2-one,
6) (R)-3-methyl-2-(2-((1-methylpyrrolidin-3-yl)methyl)-2H-pyrazolo[3,4-b]pyrazin-6-yl)-5-(trifluoromethyl)phenol,
7) (R)-4-((6-(2-hydroxy-6-methyl-4-(trifluoromethyl)phenyl)-2H-pyrazolo[3,4-b]pyrazin-2-yl)methyl)pyrrolidin-2-one,
8) 3-(6-(2-hydroxy-6-methyl-4-(trifluoromethyl)phenyl)-2H-pyrazolo[3,4-b]pyrazin-2-yl)bicyclo[1.1.1]pentane-1-carbonitrile,
9) 2-(2-((1R,5S,6S)-3-oxabicyclo[3.1.0]hexan-6-yl)-2H-pyrazolo[3,4-b]pyrazin-6-yl)-3-methyl-5-(trifluoromethyl)phenol,
10) 2-(2-((1R,5S,6R)-3-oxabicyclo[3.1.0]hexan-6-yl)-2H-pyrazolo[3,4-b]pyrazin-6-yl)-3-methyl-5-(trifluoromethyl)phenol,
11) (1S,4S)-4-(6-(2-hydroxy-6-methyl-4-(trifluoromethyl)phenyl)-2H-pyrazolo[3,4-b]pyrazin-2-yl)cyclohexane-1-carbonitrile,
12) 3-(6-(2-hydroxy-6-methyl-4-(trifluoromethyl)phenyl)-2H-pyrazolo[3,4-b]pyrazin-2-yl)-2,2-dimethylpropanenitrile,
13) 1-((6-(2-hydroxy-6-methyl-4-(trifluoromethyl)phenyl)-2H-pyrazolo[3,4-b]pyrazin-2-yl)methyl)cyclobutane-1-carbonitrile,
14) 2-(2-(3-oxabicyclo[3.1.1]heptan-1-yl)-2H-pyrazolo[3,4-b]pyrazin-6-yl)-3-methyl-5-(trifluoromethyl)phenol,
15) 3-methyl-2-(2-(1-methyl-2-oxabicyclo[3.1.1]heptan-5-yl)-2H-pyrazolo[3,4-b]pyrazin-6-yl)-5-(trifluoromethyl)phenol,
16) 2-(2-((1R,3R)-3-fluorocyclobutyl)-2H-pyrazolo[3,4-b]pyrazin-6-yl)-3-methyl-5-(trifluoromethyl)phenol,
17) 2-(2-(2-oxabicyclo[2.1.1]hexan-4-yl)-2H-pyrazolo[3,4-b]pyrazin-6-yl)-3-methyl-5-(trifluoromethyl)phenol,
18) 2-((1s,3s)-3-(6-(2-hydroxy-6-methyl-4-(trifluoromethyl)phenyl)-2H-pyrazolo[3,4-b]pyrazin-2-yl)cyclobutyl)acetonitrile,
19) (R and S)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
20) (R)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
21) (S)-3-methyl-2-(2-(1-methyl-2-oxabicyclo[3.1.1]heptan-4-yl)-2H-pyrazolo[3,4-b]pyrazin-6-yl)-5-(trifluoromethyl)phenol,
22) (1S,3R and 1R,3S)-3-(6-(2-hydroxy-6-methyl-4-(trifluoromethyl)phenyl)-2H-pyrazolo[3,4-b]pyrazin-2-yl)cyclopentane-1-carbonitrile,
23) (1S,3R)-3-(6-(2-hydroxy-6-methyl-4-(trifluoromethyl)phenyl)-2H-pyrazolo[3,4-b]pyrazin-2-yl)cyclopentane-1-carbonitrile,
24) (1R,3S)-3-(6-(2-hydroxy-6-methyl-4-(trifluoromethyl)phenyl)-2H-pyrazolo[3,4-b]pyrazin-2-yl)cyclopentane-1-carbonitrile,
25) 2-(2-((3aS,6aR and 3aR,6aS)-hexahydro-3aH-cyclopenta[b]furan-3a-yl)-2H-pyrazolo[3,4-b]pyrazin-6-yl)-3-methyl-5-(trifluoromethyl)phenol,
26) 2-(2-((3aS,6aR)-hexahydro-3aH-cyclopenta[b]furan-3a-yl)-2H-pyrazolo[3,4-b]pyrazin-6-yl)-3-methyl-5-(trifluoromethyl)phenol,
27) 2-(2-((3aR,6aS)-hexahydro-3aH-cyclopenta[b]furan-3a-yl)-2H-pyrazolo[3,4-b]pyrazin-6-yl)-3-methyl-5-(trifluoromethyl)phenol,
28) (S and R)-2-(2-(5-oxaspiro[3.5]nonan-9-yl)-2H-pyrazolo[3,4-b]pyrazin-6-yl)-3-methyl-5-(trifluoromethyl)phenol,
29) (S)-2-(2-(5-oxaspiro[3.5]nonan-9-yl)-2H-pyrazolo[3,4-b]pyrazin-6-yl)-3-methyl-5-(trifluoromethyl)phenol,
30) (R)-2-(2-(5-oxaspiro[3.5]nonan-9-yl)-2H-pyrazolo[3,4-b]pyrazin-6-yl)-3-methyl-5-(trifluoromethyl)phenol,
31) (S and R)-2-(2-(6-oxaspiro[2.5]octan-1-yl)-2H-pyrazolo[3,4-b]pyrazin-6-yl)-3-methyl-5-(trifluoromethyl)phenol,
32) (S)-2-(2-(6-oxaspiro[2.5]octan-1-yl)-2H-pyrazolo[3,4-b]pyrazin-6-yl)-3-methyl-5-(trifluoromethyl)phenol,
33) (R)-2-(2-(6-oxaspiro[2.5]octan-1-yl)-2H-pyrazolo[3,4-b]pyrazin-6-yl)-3-methyl-5-(trifluoromethyl)phenol,
34) (S and R)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
35) (S)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
36) (R)-2-(2-(1,3-dimethyl-2-oxabicyclo[2.1.1]hexan-4-yl)-2H-pyrazolo[3,4-b]pyrazin-6-yl)-3-methyl-5-(trifluoromethyl)phenol,
37) (S and R)-2-(2-(2-oxabicyclo[3.1.1]heptan-4-yl)-2H-pyrazolo[3,4-b]pyrazin-6-yl)-3-methyl-5-(trifluoromethyl)phenol,
38) (S)-2-(2-(2-oxabicyclo[3.1.1]heptan-4-yl)-2H-pyrazolo[3,4-b]pyrazin-6-yl)-3-methyl-5-(trifluoromethyl)phenol,
39) (R)-2-(2-(2-oxabicyclo[3.1.1]heptan-4-yl)-2H-pyrazolo[3,4-b]pyrazin-6-yl)-3-methyl-5-(trifluoromethyl)phenol,
40) 4-[6-[2-hydroxy-6-methyl-4-(trifluoromethyl)phenyl]pyrazolo[3,4-b]pyrazin-2-yl]bicyclo[2.1.1]hexan-1-ol,
41) 3-methyl-2-[2-(1-methyl-2-oxabicyclo[2.1.1]hexan-4-yl)pyrazolo[3,4-b]pyrazin-6-yl]-5-(trifluoromethyl)phenol,
42) 4-[6-[2-hydroxy-6-methyl-4-(trifluoromethyl)phenyl]pyrazolo[3,4-b]pyrazin-2-yl]norbornan-1-ol,
43) 3-methyl-2-[2-[(3R)-3-methyl-1,1-dioxo-thiolan-3-yl]pyrazolo[3,4-b]pyrazin-6-yl]-5-(trifluoromethyl)phenol,
44) 2-[2-[(3R)-1,1-dioxothiolan-3-yl]pyrazolo[3,4-b]pyrazin-6-yl]-3-methyl-5-(trifluoromethyl)phenol,
45) 2-[2-[(3S)-1,1-dioxothiolan-3-yl]pyrazolo[3,4-b]pyrazin-6-yl]-3-methyl-5-(trifluoromethyl)phenol,
46) (3R,4S)-4-[6-[2-hydroxy-6-methyl-4-(trifluoromethyl)phenyl]pyrazolo[3,4-b]pyrazin-2-yl]tetrahydrofuran-3-ol,
47) (3S,4R)-4-[6-[2-hydroxy-6-methyl-4-(trifluoromethyl)phenyl]pyrazolo[3,4-b]pyrazin-2-yl]tetrahydrofuran-3-ol,
48) (3R,4R)-3-[6-[2-hydroxy-6-methyl-4-(trifluoromethyl)phenyl]pyrazolo[3,4-b]pyrazin-2-yl]tetrahydropyran-4-ol,
49) (3S,4S)-3-[6-[2-hydroxy-6-methyl-4-(trifluoromethyl)phenyl]pyrazolo[3,4-b]pyrazin-2-yl]tetrahydropyran-4-ol,
50) (1R,3R)-3-(6-(2-hydroxy-6-methyl-4-(trifluoromethyl)phenyl)-2H-pyrazolo[3,4-b]pyrazin-2-yl)cyclobutane-1-carbonitrile,
51) 2-[2-[3-(methoxymethyl)-1-bicyclo[1.1.1]pentanyl]pyrazolo[3,4-b]pyrazin-6-yl]-3-methyl-5-(trifluoromethyl)phenol,
52) 2-[2-(3-methoxy-1-bicyclo[1.1.1]pentanyl)pyrazolo[3,4-b]pyrazin-6-yl]-3-methyl-5-(trifluoromethyl)phenol,
53) 2-[2-[3-(difluoromethyl)-1-bicyclo[1.1.1]pentanyl]pyrazolo[3,4-b]pyrazin-6-yl]-3-methyl-5-(trifluoromethyl)phenol,
54) (1R,4R)-4-(6-(2-hydroxy-6-methyl-4-(trifluoromethyl)phenyl)-2H-pyrazolo[3,4-b]pyrazin-2-yl)cyclohexane-1-carbonitrile,
55) 2-[2-[1-(hydroxymethyl)-2-oxabicyclo[2.1.1]hexan-4-yl]pyrazolo[3,4-b]pyrazin-6-yl]-3-methyl-5-(trifluoromethyl)phenol,
56) 2-[2-(1-bicyclo[2.1.1]hexanyl)pyrazolo[3,4-b]pyrazin-6-yl]-3-methyl-5-(trifluoromethyl)phenol,
57) 2-[2-[3-(hydroxymethyl)-1-bicyclo[1.1.1]pentanyl]pyrazolo[3,4-b]pyrazin-6-yl]-3-methyl-5-(trifluoromethyl)phenol,
58) 2-(2-((1S,4S)-4-hydroxycyclohexyl)-2H-pyrazolo[3,4-b]pyrazin-6-yl)-3-methyl-5-(trifluoromethyl)phenol,
59) 3-methyl-2-[2-(8-oxabicyclo[3.2.1]octan-3-yl)pyrazolo[3,4-b]pyrazin-6-yl]-5-(trifluoromethyl)phenol,
60) 2-[3-[6-[2-hydroxy-6-methyl-4-(trifluoromethyl)phenyl]pyrazolo[3,4-b]pyrazin-2-yl]-1-bicyclo[1.1.1]pentanyl]acetonitrile,
61) 4-[6-[2-hydroxy-6-methyl-4-(trifluoromethyl)phenyl]pyrazolo[3,4-b]pyrazin-2-yl]bicyclo[2.1.1]hexane-1-carbonitrile,
62) 2-[2-(3-fluoro-1-bicyclo[1.1.1]pentanyl)pyrazolo[3,4-b]pyrazin-6-yl]-3-methyl-5-(trifluoromethyl)phenol,
63) 2-(2-((1R,3R)-3-chlorocyclobutyl)-2H-pyrazolo[3,4-b]pyrazin-6-yl)-3-methyl-5-(trifluoromethyl)phenol,
64) 3-methyl-2-[2-[(1R,2R)-2-methylcyclopropyl]pyrazolo[3,4-b]pyrazin-6-yl]-5-(trifluoromethyl)phenol,
65) 3-methyl-2-[2-(4-methyltetrahydropyran-4-yl)pyrazolo[3,4-b]pyrazin-6-yl]-5-(trifluoromethyl)phenol,
66) 3-methyl-2-[2-[(1S,2S)-2-methylcyclopropyl]pyrazolo[3,4-b]pyrazin-6-yl]-5-(trifluoromethyl)phenol,
67) (1R,5S,6S)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
68) (1R,5S,6R)-6-(6-(2-hydroxy-6-methyl-4-(trifluoromethyl)phenyl)-2H-pyrazolo[3,4-b]pyrazin-2-yl)-3-thiabicyclo[3.1.0]hexane 3,3-dioxide,
69) 5-chloro-2-(2-((1R,3R)-3-fluorocyclobutyl)-2H-pyrazolo[3,4-b]pyrazin-6-yl)-3-methylphenol,
70) 2-(2-((1R,5S,6S)-3-oxabicyclo[3.1.0]hexan-6-yl)-2H-pyrazolo[3,4-b]pyrazin-6-yl)-5-chloro-3-methylphenol,
71) 2-(2-((1R,5S,6R)-3-oxabicyclo[3.1.0]hexan-6-yl)-2H-pyrazolo[3,4-b]pyrazin-6-yl)-5-chloro-3-methylphenol,
72) (R)-4-((6-(2-hydroxy-6-methyl-4-(trifluoromethyl)phenyl)-2H-pyrazolo[3,4-b]pyrazin-2-yl)methyl)-1-methylpyrrolidin-2-one,
73) (S)-4-((6-(2-hydroxy-6-methyl-4-(trifluoromethyl)phenyl)-2H-pyrazolo[3,4-b]pyrazin-2-yl)methyl)-1-methylpyrrolidin-2-one,
74) (R)-5-(6-(2-hydroxy-6-methyl-4-(trifluoromethyl)phenyl)-2H-pyrazolo[3,4-b]pyrazin-2-yl)-1-isopropylpiperidin-2-one,
75) (S)-5-(6-(2-hydroxy-6-methyl-4-(trifluoromethyl)phenyl)-2H-pyrazolo[3,4-b]pyrazin-2-yl)-1-isopropylpiperidin-2-one,
76) (R)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
77) (S)-1-cyclopropyl-4-((6-(2-hydroxy-6-methyl-4-(trifluoromethyl)phenyl)-2H-pyrazolo[3,4-b]pyrazin-2-yl)methyl)pyrrolidin-2-one,
78) (R)-1-ethyl-5-(6-(2-hydroxy-6-methyl-4-(trifluoromethyl)phenyl)-2H-pyrazolo[3,4-b]pyrazin-2-yl)piperidin-2-one,
79) (S)-1-ethyl-5-(6-(2-hydroxy-6-methyl-4-(trifluoromethyl)phenyl)-2H-pyrazolo[3,4-b]pyrazin-2-yl)piperidin-2-one,
80) (R)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
81) (S)-1-ethyl-4-((6-(2-hydroxy-6-methyl-4-(trifluoromethyl)phenyl)-2H-pyrazolo[3,4-b]pyrazin-2-yl)methyl)pyrrolidin-2-one,
82) (R)-3-methyl-2-(2-(1-methyl-4,5,6,7-tetrahydro-1H-benzo[d][1,2,3]triazol-6-yl)-2H-pyrazolo[3,4-b]pyrazin-6-yl)-5-(trifluoromethyl)phenol,
83) (S)-3-methyl-2-(2-(1-methyl-4,5,6,7-tetrahydro-1H-benzo[d][1,2,3]triazol-6-yl)-2H-pyrazolo[3,4-b]pyrazin-6-yl)-5-(trifluoromethyl)phenol,
84) (R)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
85) (S)-3-methyl-5-(trifluoromethyl)-2-(2-(3-(trifluoromethyl)-5,6,7,8-tetrahydro-[1,2,4]triazolo[4,3-a]pyridin-6-yl)-2H-pyrazolo[3,4-b]pyrazin-6-yl)phenol,
86) (R)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
87) (S)-2-(2-(3-ethyl-5,6,7,8-tetrahydro-[1,2,4]triazolo[4,3-a]pyridin-6-yl)-2H-pyrazolo[3,4-b]pyrazin-6-yl)-3-methyl-5-(trifluoromethyl)phenol,
88) 2-(2-((1R,3R)-3-fluoro-1-methylcyclobutyl)-2H-pyrazolo[3,4-b]pyrazin-6-yl)-3-methyl-5-(trifluoromethyl)phenol,
89) 2-(2-((1S,3S)-3-fluoro-1-methylcyclobutyl)-2H-pyrazolo[3,4-b]pyrazin-6-yl)-3-methyl-5-(trifluoromethyl)phenol,
90) 2-(2-((1R,5R)-2,2-dimethyl-3-oxabicyclo[3.1.0]hexan-1-yl)-2H-pyrazolo[3,4-b]pyrazin-6-yl)-3-methyl-5-(trifluoromethyl)phenol,
91) 2-(2-((1S,5S)-2,2-dimethyl-3-oxabicyclo[3.1.0]hexan-1-yl)-2H-pyrazolo[3,4-b]pyrazin-6-yl)-3-methyl-5-(trifluoromethyl)phenol,
92) (S)-3-methyl-2-(2-(3-methyltetrahydrofuran-3-yl)-2H-pyrazolo[3,4-b]pyrazin-6-yl)-5-(trifluoromethyl)phenol,
93) (R)-3-methyl-2-(2-(3-methyltetrahydrofuran-3-yl)-2H-pyrazolo[3,4-b]pyrazin-6-yl)-5-(trifluoromethyl)phenol,
94) (R)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
95) (S)-4-((6-(2-hydroxy-6-methyl-4-(trifluoromethyl)phenyl)-2H-pyrazolo[3,4-b]pyrazin-2-yl)methyl)-1-isopropylpyrrolidin-2-one,
96) (1R,4R)-4-(6-(2-hydroxy-6-methyl-4-(trifluoromethyl)phenyl)-2H-pyrazolo[3,4-b]pyrazin-2-yl)-1-methylphosphinane 1-oxide,
97) (1S,4S)-4-(6-(2-hydroxy-6-methyl-4-(trifluoromethyl)phenyl)-2H-pyrazolo[3,4-b]pyrazin-2-yl)-1-methylphosphinane 1-oxide,
98) 2-(2-((1R,5R)-3-oxabicyclo[3.1.0]hexan-1-yl)-2H-pyrazolo[3,4-b]pyrazin-6-yl)-3-methyl-5-(trifluoromethyl)phenol,
99) 2-(2-((1S,5S)-3-oxabicyclo[3.1.0]hexan-1-yl)-2H-pyrazolo[3,4-b]pyrazin-6-yl)-3-methyl-5-(trifluoromethyl)phenol,
100) 3-methyl-2-(2-((3R,4R)-3-methyltetrahydro-2H-pyran-4-yl)-2H-pyrazolo[3,4-b]pyrazin-6-yl)-5-(trifluoromethyl)phenol,
101) 3-methyl-2-(2-((3S,4S)-3-methyltetrahydro-2H-pyran-4-yl)-2H-pyrazolo[3,4-b]pyrazin-6-yl)-5-(trifluoromethyl)phenol,
102) 3-methyl-2-(2-((3R,4S)-3-methyltetrahydro-2H-pyran-4-yl)-2H-pyrazolo[3,4-b]pyrazin-6-yl)-5-(trifluoromethyl)phenol,
103) 3-methyl-2-(2-((3S,4R)-3-methyltetrahydro-2H-pyran-4-yl)-2H-pyrazolo[3,4-b]pyrazin-6-yl)-5-(trifluoromethyl)phenol,
104) 3-methyl-2-(2-((2R,4S)-2-methyltetrahydro-2H-pyran-4-yl)-2H-pyrazolo[3,4-b]pyrazin-6-yl)-5-(trifluoromethyl)phenol,
105) 3-methyl-2-(2-((2S,4R)-2-methyltetrahydro-2H-pyran-4-yl)-2H-pyrazolo[3,4-b]pyrazin-6-yl)-5-(trifluoromethyl)phenol,
106) 2-(2-((1R,6S)-3-oxabicyclo[4.1.0]heptan-6-yl)-2H-pyrazolo[3,4-b]pyrazin-6-yl)-3-methyl-5-(trifluoromethyl)phenol,
107) 2-(2-((1S,6R)-3-oxabicyclo[4.1.0]heptan-6-yl)-2H-pyrazolo[3,4-b]pyrazin-6-yl)-3-methyl-5-(trifluoromethyl)phenol,
108) 3-methyl-2-(2-((2S,4S)-2-methyltetrahydro-2H-pyran-4-yl)-2H-pyrazolo[3,4-b]pyrazin-6-yl)-5-(trifluoromethyl)phenol,
109) 3-methyl-2-(2-((2R,4R)-2-methyltetrahydro-2H-pyran-4-yl)-2H-pyrazolo[3,4-b]pyrazin-6-yl)-5-(trifluoromethyl)phenol,
110) (R)-2-(2-(2,2-dimethyltetrahydro-2H-pyran-4-yl)-2H-pyrazolo[3,4-b]pyrazin-6-yl)-3-methyl-5-(trifluoromethyl)phenol,
111) (S)-2-(2-(2,2-dimethyltetrahydro-2H-pyran-4-yl)-2H-pyrazolo[3,4-b]pyrazin-6-yl)-3-methyl-5-(trifluoromethyl)phenol,
112) (R)-2-(2-(3,3-dimethyltetrahydro-2H-pyran-4-yl)-2H-pyrazolo[3,4-b]pyrazin-6-yl)-3-methyl-5-(trifluoromethyl)phenol,
113) (S)-2-(2-(3,3-dimethyltetrahydro-2H-pyran-4-yl)-2H-pyrazolo[3,4-b]pyrazin-6-yl)-3-methyl-5-(trifluoromethyl)phenol,
114) (R)-2-(2-(5-oxaspiro[2.5]octan-8-yl)-2H-pyrazolo[3,4-b]pyrazin-6-yl)-3-methyl-5-(trifluoromethyl)phenol,
115) (S)-2-(2-(5-oxaspiro[2.5]octan-8-yl)-2H-pyrazolo[3,4-b]pyrazin-6-yl)-3-methyl-5-(trifluoromethyl)phenol,
116) 2-(2-((3S,4S)-3-fluorotetrahydro-2H-pyran-4-yl)-2H-pyrazolo[3,4-b]pyrazin-6-yl)-3-methyl-5-(trifluoromethyl)phenol,
117) 2-(2-((3R,4R)-3-fluorotetrahydro-2H-pyran-4-yl)-2H-pyrazolo[3,4-b]pyrazin-6-yl)-3-methyl-5-(trifluoromethyl)phenol,
118) 2-(2-((3R,4S)-3-fluorotetrahydro-2H-pyran-4-yl)-2H-pyrazolo[3,4-b]pyrazin-6-yl)-3-methyl-5-(trifluoromethyl)phenol,
119) 2-(2-((3S,4R)-3-fluorotetrahydro-2H-pyran-4-yl)-2H-pyrazolo[3,4-b]pyrazin-6-yl)-3-methyl-5-(trifluoromethyl)phenol,
120) (R)-2-(2-(3,3-difluorotetrahydro-2H-pyran-4-yl)-2H-pyrazolo[3,4-b]pyrazin-6-yl)-3-methyl-5-(trifluoromethyl)phenol,
121) (S)-5-(6-(2-hydroxy-6-methyl-4-(trifluoromethyl)phenyl)-3-methyl-2H-pyrazolo[3,4-b]pyrazin-2-yl)-1-methylpiperidin-2-one,
122) (R and S)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
123) (R)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
124) (R)-5-(6-(2-hydroxy-6-methyl-4-(trifluoromethyl) phenyl)-3-methyl-2H-pyrazolo[3,4-b]pyrazin-2-yl)-1-(4-methoxybenzyl)piperidin-2-one,
125) (R)-5-(6-(2-hydroxy-6-methyl-4-(trifluoromethyl)phenyl)-3-methyl-2H-pyrazolo[3,4-b]pyrazin-2-yl)piperidin-2-one,
126) (S)-5-(6-(2-hydroxy-6-methyl-4-(trifluoromethyl)phenyl)-3-methyl-2H-pyrazolo[3,4-b]pyrazin-2-yl)piperidin-2-one,
127) (R)-3-ethyl-5-((6-(2-hydroxy-6-methyl-4-(trifluoromethyl)phenyl)-2H-pyrazolo[3,4-b]pyrazin-2-yl)methyl)oxazolidin-2-one,
128) (R)-5-(6-(2-hydroxy-4-(trifluoromethyl)phenyl)-5-methyl-2H-pyrazolo[3,4-b]pyrazin-2-yl)-1-methylpiperidin-2-one,
129) (S)-5-(6-(2-hydroxy-4-(trifluoromethyl)phenyl)-5-methyl-2H-pyrazolo[3,4-b]pyrazin-2-yl)-1-methylpiperidin-2-one,
130) (S)-1-cyclopropyl-4-((6-(2-(ethoxymethoxy)-6-methyl-4-(trifluoromethyl)phenyl)-3-((S)-1-hydroxyethyl)-2H-pyrazolo[3,4-b]pyrazin-2-yl)methyl)pyrrolidin-2-one,
131) (S)-1-cyclopropyl-4-((6-(2-(ethoxymethoxy)-6-methyl-4-(trifluoromethyl)phenyl)-3-((R)-1-hydroxyethyl)-2H-pyrazolo[3,4-b]pyrazin-2-yl)methyl)pyrrolidin-2-one,
132) (R)-1-cyclopropyl-4-((6-(2-(ethoxymethoxy)-6-methyl-4-(trifluoromethyl)phenyl)-3-((R)-1-hydroxyethyl)-2H-pyrazolo[3,4-b]pyrazin-2-yl)methyl)pyrrolidin-2-one,
133) (R)-1-cyclopropyl-4-((6-(2-(ethoxymethoxy)-6-methyl-4-(trifluoromethyl)phenyl)-3-((S)-1-hydroxyethyl)-2H-pyrazolo[3,4-b]pyrazin-2-yl)methyl)pyrrolidin-2-one,
134) (S)-5-(6-(2-hydroxy-6-methyl-4-(trifluoromethyl)phenyl)-3-((S)-1-hydroxyethyl)-2H-pyrazolo[3,4-b]pyrazin-2-yl)-1-isopropylpiperidin-2-one,
135) (R)-5-(6-(2-hydroxy-6-methyl-4-(trifluoromethyl)phenyl)-3-((S)-1-hydroxyethyl)-2H-pyrazolo[3,4-b]pyrazin-2-yl)-1-isopropylpiperidin-2-one,
136) (R)-5-(6-(2-hydroxy-6-methyl-4-(trifluoromethyl)phenyl)-3-((R)-1-hydroxyethyl)-2H-pyrazolo[3,4-b]pyrazin-2-yl)-1-isopropylpiperidin-2-one,
137) (S)-5-(6-(2-hydroxy-6-methyl-4-(trifluoromethyl)phenyl)-3-((R)-1-hydroxyethyl)-2H-pyrazolo[3,4-b]pyrazin-2-yl)-1-isopropylpiperidin-2-one, and
138) 2-(2-((1R,3R)-3-fluorocyclobutyl)-2H-[1,2,3]triazolo[4,5-b]pyrazin-5-yl)-3-methyl-5-(trifluoromethyl)phenol.

26. The compound according to claim 1, or a pharmaceutically acceptable salt thereof, wherein the compound is selected from: 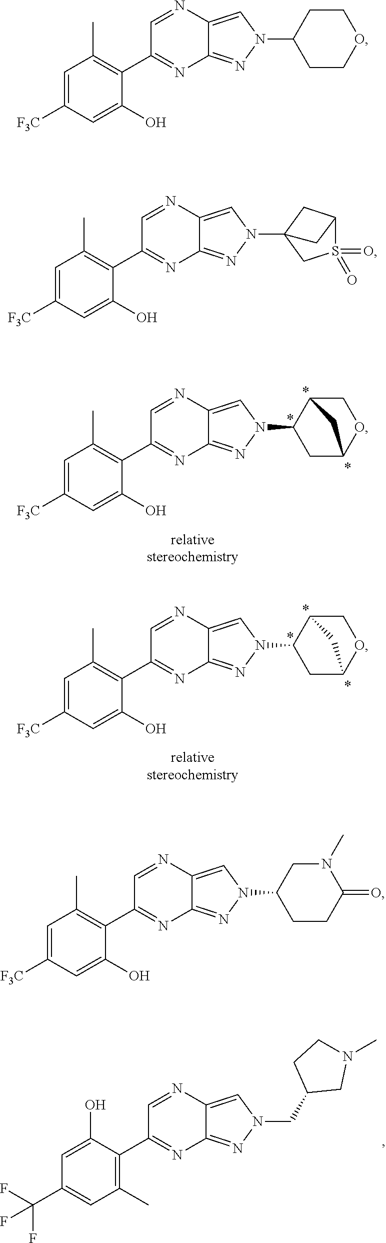 [SMILES: Cc1cc(C(F)(F)F)cc(O)c1-c1cnc2cn(C3CCOCC3)nc2n1 ; Cc1cc(C(F)(F)F)cc(O)c1-c1cnc2cn(C34CC(C3)S(=O)(=O)C4)nc2n1 ; Cc1cc(C(F)(F)F)cc(O)c1-c1cnc2cn([C@@H]3C[C@@H]4C[C@H]3CO4)nc2n1 ; Cc1cc(C(F)(F)F)cc(O)c1-c1cnc2cn([C@H]3C[C@H]4C[C@@H]3CO4)nc2n1 ; Cc1cc(C(F)(F)F)cc(O)c1-c1cnc2cn([C@H]3CCC(=O)N(C)C3)nc2n1 ; Cc1cc(C(F)(F)F)cc(O)c1-c1cnc2cn(C[C@@H]3CCN(C)C3)nc2n1] 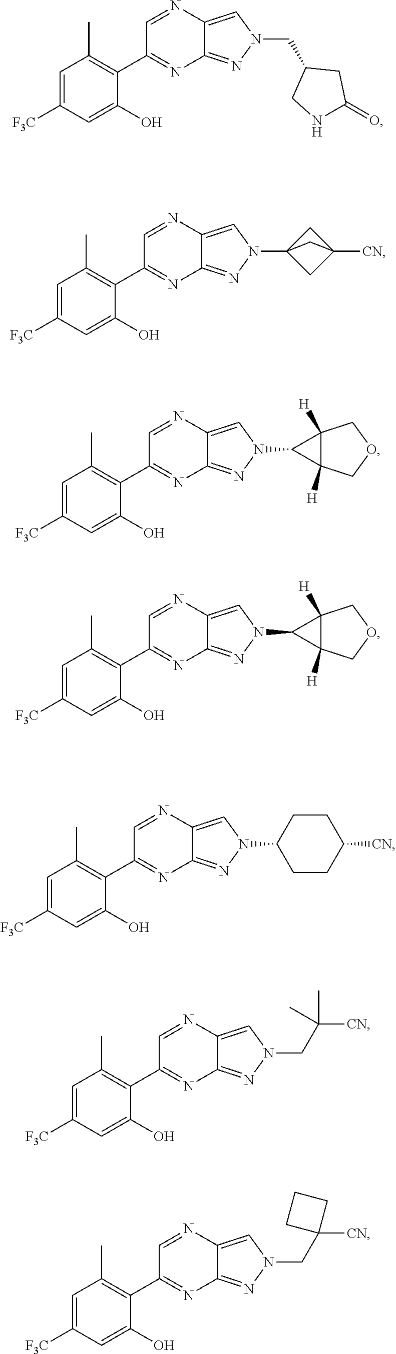 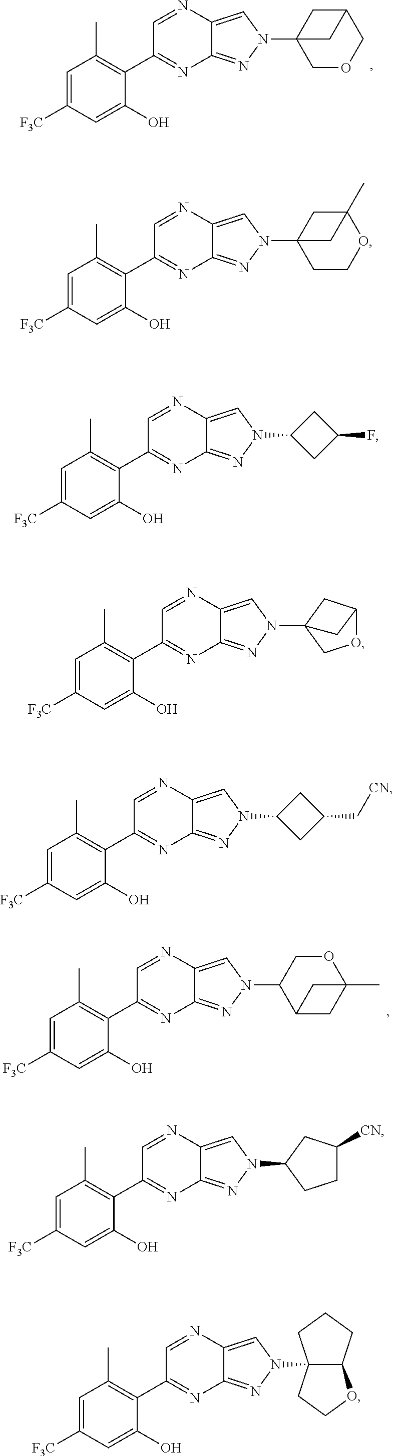 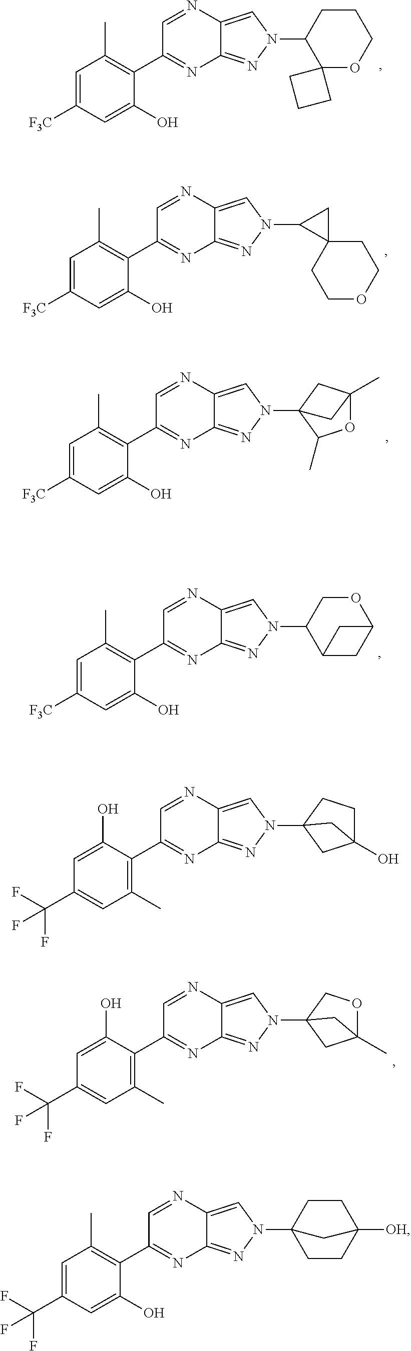 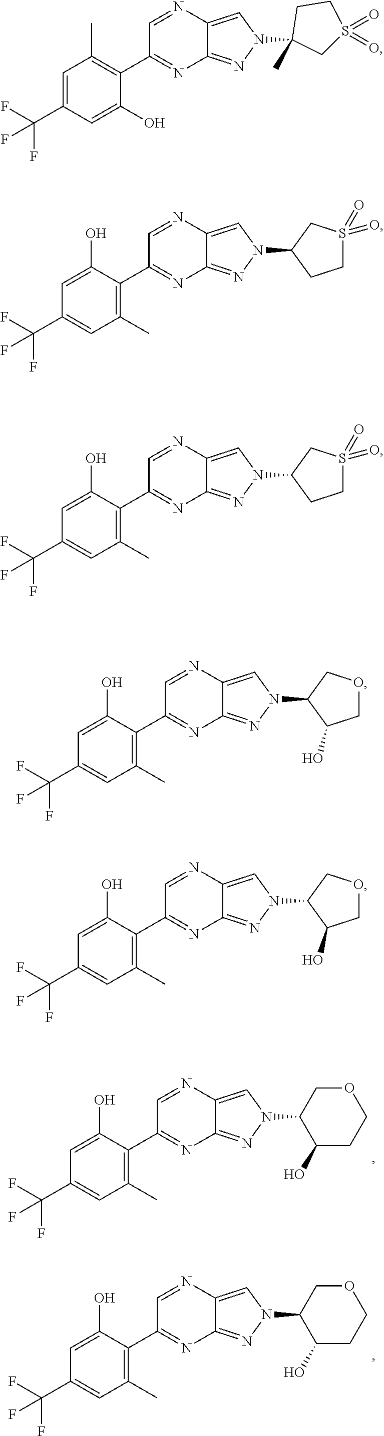 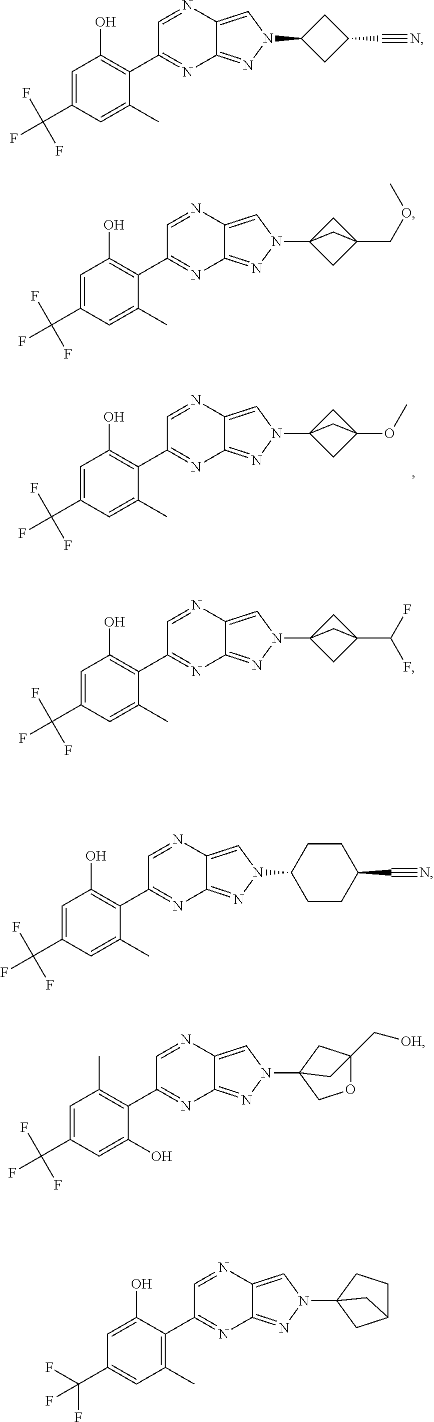 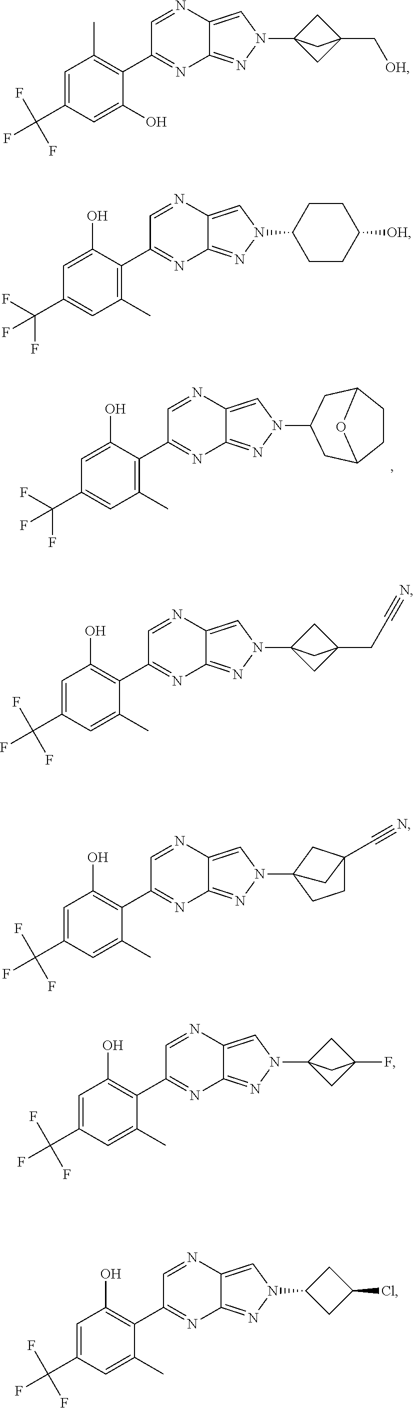 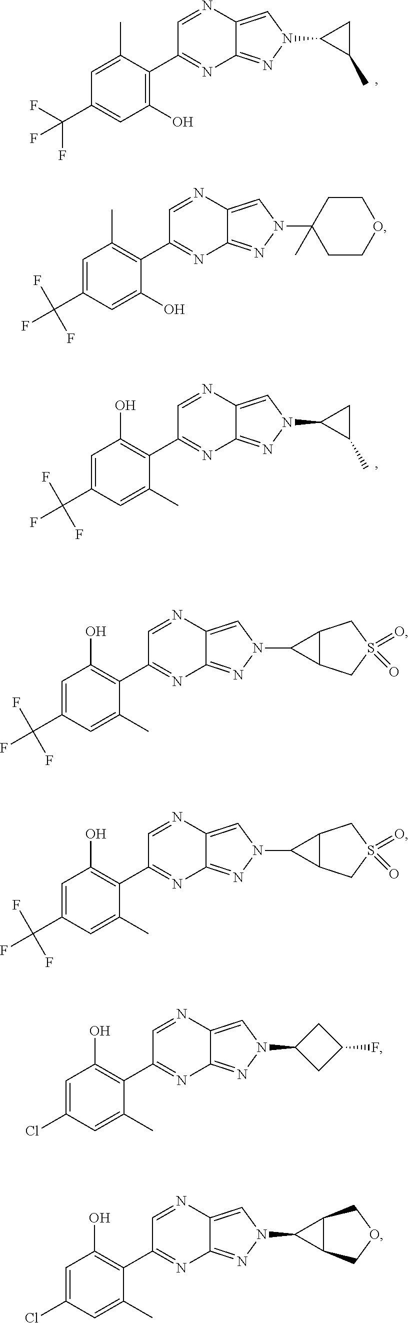 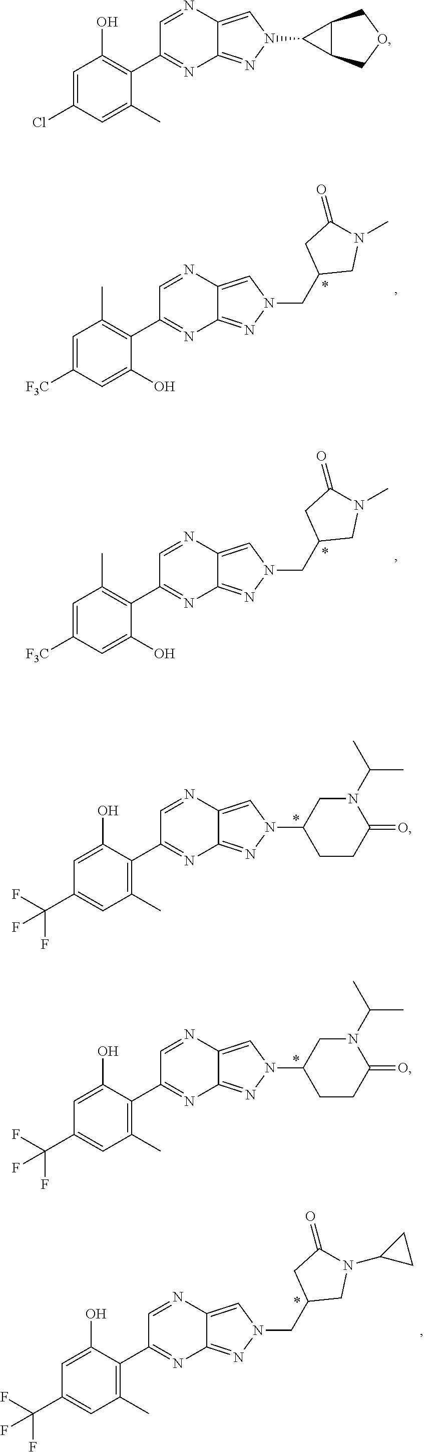 [SMILES: Cc1cc(Cl)cc(O)c1-c1cnc2cn([C@H]3[C@@H]4COC[C@@H]43)nc2n1 ; Cc1cc(C(F)(F)F)cc(O)c1-c1cnc2cn(CC3CC(=O)N(C)C3)nc2n1 ; Cc1cc(C(F)(F)F)cc(O)c1-c1cnc2cn(CC3CC(=O)N(C)C3)nc2n1 ; Cc1cc(C(F)(F)F)cc(O)c1-c1cnc2cn(C3CCC(=O)N(C(C)C)C3)nc2n1 ; Cc1cc(C(F)(F)F)cc(O)c1-c1cnc2cn(C3CCC(=O)N(C(C)C)C3)nc2n1 ; Cc1cc(C(F)(F)F)cc(O)c1-c1cnc2cn(CC3CC(=O)N(C4CC4)C3)nc2n1] 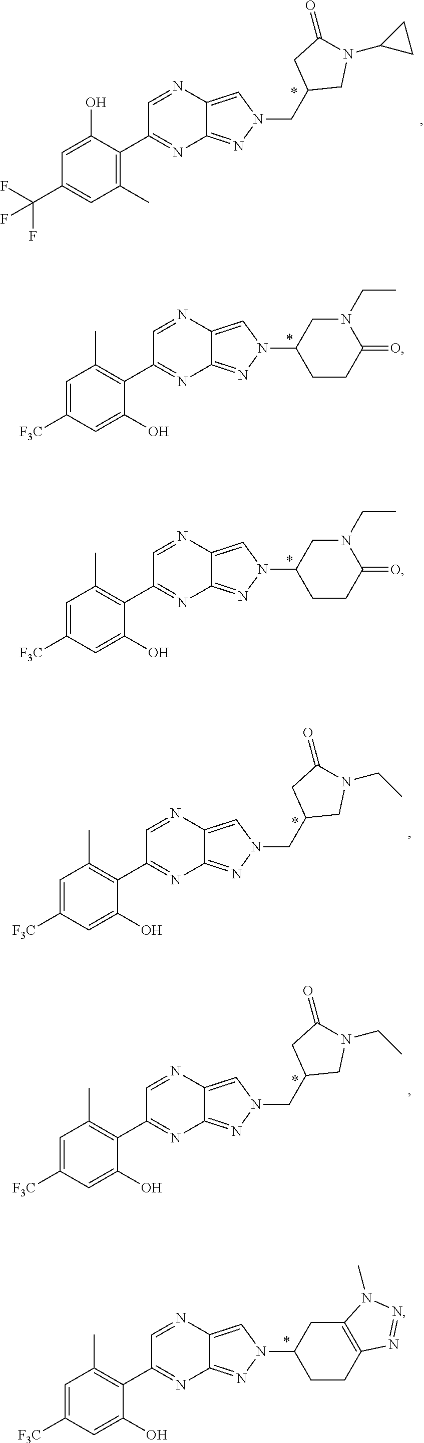 [SMILES: Cc1cc(C(F)(F)F)cc(O)c1-c1cnc2cn(CC3CC(=O)N(C4CC4)C3)nc2n1 ; CCN1CC(n2cc3ncc(-c4c(C)cc(C(F)(F)F)cc4O)nc3n2)CCC1=O ; CCN1CC(n2cc3ncc(-c4c(C)cc(C(F)(F)F)cc4O)nc3n2)CCC1=O ; CCN1CC(Cn2cc3ncc(-c4c(C)cc(C(F)(F)F)cc4O)nc3n2)CC1=O ; CCN1CC(Cn2cc3ncc(-c4c(C)cc(C(F)(F)F)cc4O)nc3n2)CC1=O ; Cc1cc(C(F)(F)F)cc(O)c1-c1cnc2cn(C3CCc4nnn(C)c4C3)nc2n1] 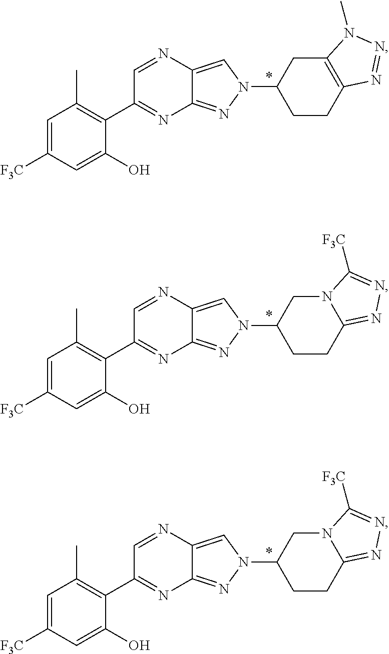 [SMILES: Cc1cc(C(F)(F)F)cc(O)c1-c1cnc2cn(C3CCc4nnn(C)c4C3)nc2n1 ; Cc1cc(C(F)(F)F)cc(O)c1-c1cnc2cn(C3CCc4nnc(C(F)(F)F)n4C3)nc2n1 ; Cc1cc(C(F)(F)F)cc(O)c1-c1cnc2cn(C3CCc4nnc(C(F)(F)F)n4C3)nc2n1] 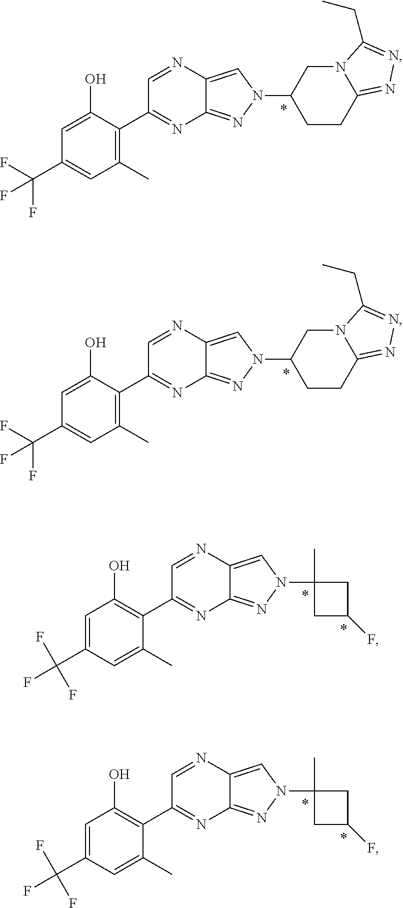 [SMILES: CCc1nnc2n1CC(n1cc3ncc(-c4c(C)cc(C(F)(F)F)cc4O)nc3n1)CC2 ; CCc1nnc2n1CC(n1cc3ncc(-c4c(C)cc(C(F)(F)F)cc4O)nc3n1)CC2 ; Cc1cc(C(F)(F)F)cc(O)c1-c1cnc2cn(C3(C)CC(F)C3)nc2n1 ; Cc1cc(C(F)(F)F)cc(O)c1-c1cnc2cn(C3(C)CC(F)C3)nc2n1] 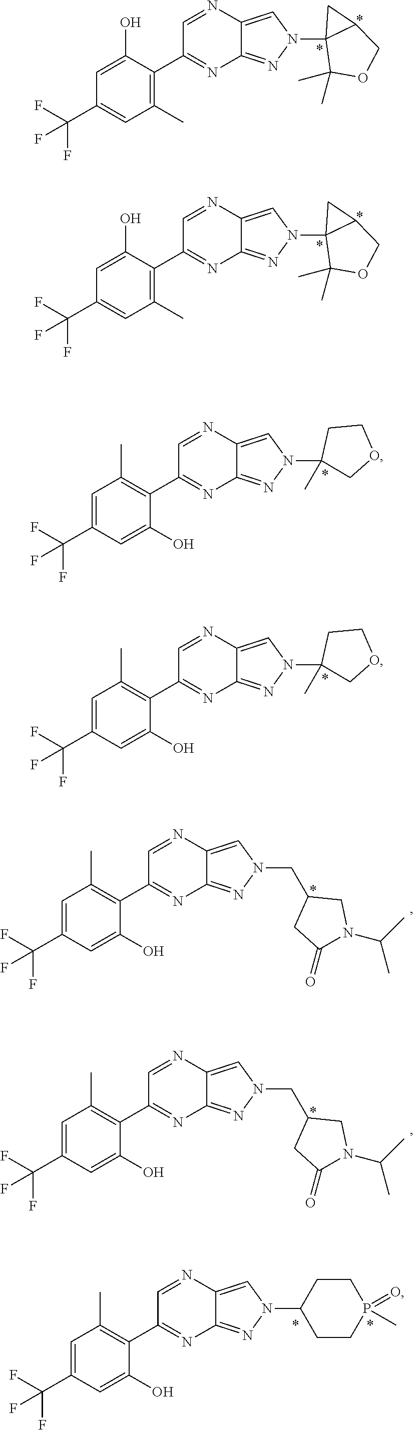 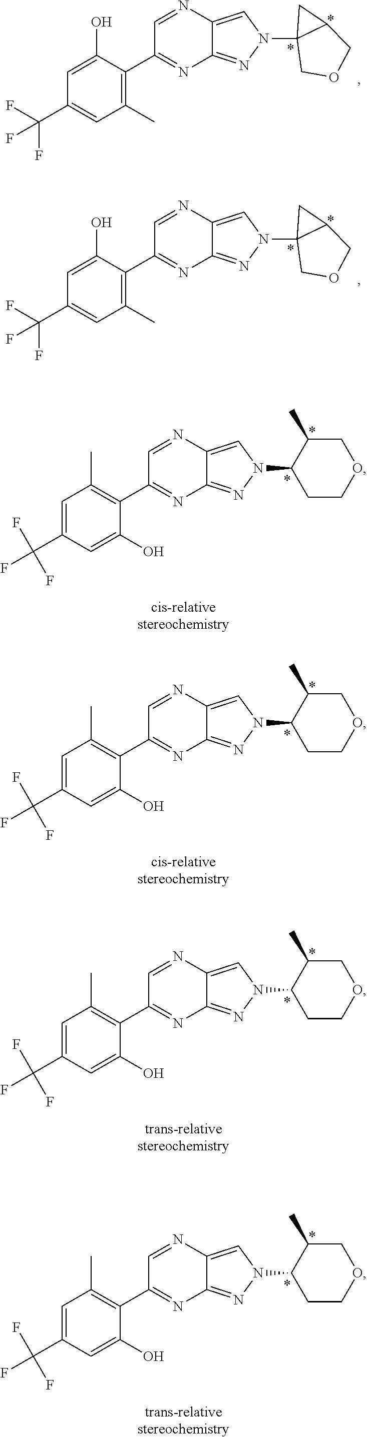 [SMILES: Cc1cc(C(F)(F)F)cc(O)c1-c1cnc2cn(C34COCC3C4)nc2n1 ; Cc1cc(C(F)(F)F)cc(O)c1-c1cnc2cn(C34COCC3C4)nc2n1 ; Cc1cc(C(F)(F)F)cc(O)c1-c1cnc2cn([C@@H]3CCOC[C@@H]3C)nc2n1 ; Cc1cc(C(F)(F)F)cc(O)c1-c1cnc2cn([C@@H]3CCOC[C@@H]3C)nc2n1 ; Cc1cc(C(F)(F)F)cc(O)c1-c1cnc2cn([C@H]3CCOC[C@@H]3C)nc2n1 ; Cc1cc(C(F)(F)F)cc(O)c1-c1cnc2cn([C@H]3CCOC[C@@H]3C)nc2n1] 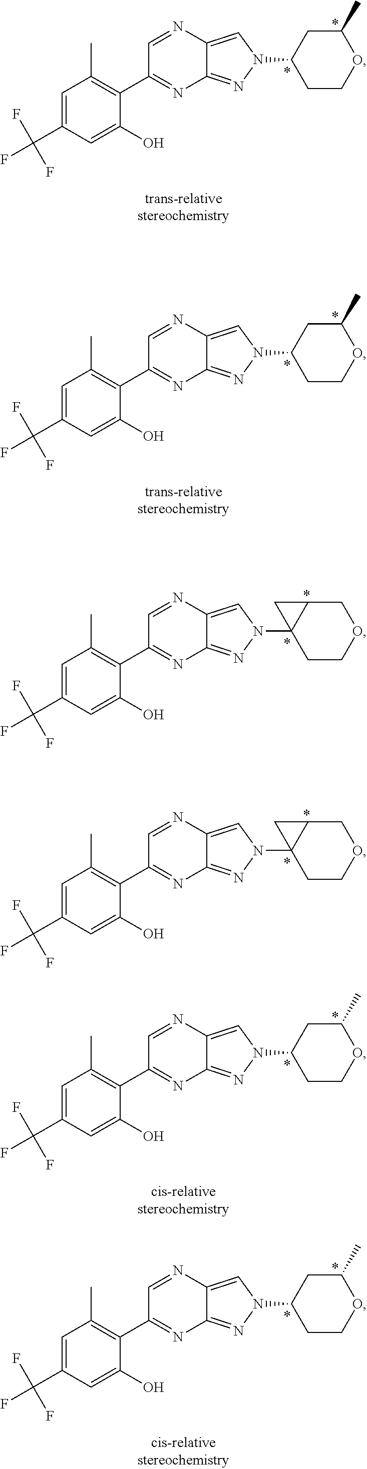 [SMILES: Cc1cc(C(F)(F)F)cc(O)c1-c1cnc2cn([C@H]3CCO[C@H](C)C3)nc2n1 ; Cc1cc(C(F)(F)F)cc(O)c1-c1cnc2cn([C@H]3CCO[C@H](C)C3)nc2n1 ; Cc1cc(C(F)(F)F)cc(O)c1-c1cnc2cn(C34CCOCC3C4)nc2n1 ; Cc1cc(C(F)(F)F)cc(O)c1-c1cnc2cn(C34CCOCC3C4)nc2n1 ; Cc1cc(C(F)(F)F)cc(O)c1-c1cnc2cn([C@H]3CCO[C@@H](C)C3)nc2n1 ; Cc1cc(C(F)(F)F)cc(O)c1-c1cnc2cn([C@H]3CCO[C@@H](C)C3)nc2n1] 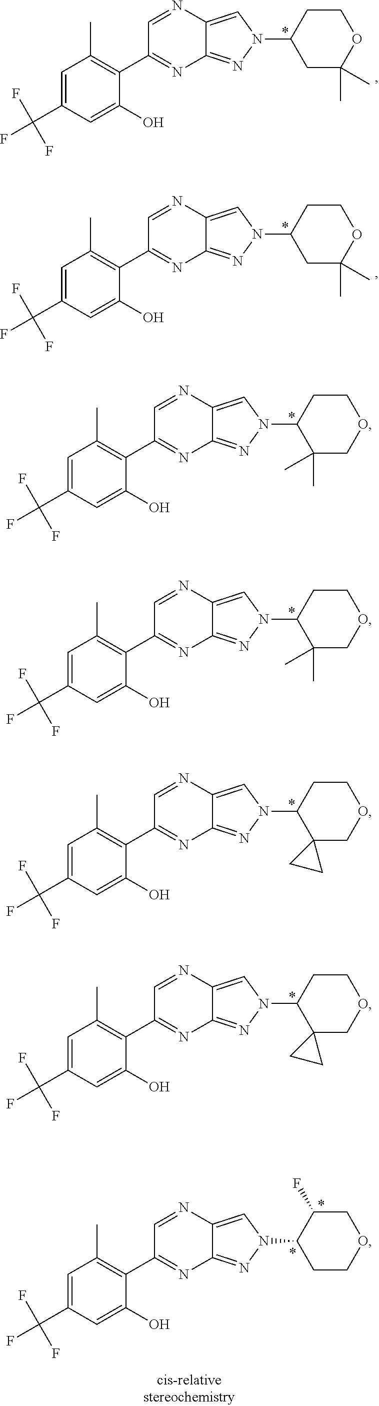 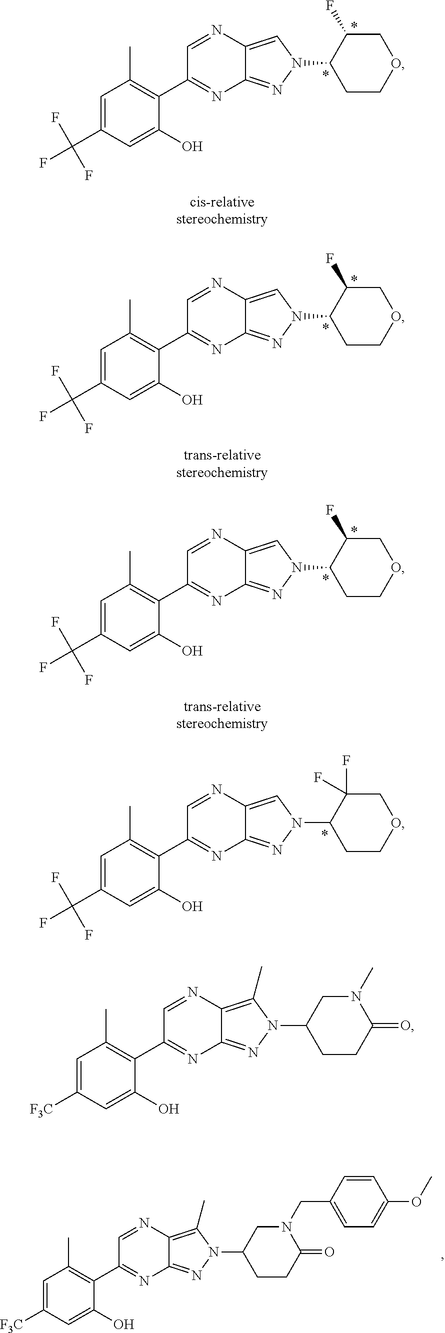 [SMILES: Cc1cc(C(F)(F)F)cc(O)c1-c1cnc2cn([C@H]3CCOC[C@H]3F)nc2n1 ; Cc1cc(C(F)(F)F)cc(O)c1-c1cnc2cn([C@H]3CCOC[C@@H]3F)nc2n1 ; Cc1cc(C(F)(F)F)cc(O)c1-c1cnc2cn([C@H]3CCOC[C@@H]3F)nc2n1 ; Cc1cc(C(F)(F)F)cc(O)c1-c1cnc2cn(C3CCOCC3(F)F)nc2n1 ; Cc1cc(C(F)(F)F)cc(O)c1-c1cnc2c(C)n(C3CCC(=O)N(C)C3)nc2n1 ; COc1ccc(CN2CC(n3nc4nc(-c5c(C)cc(C(F)(F)F)cc5O)cnc4c3C)CCC2=O)cc1] 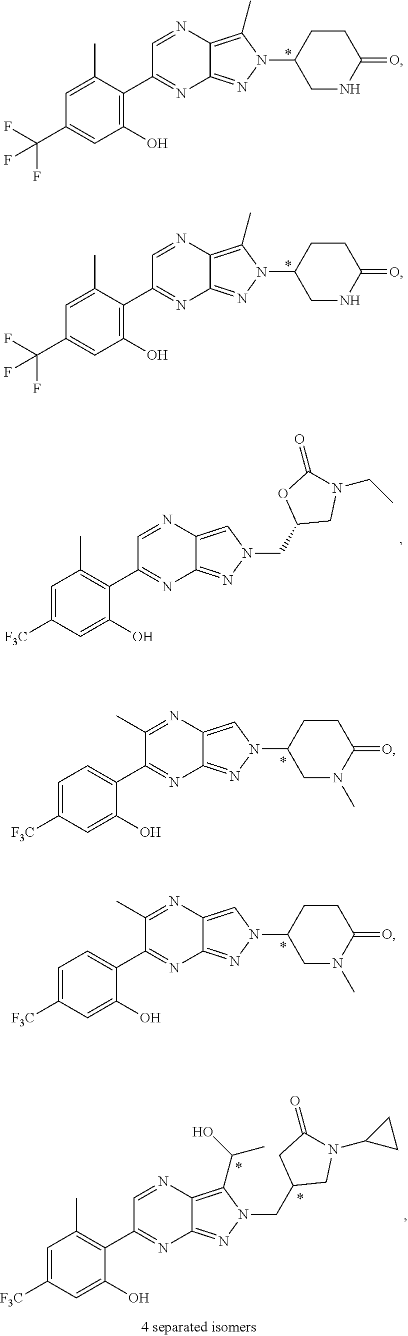 [SMILES: Cc1cc(C(F)(F)F)cc(O)c1-c1cnc2c(C)n(C3CCC(=O)NC3)nc2n1 ; Cc1cc(C(F)(F)F)cc(O)c1-c1cnc2c(C)n(C3CCC(=O)NC3)nc2n1 ; CCN1C[C@H](Cn2cc3ncc(-c4c(C)cc(C(F)(F)F)cc4O)nc3n2)OC1=O ; Cc1nc2cn(C3CCC(=O)N(C)C3)nc2nc1-c1ccc(C(F)(F)F)cc1O ; Cc1nc2cn(C3CCC(=O)N(C)C3)nc2nc1-c1ccc(C(F)(F)F)cc1O ; Cc1cc(C(F)(F)F)cc(O)c1-c1cnc2c(C(C)O)n(CC3CC(=O)N(C4CC4)C3)nc2n1] 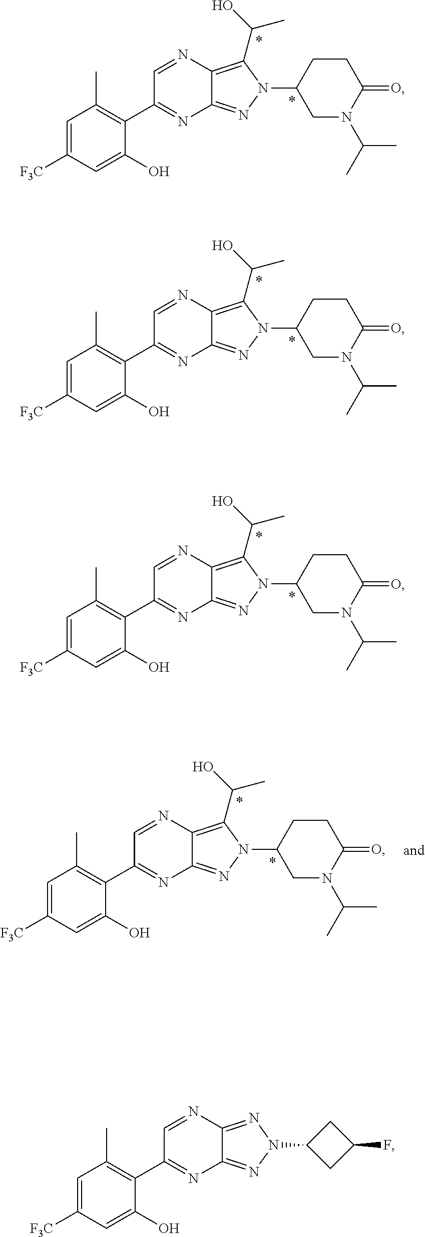 [SMILES: Cc1cc(C(F)(F)F)cc(O)c1-c1cnc2c(C(C)O)n(C3CCC(=O)N(C(C)C)C3)nc2n1 ; Cc1cc(C(F)(F)F)cc(O)c1-c1cnc2c(C(C)O)n(C3CCC(=O)N(C(C)C)C3)nc2n1 ; Cc1cc(C(F)(F)F)cc(O)c1-c1cnc2c(C(C)O)n(C3CCC(=O)N(C(C)C)C3)nc2n1 ; Cc1cc(C(F)(F)F)cc(O)c1-c1cnc2c(C(C)O)n(C3CCC(=O)N(C(C)C)C3)nc2n1 ; Cc1cc(C(F)(F)F)cc(O)c1-c1cnc2nn([C@H]3C[C@H](F)C3)nc2n1]
and pharmaceutically acceptable salts thereof.

27. A pharmaceutical composition comprising a compound according to claim 1, or a pharmaceutically acceptable salt thereof, and a pharmaceutically acceptable carrier.

28. A compound according to claim 1, or a pharmaceutically acceptable salt thereof, for use in therapy.

29. A method of treating or preventing a disorder, condition or disease that is responsive to the inhibition of NLRP3 in a patient in need thereof comprising administration to said patient of a therapeutically effective amount of a compound according to claim 1, or a pharmaceutically acceptable salt thereof.

30. The method of claim 29, wherein the disorder is selected from: an inflammatory disorder, a fibrotic disorder, a cardiovascular disorder, a metabolic disorder, or a neurodegenerative disorder.

31. The method of claim 30, wherein the disorder is an inflammatory disorder.

32. The method of claim 31, wherein the inflammatory disorder is selected from: an auto-immune disorder, an auto-inflammatory disorder, an inflammatory joint disorder, an inflammatory skin disorder, and a neuroinflammatory disorder.

33. The method of claim 29, wherein the disorder is selected from: atherosclerosis, non-alcoholic steatohepatitis, Alzheimer's disease, Parkinson's disease, and dementia with Lewy bodies.
